(19) United States
(12) Patent Application Publication
(10) Pub. No.: US 2014/0242609 A1
(43) Pub. Date: Aug. 28, 2014

(54) Diagnosis and treatment of kidney stones, methods and compositions therefor

(71) Applicant: Washington University, Saint Louis, MO (US)

(72) Inventors: Jianghui Hou, Saint Louis, MO (US); Yongfeng Gong, Saint Louis, MO (US)

(73) Assignee: WASHINGTON UNIVERSITY, Saint Louis, MO (US)

(21) Appl. No.: 14/191,356

(22) Filed: Feb. 26, 2014

Related U.S. Application Data
(60) Provisional application No. 61/914,895, filed on Dec. 11, 2013.
(60) Provisional application No. 61/769,499, filed on Feb. 26, 2013.

(51) Int. Cl. G01N 33/68

(52) U.S. Cl. CPC G01N 33/6872
USPC 435/7.21

(57) ABSTRACT

Methods of detecting, diagnosing, monitoring and treating kidney stones are disclosed. In some embodiments, methods of detecting, diagnosing or monitoring kidney stones comprise contacting a urine sample with anti-Claudin-14 antibody, and detecting quantity of a complex comprising Claudin-14 and the antibody, wherein an increase compared to control levels is diagnostic for kidney stones. In some embodiments, methods further comprise testing a second sample at a second time point to detect increased kidney stones. In some embodiments, methods of treating kidney stone disease comprise administering an miR-9 mimic or an miR-374 mimic. In some embodiments, methods comprise administering an inhibitor of CaSR signaling. In some embodiments, methods comprise administering a HDAC inhibitor. In some embodiments methods of treating hyperparathyroidism and hypercalcemia are disclosed, comprising administering an agonist of CaSR. Methods of abrogating CaSR-mediated regulation of claudin-14, microRNAs and urinary Ca++ excretion are disclosed, comprising administering a calcineurin inhibitor.

### CROSS-REFERENCE TO RELATED APPLICATIONS

This application claims the benefit of priority to U.S. Provisional Patent Application 61/769,499 filed Feb. 26, 2013 and U.S. Provisional Patent Application 61/914,895 filed Dec. 11, 2013, each of which is incorporated herein by reference in its entirety.

### STATEMENT REGARDING FEDERALLY SPONSORED RESEARCH OR DEVELOPMENT

This work received support from National Institute of Diabetes and Digestive and Kidney Diseases grants 1ROI1DK084059-01 and 5P30DK079333, National Institutes of Health Grants RO1DK084059 and P30 DK07933. The government may have certain rights in the invention.

### INCORPORATION BY REFERENCE OF SEQUENCE LISTING

The Sequence Listing, which is a part of the present disclosure, includes a computer readable form and a written sequence listing comprising nucleotide and/or amino acid sequences. The sequence listing information recorded in computer readable form is identical to the written sequence listing. The subject matter of the Sequence Listing is incorporated herein by reference in its entirety.

### INTRODUCTION

Nephrolithiasis is a common condition and difficult to cure. For many years, the only therapies for hypercalciuria were diuretics such as thiazide and amiloride. The physiological mechanism of diuretic-induced hypocalciuria is considered secondary to extracellular volume (ECV) depletion and reduced GFR, which increase paracellular Ca⁺⁺ reabsorption in the proximal tubules. (Nijenhuis, T., et al., 2005)

Three tight junction genes in the kidney, claudin-14, 16 and 19, play a role in calcium imbalance diseases including kidney stones. (Simon, D. B., et al., 1999, Konrad, M., et al., 2006, and Thorleifsson, G., et al., 2009) Autosomal recessive familial hypomagnesemia with hypercalciuria and nephrocalcinosis (FHHNC: OMIM #248250), is caused by mutations in the genes claudin-16 (Simon, D. B., et al., 1999) and claudin-19. (Konrad, M., et al., 2006)

The calcimimetic cinacalcet, which reduces serum Ca²⁺ levels and PTH levels, has been used to treat secondary hyperparathyroidism in chronic kidney disease (CKD) and dialysis patients (Lindberg et al., 2005).

NPS2143 is a Ca⁺⁺ sensing receptor (CaSR) antagonist (calcilytic) that increases serum Ca²⁺ level independent of PTH secretion. It is used as a treatment for restoring extracellular Ca²⁺ levels in primary hypoparathyroidism (Loupy et al., 2012). Renal CaSR in extracellular Ca²⁺ metabolism has been demonstrated in a kidney specific CaSR KO mouse model (Toka et al., 2012). The kidney CaSR is predominantly expressed in the TALH, co-localizing with claudin-14 (Loupy et al., 2012).

Claudin-14 (CLDN14) plays a role in the physiology of cochlear hair cells in the inner ear (Ben-Yosef et al., 2003). Mutations in CLDN14 have been linked to autosomal recessive nonsyndromic deafness (DFNB29) (Wilcox et al., 2001). Nevertheless, neither hypercalciuria nor nephrolithiasis has been found in human or transgenic knockout animals with these mutations (Wilcox et al., 2001. Ben-Yosef et al., 2003).

Dimke, H., et al., Am J Physiol Renal Physiol. 2013 Mar. 15; 304(6):F761-9 discloses that activation of CaSR increases CLDN14 expression, which in turn blocks the paracellular reabsorption of Ca²⁺. These authors hypothesize that dysregulation of the CaSR-Cldn14 axis likely contributes to the development of hypercalciuria and kidney stones. While Dimke et al., measures excretion of Ca²⁺ in urine, there is no mention of measuring CLDN14 in urine.

U.S. Pat. No. 7,426,441 “Methods for Determining Renal Toxins” of Mendrick, D., et al., lists CLDN16 as a marker for kidney injury/malfunction. U.S. Pat. No. 7,186,802, “Claudin Polypeptides” of Youakim, A., et al., discloses claudin polypeptides for ion transport disorders in the kidney, in particularly Claudin-19. Neither U.S. Pat. No. 7,426,441 nor U.S. Pat. No. 7,186,802 mentions CLDN14.

Hou, J., et al., Annu. Rev. Physiol. 75: 16.1-16.23, 2013 describes all claudins-14, -16, and -19 in relation to hyper/hypocalciuria. Toka, H. R., et al., J. Am. Soc. Nephrol. 23:1879-1890, 2012, indicates that loss-of-function mutations in Claudin-16 and Claudin-19 can cause hypercalciuria and nephrocalcinosis in humans. Toka, H. R., et al., states that renal Casr has a PTH-independent role for renal Ca2+ reabsorption that occurs in the TAL accompanied by Claudin14 downregulation, increasing paracellular Ca2+ reabsorption without significantly affecting Mg2+ reabsorption. Additionally, that they did not know if the effects of CaSR on Claudin 14 expression are independently regulated phenomena or if they are biochemically related. Neither Hou, J., et al., nor Toka, H. R., et al., mention measuring CLDN14 in urine or administering a drug to treat kidney stones.

### SUMMARY

In various embodiments, the present teachings include methods of treating hypercalciuria, nephrolithiasis, and other related disorders in a subject in need thereof. In various embodiments, these methods comprise administering to a subject a therapeutically effective amount of at least one inhibitor of histone deacetylase (HDAC).

Animal studies by the inventors show that HDAC inhibitors only affect the microRNA-CLDN14 pathway in the kidney. In various embodiments, these epigenetic inhibitors can promote positive Ca⁺⁺ homeostasis at a low dose. In various embodiments, curbing CLDN14 expression can be beneficial to kidney stone patients as an independent factor apart from CLDN14's role in reducing renal Ca⁺⁺ excretion.

In various embodiments, the present teachings include methods of treating hypercalciuria, nephrolithiasis, and other related disorders in a subject in need thereof. These methods can comprise administering to a subject a therapeutically effective amount of at least one HDAC inhibitor such as trichostatin A (TsA) or suberanilohydroxanmic acid (SAHA; approved by FDA as Vorinostat®) (Marks, P. A. et al., 2007). In some configurations. SAHA can downregulate CLDN14 mRNA levels by 38% at 0.1 μM (p<0.01, n=3 versus vehicle; FIG. 18A). In various configurations, the downregulation can persist through 0.33 μM to 3.3 μM and can reach 54% at 0.33 μM (FIG. 18A). In some configurations, at a concentration of 0.01 μM, TsA can reduce CLDN14 mRNA levels by 64% (p<0.001, n=3 versus vehicle; FIG. 18B); the reduction can be through 0.033 μM to 0.33 μM (FIG. 18B).

In various embodiments, the present teachings include methods of treating, ameliorating, and/or preventing kidney stones. In various embodiments, these methods can comprise, consist essentially of, or consist of administering an antagonist (calcilytic) of Ca⁺⁺ sensing receptor (CaSR). In some embodiments, the CaSR antagonist can be NPS2143 of structure

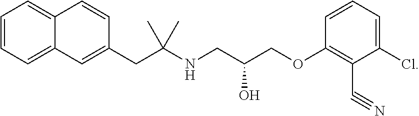 [SMILES: CC(C)(Cc1ccc2ccccc2c1)NC[C@@H](O)COc1cccc(Cl)c1C#N]

Methods of treating a disease such as hyper/hypoparathyroidism, kidney stone, osteoporosis, Alzheimer's disease or epilepsy are also disclosed. In various embodiments, these methods comprise administering an agonist of CaSR or an antagonist of CaSR. In various embodiments, an agonist of CaSR can be a calcimimetic. In some configurations, an agonist of CaSR can be cinacalcet. In various embodiments, an antagonist of CaSR can be NPS2143.

In some embodiments, the present teachings include an antibody against CLDN14, or an antigen-binding portion thereof. In various configurations, the antibody can be a monoclonal antibody, a polyclonal antibody, a single chain antibody such as a camelid antibody, or an antigen binding fragment of an antibody. In some embodiments, the antibody can be directed against the first extracellular loop of CLDN14.

In some embodiments, the present teachings include a method for diagnosing kidney stones. These methods comprise obtaining a sample comprising exosomes, such as urine sample comprising exosomes, from a subject. In various configurations, the subject can have, or can be suspected of having, kidney and/or bladder stones. In these embodiments, the sample can be contacted with an antibody against CLDN14. The presence, absence, and/or quantity of CLDN14-antibody complex can then be determined by routine detection methods such as, without limitation, ELISA or Western blot assay. A subject can be diagnosed with kidney stones if the amount of an immune complex that forms between the antibody and the CLDN14 exceeds that of healthy volunteers by a statistically significant amount. In various configurations, the subject can be a mammal such as, without limitation, a human, a rodent, a canine, a feline, a bovine, an ovine, or a porcine, or an avian such as a chicken.

The present teachings include the following non-limiting aspects.

1. A method of detecting, diagnosing or monitoring kidney stone disease in a subject, comprising:

providing a urine sample from a subject having or suspected of having kidney stone disease;

contacting the sample with an antibody that binds Claudin-14 polypeptide, under conditions sufficient for formation of a primary complex comprising the antibody and the Claudin-14 polypeptide if present:

measuring quantity of the primary complex;

comparing the quantity of the primary complex to that of a control complex formed from the antibody and a urine sample of an individual who does not have kidney stone disease; and

detecting kidney stone disease if the quantity of the primary complex from the subject is statistically significantly greater than that of an individual who does not have kidney stone disease.

2. A method of detecting, diagnosing or monitoring kidney stone disease in accordance with aspect 1, wherein the antibody is a monoclonal antibody.
3. A method of detecting, diagnosing or monitoring kidney stone disease in accordance with aspect 1, wherein the antibody is a polyclonal antibody.
4. A method of detecting, diagnosing or monitoring kidney stone disease in accordance with aspect 1, wherein the antibody is a single chain antibody.
5. A method in accordance with aspect 1, wherein the antibody is directed against the first extracellular loop of CLDN14.
6. A method of detecting, diagnosing or monitoring kidney stone disease in accordance with aspect 1, wherein the measuring comprises an immunoprecipitation assay, an ELISA, a radioimmunoassay, a Western blot assay, a dip stick assay, a microarray, or a bead assay.
7. A method of detecting, diagnosing or monitoring kidney stone disease in accordance with aspect 6, wherein the measuring comprises an ELISA or a Western blot assay.
8. A method of detecting, diagnosing or monitoring kidney stone disease in accordance with aspect 1, wherein the antibody comprises a label, and the measuring quantity of the complex comprises quantifying the label.
9. A method of detecting, diagnosing or monitoring kidney stone disease in accordance with aspect 8, wherein the label is selected from the group consisting of an enzyme, a radioisotope, a fluorogen, fluorophore, a chromogen and a chromophore.
10. A method of detecting, diagnosing or monitoring kidney stone disease in accordance with aspect 9, wherein the enzyme is selected from the group consisting of a peroxidase, a phosphatase, a galactosidase and a luciferase.
11. A method of detecting, diagnosing or monitoring kidney stone disease in accordance with aspect 9, wherein the radioisotope is selected from the group consisting of a ³²P, a ³³P, ³⁵S, a ¹⁴C, an ¹²⁵I, an ¹³¹I and a ³H.
12. A method of detecting, diagnosing or monitoring kidney stone disease in accordance with aspect 9, wherein the fluorophore is selected from the group consisting of a fluorescein, a rhodamine, an Alexa Fluor®, an IRDye®, a coumarin, an indocyanine and a quantum dot.
13. A method of detecting, diagnosing or monitoring kidney stone disease in accordance with aspect 11, wherein the label is selected from the group consisting of a biotin, a digoxygenin, and a peptide comprising an epitope.
14. A method of detecting, diagnosing or monitoring kidney stone disease in accordance with aspect 1, wherein the measuring quantity of the primary complex comprises contacting the complex with at least one secondary probe that binds the antibody that binds Claudin-14 polypeptide under conditions sufficient for formation of a second complex comprising the at least one secondary probe, the antibody and the Claudin-14 polypeptide if present; and

measuring quantity of the second complex.

15. A method of detecting, diagnosing or monitoring kidney stone disease in accordance with aspect 14, wherein the at least one secondary probe is selected from the group consisting of an antibody directed against the antibody that binds Clauin-14 polypeptide, an aptamer that binds the antibody that binds Clauin-14 polypeptide, an avimer that binds the antibody that binds Clauin-14 polypeptide, an avidin and a streptavidin.
16. A method of detecting, diagnosing or monitoring kidney stone disease in accordance with aspect 11, wherein the at least one secondary probe comprises a label, and the measuring quantity of the second complex comprises quantifying the label.
17. A method of detecting, diagnosing or monitoring kidney stone disease in accordance with aspect 16, wherein the label is selected from the group consisting of an enzyme, a radioisotope, a fluorogen, fluorophore, a chromogen and a chromophore.
18. A method of detecting, diagnosing or monitoring kidney stone disease in accordance with aspect 17, wherein the enzyme is selected from the group consisting of a peroxidase, a phosphatase, a galactosidase and a luciferase.
19. A method of detecting, diagnosing or monitoring kidney stone disease in accordance with aspect 17, wherein the radioisotope is selected from the group consisting of a ³²P, a ³³P, ³⁵S, a ¹⁴C. an ¹²⁵I, an ¹³¹I and a ³H.
20. A method of detecting, diagnosing or monitoring kidney stone disease in accordance with aspect 17, wherein the fluorophore is selected from the group consisting of a fluorescein, a rhodamine, an Alexa Fluor®, an IRDye®, a coumarin, an indocyanine and a quantum dot.
21. A method of detecting, diagnosing or monitoring kidney stone disease in accordance with aspect 16, wherein the label is selected from the group consisting of a biotin, a digoxygenin, and a peptide comprising an epitope.
22. A method in accordance with aspect 1, wherein the subject is selected from the group consisting of a human, a rodent, a canine, a feline, a bovine, an ovine, a porcine, and an avian such as a chicken.
23. A method in accordance with aspect 1, wherein the subject is a mammal.
24. A method in accordance with aspect 1, wherein the subject is a human.
25. A method of detecting, diagnosing or monitoring kidney stone disease in accordance with aspect 1, further comprising administering a kidney stone treatment.
26. A method in accordance with aspect 25, wherein the kidney stone treatment is selected from the group consisting of an miR-9 mimic, an miR-374 mimic, an agent that inhibits signaling through the CaSR pathway, an antibody against Claudin-14, an antagonist of CaSR, NPS2143, at least one HDAC inhibitor, suberanilohydroxamic acid (SAHA), trichostatin A (TsA), and a combination thereof.
27. A method of monitoring kidney stone disease in a subject, comprising:

providing a first urine sample from a subject at a first time point;

contacting the sample with an antibody that binds Claudin-14 polypeptide, under conditions sufficient for formation of a first primary complex comprising the first antibody and Claudin-14 polypeptide if present;

measuring quantity of the first primary complex;

providing a second sample from the subject at a second time point;

contacting the sample with the antibody that binds Claudin-14 polypeptide, under conditions sufficient for formation of a second primary complex comprising the second antibody and Claudin-14 polypeptide if present; and

measuring quantity of the second primary complex wherein an increase compared to the first sample is diagnostic for increased kidney stone disease.

28. A method of detecting, diagnosing or monitoring kidney stone disease in accordance with aspect 27, further comprising administering a kidney stone treatment.
29. A method of detecting, diagnosing or monitoring kidney stone disease in accordance with aspect 28, wherein the kidney stone treatment is selected from the group consisting of an miR-9 mimic, an miR-374 mimic, an agent that inhibits signaling through the CaSR pathway, an antibody against Claudin-14, an antagonist of CaSR, NPS2143, at least one HDAC inhibitor, suberanilohydroxamic acid (SAHA), trichostatin A (TsA), and a combination thereof.
30. A method of treating kidney stone disease in a subject in need thereof, comprising;

administering to a subject a therapeutically effective amount of an miR-9 mimic, an miR-374 mimic or a combination thereof.

31. A method in accordance with aspect 30, wherein the administering is intravenous administering.
32. A method of treating kidney stone disease in a subject in need thereof, comprising;

administering to a subject a therapeutically effective amount of an agent that inhibits signaling through the CaSR pathway.

33. A method in accordance with aspect 32, wherein the agent is an antibody against Claudin-14.
34. A method in accordance with aspect 32, wherein the agent is an miR-9 mimic, an miR-374 mimic or a combination thereof.
35. A method of treating a disease associated with hypocalcemia comprising:

administering to a subject in need of treatment thereof a therapeutically effective anount of an antagonist of CaSR.

36. A method in accordance with claim 35, wherein the disease is selected from the group consisting of nephrolithiasis, osteoporosis, hypoparathyroidism, Alzheimer's disease, and epilepsy.
37. A method of treating kidney stone disease comprising:

administering to a subject in need of treatment thereof a therapeutically effective amount of an antagonist of CaSR.

38. A method in accordance with aspect 35 or 37, wherein the CaSR antagonist is a calcilytic compound.
39. A method in accordance with aspect 35 or 37, wherein the CaSR antagonist is NPS2143.
40. A method in accordance with aspect 35 or 37, wherein administering is by oral administration.
41. A method in accordance with aspect 35 or 37, wherein the therapeutically effective amount of NPS2143 is about 15 mg/kg BW⁻¹, about 15 mg/kg BW⁻¹-45 mg/kg BW⁻¹, or about 45 mg/kg BW⁻¹.
42. A method in accordance with aspect 35 or 37, wherein the therapeutically effective amount of NPS2143 is 15 mg/kg BW⁻¹.
43. A method in accordance with aspect 35 or 37, wherein the therapeutically effective amount of NPS2143 is 30 mg/kg BW⁻¹.
44. A method in accordance with aspect 35 or 37, wherein the therapeutically effective amount of NPS2143 is 45 mg/kg BW⁻¹.
45. A method of treating a disease selected from the group consisting of hyperparathyroidism and hypercalcemia, comprising:

administering an agonist of CaSR.

46. A method in accordance with aspect 45, wherein the agonist of CaSR is a calcimimetic or a cinacalcet.
47. A method in accordance with aspect 45, wherein administering is by oral administration.
48. A method in accordance with aspect 45, wherein the therapeutically effective amount of cinacalcet is 30 mg/kg BW⁻¹ or about 30 mg/kg BW⁻¹.
49. A method of abrogating CaSR mediated regulation of claudin-14, microRNAs and urinary Ca++ excretion in a subject in need thereof, comprising:

administering to a subject a therapeutically effective amount of a calcineurin inhibitor.

50. A method in accordance with aspect 49, wherein the calcineurin inhibitor is cyclosporine-A.
51. A method of treating kidney stone disease in a subject in need thereof, comprising;

administering to a subject a therapeutically effective amount of at least one HDAC inhibitor.

52. A method in accordance with aspect 51, wherein the HDAC inhibitor is selected from the group consisting of suberanilohydroxamic acid (SAHA) and trichostatin A (TsA).
53. A method in accordance with aspect 52, wherein the therapeutically effective amount of SAHA is about 5 mg/kg, 5 mg/kg-25 mg/kg, or about 25 mg/kg.
54. A method in accordance with aspect 52, wherein the therapeutically effective amount of SAHA is at least 5 mg/kg or about 5 mg/kg.
55. A method in accordance with aspect 52, wherein the therapeutically effective amount of TsA is at least 1 mg/kg or about 1 mg/kg.

### BRIEF DESCRIPTION OF THE DRAWINGS

FIG. 1 illustrates CLDN14 gene localization in the kidney. (A) β-galactosidase activity in renal tubules detected by lacZ reporter assay. Bar: 100 μm. (B) Microdissected tubular segments from the mouse kidney. Microdissection criterion: proximal convoluted tubule (PCT), distal convoluted tubule (DCT) and thick ascending limb (TAL) joined at glomerulus/macula densa are dissected out (i); glomerulus (Glom) and PCT are separated from macula densa (ii): PCT is separated from glomerulus and DCT is separated from TAL free of any junctional tissue—macula densa (iii); proximal straight tubule (PST) is separated from PCT, connecting tubule/collecting duct (CNT/CD) separated from DCT and TAL according to morphological differences (iv). (C) CLDN14 and CLDN16 mRNA levels relative to f-actin mRNA levels in microdissected tubular segments. *: p<0.05, n=3.

FIG. 2 illustrates microRNA regulation of CLDN14 expression in the kidney. (A-C) The firefly/Renilla luciferase activity ratios measured in HEK293 cells transfected with pMir-Reporter-CLDN14:3′-UTRmouse and either scrambled miRNA, miR-9 or miR-374 precursor (A); with pMir-Reporter-CLDN14:3′-UTRhuman and miRNA precursor (B); with pMir-Reporter-CLDN14:3′-UTRhuman containing deletions in miRNA binding sites (C). (D) Transfection with antagomirs increases pMir-Reporter-CLDN14:3′-UTRmouse activity in MKTAL cells. (E) CLDN14 mRNA levels relative to β-actin mRNA levels in MKTAL cells transfected with antagomirs. (F) CLDN 14 protein levels relative to β-actin protein levels in MKTAL cells transfected with antagomirs. (G) Antagomir transfection increases pMir-Reporter-CLDN14:3′-UTRmouse activity in freshly isolated mouse TAL cells. (H) CLDN14 mRNA levels in freshly isolated mouse TAL cells transfected with antagomirs.

FIG. 3 illustrates that CLDN14 interacts with CLDN16. (A) Y2H assays showing interaction of CLDN14 with CLDN16 but not with CLDN19, determined by three reporter genes (HIS3, lacZ, and ADE2) in the yeast NMY51 strain. Shown are plates with selective medium lacking leucine and tryptophan (SD-LW), indicating the transforming of both bait and prey vectors; with SD-LWH and SD-LWHA, indicating the expression of reporter genes HIS3 and ADE2; and the β-galactosidase assay. (B) Quantitative β-galactosidase assay to determine the relative interaction strength of the following pairs: CLDN16-19, CLDN14-16 and CLDN14-19. (C-D) Coimmnunoprecipitation of claudins in doubly transfected HEK293 cells to determine CLDN14-16 interaction (C) and CLDN14-19 interaction (D). (E) Coimmunoprecipitation of claudins in triply transfected HEK293 cells to determine CLDN14-16-19 interaction. Antibodies used for immunoprecipitation are shown above the lanes; antibody for blot visualization is shown at left. Nonspecific 1: anti-CLDN1; Nonspecific 2: anti-occludin; *Lane shows 10% of input amount as in other lanes.

FIG. 4 ilhlstrates effects of CLDN14 on CLDN16 and CLDN19 function in combinatorial expression assays. Diffusion potential values across LLC-PK1 cell monolayers expressing CLDN14, CLDN16 and CLDN19, individually or in combinations, are shown. Positive diffusion potential reflects cation selectivity of paracellular channel while negative potential reflects anion selectivity.

FIG. 5 illustrates extracellular Ca²⁺ regulation of CLDN14 in the kidney. (A) The plasma Ca²⁺ levels in mice receiving dietary Ca²⁺ variations. (B) CLDN14 mRNA levels relative to f-actin mRNA levels in mouse kidney TALs receiving dietary Ca²⁺ variations. (C) Immunostaining on mouse kidney sagittal sections (10 μm) shows CLDN14 protein localization in the tight junctions (arrow-head) of TAL tubules from animal receiving high Ca² diet but not basal diet. Bar: 50 μm. (D) CLDN14 protein levels relative to β-actin protein levels in mouse kidney TALs receiving dietary Ca²⁺ variations. (E) CLDN14 mRNA levels in MKTAL cells cultured in 0-3 mM Ca²⁺. (F) Quantitation of CLDN14 protein levels in MKTAL cells cultured in 3 mM Ca²⁺ versus 0.1 mM Ca². (G) CLDN14 mRNA levels in MKTAL cells cultured in 0-100 nM PTH.

FIG. 6 illustrates extracellular Ca²⁺ regulation of microRNAs in the kidney. (A) miR-9, (B) miR-374 transcript levels relative to U6 snRNA transcript levels in microdissected tubular segments. *: p<0.05, n=3. (C) miR-9, (D) miR-374 transcript levels in mouse kidney TALs receiving dietary Ca²⁺ variations. (E) miR-9, (F) miR-374 transcript levels in MKTAL cells cultured in 0-3 mM Ca²⁺.

FIG. 7 illustrates a role of CaSR in extracellular Ca²⁺ regulation of CLDN14 and microRNAs. (A) CLDN14 mRNA, (B) CLDN14 protein, (C) miR-9 transcript, (D) miR-374 transcript levels in MKTAL cells transfected with either CaSR siRNA or scrambled siRNA, followed by induction with 3 mM Ca²⁺ versus 0.1 mM Ca²⁺.

FIG. 8 illustrates NPS2143 and cinacalcet regulation of claudin-14 in the kidney. (A-B) Time response of NPS2143 (A) and cinacalcet (B) effects on claudin-14 mRNA levels. (C-D) Dose response of NPS2143 (C) and cinacalcet (D) effects on claudin-14 mRNA levels. (E) Claudin-14 protein levels in NPS2143, vehicle or cinacalcet treated mouse kidneys. (F) Immunostaining on mouse kidney sagittal sections shows claudin-14 protein localization in the tight junctions of TALH tubules from animal receiving NPS2143, vehicle or cinacalcet. Bar: 20 μm. (G) Effects of 12 hr pretreatment with NPS2143, vehicle or cinacalcet on claudin-14 mRNA levels. (H-I) Effects of 6d treatment with NPS2143, vehicle or cinacalcet on claudin-14 mRNA (H) and protein (I) levels.

FIG. 9 illustrates Claudin-14 gene regulation and PTH secretion. (A-C) Plasma Ca²⁺ (A), plasma PTH (B) and renal claudin-14 mRNA (C) levels in animals fed with low Ca²⁺ diet or basal diet. (D-F) Plasma Ca²⁺ (D), plasma PTH (E) and renal claudin-14 mRNA (F) levels in animals operated with TPTX or sham. (G) Effects of NPS2143, vehicle or cinacalcet on claudin-14 mRNA levels in TPTX operated mice.

FIG. 10 illustrates plasma and urine Ca²⁺ and Mg²⁺ levels in WT and claudin-14 KO mice. (A-E) Effects of NPS2143, vehicle or cinacalcet on plasma Ca²⁺ (A), fractional excretion of Ca²⁺ (B), plasma Mg²⁺ (C), fractional excretion of Mg²⁺ (D) and plasma PTH (E) levels in WT mice at 2 hrs versus 8 hrs. (F-J) Effects of NPS2143, vehicle or cinacalcet on plasma Ca²⁺ (F), fractional excretion of Ca²⁺ (G), plasma Mg²⁺ (H), fractional excretion of Mg2+ (I) and plasma PTH (J) levels in WT versus KO mice at 2 hrs.

FIG. 11 illustrates characterization of claudin-14 TG mice. (A) Diagram of claudin-14 WT allele and TG allele. (B-C) Overall claudin-14 (B) and endogenous claudin-14 (C) mRNA levels in TG versus WT mouse kidneys. (D) Claudin-14 protein levels in TALH tubules from TG versus WT mouse kidneys. (E) Immunostaining on sagittal sections of TG versus WT mouse kidneys. Bar: 20 μm. (F-I) Plasma Ca²⁺ (F), Mg²⁺ (G), fractional excretion of Ca²⁺ (H) and Mg²⁺ (I) levels in TG versus WT mice.

FIG. 12 illustrates NPS2143 and cinacalcet regulation of microRNA in the kidney. (A-D) Effects of NPS2143, vehicle or cinacalcet on pri-miR-9-1 (A), pri-miR-9-2 (B), pri-miR-9-3 (C) and pri-miR-374 (D) mRNA levels in TALH tubules from WT mouse kidneys at 2 hr versus 8 hr. (E-F) Effects of NPS2143, vehicle or cinacalcet on pri-miR-9-1 (E) and pri-miR-374 (F) mRNA levels in TPTX operated mice. (G-H) Effects of cinacalcet on lacZ mRNA (G) and protein activity (H) levels in claudin-14+/lacZ reporter mouse kidneys.

FIG. 13 illustrates cyclosporin effects on claudin-14, microRNA and renal Ca²⁺ excretion. (A-C) Effects of NPS2143, vehicle or cinacalcet on claudin-14 (A), pri-miR-9-1 (B) and pri-miR-374 (C) mRNA levels in cyclosporine pre-treated mouse kidneys. (D-H) Effects of NPS2143, vehicle or cinacalcet on plasma Ca²⁺ (D), fractional excretion of Ca²⁺ (E), plasma Mg²⁺ (F), fractional excretion of Mg²⁺ (G) and plasma PTH (H) levels at 2 hrs in WT mice pre-treated with cyclosporine.

FIG. 14 illustrates NFAT regulation of microRNA promoter. (A) RT-PCR of NFATc1-c4 mRNA levels in WT mouse TALH tubules. (B) Effects of NFAT knockdown on claudin-14 mRNA levels. (C-G) Effects of NFATc1nuc transfection on claudin-14 (C), pri-miR-9-1 (D), pri-miR-9-2 (E), pri-miR-9-3 (F) and pri-miR-374 (G) mRNA levels. (H-I) Effects of NFATc1nuc transfection on miR-9-1 (H) and miR-374 (I) promoter activities. (J-M) Effects of NFATc1nuc transfection on promoter binding (J) and histone acetylation (K) levels for miR-9-1 and miR-374 (L-M) genes. (N-Q) Effects of NPS2143, vehicle or cinacalcet on promoter binding (N) and histone acetylation (O) levels for miR-9-1 and miR-374 (P-Q) genes.

FIG. 15 illustrates anti-microRNA treatments in mouse kidney. (A) Transfection with anti-miR-9 or anti-miR-374 increased claudin-14 gene expression in freshly isolated mouse TALH tubular cells. (B) Intravenous injection with anti-microRNA significantly reduced the plasma Ca²⁺ levels in adult mice. (C) Intravenous injection with anti-microRNA significantly increased the urinary excretion levels of Ca²⁺ in adult mice.

FIG. 16 illustrates tight junction localization of CLDN14, 16 and 19 in kidney epithelial cells. Confocal microscopy shows the localization of CLDN14, 16 and 19 in triply transfected LLC-PK1 cells. Over 90% of transfected cells co-express CLDN14, 16 and 19. All three claudins are correctly localized to the tight junction. Bar: 20 μm.

FIG. 17 illustrates tight junction localization of CLDN16 and CLDN19 in the kidney. Cryostat sagittal sections (10 μm) from the mouse kidneys show CLDN16 and CLDN19 localization in the tight junctions of TAL tubules. The localization patterns of CLDN16 and CLDN19 are not affected by dietary Ca²⁺ variations. Bar: 50 μm.

FIG. 18 illustrates HDAC inhibitor regulation of CLDN14 and microRNA gene expression in the kidney. (A-B) The dose response of HDAC inhibitor—SAHA (A) and TsA (B) effects on CLDN14 mRNA levels in primary cultures of mouse TALH. (C) The time response of SAHA effects on CLDN14 mRNA levels in mouse kidneys. (D) The dose response of SAHA effects on CLDN14 mRNA levels in mouse kidneys. (E) CLDN14 proteins levels in SAHA or vehicle treated freshly isolated mouse TALH tubules. (F) The effects of TsA on CLDN14 mRNA levels in mouse kidneys. (G) The effects of SAHA on lacZ mRNA levels in CLDN14^{lacZ/+} reporter mouse kidneys. (H-I) The effects of SAHA on microRNA transcription measured for pri-miR-9-1 (H) and pri-miR-374 (I) levels. (J-K) The effects of SAHA on histone acetylation levels over the microRNA gene promoters measured for miR-9-1 (J) and miR-374 (K) genes with chromatin imnuunoprecipitation (ChIP). *p<0.05; **p<0.01; ***p<0.001.

FIG. 19 illustrates plasma and urine electrolyte levels in WT and CLDN14 KO mice treated with SAHA and/or furosemide. (A-B) The time response of SAHA effects on urinary excretion rates for Ca⁺⁺ (A) and Mg⁺⁺ (B) in WT mice with spot urine collections and shown as ratios to urinary creatinine levels. (C-D) The effects of SAHA on fractional excretion rates for Ca⁺⁺ (C) and Mg⁺⁺ (D) in WT versus CLDN14 KO mice or mice pretreated with furosemide with 24 hr urine collections. *p<0.05; **p<0.01.

FIG. 20 illustrates renal effects of antagomir treatments. (A) CLDN14 mRNA levels in primary TALH cultures transfected with antagomirs against miR-9 or miR-374. (B) CLDN14 mRNA levels in mouse kidneys treated with antagomirs against miR-9 or miR-374. (C) CLDN14 protein levels in freshly isolated TALHs from mouse kidneys treated with anti-miR-374 versus scrambled antagomir. (D) Mouse kidney sagittal sections immunostained for CLDN14 from anti-miR-374 versus scrambled treatments. Bar: 10 μm. (E-G) Plasma Mg⁺⁺ (E), fractional excretion of Ca⁺⁺ (F) and Mg (G) levels in anti-miR-374 versus scrambled animal groups. *p<0.05; **p<0.01; ***p<0.001.

FIG. 21 illustrates CLDN14 protein levels in human urinary exosomes. Representative immunoblot images (A) and quantitative densitometric data (B) (n=8 in SF group: n=7 in HV group) showing levels of CLDN14 and THP proteins and their abundance ratios in human urinary exosomes from kidney stone patients versus location matched healthy controls. SF: stone former, HV: healthy volunteer.

FIG. 22 illustrates epigenetic effects on claudin-14 mRNA levels in primary cultures of mouse TALH cells. The mouse TALH cells were cultured for 16 hrs in presence of 5μ M 5-Aza-2′-deoxycytidine (AZA), 0.1μ M BIX 01294 and 10μ M tranylcypromine respectively, assayed for CLDN14 mRNA levels with real-time PCR, normalized to β-actin mRNA, and compared to vehicle treatments (N=3).

FIG. 23 illustrates SAHA effects on NFATc1 binding to microRNA promoters. Wild-type C57BL/6 mice were treated with 25 mg/kg BW-1 SARA for 4 hrs and assayed for NFATc1 binding affinity to miR-9-1 (A) and miR-374 (B) gene promoters in the kidney with ChiP.

FIG. 24 illustrates circulating hormonal levels in SAHA versus vehicle treated mouse kidneys. Serum PTH (A), 1,25-(OH)2-vitD3 (B) and FGF23 (C) levels were measured with ELISA and shown as mean±SEM, n=5.

FIG. 25 illustrates gene expression analyses of renal ion transporters in SAHA versus vehicle treated mouse kidneys by real-time PCR. Values are shown as fold ratios relative to the vehicle level (as mean±SEM; n=5).

FIG. 26 illustrates NPS2143 and cinacalcet regulation of claudin-14 in the kidney. (A-B) Time response of NPS2143 (A) and cinacalcet (B) effects on claudin-14 mRNA levels. (C-D) Dose response of NPS2143 (C) and cinacalcet (D) effects on claudin-14 mRNA levels. (E) Claudin-14 protein levels in NPS2143, vehicle or cinacalcet treated mouse kidneys. (F) Immunostaining on mouse kidney sagittal sections shows claudin-14 protein localization in the tight junctions of TALH tubules from animal receiving NPS2143, vehicle or cinacalcet. Bar: 20 μm. (G) Effects of 12 hr pretreatment with NPS2143, vehicle or cinacalcet on claudin-14 mRNA levels. (H-I) Effects of 6d treatment with NPS2143, vehicle or cinacalcet on claudin-14 mRNA (H) and protein (I) levels. (J-K) Effects of NPS2143, vehicle or cinacalcet on claudin-14 mRNA levels in TPTX operated mice (J) relative to its basal level in sham-operated animals (K). *: p<0.05: **: p<0.01.

FIG. 27 illustrates plasma and urine Ca⁺⁺ and Mg⁺⁺ levels in WT and claudin-14 KO mice. (A-E) Effects of NPS2143, vehicle or cinacalcet on plasma Ca⁺⁺ (A), fractional excretion of Ca⁺⁺ (B), plasma Mg⁺⁺ (C), fractional excretion of Mg⁺⁺ (D) and plasma PTH (E) levels in WT mice at 2 hrs versus 8 hrs. (F-J) Effects of NPS2143, vehicle or cinacalcet on plasma Ca⁺⁺ (F), fractional excretion of Ca⁺⁺ (G), plasma Mg⁺⁺ (H), fractional excretion of Mg⁺⁺ (I) and plasma PTH (J) levels in WT versus KO mice at 2 hrs.

FIG. 28 illustrates characterization of claudin-14 TG mice. (A) Diagram of claudin-14 WT allele and TG allele. (B-C) Overall claudin-14 (B) and endogenous claudin-14 (C) mRNA levels in TG versus WT mouse kidneys. (D) Claudin-14 protein levels in TALH tubules from TG versus WT mouse kidneys. (E) Immunostaining on sagittal sections of TG versus WT mouse kidneys. Bar: 20 μm. (F-I) Plasma Ca⁺⁺ (F), Mg⁺⁺ (G), fractional excretion of Ca⁺⁺ (H) and Mg⁺⁺ (I) levels in TG versus WT mice.

FIG. 29 illustrates NPS2143 and cinacalcet regulation of microRNA in the kidney. (A-D) Effects of NPS2143, vehicle or cinacalcet on pri-miR-9-1 (A), pri-miR-9-2 (B), pri-miR-9-3 (C) and pri-miR-374 (D) mRNA levels in TALH tubules from WT mouse kidneys at 2 hr versus 8 hr. (E-F) Effects of NPS2143, vehicle or cinacalcet on pri-miR-9-1 (E) and pri-miR-374 (F) mRNA levels in TPTX operated mice. (G-H) Effects of cinacalcet on lacZ mRNA (G) and protein activity (H) levels in claudin-14^{+/lacZ} reporter mouse kidneys.

FIG. 30 illustrates cyclosporin effects on claudin-14, microRNA and renal Ca⁺⁺ excretion. (A-C) Effects of NPS2143, vehicle or cinacalcet on claudin-14 (A), pri-miR-9-1 (B) and pri-miR-374 (C) mRNA levels in cyclosporine pre-treated mouse kidneys. (D-H) Effects of NPS2143, vehicle or cinacalcet on plasma Ca⁺⁺ (D), fractional excretion of Ca⁺⁺ (E), plasma Mg⁺⁺ (F), fractional excretion of Mg⁺⁺ (G) and plasma PTH (H) levels at 2 hrs in WT mice pre-treated with cyclosporine.

FIG. 31 illustrates NFAT regulation of microRNA promoter. (A) Effects of NFAT knockdown on claudin-14 mRNA levels. (B-F) Effects of NFATc1nuc transfection on claudin-14 (B), pri-miR-9-1 (C), pri-miR-9-2 (D), pri-miR-9-3 (E) and pri-miR-374 (F) mRNA levels. (G-H) Effects of NFATc1nuc transfection on miR-9-1 (G) and miR-374 (H) promoter activities. (I-L) Effects of NFATc1nuc transfection on promoter binding (I) and histone acetylation (J) levels for miR-9-1 and miR-374 (K-L) genes. (M-P) Effects of NPS2143, vehicle or cinacalcet on promoter binding (M) and histone acetylation (N) levels for miR-9-1 and miR-374 (O-P) genes.

FIG. 32 illustrates effects of NPS2143 and cinacalcet on claudin-14 gene expression in FK506 pre-treated mouse kidneys. FK506 abolished NPS2143 induced downregulation while attenuated cinacalcet induced upregulation of claudin-14 mRNA levels. *: p<0.05.

FIG. 33 illustrates RT-PCR of NFATc 1-c4 mRNA levels in mouse TALH tubules. The TALH tubular cells were inuunoisolated from pooled mouse kidneys with the TALH specific antibody—anti-THP (Tamm-Horsfall protein) for RNA extraction and RT-PCR analyses.

FIG. 34 illustrates SiRNA mediated knockdown of NFATs in mouse TALH cells. Pre-validated Silencer Select siRNAs (Ambion) against NFATc1-4 were transfected into primary cultures of mouse TALH cells with Lipofectamine-LTX & Plus reagent. 24 hr after transfection, the mRNA levels of NFATc1-4 were quantified with real-time PCR. The knockdown efficacy was between 50-60% for NFAT siRNAs, likely corresponding to the transfection efficiency at similar levels. The basal expression level of each NFAT was set at 1.0, mindful that NFATc4 was expressed far lower (<1/10) than other NFATs. **: p<0.01.

### DETAILED DESCRIPTION

### Abbreviations

ANOVA: Analysis of variance
CaSR: Ca++ sensing receptor
ChIP: Chromatin immunoprecipitation
CKD: Chronic kidney disease
Ct: Cycle threshold

### DMEM: Dulbecco's Modified Eagle Medium

DMSO: Dimethyl sulfoxide
ECV: Extracellular volume
FBS: Fetal bovine serum
FE: Fractional excretion
FITC: Fluorescein isothiocyanate
FHH: Familial hypocalciuric hypercalcemia
FHHNC: Familial hypomagnesemia with hypercalciuria and nephrocalcinosis

### Furo: Furosemide

GFR: Glomerular filtration rate
GPCR: G protein-coupled receptors
GWAS: Genome-wide association study
HDAC: Histone deacetylases
HEK293: Human Embryonic Kidney 293 cells
HV: Healthy volunteers
IP injection or I.P. injection: Intraperitoneal injection

### KO: Knock out

LNA: Locked nucleic acid
ncRNA: Non-coding RNA
NFAT: Nuclear factor of activated T-cells
NSHPT: Neonatal severe hyperparathyroidism

### OMIM: Online Mendelian Inheritance in Man

PBS: Phosphate buffered saline
P_{ca}: Plasma calcium concentration
PCR: Polymerase chain reaction
P_{Mg}: Plasma magnesium concentration
PTH: Parathyroid hormone
SAHA: Suberanilohydroxamic acid
SEM: Standard error of the mean
SF: Stone formers
TALH: Thick ascending limb of Henle's loop
THP: Tamm-Horsfall protein

### TIFF: Tagged Image File Format

TJ: Tight junction

### TPTX: Thyroparathyroidectomy

### TsA: Trichostatin A

UTR: Untranslated region
UV: Urine volume

### Veh: Vehicle

WT: Wild type
Y2H: Yeast two-hybrid

The inventors demonstrated that claudin-14 fulfills the transport role for Ca²⁺ in the kidney. The mRNA, protein and TJ localization levels of claudin-14 in the kidney can be regulated within hours by systemic administration of calcimimnetic and calcilytic compounds.

Using a knockout (KO) strategy and time-controlled renal clearance measurements, the inventors have demonstrated a functional role of claudin-14 in CaSR-induced calciuretic and magnesiuretic responses. The experiments were carried out in intact animals and PTH levels were simultaneously monitored and found similar in WT and claudin-14 KO following calcimimetic and calcilytic administration, arguing against the role of PTH in CaSR mediated renal function. PTH appeared to set a “floor” level defending against hypocalcemia, mindful that KO of claudin-14 could not correct the hypocalcemia induced by cinacalcet. This phenomenon has also been observed by Kantham et al., 2009 showing that the presence of CaSR in the setting of PTH KO could not rescue the CaSR/PTH double KO from hypocalcemia.

The inventors discovered a transcriptional program in the TALH of the kidney that comprises NFATc 1-microRNA and plays a functional role through the regulation of claudin-14. Our data can explain how calcineurin inhibitor cyclosporine can abolish the NPS2143 effect both on the levels of claudin-14 gene regulation and calciuretic response. Consistent with NPS2143 eliciting profound decreases in claudin-14 gene expression, NFATc1 binding to miR-9-1 and miR-374 promoters was concomitantly increased, inducing local histone H3 acetylation and stimulating microRNA transcription. Since claudin-14 transcripts are already present intracellularly, alteration of microRNA transcription allows rapid translational regulation of its target protein, claudin-14, ensuring timely functional regulation for the signaling pathway. The cinacalcet effect can be attenuated but not abolished by cyclosporine. There was not a significant change in NFATc 1 binding to microRNA promoter or histone acetylation.

The inventors demonstrate that claudin-14 inhibits claudin-16 permeability and integrates into the claudin-16/-19 channel complex using several biochemical, biophysical and cellular approaches. The inventors present in vivo evidence that overexpression of claudin-14 in the TALH of the kidney generates a renal phenotype characteristic with hypomagnesemia and hypercalciuria, and characteristic with claudin-16 KO phenotype (Hou et al., 2007). The inventors discovered that claudin-14 is a regulatory molecule for CaSR. Accumulating data demonstrated that paracellular Ca²⁺ reabsorption in the TALH can be directly regulated by CaSR during hypercalcemia (Desfleurs et al., 1998; Motoyama and Friedman, 2002). Through physical interactions, claudin-14 blocks the paracellular channel made of claudin-16 and -19, suggesting a mechanism for its role in nephrolithiasis. Tight junction (TJ) proteins were previously considered to be constitutive and structural molecules. Claudin-14 is the first TJ molecule of which the expression can be rapidly regulated in response to physiological changes.

In this study, The inventors demonstrate that extracellular Ca²⁺, through activation of CaSR, regulates the expression levels of two microRNAs: miR-9 and miR-374, which in turn transduce the extracellular signal to CLDN14 through microRNA mediated gene silencing. CLDN14 relays the extracellular Ca²⁺ signal to CLDN16-19, the final effector of Ca 2, transport in the kidney, through direct functional modulation of their permeabilities.

Under normal physiological conditions, miR-9 and miR-374 tightly regulate the gene expression level of CLDN14 and protect CLDN16-19 channel function. The observed association between CLDN14 and hypercalciuric nephrolithiasis (Thorleifsson et al., 2009) can be explained by CLDN14 deregulation that escapes microRNA suppression, inhibits CLDN16-19 channel permeabilities, and phenocopies FHHNC to variable degrees. FHHNC patients (Konrad et al., 2006; Weber et al., 2001) and animal models (Hou et al., 2007: 2009) with CLDN16 or CLDN19 mutations are known to have hypercalciuria, nephrocalcinosis and nephrolithiasis. CLDN14 deregulation has two distinct origins: cis- or trans-acting. Cis-acting variants include changes in promoters, splicing sites and microRNA target sites. The miR-9 and miR-374 target sites in the CLDN14 gene are surrounded by four synonymous SNPs that associate with hypercalciuric nephrolithiasis (Thorleifsson et al., 2009), suggesting the linkage disequilibrium (LD) block may contain susceptible genetic variations related to microRNA regulation. Allelic variation in mRNA levels presents another explanation for association of exonic SNPs. Two of the four identified SNPs are located in the last exon of CLDN14 gene, which may be associated with abnormally higher levels of CLDN14 mRNA in the kidney (Yan et al., 2002). Trans-acting variants are located in distant genes that alter the transcript levels of a target gene. miR-9 and miR-374 are trans-acting effectors of the CLDN14 gene. The gene transcription level of miR-9 itself is regulated by the myc and ras oncoproteins (Ma et al., 2010), implicating a potential tumorigenic role for CLDN14 as reported for CLDN2 (Buchert et al., 2010) and CLDN7 (Nilbel et al., 2009).

A role of CaSR in the kidney is the regulation of Ca²⁺ reabsorption in the thick ascending limb (TAL). While mostly found in the luminal membrane elsewhere of the nephron, CaSR is located in the basolateral membrane of the TAL, where it senses peritubular Ca²⁺ changes and regulates both transcelhdlar and paracelular electrolyte transports (Riccardi and Brown, 2010). An extensive literature testifies to the suppression of paracellular Ca²⁺ reabsorption by CaSR activation during hypercalcemia (Desfleurs et al., 1998; Motoyama et al, 2002). CaSR activation also inhibits ROMK channels (Wang et al., 1996; 1997), diminishes transcellular NaCl reabsorption and produces a “Bartter-like” phenotype (Vargas-Poussou et al., 2002). Several signaling pathways have been revealed underpinning CaSR regulation of transcellular channels, including P-450 metabolites and prostaglandins (Hebert et al., 2007). Owing to the short seed sequence (nucleotides: 2-7) of microRNA, a cognate microRNA regulates multiple target genes.

Although this study reported miR-9 and miR-374 convergence onto CLDN14, they could extend CaSR signaling to cellular functions beyond the paracellular channel and organ functions beyond the kidney. The regulation of microRNA by CaSR signaling may occur on several layers: microRNA transcription, processing or degradation (Krol et al., 2010). The promoters of both miR-9 (miR-9-3 locus) and miR-374 genes contain a canonical myc-binding site (E-box: CACGTG). The transcription of miR-9-3 is upregulated by myc in human breast cancer cells (Ma et al., 2010); miR-421/-374 cluster is upregulated by myc in Hela cells although miR-374 itself has not been measured (Hu et al., 2010). Genetic ablation of Dicer, a nuclease required for microRNA processing, in the proximal tubule (Wei et al., 2010) and the glomerular podocyte (Harvey et al., 2008) demonstrated a role for microRNA processing in renal pathophysiology. It is not known whether myc or Dicer is directly involved in CaSR signaling.

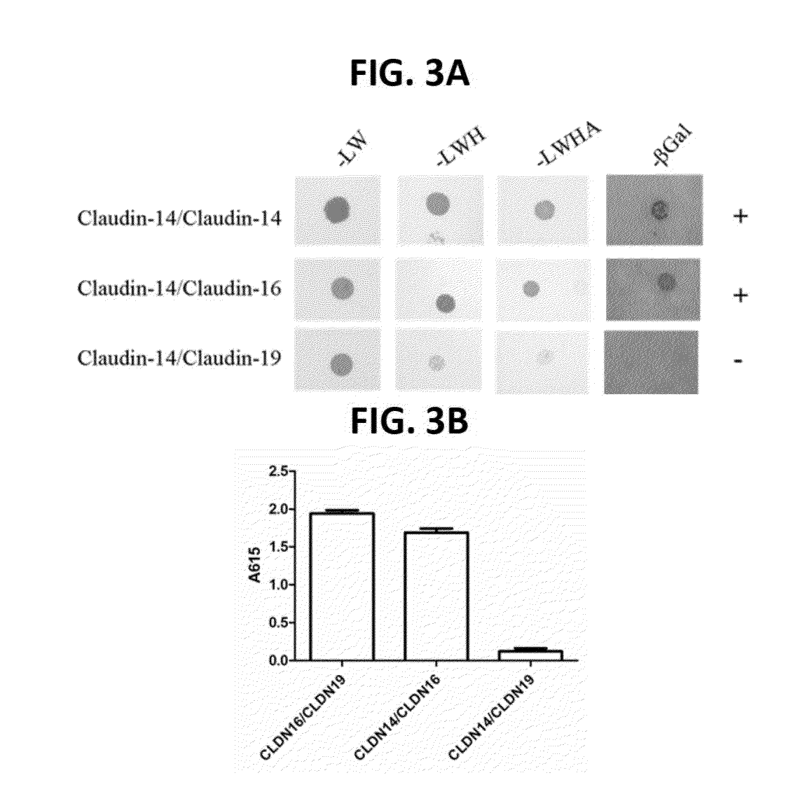

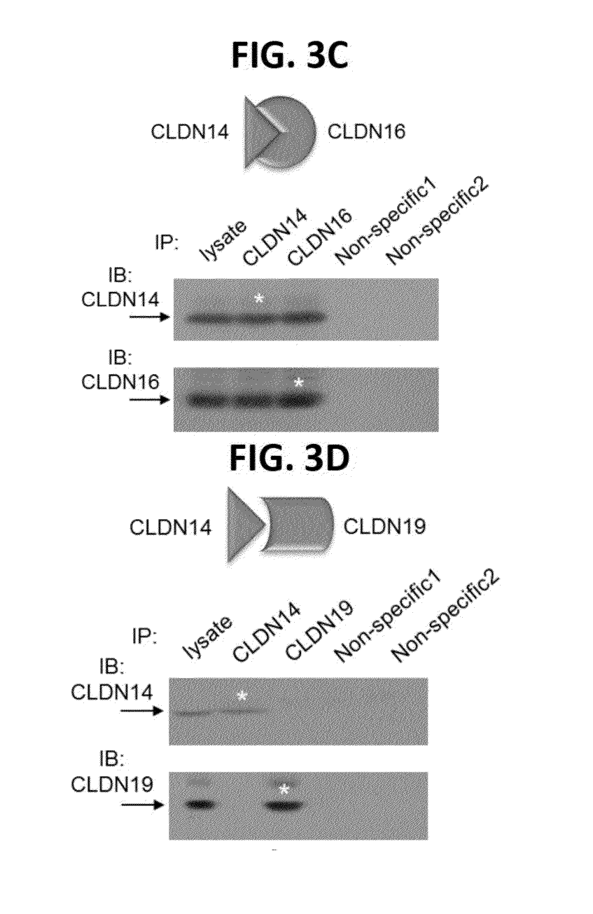

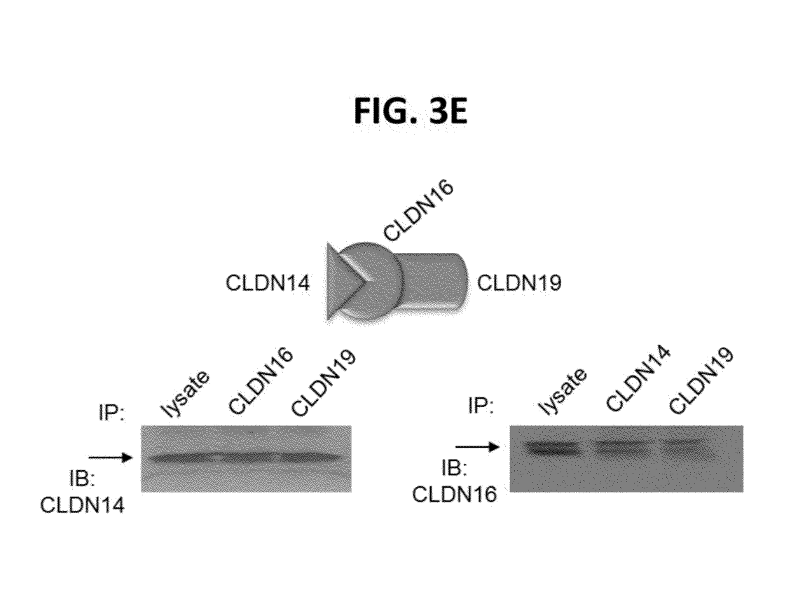

CLDN14 physically interacts with CLDN16 and directly blocks its cation permeability. Our data is consistent with a previous finding of CLDN14 as a non-selective cation blocker in kidney MDCK cells (Ben-Yosef et al., 2003). The paracellular barrier function of CLDN14 also underlies its physiological role in the inner ear (Ben-Yosef et al., 2003). CLDN14 showed strong homomeric interaction on our yeast 2-hybrid reporter assays (FIG. 3A). Freeze-fracture replicas showed well-developed networks of TJ strands in CLDN14-expressing fibroblast cells (Van Itallie et al., 2005), suggesting CLDN14 is capable of self-assembly into tight junction.

Although CLDN14 does not physically interact with CLDN19, our biochemical data suggest that CLDN14 integrates into CLDN16-19 channel to form a higher oligomeric complex with novel physiological signature. Using Brownian dynamics simulations, a single-pore model has been suggested for claudin channel structure (Yu et al., 2009). The channel pore is formed by two hemi-channels located in the TJ of adjacent cells which has a 6.5-Å diameter cylindrical shape and charged side chain on the conserved residue-65. The channel conductance varies with the effective charge valence on the side chain of residue-65 (Yu et al., 2009). CLDN14 has a non-charged residue-glutamine at position-65, which will reduce the effective charge density of CLDN16 channel pore (D65: −1e→0) once CLDN14 cis-associates with CLDN16, leading to decreases in cation permeation.

Gene regulation has two distinct origins: cis- or trans-acting. Cis-acting elements include promoter, splicing sites, ribosome entry sites and microRNA target sites. Trans-acting components include transcriptional factors, epigenetic modulators and non-coding RNAs (ncRNAs). MicroRNAs are single-stranded, ncRNA molecules of 19-25 nt in length, which are generated from endogenous hairpin-shaped transcripts. (Krol J, et al., 2010) base pair with their target mRNAs and induce either translational repression or mRNA destabilization. (Berezikov E. 2011) Our previous work identified two microRNA molecules from the kidney that target the 3′-UTR of claudin-14 gene: miR-9 and miR-374. (Gong Y, et al., 2009) Here, The inventors show that the transcriptional levels of both microRNAs are directly regulated by CaSR in the kidney, while the promoter activity of claudin-14 itself is not affected. A bioinformatic search found strong NFAT binding sites within the proximal promoter region of both miR-9 and miR-374 genes. Using ChIP analyses, the inventors demonstrated direct binding of NFATc1 to miR-9 and miR-374 promoters. The calcineurin inhibitor—cyclosporine abrogates CaSR mediated regulation of claudin-14, microRNAs and urinary Ca++ excretion in the kidney. The promoter binding of NFATc1 is regulated by CaSR and found to induce local histone acetylation, providing a mechanism to integrate CaSR based extracellular Ca++ signaling with calcineurin based intracellular Ca++ signaling pathways.

Using CaSR-specific pharmacological reagents, the inventors demonstrated that CaSR regulates the gene expression of claudin-14 in the kidney transiently. Using a transgenic approach, the inventors show that gain of claudin-14 function in the kidney induces renal Mg⁺⁺ and Ca⁴⁺ losses, demonstrating a physiological origin of kidney stone disease. The mRNA, protein and TJ localization of claudin-14 peaked at 2-4 hrs that coincided with maximal Ca⁺⁺ transport levels. Knockout of claudin-14 abolished the renal Ca⁺⁺ transport induced by CaSR.

Claudins form a class of channels oriented perpendicular to the membrane plane and serving to join two extracellular compartments, known as the paracellular channel. (Hou, J., et al., 2013) The paracellular Ca⁺⁺ transport in the TALH of the kidney involves the functional interplay of three claudin genes: claudin-14, -16 and -19, all of which are associated with human kidney diseases of hypercalciuria, nephrolithiasis and bone mineral loss. (Hou, J. 2012) In a previous report, the inventors demonstrated that claudin-14 inhibits claudin-16 permeability and integrates into the claudin-16/-19 channel complex in vitro. (Gong, Y., et al., 2012)

The inventors now present in vivo evidence that overexpression of claudin-14 in the TALH of the kidney generates a renal phenotype characteristic with hypomagnesemia and hypercalciuria, an exact phenocopy of claudin-16 KO. (Hou, J. et al., 2007) Our data have demonstrated that paracellular Ca⁺⁺ reabsorption in the TALH can be directly regulated by CaSR during hypercalcemia. (Desfleurs, E., et al., 1998: Motoyama, H. I., et al., 2002) Through physical interactions, claudin-14 inhibits the paracellular channel made of claudin-16 and -19. Also, tight junction proteins were previously considered to be static and structural molecules. Claudin-14 is the first TJ molecule the expression level of which can be regulated within hours in response to physiological changes. The changes in its mRNA levels can be captured as early as 1 hrs following NPS2143 or cinacalcet treatment, while the changes in protein levels slightly lagged behind and became apparent by 2 hrs.

Evidence supports that claudins are regulatory molecules with particularly fast protein turnover rate of less than 60 min in the colon (Buzza, M. S., et al., 2010) and the blood vessel. (Mandel, I., et al., 2012) Additional mechanisms such as phosphorylation, palmitoylation and trafficking may also exist for claudin-14, -16 and -19 that allow explaining more transient changes (within minutes) in paracellular Ca⁺⁺ transport during some in vitro recordings in perfised TALH tubules. (Loupy, A., et al., 2012; Mandel, I., et al., 2012)

CaSR is a G protein-coupled receptor (GPCR) that can be activated by an extracellular ion—Ca⁺⁺. Its function has been demonstrated to play a role in many physiological processes—including sperm generation, embryonic development, Ca⁺⁺ metabolism, neuronal excitability etc.; underlying various diseases, such as hyper/hypoparathyroidism, kidney stone, osteoporosis, Alzheimer's disease, epilepsy etc. (Riccardi, D., et al., 2012) The classic CaSR signaling pathway involves its binding to the G proteins—G_{q/11}, Gᵢ and G_{12/13} that in turn stimulate the phospholipase C (PLC), producing diacylglycerol (DAG) and inositol 1,4,5-trisphosphate (IP₃) and increasing intracellular Ca⁺⁺ levels (Caᵢ⁺⁺). (Riccardi, D., et al., 2010) The calcineurin-NFAT pathway, a canonical intracellular Ca⁺⁺ signaling mechanism, responds to changes in Caᵢ⁺⁺ by turning on or off a variety of genes in different tissues. (Crabtree, G. R., et al., 2002) In the TALH of the kidney, there have been reports showing a generic increase in calcineurin activity upon CaSR activation that played a role in the production of tumor necrosis factor (TNF) and prostaglandins. (Abdullah, H. I., et al., 2008; Herbert, S. C., et al., 2007)

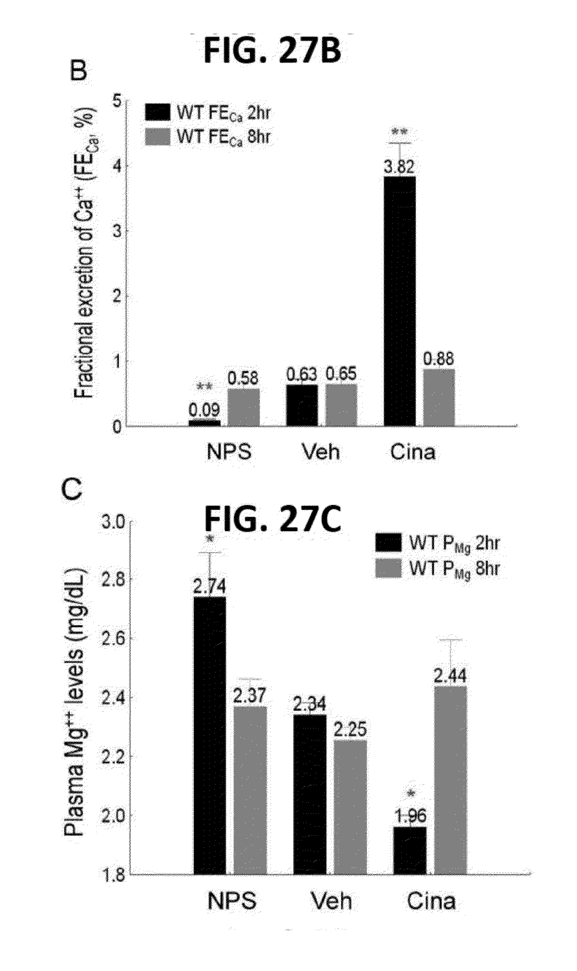

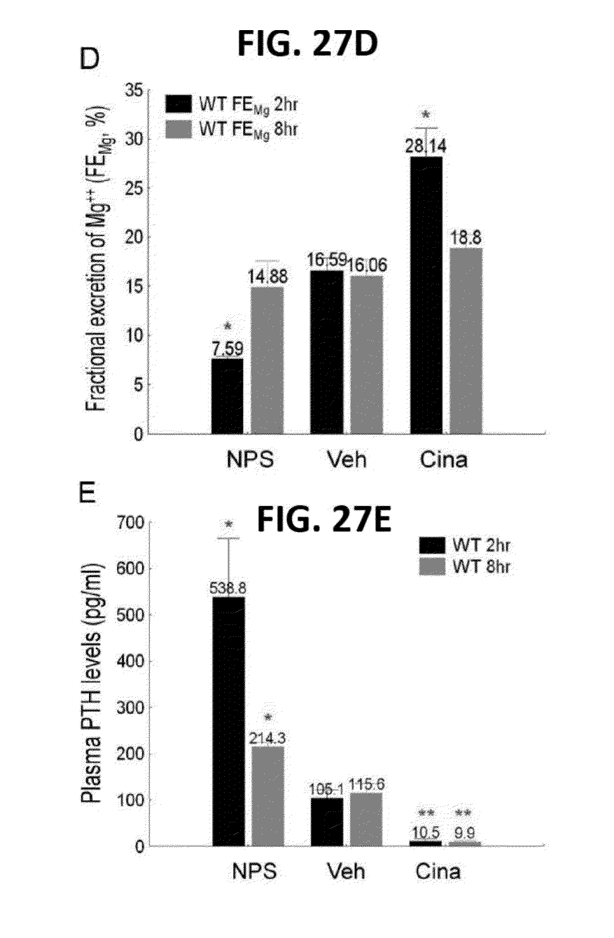

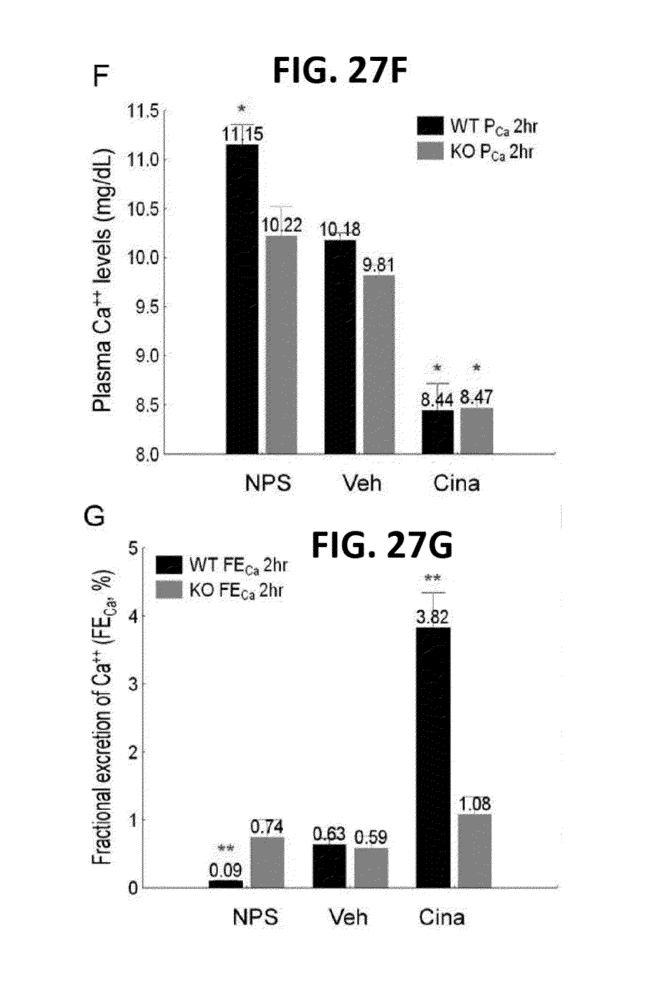

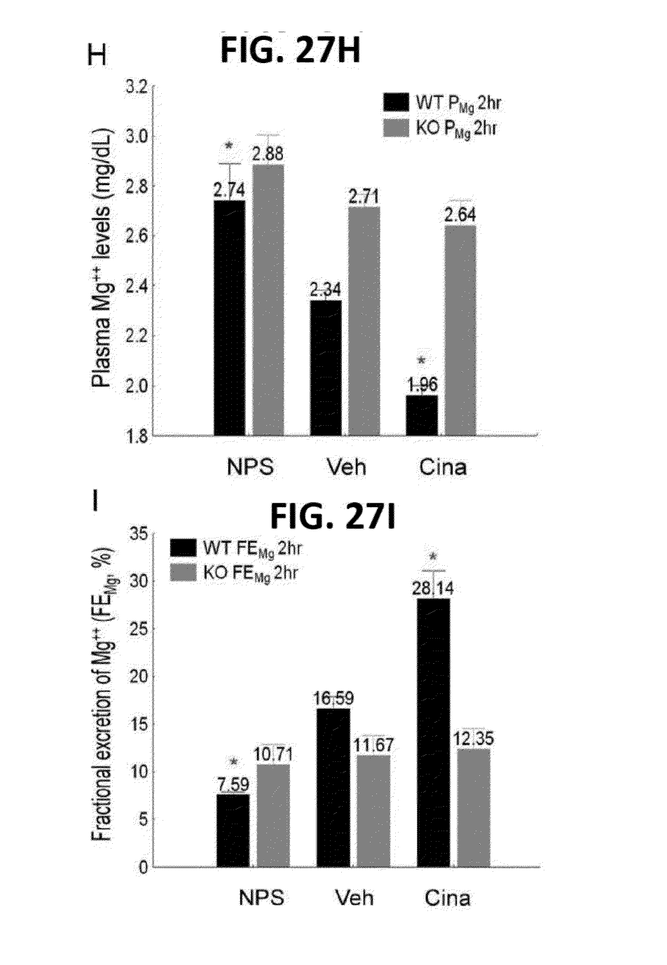

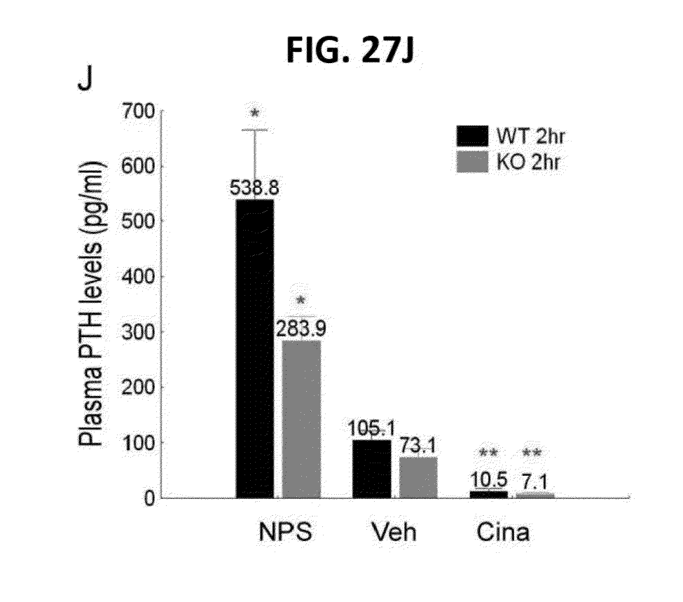

The inventors discovered a transcriptional program in the TALH of the kidney that comprises NFATc1-microRNA and plays a functional role through the regulation of claudin-14. Our data explained how calcineurin inhibitor—cyclosporine abolished the NPS2143 effect both on the levels of claudin-14 gene regulation and calciuretic response. Consistent with NPS2143 eliciting profound decreases in claudin-14 gene expression, NFATc1 binding to miR-9-1 and miR-374 promoters was concomitantly increased, inducing local histone H3 acetylation and therefore stimulating microRNA transcription. Because claudin-14 transcripts were already present intracellularly, alteration of microRNA transcription allowed rapid regulation of the transcript stability and the translational efficiency of its target gene—claudin-14, ensuring timely functional regulation for the signaling pathway. The cinacalcet effect was attenuated but not abolished by cyclosporine, nor was there a significant change in NFATc1 binding to microRNA promoter or histone acetylation. It is not known what underlies the binary signal of CaSR regulation. Notably, the resetting mechanism for CaSR signaling appears to be quite different between the kidney and the parathyroid glands. The renal CaSR signaling was reset within 8 hrs, leading to rapid recovery in the levels of claudin-14 gene expression and calciuretic response, by which time the calcialytic/calcimimetic effects on serum PTH levels persist (FIG. 27E) even though the half-life of PTH itself is within minutes. The resetting in the kidney could be due to many mechanisms such as regulation at the level of receptor or through pharmacokinetic regulation of calcialytic/calcimimetic metabolism. The maximal serum concentration of cinacalcet after oral administration is believed to be achieved in approximately 1.5-3 hrs, (Nemeth, E. F., et al., 2004) which coincides with the maximal expression level of claudin-14. The half-life of absorbed cinacalcet is 30-40 hrs in serum, which however far exceeds the time needed to reset the kidney. NPS2143 is expected to have similar pharmacokinetic profiles. Resetting at the level of receptor can involve regulation of its gene expression, protein trafficking and endocytosis, (Grant, M. P., et al., 2011) or interaction with adaptor proteins such as filamin A (Huang, C., et al., 2007) and AP2S1. (Nesbit, M. A., et al., 2013)

The renal role of CaSR in calcium metabolism has been difficult to delineate owing to its effects on PTH secretion. A constitutive KO of CaSR in the mouse has failed to provide further insight, largely owing to early lethality and its association with hyperparathyroidism. (Ho, C., et al., 1995) Kos, C. H., et al., 2003 and Tu, Q., et al., 2003 have bred CaSR^{-/-} mice with mice lacking the parathyroid hormone (PT^{-/-}). These mice demonstrated that suppression of PTH is not required for robust defense against various hypercalcemic challenges. (Kanthanr L., et al., 2009) Instead, the kidney plays a primary role in this regard. CaSR⁺PTH⁻ and CaSR⁺PTH⁺ mice showed similar increases in urinary Ca excretion levels when fed with high Ca⁺⁺ diet, while CaSR⁻PTH⁻ mice were unable to excrete excess Ca⁺⁺ in face of hypercalcemia. (Kantham, L., et al., 2009) With a kidney specific CaSR KO mouse model, authors have elegantly shown that the KO mice excrete less Ca⁺⁺ in face of hypercalcemia even though their PTH secretion was intact (Toka, H. R., et al., 2012). Loupy, A., et al., have studied the renal role of CaSR in cases of hypercalcemia induced by the calcilytic compound—NPS2143. (Loupy, A., et al., 2012) In TPTX rats supplemented with a constant PTH level, NPS2143 administration elicited a decrease in urinary Ca⁺⁺ excretion without affecting net Ca⁺⁺ release from the bone or intestinal Ca⁺⁺ absorption. These earlier efforts fell short of providing a molecular mechanism underlying the renal function of CaSR.

The inventors discovered a molecule, claudin-14, that fulfills the transport role for Ca⁺⁺ in the kidney. The mRNA, protein and TJ localization levels of claudin-14 in the kidney can all be rapidly regulated within hours by systemic administration of calcimimetic and calcilytic compounds. Without being limited by theory, these findings fulfill a signaling role for CaSR. Aided by KO strategy and time-controlled renal clearance measurements, the inventors demonstrated the functional role of claudin-14 in CaSR induced calciuretic and magnesiuretic responses. Because the experiments were carried out in intact animals, PTH levels were simultaneously monitored and found similar in WT and claudin-14 KO following calcimimetic and calcilytic administration, arguing against the role of PTH in CaSR mediated renal function. Nevertheless, PTH appeared to set a “floor” level defending against hypocalcemia, mindful that KO of claudin-14 could not correct the hypocalcemia induced by cinacalcet. This phenomenon has also been observed by Kantham et al. showing that the presence of CaSR in the setting of PTH KO could not rescue the CaSR/PTH double KO from hypocalcemia. (Kantham, L., et al., 2009)

### Methods

The methods and compositions described herein utilize laboratory techniques well known to skilled artisans, and can be found in laboratory manuals such as Sambrook. J., et al., Molecular Cloning: A Laboratory Manual, 3rd et al., Cold Spring Harbor Laboratory Press, Cold Spring Harbor, N.Y., 2001; Spector, D. L. et al., Cells: A Laboratory Manual, Cold Spring Harbor Laboratory Press, Cold Spring Harbor, N.Y., 1998: Nagy, A., Manipulating the Mouse Embryo: A Laboratory Manual (Third Edition), Cold Spring Harbor, N.Y., 2003 and Harlow, E., Using Antibodies: A Laboratory Manual, Cold Spring Harbor Laboratory Press, Cold Spring Harbor, N.Y., 1999. Methods of administration of pharmaceuticals and dosage regimes, can be determined according to standard principles of pharmacology well known skilled artisans, using methods provided by standard reference texts such as Remington: the Science and Practice of Pharmacy (Alfonso R. Gennaro ed. 19th ed. 1995); Hardman, J. G., et al., Goodman & Gilman's The Pharmacological Basis of Therapeutics, Ninth Edition, McGraw-Hill, 1996; and Rowe, R. C., et al., Handbook of Pharmaceutical Excipients, Fourth Edition, Pharmaceutical Press, 2003. As used in the present description and the appended claims, the singular forms “a”, “an” and “the” are intended to include the plural forms as well, unless the context indicates otherwise.

The present teachings include descriptions provided in the examples that are not intended to limit the scope of any aspect or claim. Unless specifically presented in the past tense, an example can be a prophetic or an actual example. The following non-limiting examples are provided to further illustrate the present teachings. Those of skill in the art, in light of the present disclosure, will appreciate that many changes can be made in the specific embodiments that are disclosed and still obtain a like or similar result without departing from the spirit and scope of the present teachings.

### The Following Methods are Applicable to Examples 1-16

### Reagents, Antibodies, Cell Lines and Animals

Reagents, kits, and antibodies are listed in Table 1. The following antibodies were used in this study rabbit polyclonal anti-Tamm-Horsfall protein (THP) (Biomedical Technologies), rabbit polyclonal anti-Thiazide-sensitive NaCl cotransporter (NCC) (Chemicon); rabbit polyclonal anti-CLDN1 (Zymed Laboratories); rabbit polyclonal anti-CLDN14; rabbit polyclonal anti-CLDN16 (against SYSAPRTETAKMYAVDTRV) (SEQ ID NO: 5); rabbit polyclonal anti-CLDN19 (against NSIPQPYRSGPSTAAREYV) (SEQ ID NO: 6); goat polyclonal anti-aquaporin-2 (AQP2) (Santa Cruz Biotech): and mouse monoclonal anti-occludin antibodies (Zymed Laboratories). Mouse MKTAL cells and human HEK293 cells were cultured in DMEM (Invitrogen) supplemented with 10% FBS, penicillin/streptomycin, and 1 mM sodium pyruvate. WT C57BL6 mice were from Charles River Laboratory. The CLDN14^{+/lacZ} reporter/knockout mice were back-crossed to C57BL/6 background for 7 generations.

TABLE 1
Reagents and Experimental Materials

| | Name | Supplier | Cat No |
|---|---|---|---|
| Chemicals | TRIzol ® Reagent | Invitrogen | 15596-018 |
| | (2-Hydroxypropyl)-β-cyclodextrin | Sigma | 332607 |
| | Advantage ® RT-for-PCR Kit | Clontech | 639506 |
| | iQ ™ SYBR ® Green Supermix | Bio Rad | 170-8882 |
| | Cinacalcet HCL | Amegen | |
| | NPS2143 hydrochloride | Sigma | SML0362-25MG |
| | Cremophor EL | Sigma | C5135-500G |
| | FK-506 monohydrate | Sigma | F4679-5MG |
| | Sandimmune Injection (cyclosporine | Novartis | |
| | injection) | | |
| | Inulin-FITC | Sigma | F3272 |
| | Lipofectamine ® LTX & Plus Reagent | Invitrogen | 15338-100 |
| | Lipofectamine ® 2000 Transfection | Invitrogen | 11668019 |
| | Reagent | | |
| | Silencer ® Select Negative Control No. 1 | Invitrogen | 4390843 |
| | siRNA | | |
| | NFAT Silencer Selected siRNA | Invitrogen | 4390771 |
| | Collagenase | Worthington | LS004196 |
| | MAGnify ™ Chromatin | Invitrogen | 49-2024 |
| | Immunoprecipitation System | | |
| | β-Gal Assay Kit | Invitrogen | K1455-01 |
| | Dual-Glo Luciferase Assay System | Promega | E2940 |
| | DC Protein Assay Kit II | Bio Rad | 500-0112 |
| | Precision Plus Protein ™ WesternC ™ Pack | Bio Rad | 161-0385 |
| | MOUSE PTH 1-84 ELISA KIT | IMMUTOPICS | 60-2305 |
| | QuikChange Lightning Multi Site-Directed | Agilent | 210518 |
| | Mutagenesis | Technologies | |
| | Kit | | |
| Tissue | Tet System Approved FBS | Clontech | 631106 |
| Culture | FBS Advantage | Atlanta Bio | S11050 |
| | Penicillin-Streptomycin | Invitrogen | 15140-122 |
| | Trypsin EDTA | Cellgro | 25-053-Cl |
| | Dulbecco's Modification of Eagle's | Cellgro | 10-013-CV |
| | Medium (DMEM) | | |
| Antibody | Anti-acetyl-Histone H3 Antibody | Millipore | 06-599 |
| | Anti-NF-ATc1 Antibody | BD Bioscience | 556602 |
| | Anti-cldn14 | Lab Generated | |
| | anti-Tamm-Horsfall Glycoprotein | Biomedical | BT-590 |
| | | Technologies | |

### Pharmacological and Dietary Manipulation in Experimental Animals

NPS2143 and cinacalcet (Table 1) were dissolved in vehicle—20% (w/v) (2-Hydroxypropyl)-β-cyclodextrin solution and fed to mice with gavage syringe. Cyclosporin-A (Sandimmune; Table 1) was diluted in 0.9% saline and I.P. injected to animals. Control animals received vehicle injection (13% wiv Cremophor EL and 32.9% ethanol). For dietary Ca²⁺ manipulation, animals were fed with the following diets for six consecutive days: basal diet: 0.61% Ca²⁺ (TestDiet #5755); low Ca²⁺ diet: 0 Ca⁺⁺ (TestDiet #5855); high Ca²⁺ diet: 5% Ca²⁺ (TestDiet #5AVB). All animals had free access to water and were housed under a 12 hr light cycle. Blood samples were taken by cardiac puncture rapidly after initiation of terminal anesthesia and centrifuged at 4° C. for 10 min. Kidney were dissected out and immediately frozen at −80° C.

### Surgical Protocols and Renal Clearance

The method for performing renal clearance measurements in the mouse has been described by Hou et al., (2007; 2009). Mice were anesthetized by i.p. injection of Inactin (Sigma; 100 mg/kg). The jugular vein was catheterized for i.v. infusion of 0.9% saline at 2 μL/min, with 1% FITC-inulin included in the infusate. After an equilibration period of 60 min, renal clearance measurements were carried out for a 60 min period. Urine was collected under mineral oil, and 30 μL blood sample was taken at hourly intervals. Urine and plasma Ca²⁺ and Mg²⁺ concentrations were measured by atomic flame absorption spectrophotometer (PerkinElmer). Urine and plasma FITC-inulin levels were measured in 100 mM HEPES buffer (pH7.0) with fluorescence spectrophotometer (BioTek). The fractional excretion of electrolytes was calculated using the following equation FEᵢₒₙ=V×Uᵢₒₙ/(GFR×Pᵢₒₙ), where GFR was calculated according to the clearance rate of FITC-inulin (GFR=V×Uᵢₙᵤₗᵢₙ/Pᵢₙᵤₗᵢₙ).

### Animal Metabolic Studies

Age (9-10 weeks old) and sex (male) matched wild-type and CLDN14 KO mice were housed individually in metabolic cages (Harvard Apparatus) with free access to water and food for 24 hr. Urine was collected under mineral oil. The plasma and urine Ca⁺⁺ and Mg²⁺ levels were determined with atomic flame absorption spectrophotometer (PerkinElmer). The plasma (P) and urine (U) electrolyte levels were determined with the Roche Cobas clinical analyzer (Roche Diagnostics). Creatinine levels were measured with an enzymatic method independent of plasma chromogens (Himnerkus et al., 2008). The fractional excretion of electrolytes was calculated using the following equation: FEᵢₒₙ=V×Uᵢₒₙ/(GFR×Pᵢₒₙ), where GFR was calculated according to the clearance rate of creatinine (GFR=V×U_{creatinine}/P_{creatinine}).

### Generation of Transgenic Mice

The coding sequence of mouse claudin-14 gene was cloned into a bicistronic pIRES-GFP vector (Clontech). A 3.7 kb mouse Tamm-Horsfall protein (THP) promoter (kindly provided by Dr Donald Kohan: Stricklett et al., 2003) was cloned into the pIRES-GFP vector to replace the CMV promoter. Inclusion of GFP allowed rapid screening of transgene expression in the kidney. To generate claudin-14 overexpression transgenic (TG) mice, female donor mice (C57BL/6×CBA hybrid strain) were superovulated with a combination of pregnant mare serum (5 units) and human CG (5 units). The transgenic vector was injected into the pronucleus of single-cell mouse embryos and allowed to develop to two-cell embryo stage. Injected embryos were implanted into pseudopregnant females and carried to term. The transgenic founder mice were crossed to WT C57BL/6 mice, and F1 progeny were analyzed. Littermate WT mice were used as controls. Out of 41 transgenic founders, 9 had germ-line transmission of transgene; 4 had detectable transgene expression in the kidney.

### Establishing Primary Cultures of Mouse Thick Ascending Limb Cells

An immunomagnetic separation method was used to isolate the TAL cells from the mouse kidney (Hou et al., 2009). Antibodies against the TAL cell specific surface antigen, Tamm-Horsfall protein (THP), were coated onto the paramagnetic polystyrene beads (Dynabeads M-280; Dynal), allowing immunoprecipitation of the TAL cells from collagenase digested mouse kidneys. The isolated cells were plated in DMEM medium supplemented with 10% FBS, penicillin/streptomycin, and 1 mM sodium pyruvate for 16 hours, followed by immediate transfection with NFAT or antagomirs.

### Preparation of Low Ca²⁺ Cell Culture Medium

Earle's Balanced Salt Solution (EBSS; Invitrogen) containing (117.24 mM NaCl, 5.33 mM KCl, 26.19 mM NaHCO₃, 1.01 mM NaH₂PO₄, 0.70 mM MgCl₂ and 5.56 mM glucose) was supplemented with the MEM Amino Acids solution, the MEM Non-Essential Amino Acids solution, the MEM Vitamin solution, L-Glutamine, 1 mM Sodium Pyruvate, penicillin/streptomycin and 1% FBS. For determining Ca²⁺ effects on gene expression, MKTAL cells were cultured in EBSS supplemented with the indicated concentrations of Ca²⁺ for 16 hrs. For examining PTH effects, MKTAL cells were cultured in EBSS supplemented with the indicated concentrations of human recombinant PTH (1-84; Sigma) for 16 hrs.

Bioinformatical Analyses of mRNA and miRNA

Transcripts of CLDN14 gene are alternatively spliced, generating 5 mRNA variants in human: 1—NM_{—}144492, 2—NM_{—}012130, 3—NM_{—}001146077, 4—NM_{—}001146078 and 5—NM_{—}001146079; 3 variants in mouse: 1—NM_{—}019500, 2—NM_{—}001165925 and 3—NM_{—}001165926; and 1 variant in rat: NM_{—}001013429. For predictions of miRNAs targeting CLDN14:3′-UTR (mouse: 171 bp [AF314089](SEQ ID NO: 1); human: 183 bp [AF314090](SEQ ID NO: 2) based on sequence complementarities and cross-species conservation, four algorithms were used: TargetScan, miRanda. Diana microT and MirTarget2.

Mouse Claudin-14 [AF314089]
(SEQ ID NO: 1)
GTTCCTTCCCCGGGCTTCTGCCAGGGATGCTGGGCCCCAAAGGACCAATG
ATGGATGTGGGAAGGATGCAGAGAGCAAGCCCGGAACACAGGGAAGGAGG
TGCTCTTCAAAGCAAAGACTTCTAAAAAGTGCTGGTTTTTTATTTATTAT
ATGTATTTATGCGGGTGGCTT
Human Claudin-14 [AF314090]
(SEQ ID NO: 2)
AGCACAAAGTTTACTTCTGGGCAATTTTTGTATCCAAGGAAATAATGTGA
ATGCGAGGAAATGTCTTTAGAGCACAGGGACAGAGGGGGAAATAAGAGGA
GGAGAAAGCTCTCTATACCAAAGACTGAAAAAAAAAATCCTGTCTGTTTT
TGTATTTATTATATATATTTATGTGGGTGATTT

MicroRNA and mRNA Quantification

Total RNA including microRNA was extracted using Trizol (Invitrogen). Reverse transcription was performed on 1 μg of total RNA using miRNA specific RT primer and TaqMan miRNA reverse transcription kit (Invitrogen). Real-time PCR amplification was performed on reverse transcribed miRNA using TaqMan Universal PCR Master Mix, No AmpErase UNG (Invitrogen) and Eppendorf Realplex 2S real-time PCR system. Results were expressed as 2^{-ΔCt} values with ΔCT=Ct_{mRNA}−Ct_{U6snRNA}. Cellular mRNA was reverse transcribed using the Superscript-III kit (Invitrogen), followed by real-time PCR amplification using SYBR Green PCR Master Mix (Bio-Rad) with the primer pairs: CLDN14, ACCCTGCTCTGCTTATCC (SEQ ID NO: 7) and GCACGGTTGTCCTGTAG (SEQ ID NO: 8); CLDN16. CAAACGCTTTTIGATGGGATTC (SEQ ID NO: 9) and TTTGTGGGTCATCAGGTAGG (SEQ ID NO: 10); β-actin, CGTTGACATCCGTAAAGAC (SEQ ID NO: 11) and TGGAAGGTGGACAGTGAG (SEQ ID NO: 12). Results were expressed as 2^{-ΔCt} values with ΔCT=Ct_{CLDN}−Ct_{β-actin}.

### Real-Time PCR Quantification

Total RNA including microRNA was extracted using Trizol (Invitrogen). Cellular mRNA was reverse transcribed using the Superscript-III kit (Invitrogen), followed by real-time PCR amplification using SYBR Green PCR Master Mix (Bio-Rad) and Eppendorf Realplex2S system. The design of claudin-14 primers was described above. The design of pri-miRNA primers was according to Ma et al., (2010). The PCR primers are listed in Table 2. Results were expressed as 2^{-ΔCt} values with ΔCT=Ct_{gene}−Ct_{β-actin}.

TABLE 2
PCR Primers

| Name. | Sequence |
|---|---|
| β-actin | CGTTGACATCCGTAAAGAC |
| | (SEQ ID NO: 11) |
| | TGGAAGGTGGACAGTGAG |
| | (SEQ ID NO: 12) |
| claudin-14 orf | CACACCCGCCAAGACCACCT |
| | (SEQ ID NO: 13) |
| | AGGTACAGGGCCTGGCCGAT |
| | (SEQ ID NO: 14) |
| claudin-14 utr | GAAGTTTGAGATTGGCCAGGC |
| | (SEQ ID NO: 15) |
| | GTGCGGTGTTTGCAGTGGTC |
| | (SEQ ID NO: 16) |
| pri-miR-9-1 | GGAGGAGGGAGTGGGAAATG |
| | (SEQ ID NO: 17) |
| | ACCTCACGCACTATCACGAC |
| | (SEQ ID NO: 18) |
| pri-miR-9-2 | CAGCTTGCTGCACCTTAGTC |
| | (SEQ ID NO: 19) |
| | TGTGCGGCTAGAACATCCAA |
| | (SEQ ID NO: 20) |
| pri-miR-9-3 | GGGGAACTGGGGATGGAGAA |
| | (SEQ ID NO: 21) |
| | CAGAGGGGACCGACAGACA |
| | (SEQ ID NO: 22) |
| pri-miR-374 | ACTCGATGATTCTTCCATTCATGC |
| | (SEQ ID NO: 23) |
| | CGGCTTCAGGTCAACTGTGA |
| | (SEQ ID NO: 24) |
| lacZ primer | ATGGGTAACAGTCTTGGCGG |
| | (SEQ ID NO: 25) |
| | GGCGTATCGCCAAAATCACC |
| | (SEQ ID NO: 26) |
| ChIP miR-374 | GTTTTTGGAATAGCTGACAATG |
| | SEQ ID NO: 27) |
| | CTGTTCAGGAACTGGCTTTG |
| | (SEQ ID NO: 28) |
| ChIP miR-9-1 | CCTGGIGCTGAGATTACAGA |
| | (SEQ ID NO. 29) |
| | CTGCCACGTCTATAATCCAT |
| | (SEQ ID NO: 30) |
| NFATc1 | GTGTTCCCAGCGGTCAGCCC |
| | (SEQ ID NO: 31) |
| | GTGGAGCTTCGGGCTTGCGT |
| | (SEQ ID NO: 32) |
| NFATc2 | AGGACGACAGCATCCCCGCT |
| | (SEQ ID NO: 33) |
| | ACGGCTGCCCTCCGTCTCAT |
| | (SEQ ID NO: 34) |
| NFATc3 | GCAGCTTCCCCAGCAGCCTC |
| | SEQ ID NO: 35) |
| | CCGGACCCGGGAGAGGAGTG |
| | SEQ ID NO: 36) |
| NFATc4 | GCAGGCAGCATCAGTGCCCA |
| | (SEQ ID NO: 37) |
| | TCCTGCTCCGAAGCCCCCTC |
| | (SEQ ID NO: 38) |

### Manipulation of NFAT Gene Expression

Pre-validated Silencer Select siRNAs were designed and synthesized by Ambion. A pool of 3 target-specific 21 nt siRNA duplexes was designed against the coding region of mouse NFATc 1-c4 genes. A scrambled siRNA duplex was used as negative control. The NFATc1nuc expression vector (in pcDNA3.1 backbone) was generated by the Crabtree lab and obtained through Dr. Feng Chen. Transfection of siRNAs or NFATc1nuc was carried out with Lipofectamine LTX & Plus Reagent for primary cultures.

### Luciferase Reporter Assay

The pMir-Reporter construct was generated by inserting the mouse CLDN14:3′-UTR sequence (AF314089: 171 bp from the stop codon to the first polyA site) (SEQ ID NO: 1) or human CLDN14:3′-UTR sequence (AF314090: 183 bp from the stop codon to the first polyA site) (SEQ ID NO: 2) into the pMIR-REPORT-Luciferase vector (Clontech) downstream of the luciferase gene using Spe1 and Sac1. Deletions of miR-9 (CCAAAG) and miR-374 (AUUAUA) binding sites in human CLDN14:3′-UTR were generated using site-directed nmtagenesis (Stratagene). The pMir-Reporter (500 ng), the pGL4.74 Renilla luciferase control vector (500 ng; Promega) and 30 pmol of either scrambled miRNA, miR-9 or miR-374 precursor (Invitrogen) were co-transfected to HEK293 cells in 12-well culture dishes using Lipofectamine-2000 (Invitrogen). The miR-9-1 and miR-374 gene promoters were cloned into pGL4.10 luciferase reporter (Promega) with Sfi1 sites. Deletion of the NFAT binding sites (AGGAAAAT) in miR-9-1 and miR-374 promoters were generated using site-directed mutagenesis (Stratagene). The pGL4.10 reporter (500 ng), the pGL4.74 Renilla luciferase control vector (500 ng; Promega) and pcDNA3.1-NFATc1nuc vector were co-transfected to HEK293 cells in 12-well culture dishes using Lipofectamine-2000 (Invitrogen). Twenty-four hours after transfection, firefly and renilla luciferase activities were measured with a chemilhunminescence reporter assay system-Dual Glo (Promega) in a GLOMAX Luminometer (Promega).

### Antagomir Assay

Antagomirs for miR-9, miR-374 and scrambled control miRNA were designed and synthesized by Exiqon using locked nucleic acids (LNA). 60 pmol of each antagomir (or 30 pmol anti-miR-9+30 pmol anti-miR-374 in synergistic assays) were transfected to MKTAL cells in 12-well culture dishes using Lipofectamine-2000 (Invitrogen). Twenty-four hours after transfection, total cellular RNAs or membrane proteins were collected.

CaSR Knockdown by siRNA

Pre-validated Silencer Select siRNAs were designed and synthesized by Ambion. A pool of 3 target-specific 21 nt siRNA duplexes were designed against the coding region of mouse CaSR gene (AF110178). A scrambled siRNA duplex was used as negative control. Twenty-four hours prior to Ca²⁺ activation, 50 pmol of either CaSR siRNA or scrambled siRNA was transfected to MKTAL cells in 12-well culture dishes with Lipofectmine-2000. To improve transfection efficiency, DMEM medium was used during transfection. At the end of transfection, cells were washed for three times in PBS and switched to the low Ca²⁺ medium (EBSS) followed by Ca²⁺ activation.

### Coimmunoprecipitation

HEK293 cells expressing CLDN14 with CLDN16 or CLDN19 were lysed in 50 mM Tris (pH 8.0) by 25-30 repeated passages through a 25-gauge needle, followed by centrifugation at 5,000 g. The membranes of lysed cells were extracted using CSK buffer (150 mM NaCl; 1% Triton X-100; 50 mM Tris, pH 8.0; and protease inhibitors). The membrane extract was precleared by incubation with protein A/G-sepharose (Sigma-Aldrich) prior to coimmunoprecipitation. The precleared membrane extract was incubated for 16 h at 4° C. with anti-CLDN14, anti-CLDN16, anti-CLDN19, anti-CLDN1 and anti-occludin antibodies. Antibody-bound material was pelleted with protein A/G-sepharose, washed 3 times with CSK buffer, and detected by immunoblotting.

### LacZ Reporter Assay in Mouse Kidney

CLDN14^{+/lacZ} mouse kidneys were fixed with 4% paraformaldehyde at 4° C. overnight, washed three times in PBS, cryoprotected for 24 h in 30% sucrose in PBS, and embedded in OCT prior to cryostat sectioning. Cryostat sections (10 μm) were stained for β-galactosidase activity using the β-Gal staining kit (Invitrogen), followed by incubation with anti-THP antibody (diluted 1:200) and FITC-labeled secondary antibody (diluted 1:200). Epifluorescence images were taken with a Nikon 80i photomicroscope equipped with a DS-Qi1Mc digital camera. All images were converted to JPEG format and arranged using Photoshop CS4 (Adobe).

### Y2H Membrane Protein Interaction Assay

The Y2H membrane protein interaction assay (MoBiTec Molecular Biotechnology) for analyzing the specific claudin interactions [mouse CLDN14 (AF314089) (SEQ ID NO: 1), human CLDN16 (AF152101) (SEQ ID NO: 3) and human CLDN19 (BC030524) (SEQ ID NO: 4) has been described by Hou, J. et al., (2008). The assay was performed by transforming the yeast strain NMY51 with 1.5 μg of bait and prey vectors. Transformed yeast cells were plated on drop-out media lacking leucine and tryptophan (SD-LW) and incubated for growth of positive transformants. Three to six independent positive transformants were then selected and resuspended in 50 ml of 0.9% NaCl buffer: 5 μl of each suspension was spotted on SD-LWHA media. Growth of colonies on the selective medium was scored as positive for interaction. To further verify the positive interactions, β-galactosidase activity was performed using a filter lift assay (MoBiTec GmbH).

Human Claudin-16 [AF152101]
(SEQ ID NO: 3)
CCCCACCCGAAACACACTCAGCCCTTGCACTGACCTGCCTTCTGATTGGA
GGCTGGTTGCTTCGGATAATGACCTCCAGGACCCCACTGTTGGTTACAGC
CTGTTTGTATTATTCTTACTGCAACTCAAGACACCTGCAGCAGGGCGTGA
GAAAAAGTAAAAGACCAGTATTTTCACATTGCCAGGTACCAGAAACACAG
AAGACTGACACCCGCCACTTAAGTGGGGCCAGGGCTGGTGTCTGCCCATG
TTGCCATCCTGATGGGCTGCTTGCCACAATGAGGGATCTTCTTCAATACA
TCGCTTGCTTCTTTGCCTTTTTCTCTGCTGGGTTTTTGATTGTGGCCACC
TGGACTGACTGTTGGATGGTGAATGCTGATGACTCTCTGGAGGTGAGCAC
AAAATGCCGAGGCCTCTGGTGGGAATGCGTCACAAATGCTTTTGATGGGA
TTCGCACCTGTGATGAGTACGATTCCATACTTGCGGAGCATCCCTTGAAG
CTGGTGGTAACTCGAGCGTTGATGATTACTGCAGATATTCTAGCTGGGTT
TGGATTTCTCACCCTGCTCCTTGGTCTTGACTGCGTGAAATTCCTCCCTG
ATGAGCCGTACATTAAAGTCCGCATCTGCTTTGTTGCTGGAGCCACGTTA
CTAATAGCAGGTACCCCAGGAATCATTGGCTCTGTGTGGTATGCTGTTGA
TGTGTATGTGGAACGTTCTACTTTGGTTTTGCACAATATATTTCTTGGTA
TCCAATATAAATTTGGTTGGTCCTGTTGGCTCGGAATGGCTGGGTCTCTG
GGTTGCTTTTTGGCTGGAGCTGTTCTCACCTGCTGCTTATATCTTTTTAA
AGATGTTGGACCTGAGAGAAACTATCCTTATTCCTTGAGGAAAGCCTATT
CAGCCGCGGGTGTTTCCATGGCCAAGTCATACTCAGCCCCTCGCACAGAG
ACGGCCAAAATGTATGCTGTAGACACAAGGGTGTAAAATGCACGTTTCAG
GGTGTGTTTGCATATGATTTAATC
Human Clandin-19 [BC030524]
(SEQ ID NO: 4)
CTGGCCATGACCAAAGCCCCTGCTGGCACCCTGGCCCAGCTCTGAGTCCT
GGGACCCTCGGTCCTCTCTCCTGGGCCATGGCCAACTCAGGCCTCCAGCT
CCTGGGCTACTTCTTGGCCCTGGGTGGCTGGGTGGGCATCATTGCTAGCA
CAGCCCTGCCACAGTGGAAGCAGTCTTCCTACGCAGGCGACGCCATCATC
ACTGCCGTGGGCCTCTATGAAGGGCTCTGGATGTCCTGCGCCTCCCAGAG
CACTGGGCAAGTGCAGTGCAAGCTCTACGACTCGCTGCTCGCCCTGGACG
GTCACATCCAATCAGCGCGGGCCCTGATGGTGGTGGCCGTGCTCCTGGGC
TTCGTGGCCATGGTCCTCAGCGTAGTTGGCATGAAGTGTACGCGGGTGGG
AGACAGCAACCCCATTGCCAAGGGCCGTGTTGCCATCGCCGGGGGAGCCC
TCTTCATCCTGGCAGGCCTCTGCACTTTGACTGCTGTCTCGTGGTATGCC
ACCCTGGTGACCCAGGAGTTCTTCAACCCAAGCACACCTGTCAATGCCAG
GTATGAATTTGGCCCAGCCCTGTTCGTGGGCTGGGCCTCAGCTGGCCTGG
CCGTGCTGGGCGGCTCCTTCCTCTGCTGCACATGCCCGGAGCCAGAGAGA
CCCAACAGCAGCCCACAGCCCTATCGGCCTGGACCCTCTGCTGCTGCCCG
AGAGTACGTCTGAGCTCTGCCTGCCCTGGCCAGCCCCCCACCCAGTGGCC
CCCTTGCCCAGCATCCAGCCAGCCTCGCAGCACCCTGGGCAGGGCCACTG
GGGCATAGGATGGGCATAGGTGCTCTGAGCAGCTTGTCCTCAACACAAGC
ACCCACCCTGCAATCTGAGACCCAGATCCTCAGAGAGACACCAGAGGCAG
GACCCAGCCCCCAGGCATACACACAGATGCAGGTCCAGGCACGGTCTTGT
CTGCACAGCCTGGTGGGCACCAGCATGCATCCCTGGAGACAGGCCCTCAG
GCACCAGCCCGGCTGTTTACTCACTGAAGAGCTGCGTGGGTGTCTGCTAC
GTGCTGGGCCCTAGAGATAGAGCAGTGGCCAAGACGTACCTTAGTACCCA
GGTCCTTGGGGTGAGCAGAAACCTTCACCCTCCCCAGTCCCATGGGCTCC
TCACAGCAACCCCACAAGGGCAGTGCCGGGATGCTGAACGTTCACACAAG
GACAGGGAGGGTCTGAGTTTAGGTCTCAGGTTCTTCCAGTGCGCCCAGGG
CTGGGGGCCACCTACACAGATGGTGAGGTCGGACCATGGCGCCCCTGCCC
CCGGGAATGGGCCCCAGGCAGGGCTGCTGTGAGGGCCAAGGTCTGGCCAC
GCTGGCCAGTACCCATGTCCGGGCCTGAATGCACAGCCCCTGCCCCCGAC
CCCACAGCTCACTCCACTAACCAGCTCTCTCTCTTTTGACTTTCAGACCA
GTTGTTAAATTGCCCGCCTCCGCCAAGGGCCCCCTGGGTGTGTAATGTCC
AGTCCCCAGCCAGGCTCTGTCCCCTGCCATACCTAGACTGTGTGTTTCAT
ATTTTTTTGGAAAGAGAAGTGAACATCCAGCCCCAAAAAAAAAAAAAAAA
AAAAAAAAAAAAAAAA

### Retroviral Expression of Claudins

The following full-length mammalian claudins were cloned into the retroviral vector pQCXIN (Clontech): mouse CLDN14 (AF314089) (SEQ ID NO: 1), human CLDN16 (AF152101) (SEQ ID NO: 3) and human CLDN19 (BC030524) (SEQ ID NO: 4). VSV-G pseudotyped retroviruses were produced in HEK293 cells and used to infect LLC-PK1 cells at a fixed titer of 1×10⁶ CFU/ml, as described previously (Hou et al., 2008). Cells co-expressing CLDN14, 16 and 19 were generated with sequential viral infections. Infected LLC-PK1 cells were seeded onto Transwell plates to allow polarization. On day 9 post polarization, cell monolayers were subjected to electrophysiological measurements and immunostained to visualize claudin localization.

### Electrophysiological Measurements

Electrophysiological recordings were performed on epithelial monolayers in an Ussing chamber (Harvard Apparatus #U9926/T) that had been modified to adapt Transwells (Hou et al., 2005; 2008). Voltage and current clamps were performed using the EC-800 epithelial amplifier (Warner Instruments) with Ag/AgCl electrodes and an Agarose bridge containing 3M KCl. The transepithelial resistance (TER) was measured under the “Resistance” mode by passing a constant bipolar current pulse (Iₒ) of 10 μA (<2 kΩ) or 1 μA (>2 kΩ) through the epithelium and recording voltage deflection (Vₒ). Ohm's law was used to calculate TER from Vₒ and Iₒ. The series resistance (Rₛ) was measured in absence of the epithelium and subtracted from TER. Dilution potentials (PD) were measured under the “Current Clamp” mode by clamping the transepithelial current to zero and recording the equilibrium voltage generated by NaCl diffusion. All experiments were conducted at 37° C. Electrical potentials obtained from blank inserts were subtracted from those obtained from inserts with epithelial monolayers. 1 mM ouabain was included in the basolateral perfusant to inhibit transcellular ion conductance. The ion permeability ratio (η) for the monolayer was calculated from the dilution potential using the Goldman-Hodgkin-Katz equation. The absolute permeabilities of Na⁺⁺ (P_{Na}) and Cl⁻ (P_{Cl}) were calculated by using the Kimizuka-Koketsu equation. The relative permeability of other cations (M⁺) to Na⁺⁺ (γ=P_{M}/P_{Na}) was then calculated from the bi-ionic potential, according to the following equation,

P_{Li}/P_{Na}=1/e^{ψ}.

P_{Ca}/P_{Na}=(1+e^{ψ})/(2e^{2ψ}),

where ψ=eΨ/kT (Ψ is the bionic potential).

### Chromatin Immunoprecipitation (ChIP)

Freshly isolated TALH cells or primary TALH cell cultures were cross-linked in 1% paraformaldehyde (pH7.0) for 10 min at room temperature. The cross-linked cells were lysed following to instruction of MAGnify ChIP system (Invitrogen). Chromatin was sonicated into 500 bp fragments with Bioruptor UCD-200 (High power, 15 cycles of 30 seconds ON, 30 seconds OFF; Diagenode). Sheared chromatin was precipitated with antibody-bound Dynabeads (Invitrogen). Following reverse crosslink and DNA extraction, antibody-bound DNA was eluted and analyzed with real-time PCR. The primers for ChIP analyses were listed in Table 2. Two criteria for normalization were applied: fold enrichment over anti-IgG background signal and input control over chromatin input signal.

### Immunolabeling and Fluorescence Microscopy

For viewing claudin localization in the kidney, fresh cryostat sections (10 μm) were fixed with cold methanol at −20° C., followed by blocking with PBS containing 10% FBS, incubation with primary antibody (1:300) and FITC-labeled secondary antibody (1:200). After washing with PBS, slides were mounted with Mowiol (Calbiochem). Epifluorescence images were taken with a Nikon 80i photomicroscope equipped with a DS-QilMc digital camera. All images were converted to TIFF format and arranged using Photoshop CS4 (Adobe).

### Statistical Analyses

The significance of differences between groups was tested by ANOVA (Statistica 6.0; Statsoft). When the all-effect F value was significant (p<0.05), post hoc analysis of differences between individual groups was made with the Newman-Keuls test. Values were expressed as mean±SEM, unless otherwise stated.

### EXAMPLES 1-16

### Example 1

This example illustrates renal localization of CLDN14 promoter, mRNA and protein.

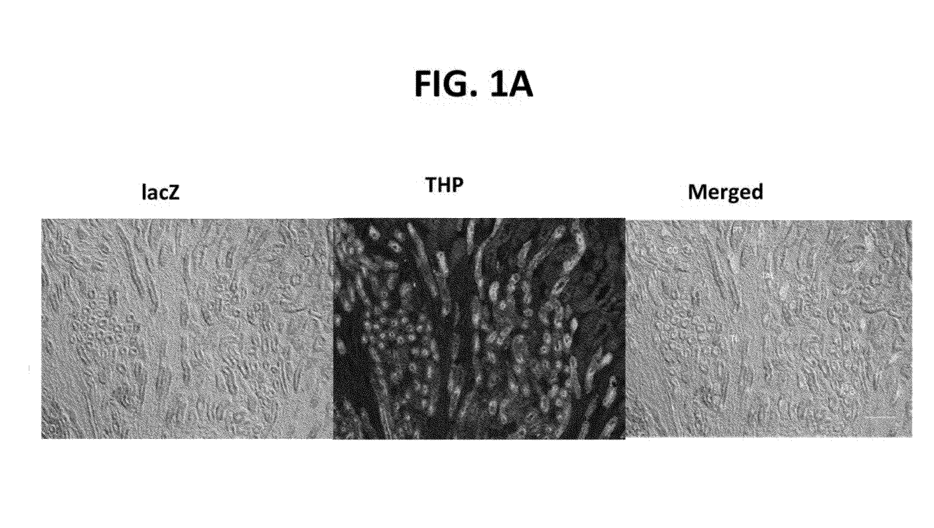

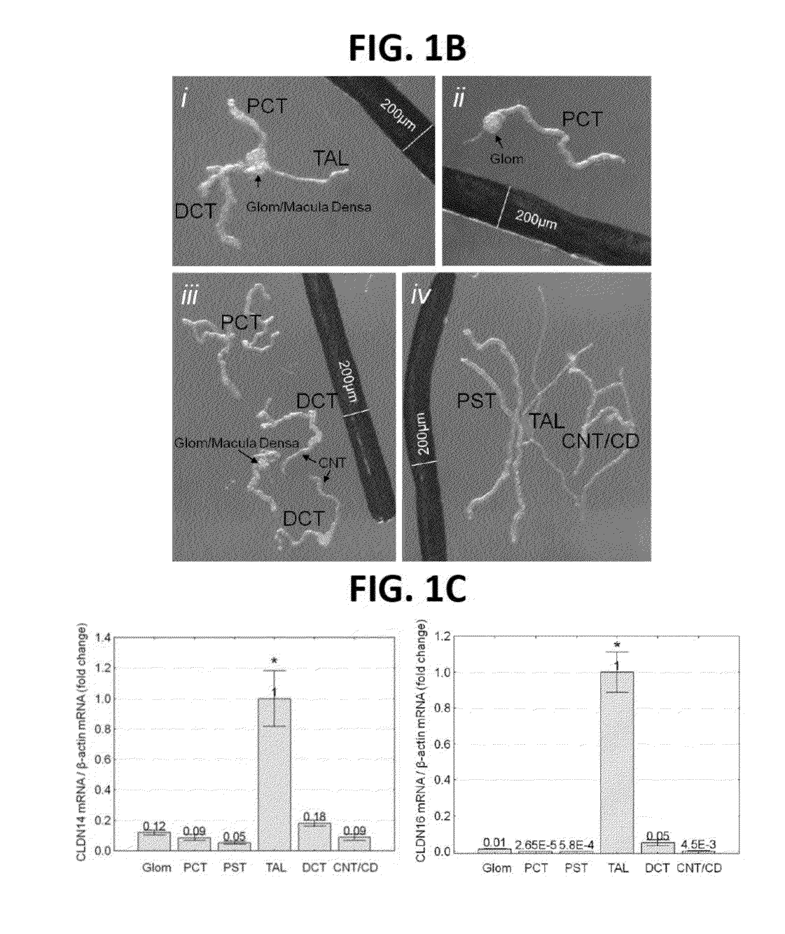

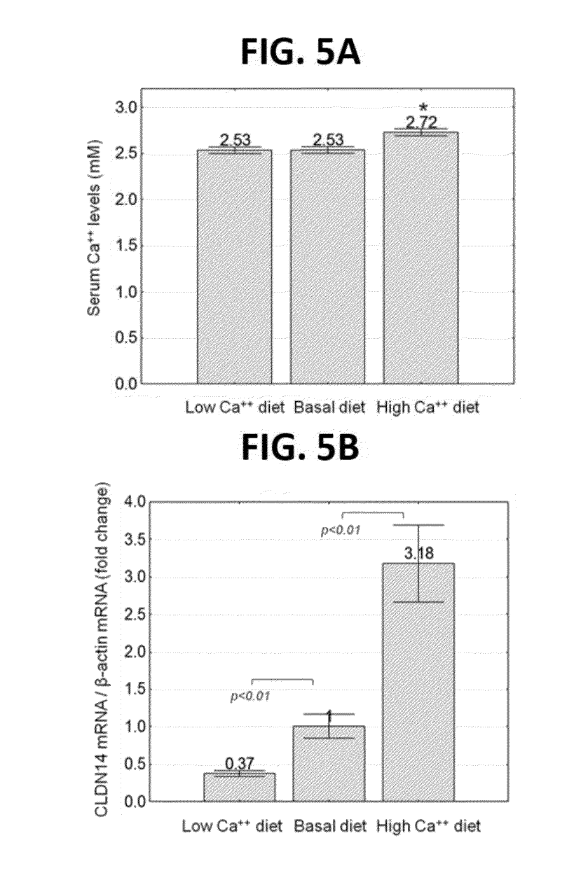

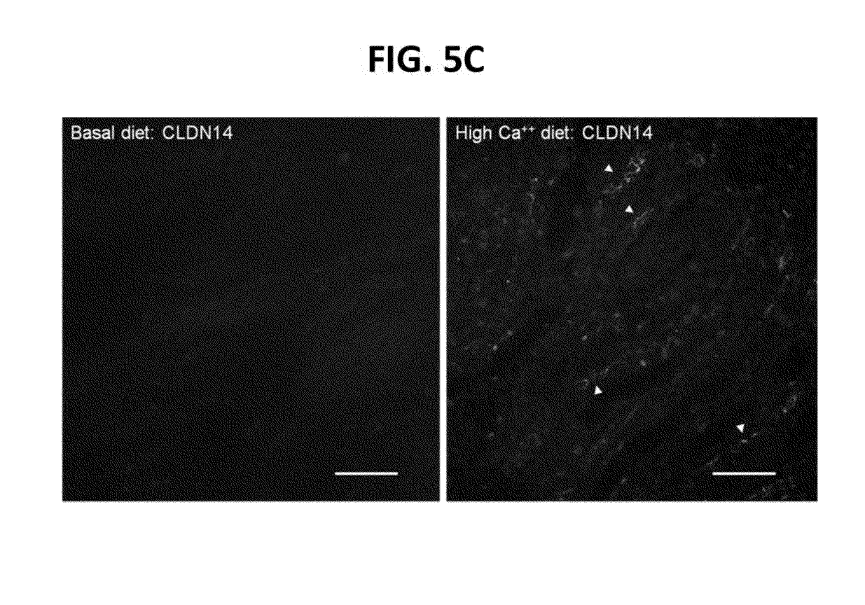

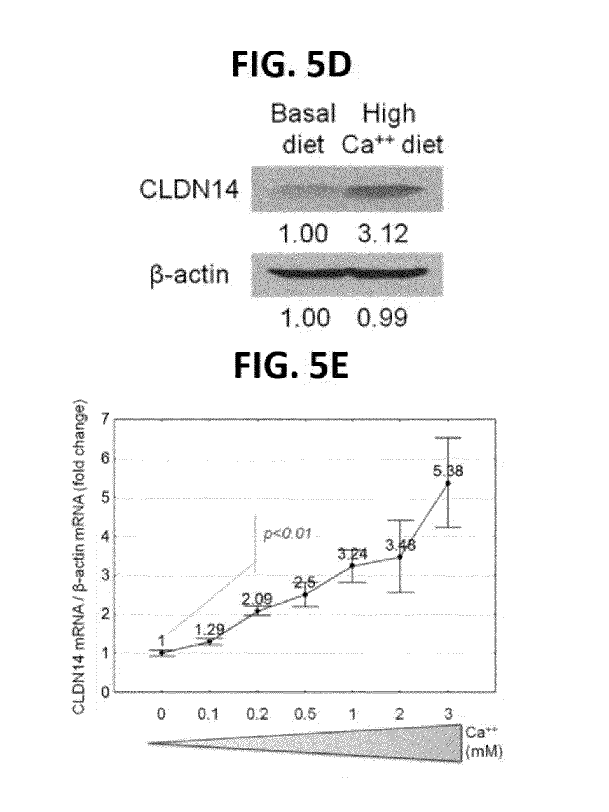

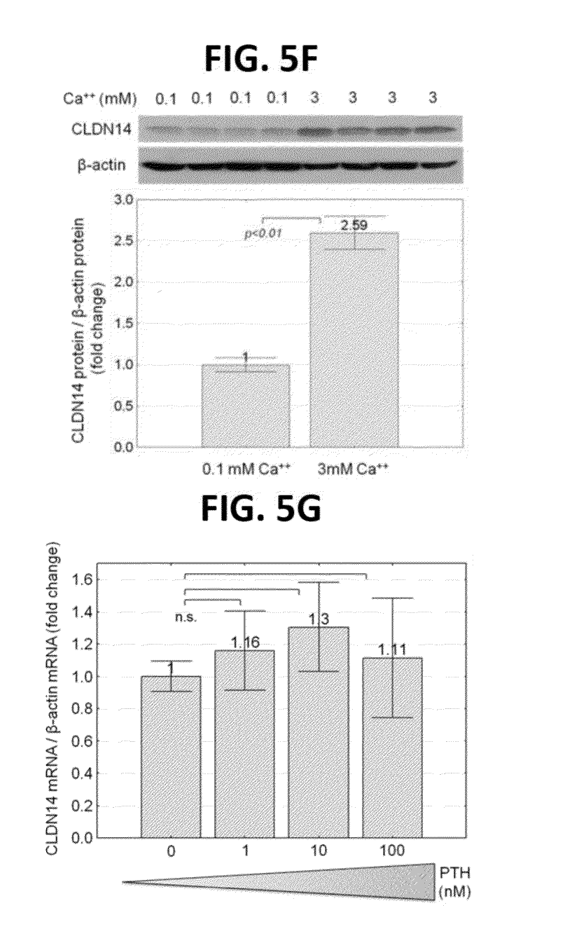

To demonstrate CLDN14 gene expression in the kidney, regardless of regulatory mechanisms that may affect its mRNA or protein level (vide infra), the inventors determined CLDN14 promoter activities in the mouse kidney. The CLDN14 promoter activity was assessed in vivo with a CLDN14-lacZ reporter mouse line (Ben-Yosef et al., 2003) in which the lacZ reporter gene replaced the CLDN14 gene under control of the endogenous CLDN14 promoter. Through colocalization analyses, the inventors found that in CLDN14+/lacZ mouse kidneys, the β-galactosidase activity was detected in tubules that co-expressed the Tamm-Horsfall protein (THP: a TAL marker) (FIG. 1A), but not in glomerulus (FIG. 5I) or in tubules that were labeled with the Lotus tetragonolobus lectin (LTL: a proximal convoluted/straight tubule [PCT/PST] marker) or that co-expressed the thiazide-sensitive Na+/Cl− cotransporter (NCC: a distal convoluted tubule [DCT] marker) or aquoporin-2 (AQP2: a connecting tubule/collecting duct [CNT/CD] marker) (FIG. 5I).

To determine CLDN14 mRNA levels in the kidney, the inventors microdissected each nephron segment from the mouse kidney (FIG. 1B; legend) and quantified CLDN14 transcript levels (normalized to 15-actin mRNA levels) with real-time PCR. TAL expressed the highest levels of CLDN14 mRNA (FIG. 1C), which were over 5-fold higher than in the glomerulus or DCT (p<0.05, n=3); over 10-fold higher than in PCT/PST or CNT/CD (p<0.01, n=3). These microdissected nephron segments were analyzed for the mRNA levels of a TAL marker gene-CLDN 6. CLDN16 transcripts were only found in the TAL (FIG. 1C), ruling out the possibility of tubular cross-contamination during the microdissection process. To look for CLDN14 proteins in the kidney, the inventors immunostained mouse kidney sections under normal dietary condition but found no detectable signal in the kidney (FIG. 5C).

### Example 2

This example illustrates CLDN14 gene expression can be suppressed by miR-9 and miR-374 in the kidney.

The apparent suppression of CLDN14 proteins in the kidney prompted us to look for underlying regulatory mechanisms. The strong CLDN14 promoter activity in the kidney suggested post-transcriptional mechanisms for CLDN14 gene regulation. The CLDN14 gene produces alternatively spliced transcripts in the human (5 variants), the mouse (3 variants) and the rat (1 variant) (see Methods). The splicing variants differ in their 5′-UTR sequences while the coding region and 3′-UTR sequences are conserved in all variants. For predictions of microRNAs targeting CLDN14:3′-UTR based on sequence complementarities and cross-species conservation, four algorithms were used: TargetScan, miRanda, Diana microT, and MirTarget2 (Methods). Because CLDN14:3′-UTR is short, 183 bp in human (#AF314090) (SEQ ID NO: 2), a total of 11 microRNAs were identified targeting the human sequence. MiR-9 consistently appeared in all four algorithms and miR-374 was reported by two independent algorithms. A cross-species search of the 11 microRNA binding sites in mouse CLDN14:3′-UTR (171 bp; AF314089) (SEQ ID NO: 1) reported only the miR-374 binding site conserved in human and mouse sequences. Human miR-374 has two isoforms, a and b, both sharing the same seed sequence. Mature human miR-374b (hsa-miR-374b) is identical to mouse and rat miR-374 (mmu-miR-374 and rno-miR-374 respectively). The miR-374 binding site (AUUAUA matching the seed sequence of miR-374) within CLDN14:3′-UTR was conserved across species. Three pre-miR-9 genes (miR-9-1, -2 and -3) encode the same mature miR-9. Sequence alignment identified a conserved miR-9 binding site within human CLDN14:3′-UTR (CCAAAG), which was however mutated in mouse and rat. A second miR-9 binding site was found in mouse and rat CLDN14:3′-UTRs, located 69 bp upstream of the conserved site.

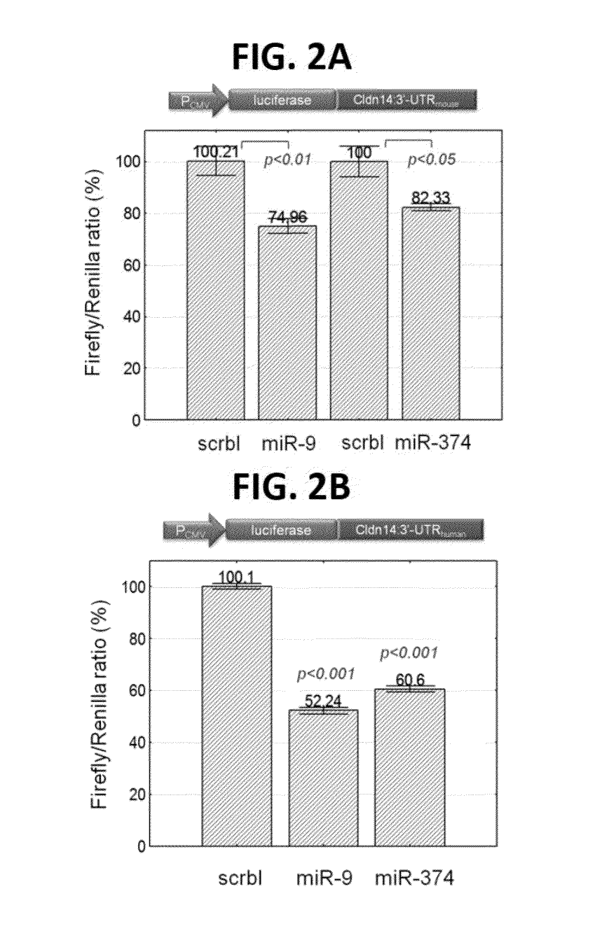

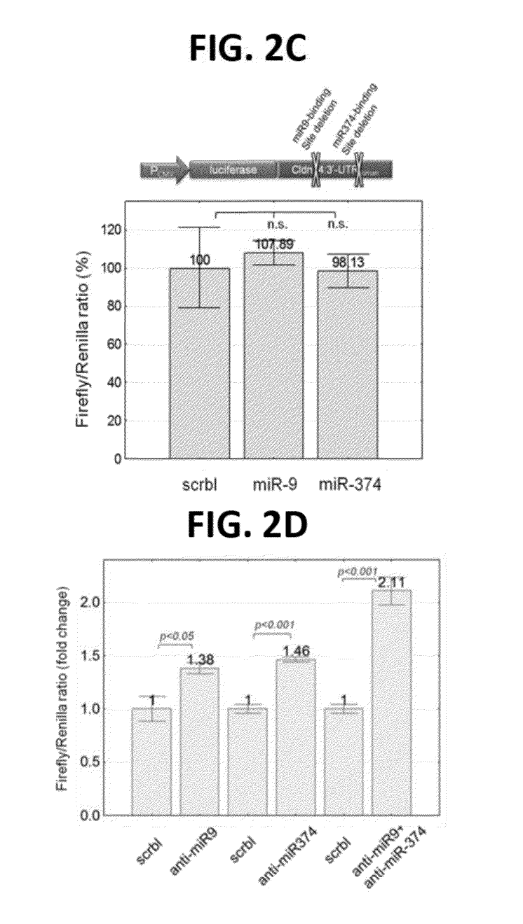

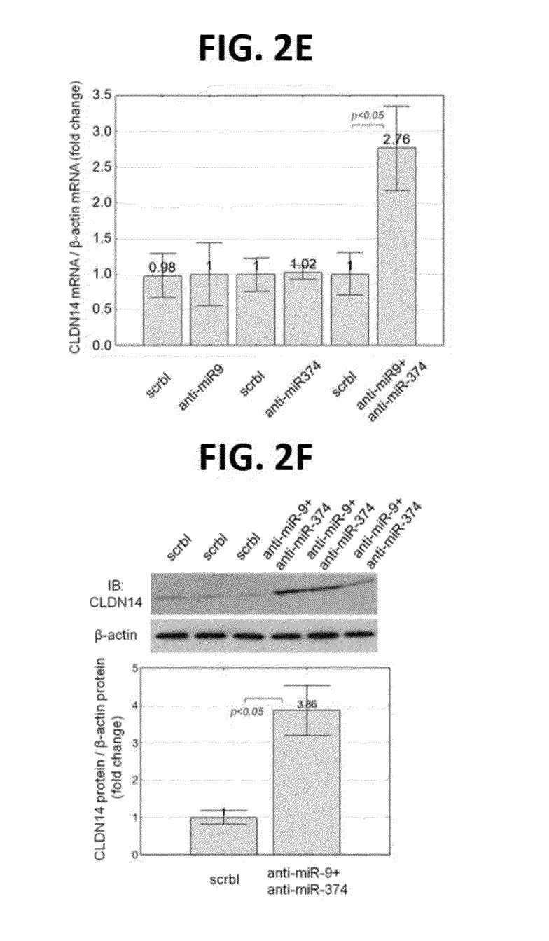

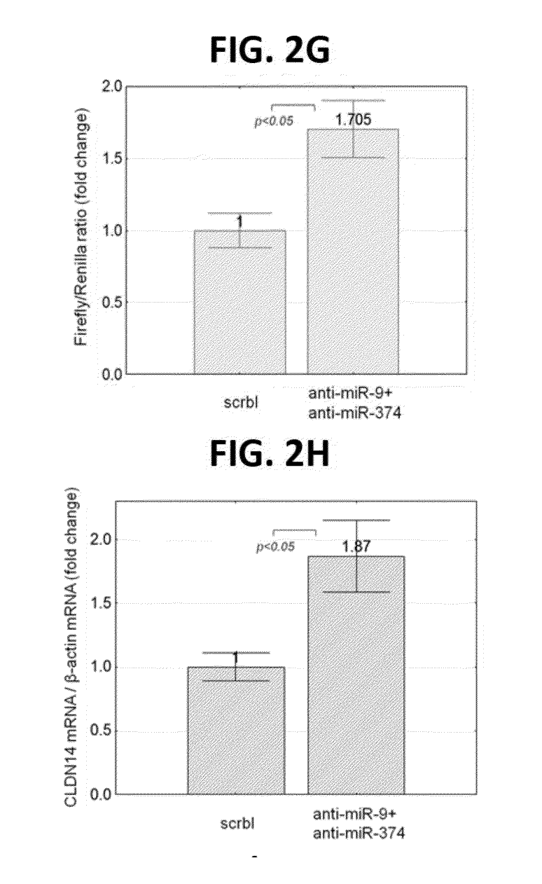

To determine whether miR-9 or miR-374 targets CLDN14:3′-UTR directly, the inventors generated the reporter constructs (pMir-Reporter; Methods) that had the mouse CLDN14:3′-UTR sequence (171 bp) or human CLDN14:3′-UTR sequence (183 bp) cloned downstream of the firefly luciferase gene, termed as pMir-Reporter-CLDN14:3′-UTRmouse or pMir-Reporter-CLDN14:3′-UTRhuman respectively. The pMir-Reporter was transfected with either scrambled miRNA, miR-9 or miR-374 precursor to HEK293 cells. A 25% decrease in firefly luciferase activity (p<0.01, n=4; normalized to Renilla luciferase activity, FIG. 2A) was found for miR-9 when assayed with pMir-Reporter-CLDN14:3′-UTRmouse; an 18% decrease (p<0.05, n=4) for miR-374, compared to scrambled miRNA. The human CLDN14:3′-UTR was more sensitive to microRNA suppression, with miR-9 suppressing pMir-Reporter-CLDN14:3′-UTRhuman activity by 48%; miR-374 by 39% (p<0.001, n=4; FIG. 2B). MiR-9 showed higher efficacy than miR-374 on both pMir-Reporters. Deletion of miR-9 and miR-374 binding sites (Methods) in CLDN14:3′-UTR abolished the miR-9 or miR-374 precursor effect (FIG. 2C).

To demonstrate whether microRNAs are required for CLDN14 regulation in vivo, the inventors adapted the antagomir method (Kruntzfeldt et al., 2005) to repress mature microRNA function in a cultured TAL cell model—MKTAL. The MKTAL cells derived from microdissected mouse TAL tubules and expressed the TAL specific genes, THP and NKCC2 (Bourgeois et al., 2003). The inventors assessed the efficacy of antagomir knockdown of microRNAs by the pMir-Reporter assay. Transfection with anti-miR-9 or anti-miR-374 but not scrambled antagomir increased pMir-Reporter-CLDN14:3′-UTRmouse activity by 1.38-fold (p<0.05, n=4; FIG. 2D) or 1.46-fold (p<0.001, n=4) respectively in MKTAL cells. Co-transfection with anti-miR-9 and anti-miR-374 (at molar ratio 1:1) increased luciferase activity by 2.11-fold (p<0.001, n=4), suggesting a functional synergy between these two microRNAs. This synergy was more evident in their regulation of endogenous CLDN14 expression in MKTAL cells. While neither anti-miR-9 nor anti-miR-374 alone affected CLDN14 mRNA levels, transfection with both antagomirs upregulated CLDN14 mRNA levels by 2.76-fold (p<0.05, n=6: normalized to β-actin mRNA; FIG. 2E) in MKTAL cells.

Concomitant CLDN14 translation assays demonstrated more pronounced microRNA effects. The repression of CLDN14 protein levels was relieved by co-transfection with anti-miR-9 and anti-miR-374, reflected by a 3.86-fold (p<0.05, n=3; normalized to O-actin protein; FIG. 2F) increase on densitometric scale, but not by either antagomir alone. The synergistic effect of multiple microRNAs on their common cognate target has been demonstrated by several studies (Gregory et al., 2008; Yoo et al., 2009). MicroRNA, when bound to its target, triggers mRNA deadenylation through a common deadenylase complex (Huntzinger and Izaurralde, 2011).

To determine whether microRNAs regulated CLDN14 gene expression in the mouse kidney, the inventors isolated mouse TAL cells with an immunomagnetic separation method described before (Methods) (Hou et al., 2009). The isolated cells were viable and express the TAL specific genes, THP and CLDN16 (Hou et al., 2009). Once isolated, they were plated in culture medium for less than 16 hrs followed by immediate antagomir transfection. Antagomir transfection into freshly isolated mouse TAL cells increased pMir-Reporter-CLDN14:3′-UTRmouse activity by 1.71-fold (anti-miR-9+ anti-miR-374 versus scrambled antagomir: p<0.05, n=3; FIG. 2G). The endogenous CLDN14 expression levels in isolated TAL cells were assessed with real-time RT-PCR. Transfection with anti-miR-9+ anti-miR-374 significantly upregulated CLDN14 transcript levels by 1.87-fold (p<0.05, n=4: FIG. 2H) compared to scrambled antagomir. These data demonstrate that miR-9 and miR-374 synergistically regulate CLDN14 gene expression in the thick ascending limb of the kidney.

### Example 3

This example illustrates that CLDN14 interacts with CLDN16 in the kidney.

Since CLDN16 and CLDN19 are colocalized in the TAL (Hou et al., 2008) and the colocalization requires their interaction (Hou et al., 2009), the TAL localization of CLDN14 prompted us to seek for evidence of CLDN14 interaction with CLDN16 or CLDN19. Claudins cis interact within the plane of the membrane to form dimers, or higher oligomeric state, followed by trans interactions between claudins in adjacent cells and additional cis interactions to assemble claudin oligomers into intramembrane tight junction (TJ) strands (Futuse et al., 1999). Because the oligomeric nature of the TJ structure denies an unambiguous study of any selected claudin interaction within the TJ matrix (Stevenson and Goodenough, 1984), cell systems with no TJ (e.g. yeast and embryonic HEK293 cells) were used to probe direct claudin interactions. To determine the cis interactions of CLDN14 with CLDN16 or 19, the inventors used the split-ubiquitin yeast 2-hybrid (Y2H) membrane protein interaction assay (Hou et al., 2008).

Our data show that CLDN14 interacted with itself and with CLDN16, as assayed with all three reporters (HIS3, lacZ, and ADE2) in the yeast NMY51 strain (FIG. 3A). No interaction was evident for CLDN14 and CLDN19 on any of these reporter assays (FIG. 3A). The level of interaction strength between CLDN14 and CLDN16 was equal to that of CLDN16-19 interaction, as quantified with the lacZ reporter (FIG. 3B).

To directly document CLDN14-CLDN16 interaction in epithelial cells, the inventors performed coimmunoprecipitation of CLDN14 and CLDN16 in doubly transfected HEK293 cells, an embryonic cell line with no endogenous claudin or transepithelial resistance (TER). At low cell density, which minimizes cell-cell contacts and trans interactions, interactions between CLDN14 and CLDN16 will be mostly cis. Imnmunoblotting showed that anti-CLDN 4 antibody coprecipitated CLDN16, whereas anti-CLDN16 antibody reciprocally precipitated CLDN14 (FIG. 3C). Other TJ protein antibodies, such as anti-CLDN1 and anti-occludin antibody (as nonspecific binding controls), precipitated neither CLDN14 nor CLDN16 (FIG. 3C). Anti-CLDN19 antibody was not able to precipitate CLDN14; nor was anti-CLDN14 antibody to precipitate CLDN19 (FIG. 3D). In triply transfected cells expressing three claudins at the molecular ratio of 1:1:1, anti-CLDN19 antibody now precipitated CLDN14 with similar affinity to anti-CLDN16 antibody (FIG. 3E), suggesting (1) CLDN119 acted through CLDN16 to interact with CLDN14; (2) CLDN14 integrated into the CLDN16-19 complex instead of forming an independent CLDN14-16 complex. Anti-CLDN14 antibody precipitated the same amount of CLDN16 as anti-CLDN19 antibody (FIG. 3E), suggesting (1) CLDN19-16 interaction was not affected in the presence of CLDN14: (2) the interaction strength of CLDN14-16 was equal to that of CLDN19-16, consistent with the Y2H studies (FIG. 3B). These data indicate that CLDN14, 16 and 19 form an oligomer by utilizing two separate binding sites in CLDN16 to anchor CLDN14 and CLDN19 respectively.

### Example 4

This example illustrates that CLDN14 abolishes the cation selectivity of CLDN16-19 heteromeric channel through repression of CLDN16.

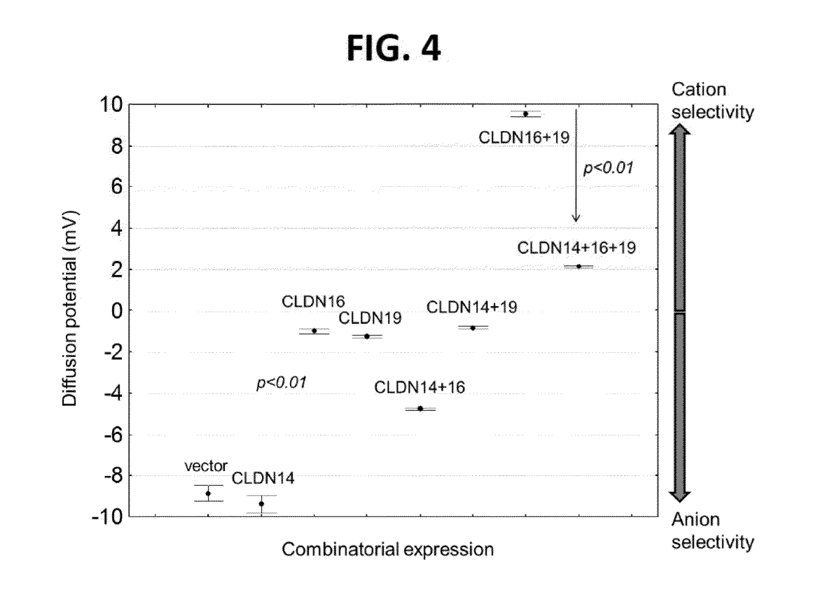

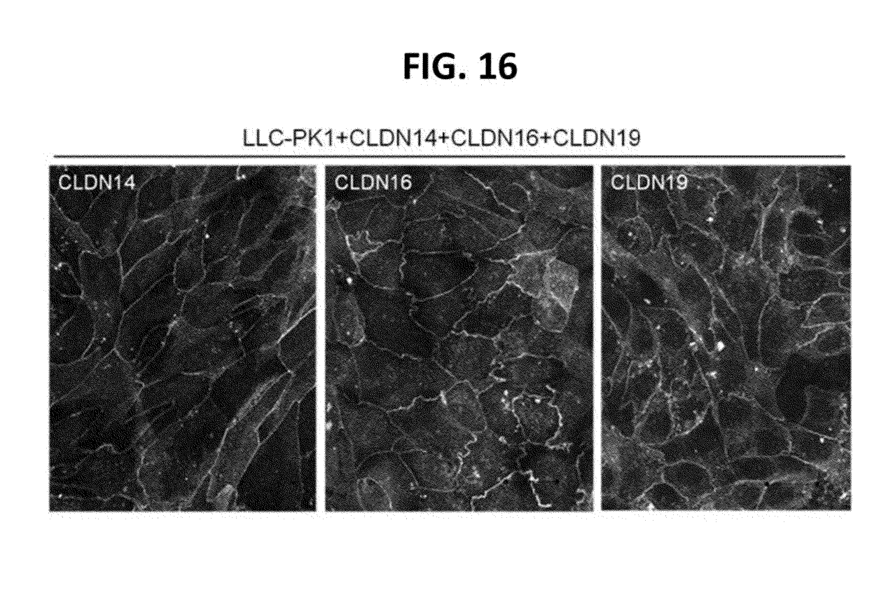

To determine the functional role of CLDN14 in the kidney, the inventors stably expressed CLDN14 in the well-established epithelial cell model LLC-PK1 cells (Hou et al., 2005) that lack the endogenous expression of CLDN14, 16 or 19. As the inventors aimed to have cells expressing CLDN14 over a prolonged period so that they could become fully polarized and form tight junctions (TJ), the inventors used a previously described retroviral expression system (Methods) to drive exogenous CLDN14 expression (Hou et al., 2005; 2008). In LLC-PK1 cells, transfected CLDN16 permeated cations (Hou et al., 2005) while CLDN19 blocked anion permeation (Hou et al., 2008). Coexpression of CLDN16 and CLDN19 generated cation selectivity of the TJ in a synergistic manner (Hou et al., 2008) and resembling the recorded levels in perfused TAL tubules (Hou et al., 2007). With a modified Ussing chamber recording method, the inventors found CLDN14 expression alone in LLC-PK1 cells was without any significant effect on ion selectivity (P_{Na}/P_{Cl}; Table 3). Coexpression of CLDN14 with CLDN16 and CLDN19 abolished their cation selectivity (P_{Na}/P_{Cl}; from 4.486±0.087 in CLDN16+19 reduced to 1.336±0.006 in CLDN14+16+19; Table 3), reflected by a significant decrease in junctional diffusion potential (PD) (FIG. 4; CLDN16+19; 9.53±0.09 versus CLDN14+16+19: 2.13±0.03 mV; p<0.01, n=3). The decrease in P_{Na}/P_{Cl} was attributable to a 2.43-fold decrease in absolute P_{P} (Table 3; CLDN16+19; 23.337±0.082 versus CLDN14+16+19; 9.603±0.019×10⁻⁶ cm/s; p<0.01, n=3), whereas P_{Cl} was mildly affected. The TJ localization of CLDN16 or CLDN19 was not affected by CLDN14 expression (FIG. 16). Since CLDN14 interacts with CLDN16, the inventors investigated whether CLDN14 affected CLDN16 permeability. Expression of CLDN14 in CLDN16 cell background significantly reduced P_{Na} by 2.03-fold (Table 3; CLDN16; 17.013±0.072 versus CLDN14+16; 8.395±0.028×10⁻⁶ cm/s; p<0.01, n=3), resulting in a significant decrease in PD (FIG. 4) and P_{Na}/P_{Cl} (Table 3). Complete substitution of Na⁺ by Li⁺ or Ca²⁺ on one side of the epithelium produced bi-ionic potentials from which the relative permeabilities could be calculated (Hou et al., 2007). CLDN14 expression produced no difference in CLDN16 channel bi-ionic potentials recorded for P_{Li}/P_{Na} or P_{Ca}/P_{Na} (Table 3: footnotes), indicating CLDN14 is a non-selective cation blocker. CLDN14 had no significant effect on CLDN19 function, compatible with the observation that CLDN14 did not interact with CLDN19. Therefore, CLDN14 abolished the cation selectivity of CLDN16-19 heteromeric channel by repressing CLDN16 permeability.

TABLE 3
Combinatorial Expression Analyses of Claudin Channel Permeabilities

| | | Diffusion | | | |
|---|---|---|---|---|---|
| | TER | potential | | P_{Na} | P_{Cl} |
| Group | (Ω · cm²) | (mV) | P_{Na}/P_{Cl} | (10⁻⁶ cm/s) | (10⁻⁶ cm/s) |
| LLC-PK1 + vector | 68.0 ± 3.6 | −8.87 ± 0.23 | 0.257 ± 0.012 | 5.510 ± 0.206 | 21.480 ± 0.204 |
| LLC-PK1 + CLDN14 | 83.3 ± 1.2 | −9.40 ± 0.26 | 0.230 ± 0.013 | 4.114 ± 0.190 | 17.920 ± 0.191 |
| LLC-PK1 + CLDN16 | 50.3 ± 2.3 | −1.00 ± 0.06 | 0.874 ± 0.007^{†} | 17.013 ± 0.072 | 19.473 ± 0.072 |
| LLC-PK1 + CLDN19 | 159.0 ± 2.3 | −1.27 ± 0.03 | 0.843 ± 0.004 | 5.269 ± 0.013 | 6.252 ± 0.013 |
| LLC-PK1 + CLDN14 + CLDN16 | 74.3 ± 2.2 | −4.77 ± 0.03* | 0.515 ± 0.003*^{†} | 8.395 ± 0.028* | 16.310 ± 0.030 |
| LLC-PK1 + CLDN16 + CLDN19 | 169.7 ± 3.4 | −0.83 ± 0.03 | 0.894 ± 0.004 | 5.104 ± 0.012 | 5.711 ± 0.012 |
| LLC-PK1 + CLDN16 + CLDN19 | 64.3 ± 1.2 | 9.53 ± 0.09 | 4.486 ± 0.087 | 23.337 ± 0.082 | 5.206 ± 0.082 |
| LLC-PK1 + CLDN14 + CLDN16 + | 109.3 ± 3.9 | 2.13 ± 0.03^{†} | 1.336 ± 0.006^{†} | 9.603 ± 0.019^{†} | 7.189 ± 0.019 |
| CLDN19 | | | | | |

*p < 0.01 relative to LLC-PK1 + CLDN16 cells, n = 3.
^{†}p < 0.01 relative to LLC-PK1 + CLDN16 + CLDN19 cells, n = 3.
^{‡}P_{Li}/P_{Na}: 1.086 ± 0.008 in LLC-PK1 + CLDN16 cells versus 1.102 ± 0.011 in LLC-PK1 + CLDN14 + CLDN16, n = 3, not significant;
P_{Ca}/P_{Na}: 0.724 ± 0.009 in LLC-PK1 + CLDN16 cells versus 0.705 ± 0.008 in LLC-PK1 + CLDN14 + CLDN16, n = 3, not significant.

### Example 5

This example illustrates regulation of CLDN14 gene expression by extracellular Ca²⁺ in the kidney.

The inventors investigated whether CLDN14 played a physiological role in renal Ca²⁺ homeostasis. If CLDN14 is required for renal excretion of Ca²⁺, manipulating the dietary intake would induce changes in its expression, compatible with its role in regulating CLDN16 and 19 functions. To manipulate dietary calcium intakes in animals, age (7-8 weeks old) and sex (female) matched mice were segregated into three groups (I-III) and fed with the basal diet (I; Ca²⁺: 0.61%), low Ca²⁺ diet (II; Ca²⁺: 0) or high Ca²⁺ diet (III; Ca²⁺: 5%) respectively for six consecutive days (Methods). While the circulating Ca² level was well defended under low Ca²⁺ diet, high Ca²⁺ diet induced significant hypercalcemia in animal group III (plasma Ca²⁺: 2.72 mM versus 2.53 mM in basal group I; p<0.01, n=5; FIG. 5A).

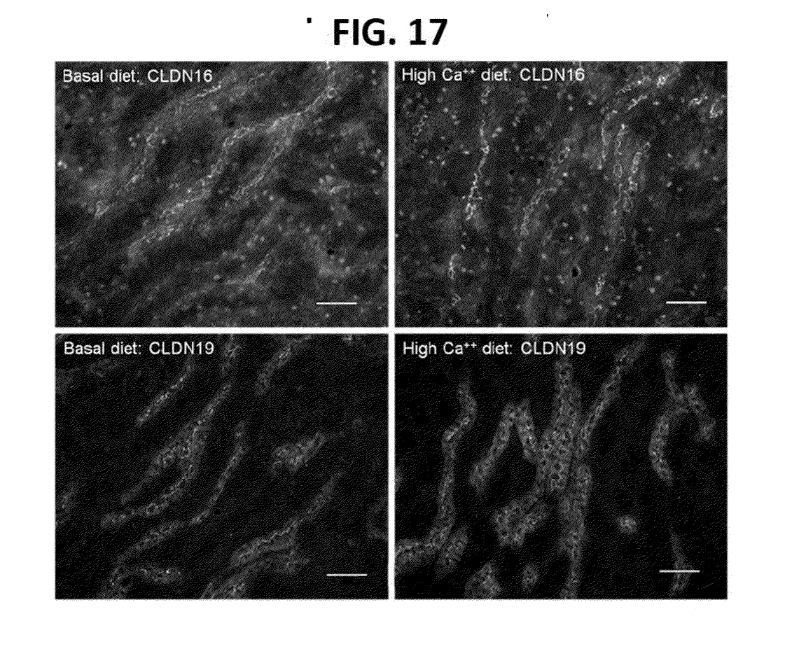

To determine the gene expression levels of CLDN14 in mouse kidneys receiving dietary treatments, the inventors isolated kidney TAL cells at the end of each treatment with the immunomagnetic separation method described elsewhere in this study and quantified CLDN14 mRNA levels (normalized to 13-actin mRNA) with real-time RT-PCR. While low Ca² diet (II) significantly downregulated CLDN14 mRNA levels to 37% of the basal level (I) (p<0.01, n=5; FIG. 5B), high Ca²⁺ diet (III) significantly upregulated CLDN14 by 3.18-fold (p<0.01, n=5; FIG. 5B) compared to the basal diet (I). CLDN14 proteins were immunostained in mouse kidneys to show changes in protein levels. While the staining signal for CLDN14 was not detectable in the basal group (I) (FIG. 5C), high Ca²⁺ diet (III) profoundly upregulated CLDN14 protein levels that now showed strong staining in tight junctions of the TAL tubules (FIG. 5C: arrow-head). The TJ localization of CLDN16 or CLDN19 in mouse kidneys was not affected by dietary Ca²⁺ variations (FIG. 17). Because the immunomagnetic separation method generated limited TAL material from a mouse kidney, the inventors pooled the TAL cells from all animals (n=5) within each dietary group and quantified CLDN14 protein levels by Western Blot. High Ca²⁺ diet (III) increased CLDN14 protein levels by 3.12-fold on densitometric scale (FIG. 5D; normalized to β-actin protein) compared to the basal diet (I). To prove that CLDN14 gene expression is regulated by extracellular Ca²⁺ per se independent of any hormonal molecule (e.g. the PTH), the inventors directly manipulated extracellular Ca²⁺ levels in a cultured TAL model—MKTAL (Methods).

Strongly correlated with the elevation of extracellular Ca²⁺ concentration (supplemented to an EBSS based low Ca²⁺ culture medium; Methods), there was a progressive induction of CLDN14 mRNA level (FIG. 5E), with statistical significance first reached at 0.2 mM Ca²⁺ (p<0.01, n=3). The induction of CLDN14 mRNA level was dose-dependent on and linear within the physiological range of extracellular Ca²⁺ (0-3 mM) (FIG. 5E). Maximal induction of CLDN14 mRNA level was observed by 5.38-fold with 3 mM Ca²⁺ (p<0.01, n=3: FIG. 5E). Because extracellular Ca²⁺ is required for TJ assembly and affects TJ protein maturation (Wong, 1997; Zheng and Cantley, 2007), the inventors used 0.1 mM Ca²⁺ in culture medium as the control condition for protein assays. CLDN14 protein levels were significantly increased by 2.59-fold in 3 mM Ca²⁺ (p<0.01, n=4; FIG. 5F) compared to control. PTH, however, when supplemented over a range of doses (1 nM, 10 nM and 100 nM), had no significant effect on CLDN14 gene expression in MKTAL cells (n=3: FIG. 5G). These results indicate that CLDN14 gene expression in the kidney is directly regulated by extracellular Ca²⁺.

### Example 6

This example illustrates that CLDN14 is required for regulating Ca²⁺ excretion in the kidney.

While targeted deletion of CLDN14 in animals initially focused on its role in the inner ear, subsequent physiological studies showed normal renal function in CLDN14 knockout (KO) mice under basal dietary condition (Ben-Yosef et al., 2003: Elkouby-Naor et al., 2008). The rare loss-of-function mutations (398delT and T254A) associated with recessive deafness DFNB29 caused no renal abnormality in affected homozygous individuals (Wilcox et al., 2001). The lack of renal phenotype in CLDN14 KO animal or DFNB29 patient was compatible with our findings that CLDN14 gene expression was endogenously suppressed in absence of high Ca²⁺ intakes. The inventors previously described a CLDN16 knockdown (KD) and CLDN19 KD animal model that both recapitulated human autosomal recessive familial hypomagnesenia with hypercalciuria and nephrocalcinosis (FHHNC) phenotypes (Hou et al., 2007; 2009). Because CLDN14 inhibited the CLDN16-19 channel, knockout of CLDN14 generates a renal phenotype opposite to that in CLDN16 KD and CLDN19 KD.

To demonstrate in vive role of CLDN14 in the kidney, the inventors performed 24 hr urinalysis on CLDN14 KO mice and their littermate WT controls (Methods). Age and sex matched animals from each group were fed with high Ca²⁺ diet (III; Ca²⁺: 5%) for six consecutive days. The plasma Mg²⁺ level in CLDN14 KO mice was significantly higher (by 15%: p<0.05, n=6; Table 4) than in WT, whereas the circulating Ca²⁺ level was not significantly altered. CLDN14 KO mice showed defects in adjusting renal tubular absorption rate to excrete excess quantities of filtered Ca²⁺ and Mg²⁺. The fractional excretion rate for Ca²⁺ (FECa) in CLDN14 KO animals was 32% of the level in WT (p<0.01, n=6; Table 4); FEMg in CLDN14 KO was 45% of WT (p<0.05, n=6: Table 4). The glomerular filtration rate (GFR) was increased by 1.84-fold in CLDN14 KO animals (p<0.01, n=6: Table 4) compared to WT; whereas the urinary volume (UV) was not significantly different between CLDN14 KO and WT. A plausible explanation for GFR increases in CLDN14 KO mice would be stimulation of the tubuloglomerular feedback (TGF) owing to higher Na⁺ absorption through the CLDN16-19 channel in the TAL. The paracellular Na⁺ absorption accounts for one half of total Na⁺ absorption in the TAL (Hebert and Andreoli, 1986). These data demonstrate that CLDN14 regulates renal Ca²⁺ excretion to counterbalance dietary changes.

TABLE 4
Analyses of Plasma and Urine Electrolytes in WT and CLDN14
KO Mice Under High Ca²⁺ Dietary Condition

| | | CLDN14 | |
|---|---|---|---|
| | WT | KO | Significance |
| Weight, g | 19.04 ± 0.58 | 19.36 ± 2.56 | NS |
| P_{Ca}, mM | 2.74 ± 0.02 | 2.75 ± 0.05 | NS |
| P_{Mg}, mM | 0.93 ± 0.03 | 1.07 ± 0.03 | p < 0.05 |
| UV, μl/24 hr/g | 45.58 ± 7.09 | 40.82 ± 13.32 | NS |
| GFR, ml/24 hr/g | 3.91 ± 0.21 | 7.20 ± 0.56 | p < 0.01 |
| FE_{Ca}, % | 1.65 ± 0.08 | 0.53 ± 0.15 | p < 0.01 |
| FE_{Mg}, % | 7.03 ± 1.00 | 3.14 ± 0.44 | p < 0.05 |

Values are expressed as means ± SEM; n = 6, the number of animals; sex, male; age, 9-10 weeks.
UV, urine volume; NS, not significant; GFR, glomerular filtration rate; P_{Ca}, P_{Mg}, plasma Ca²⁺ and Mg²⁺ concentrations; FE_{Ca}, FE_{Mg}, fractional excretion of Ca²⁺ and Mg²⁺.

### Example 7

This example illustrates regulation of miR-9 and miR-374 gene expression by extracellular Ca²⁺ in the kidney.

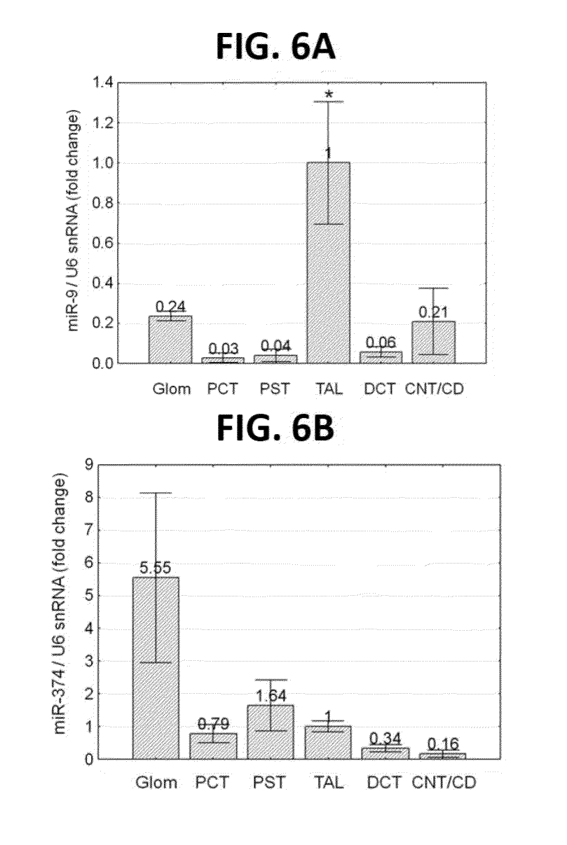

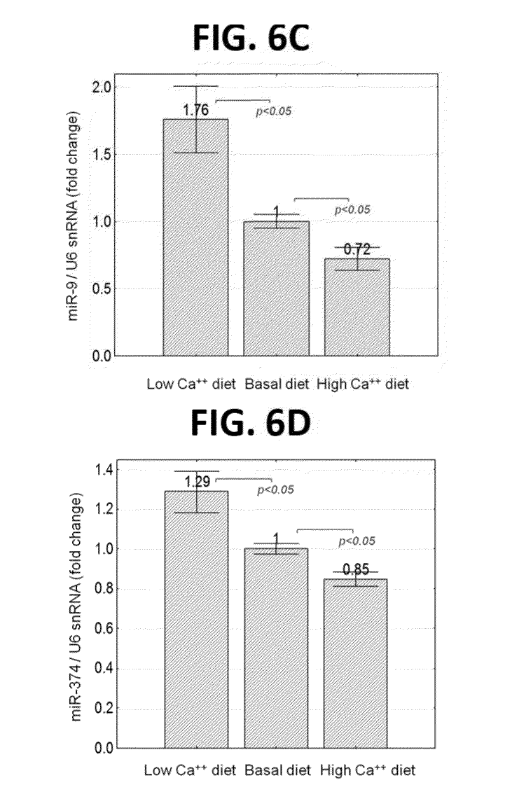

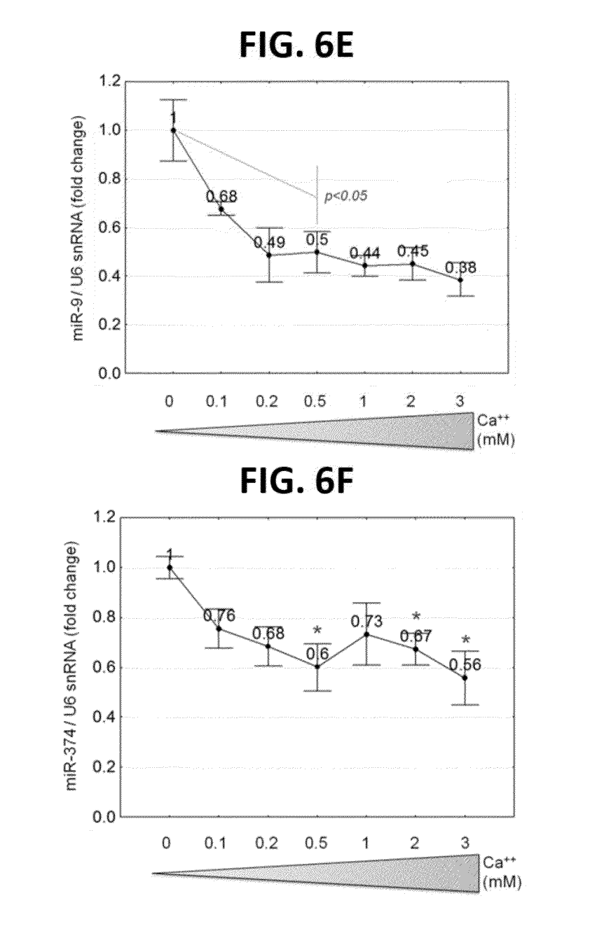

The inventors investigated whether microRNAs themselves were regulated by extracellular Ca²⁺, which may suggest a physiological role for microRNAs in renal handling of Ca²⁺. Because CLDN14 gene expression was exclusively localized in the TAL, the inventors investigated where miR-9 and miR-374 were expressed in the kidney. Quantitative PCR measurements (normalized to U6 snRNA transcript) from micro-dissected nephron segments (FIG. 1B) showed that miR-9 had the highest expression level in the TAL (p<0.05, n=3; relative to other segments; FIG. 6A); while miR-374 expression levels were: glomerulus>PCT≈PST≈TAL>DCT>CNT/CD (FIG. 6B). If observed Ca²⁺ regulation of CLDN14 was mediated through microRNAs, the experimental conditions used to elicit changes in CLDN14 gene expression would cause reciprocal changes in microRNA expression.

In the TAL of mouse kidneys receiving dietary Ca²⁺ variations, low Ca²⁺ diet (II) significantly upregulated miR-9 and miR-374 transcript levels by 1.76-fold and 1.29-fold respectively (p<0.05, n=5; relative to basal diet I; FIG. 6C-D); while high Ca²⁺ diet (III) significantly downregulated miR-9 and miR-374 by 28% and 15% respectively (p<0.05, n=5; relative to basal diet I; FIG. 6C-D). The inverse correlation between microRNA and CLDN14 expression was also evident in cultured MKTAL cells receiving direct extracellular Ca²⁺ variations. Increasing extracellular Ca²⁺ levels progressively repressed the transcript levels of both miR-9 (FIG. 6E) and miR-374 (FIG. 6F), with statistical significance first reached at 0.5 mM Ca²⁺ (p<0.05, n=3). Maximal repression for both microRNAs was found with the concentration of 3 mM Ca²⁺ (by 62% for miR-9 and by 44% for miR-374; FIG. 6E-F), which coincided with maximal induction of CLDN14. These results demonstrate that extracellular Ca²⁺ regulation of CLDN14 gene expression in the kidney is mediated through changes in miR-9 and miR-374 gene expression.

### Example 8

This example illustrates that CaSR is required for extracellular Ca²⁺ regulation of CLDN14 and microRNAs.

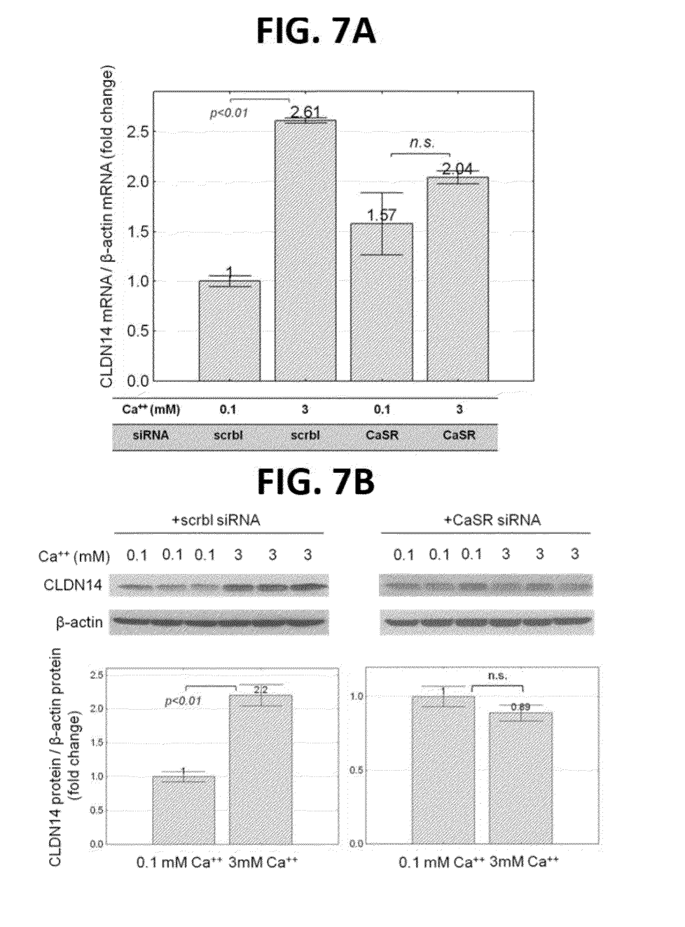

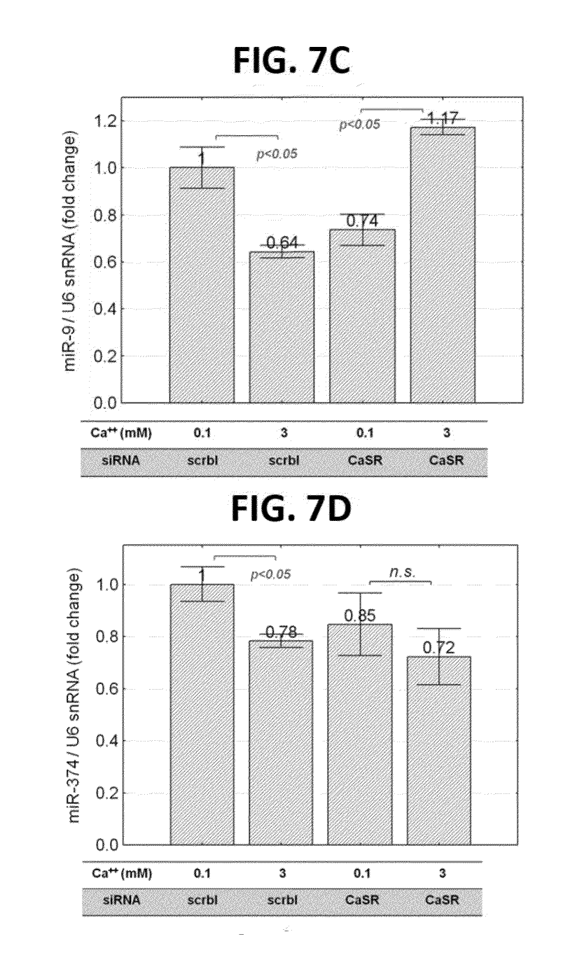

CaSR is a molecule that plays a role in the TAL that senses circulating Ca²⁺ changes and regulates urinary excretion. To provide direct evidence that CaSR was required for Ca²⁺ regulation of CLDN14 and microRNAs in the TAL, the inventors knocked down endogenous CaSR expression in MKTAL cells by RNA interference and sought for deregulation of CLDN14 and microRNAs. A pre-validated siRNA reagent, containing a pool of 3 target-specific 21 nt siRNA duplexes designed against the mouse CaSR gene (Methods), was transfected into MKTAL cells prior to Ca²⁺ activation. A scrambled siRNA duplex was transfected as negative control. While extracellular Ca²⁺ (3 mM) increased CLDN14 mRNA levels (FIG. 7A) and protein levels (FIG. 7B) by 2.61-fold and 2.20-fold respectively (versus 0.1 mM Ca²⁺; p<0.01, n=3) in scrb1-siRNA cells, CaSR-siRNA completely abolished the Ca²⁺ inducing effect on CLDN14 expression (FIG. 7A-B).

The transcript levels of miR-9 and miR-374 were significantly decreased by Ca²⁺ treatments in scrb1-siRNA cells (miR-9: by 36%, FIG. 7C; miR-374: by 22%, FIG. 7D: p<0.05, n=3), consistent with their role of silencing CLDN14. CaSR knockdown decreased the basal levels of both microRNAs. In CaSR-siRNA cells, neither microRNA was further down-regulated by Ca²⁺ treatments (FIG. 7C-D). While extracellular Ca²⁺ failed to induce any significant effect on miR-374, miR-9 was upregulated by Ca²⁺ in absence of CaSR. The fold of upregulation was small (1.58-fold) but significant (p<0.05, n=3; FIG. 7C) for miR-9. These results indicate that CaSR is required for extracellular Ca²⁺ signaling to gene regulation of the microRNA-CLDN14 axis.

### Example 9

This example illustrates that pharmacological manipulation with calcimimetic and calcilytic compounds demonstrates CaSR dependent regulation of claudin-14 gene expression in the kidney.

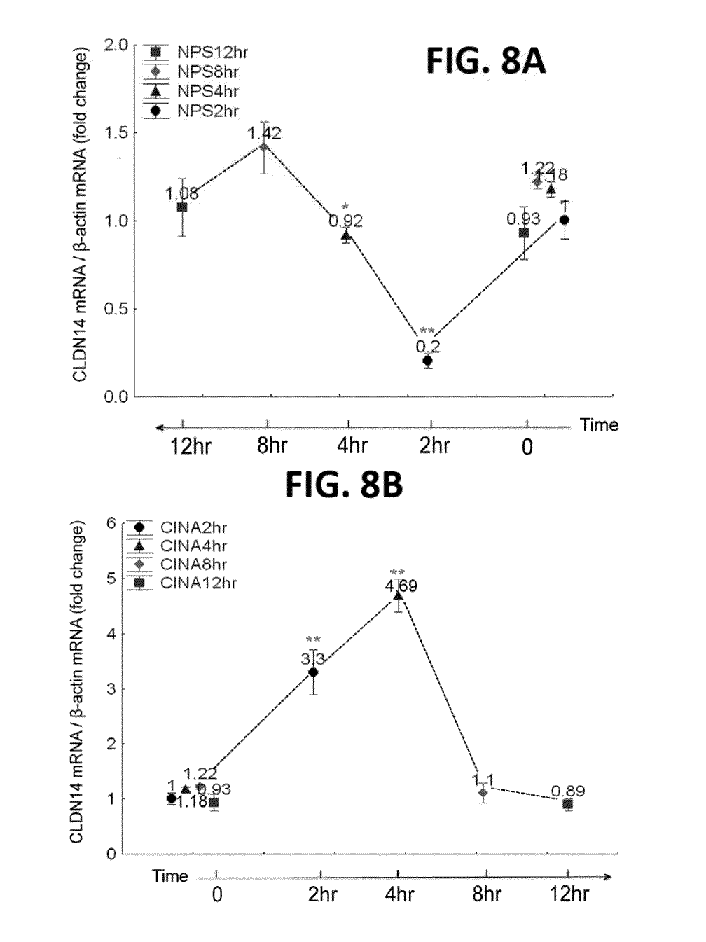

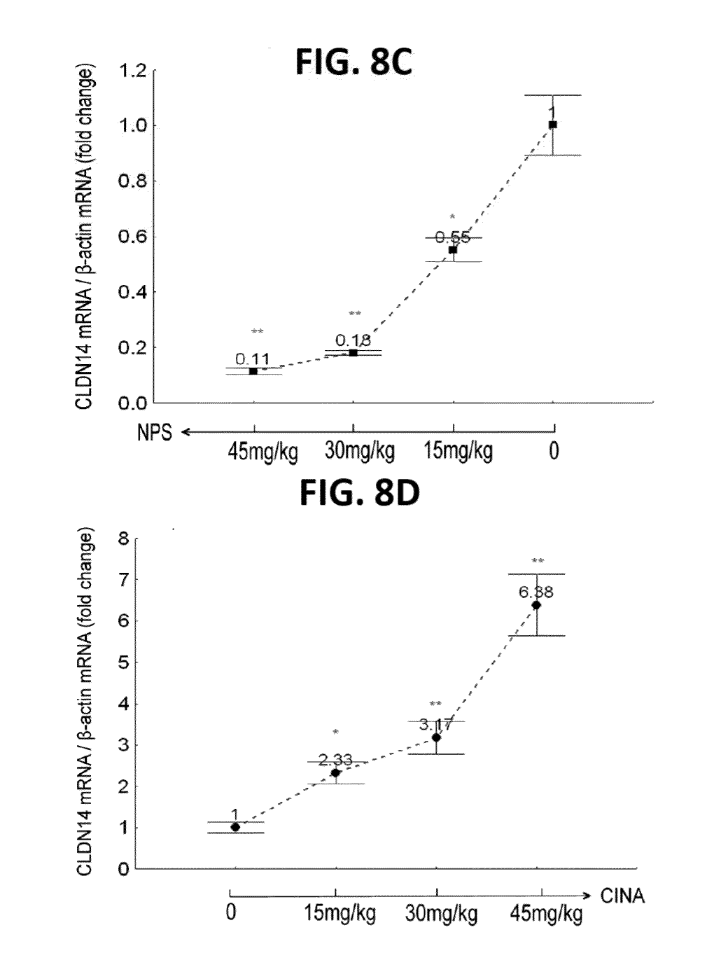

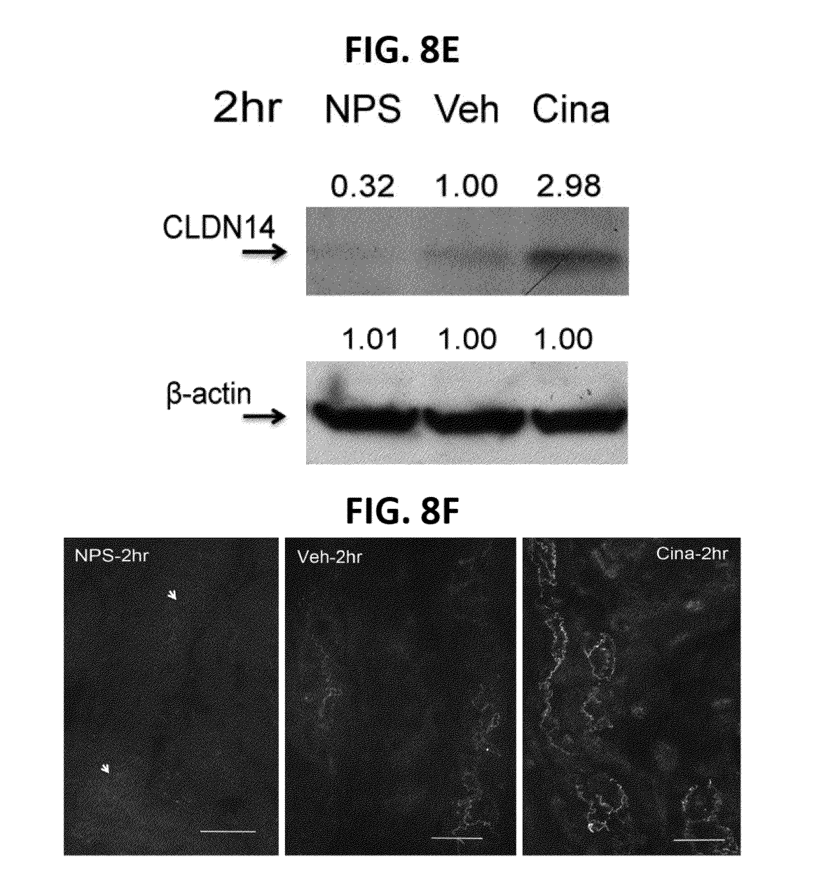

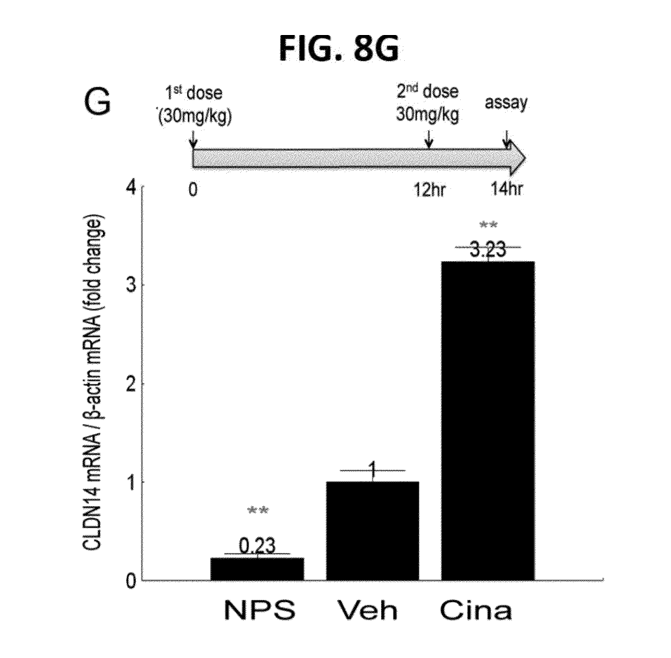

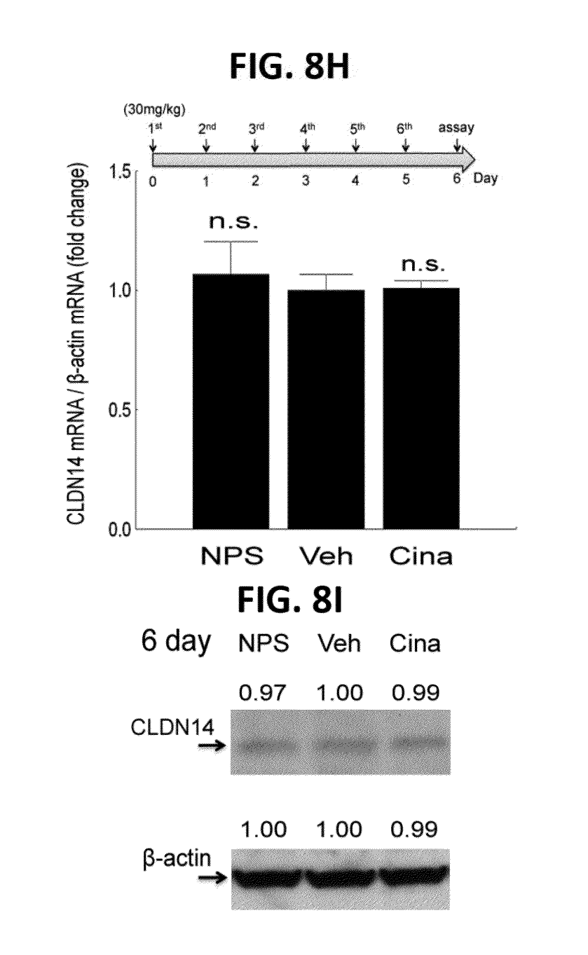

To investigate how CaSR regulated claudin-14 in vivo in the kidney, age (8-10 weeks old) and sex (male) matched mice (strain: C57BL/6) were treated with NPS2143 and cinacalcet over a range of doses and durations, isolated kidneys at the end of each treatment and quantified claudin-14 mRNA and protein levels with real-time PCR and western blot respectively. Both NPS2143 and cinacalcet rapidly regulated the mRNA and protein levels of claudin-14 in the kidney. A single oral dose of NPS2143 at 30 mg/kg BW⁻¹ significantly downregulated the mRNA level of claudin-14 by 80% (normalized to β-actin mRNA) at 2 hrs (p<0.01, n=3 versus vehicle level at 2 hrs: FIG. 8A): the downregulation persisted through to 4 hrs although claudin-14 mRNA recovered to 78% of its control level (p<0.05, n=3 versus vehicle level at 4 hrs; FIG. 8A); by 8 hrs and through to 12 hrs, claudin-14 mRNA completely recovered to its control level.

Cinacalcet at a single oral dose of 30 mg/kg BW⁻¹, on the other hand, significantly upregulated the mRNA level of claudin-14 by 3.3-fold at 2 hrs (p<0.01, n=3 versus vehicle level at 2 hrs: FIG. 8B): by 4 hrs the claudin-14 mRNA level reached the ceiling of 3.97-fold (p<0.01, n=3 versus vehicle level at 4 hrs; FIG. 8B); by 8 hrs and through to 12 hrs, claudin-14 mRNA recovered to its control level. To determine the dosage effect of NPS2143 and cinacalcet, the time point was selected for both drugs at 2 hrs. Across the doses of 15, 30 and 45 mg/kg BW⁻¹, NPS2143 treatments generated progressive reduction of claudin-14 mRNA levels, by 45%, 82% and 89% respectively (p<0.05, n=4 versus vehicle; FIG. 8C); while cinacalcet progressively induced claudin-14 expression by 2.33, 3.17 and 6.38-fold respectively (p<0.05, n=4 versus vehicle; FIG. 8D). The regulatory trajectory for both drugs appeared to be linear within 30 mg/kg BW⁻¹. Changes in claudin-14 protein levels were captured as early as 2 hrs following a single oral dose of NPS2143 or cinacalcet (30 mg/kg BW⁻¹) treatment.

Because of the low abundance of claudin-14 proteins in the whole kidney, an immunomagnetic separation method was adapted to freshly isolate the TALH tubular cells from the kidneys of each treated mouse, pooled the TALH cells from all animals (N=5) within each treatment group and quantified claudin-14 protein levels by western blot. While NPS2143 downregulated claudin-14 protein levels to 32% of the vehicle treatment on densitometric scale (FIG. 8E: normalized to β-actin protein), cinacalcet upregulated claudin-14 protein levels by 2.98-fold (FIG. 8E). Claudin-14 proteins were immunostained in mouse kidneys to investigate changes in TJ localization. Claudin-14 proteins were detected in tight junctions of vehicle treated mice that showed an interdigitated pattern characteristic of the TALH tubule (FIG. 8F). While NPS2143 reduced the staining signal for claudin-14 to punctate foci (FIG. 8F; arrow head) apically located reminiscent of dissolved TJ strands, cinacalcet profoundly upregulated claudin-14 protein levels in the tight junction that now showed reinforced staining in the TALH tubules (FIG. 8F).

To determine the long-term effects of NPS2143 and cinacalcet on claudin-14 gene expression and knowing that a first dose of NPS2143 or cinacalcet (30 mg/kg BW⁻¹) generated the most pronounced gene regulation of claudin-14 at 2 hrs that dissipated completely by 12 hrs, animals were given a second dose of NPS2143 or cinacalcet (30 mg/kg BW⁻¹) at 12 hrs and measured claudin-14 gene expression at 14 hrs. The second dose of NPS2143 reduced claudin-14 mRNA levels by 77% (p<0.01, n=3 versus vehicle, FIG. 8G), to a similar extent as a single dose treatment (FIG. 8A): whereas the second cinacalcet treatment increased claudin-14 mRNA levels by 3.23-fold (p<0.01, n=3 versus vehicle; FIG. 8G), not different from the first dose (FIG. BB).

To distinguish from the observed short-term effects, animals were given an oral dose of NPS2143 or cinacalcet (30 mg/kg BW⁻¹) per day for 6 days and measured claudin-14 gene expression 24 hrs following the last treatment. Neither NPS2143 nor cinacalcet caused changes in claudin-14 mRNA (FIG. 8H) or protein levels (FIG. 8I) during the 6-day treatment. According to Amgen, maximum serum concentration of cinacalcet after oral administration is achieved in approximately 1.5-3 hrs (Nemeth et al., 2004). The half-life of absorbed cinacalcet is 30-40 hrs. NPS2143 will likely have similar pharmacokinetic profiles. The rapid deregulation of claudin-14 following NPS2143 or cinacalcet treatment (within 8 hrs; FIGS. 8A-B) suggests a desensitization mechanism on the CaSR level. The lack of long-term effects from NPS2143 or cinacalcet treatment (FIGS. 8H-I) suggests the intracellular CaSR signals are not accumulated over time. These results indicate that the CaSR regulation events of claudin-14 in the kidney are organized in pulses that peak during 2-4 hrs after each calcimimetic or calcilytic treatment. Each pulse is independent and carries no signal to the pulses afterwards.

### Example 10

This example illustrates the gene regulation of claudin-14 by CASR is independent of the parathyroid hormone.

The inventors investigated whether CaSR regulation of claudin-14 depended upon the PTH secretion. In a previous report showed that, PTH, when supplemented to a cultured TALH cell model, had no effect on claudin-14 gene expression in vitro (see above). To test the PTH dependence of renal CaSR effects in vivo, the inventors applied the following three criteria.

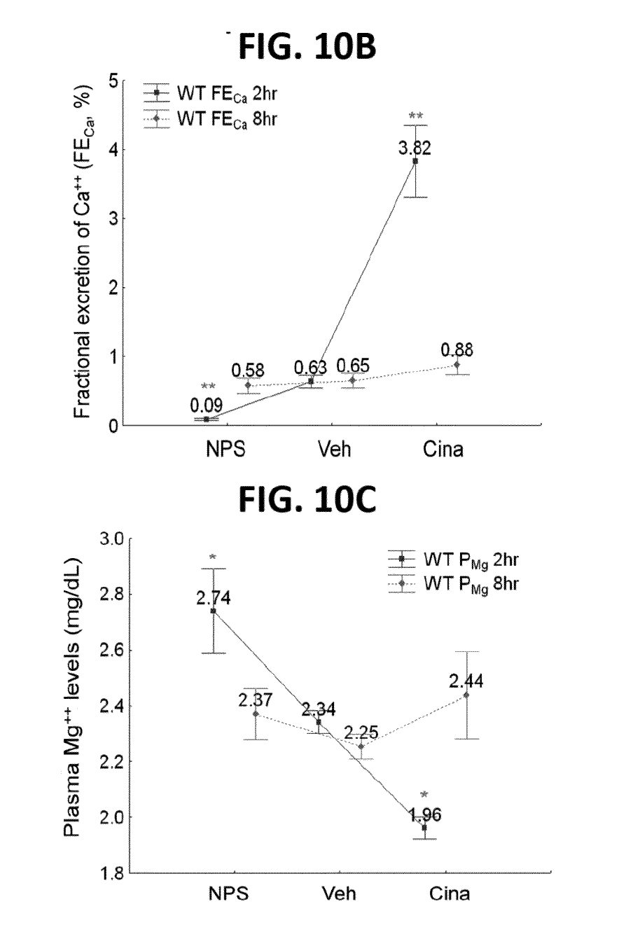

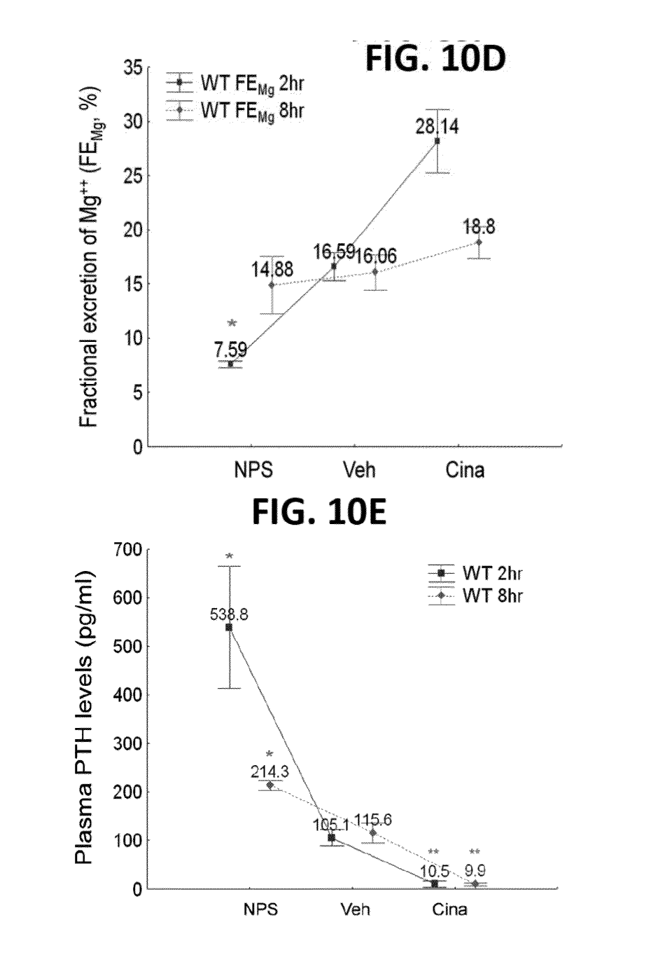

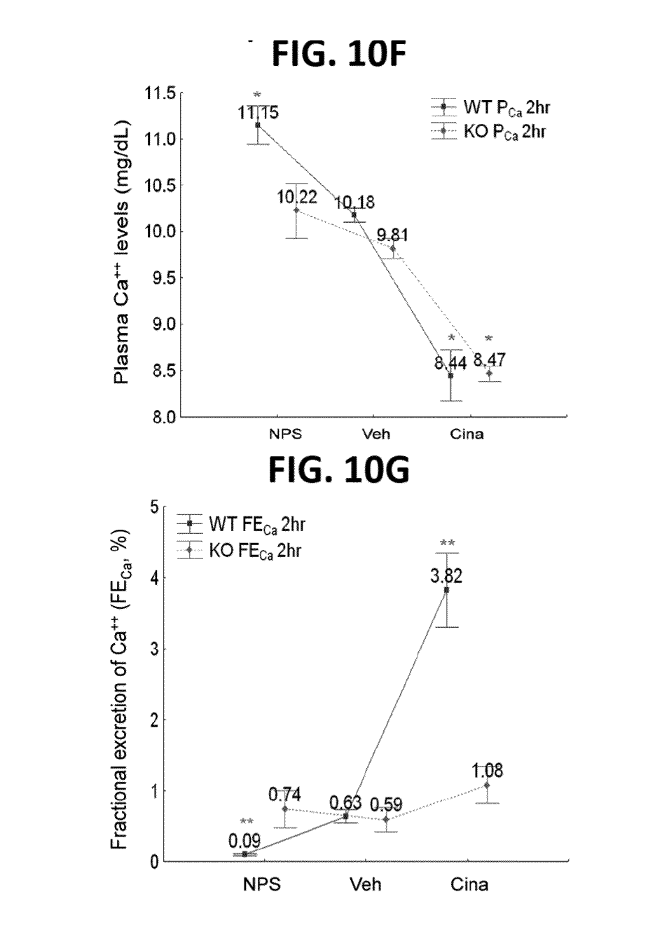

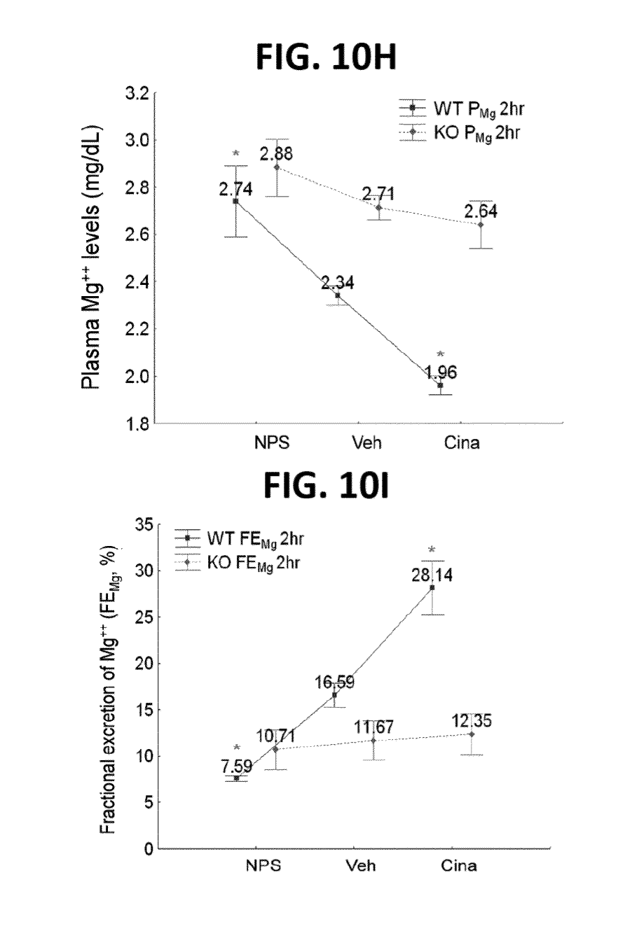

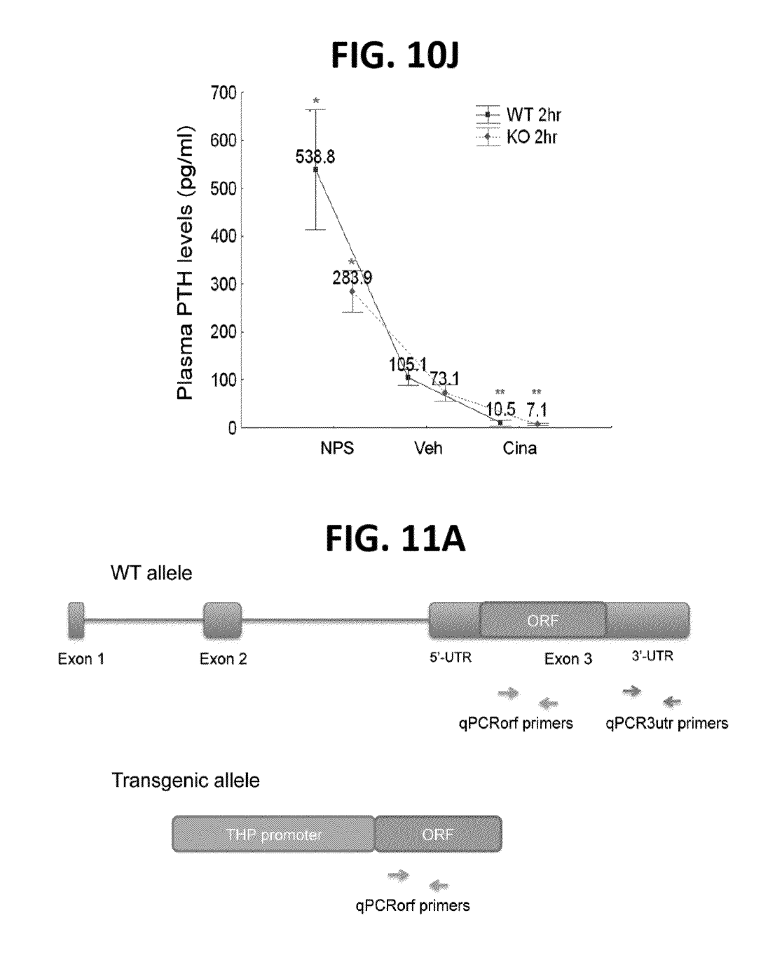

(1) Changes in PTH alone did not induce claudin-14 gene regulation. In intact mice, there was no significant change in claudin-14 gene expression 8 hrs following a single dose of NPS2143 or cinacalcet (FIG. 8A-B) despite significant differences in plasma PTH levels (FIG. 10E) at the same time point.

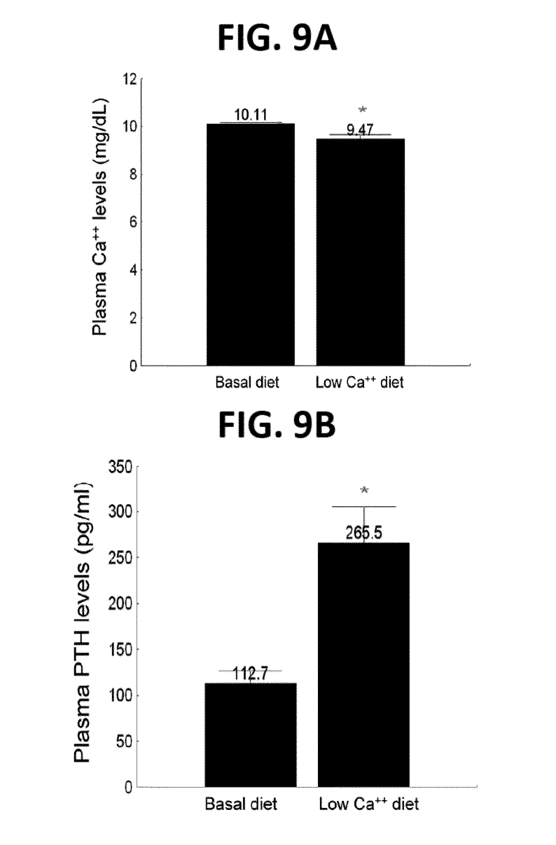

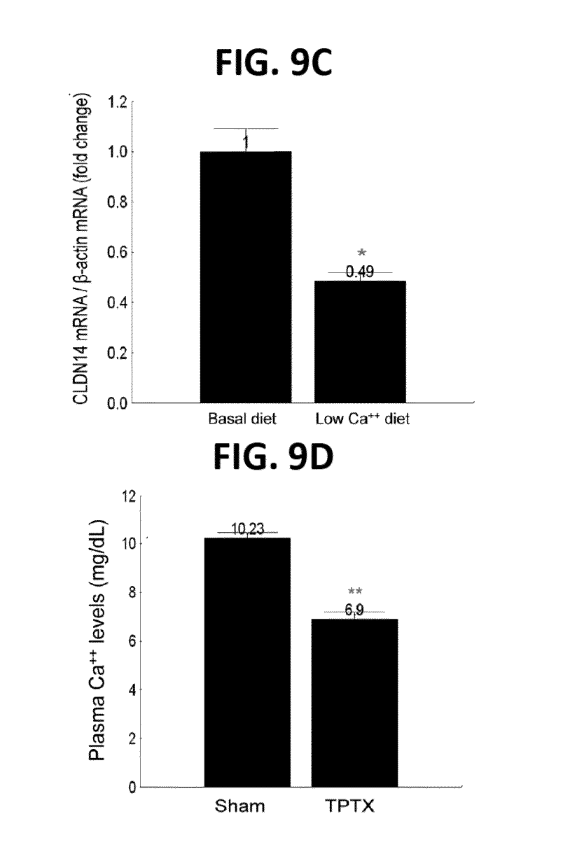

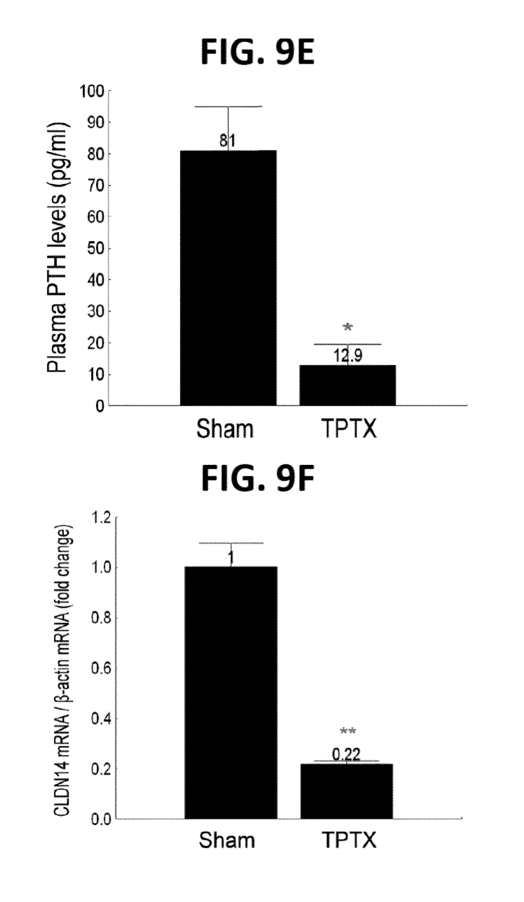

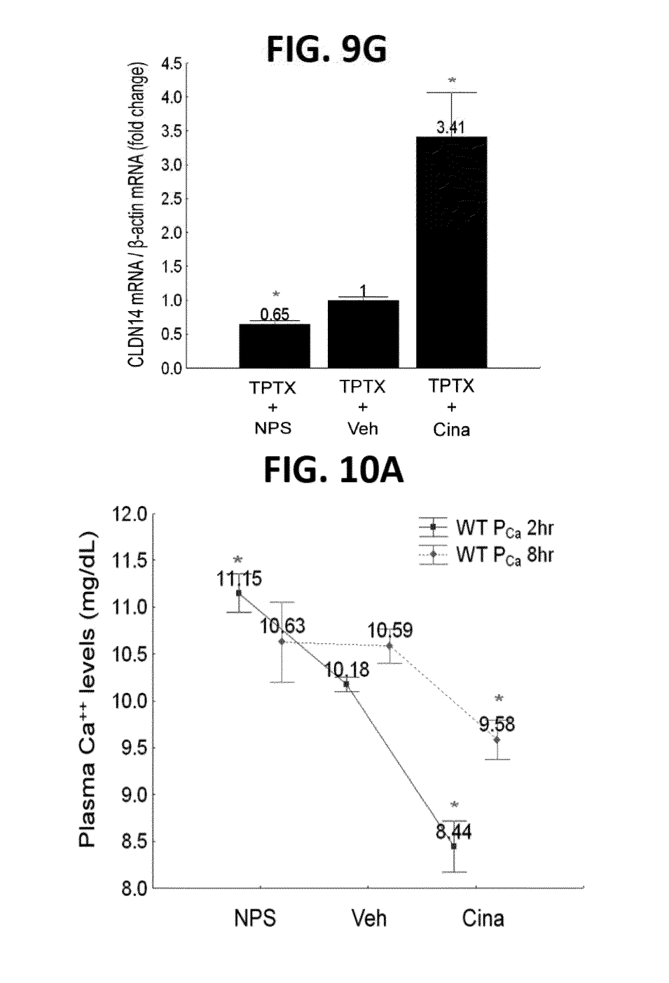

(2) Hypocalcemia regulated claudin-14 gene expression independent of plasma PTH levels. Mice fed with low Ca²⁺ diet for 6 days (see Methods) showed hypocalcemia (p<0.05, n=5: FIG. 9A), elevated plasma PTH levels (p<0.05, n=5; FIG. 9B) and 51% downregulation of claudin-14 mRNA levels in the kidney (p<0.05, n=5; FIG. 9C), compared to animals fed with basal diet for the same duration. Thyroparathyroidectomy (TPTX, see Methods), on the other hand, significantly reduced plasma PTH levels by 84% in mice (p<0.05, n=5; FIG. 9E), causing severe hypocalcemia (6.90±0.30 mg/dL versus 10.23-0.24 mg/dL in sham control; p<0.01, n=5; FIG. 9D) and even more pronounced claudin-14 downregulation in the kidney (by 78%, p<0.01, n=5; FIG. 9F), compared to sham-operated animals.

(3) PTH was not required for CaSR regulation of claudin-14 in the kidney. In TPTX treated mice with nominal presence of circulating PTH, cinacalcet at a single dose of 30 mg/kg BW⁻¹ significantly upregulated claudin-14 expression levels by 3.41-fold at 2 hrs (p<0.05, n=5 versus vehicle: FIG. 9G), by which time cinacalcet induced a comparable 3.3-fold upregulation in intact mice (FIG. 8B). NPS2143 was able to further downregulate claudin-14 gene expression by 35% at 2 hrs after a single dose of 30 mg/kg BW⁻¹ (p<0.05, n=5 versus vehicle; FIG. 8G) in TPTX treated mice, mindful that the basal level of claudin-14 in TPTX animals was already 78% lower than in sham controls (FIG. 8F). Notably, the NPS2143 effect appeared to be diminished in TPTX mice compared to that in intact animals (FIG. 8A).

These data suggest that the renal regulation of claudin-14 by CaSR will not require its endocrine role in the parathyroid gland.

### Example 11

This example illustrates that the renal calcium excretion response to calcimimetic and calcilytic treatment is abolished in claudin-14 knockout animals.

The inventors investigated whether claudin-14 underlays the renal role of CaSR in urinary Ca²⁺ handling. The claudin-14 KO mouse model that showed hypomagnesiuria and hypocalciuria under high Ca²⁺ dietary condition (described above). To investigate the functional role of claudin-14 in CaSR signaling, the inventors treated age (10-12 weeks old) and sex (male) matched claudin-14 KO mice (established on C57BL/6 strain: see Methods) and their littermate WT controls with NPS2143 or cinacalcet at a single dose of 30 mg/kg BW⁻¹ (N=5-7 for each treatment group). The temporal phase of CaSR mediated claudin-14 gene regulation (FIG. 8A-B) suggests that the urinary Ca²⁺ excretion followed the same trajectory, i.e. peaking at 2 hr but dissipating by 8 hr after NPS2143 or cinacalcet treatment.

To capture the time sensitive changes in renal function, the inventors performed 1 hr renal clearance measurements on mice infused with FITC-inulin (see Methods) starting at 2 hrs and 8 hrs respectively. At 2 hrs, NPS2143 treatment significantly increased the plasma Ca²⁺ level (PCa) to 11.15±0.21 mg/dL in WT mice (versus 10.18±0.08 mg/dL in vehicle treated animals; p<0.05; FIG. 10A), accompanied by a dramatic decrease in urinary excretion of Ca² (FE_{Ca}: 0.09±0.01% versus 0.63±0.09% in vehicle group; FIG. 10B); whereas cinacalcet significantly decreased PCa (8.44±0.27 mg/dL; p<0.05 versus vehicle; FIG. 10A) and increased FE_{Ca} (3.82±0.52%; p<0.01 versus vehicle; FIG. 10B) in WT animals. At 8 hrs, the plasma Ca²⁺ level was still significantly lower in cinacalcet group but returned to normal in NPS2143 treated animals (FIG. 10A). The urinary Ca²⁺ excretion levels at 8 hrs were not different across NPS2143, vehicle and cinacalcet groups (FIG. 10B). Renal handling of Mg²⁺ was similar to that of Ca²⁺ in WT mice. The plasma Mg²⁺ level (P_{Mg}) was significantly altered by NPS2143 and cinacalcet at 2 hrs but returned to baseline at 8 hrs (FIG. 10C). The changes in urinary excretion of Mg²⁺ (FE_{Mg}) were inversely correlated with that in P_{Mg}, which was significant at 2 hrs (p<0.05 for NPS2146 and cinacalcet versus vehicle) but dissipated at 8 hrs (FIG. 10D).

Plasma PTH levels were significantly affected by NPS2143 or cinacalcet at both 2 hrs and 8 hrs (p<0.05 versus vehicle; FIG. 10E). At 8 hrs, the PTH level in NPS2143 group started to return towards baseline (p=0.08 versus 2 hr) while cinacalcet continued to suppress plasma PTH levels through 2 hrs to 8 hrs, which likely explained the observed hypocalcemia at 8 hrs despite normocalciuria. The claudin-14 KO animals were refractory towards CaSR mediated renal Ca²⁺ regulation. At 2 hrs, NPS2143 induced hypercalcemia (PCa; FIG. 10F) and hypocalciuria (FE_{Ca}; FIG. 10G) were abolished in KO animals despite significant increases in plasma PTH levels (p<0.05 versus vehicle; FIG. 10J). Cinacalcet-induced hypocalcemia was still present in KO animals (p<0.05 versus vehicle, FIG. 10F), which appeared to have originated from extrarenal sources due to suppressed PTH levels (FIG. 10J), because their urinary Ca²⁺ excretion was not different from the vehicle group (p=0.19; FIG. 10G).

The baseline plasma Mg²⁺ level was significantly higher in KO animals compared to that in WT animals (vehicle group; P_{Mg}: 2.71-0.05 mg/dL in KO versus 2.34±0.04 mg/dL in WT; p<0.01; FIG. 10H), accompanied by a trend towards lower baseline urinary excretion of Mg²⁺ (FE_{Mg}: 11.67±2.11% in KO versus 16.59±1.31% in WT; p=0.09; FIG. 10I). This is consistent with the in vitro recordings that claudin-14 is a negative regulator of the paracellular channel reabsorbing divalent cations in the kidney (described above).

Both NPS2143 and cinacalcet dependent regulation of Pus (FIG. 10H) and FE_{Mg} (FIG. 10I) was abrogated in KO animals comparable to their effects on Ca²⁺ handling. The deregulation of urinary Ca²⁺ metabolism in claudin-14 KO animals following CaSR modulation suggests that claudin-14 is the functional “effector” underpinning the renal signaling pathway of CaSR.

### Example 12

This example illustrates transgenic overexpression of claudin-14 in the kidney induces hypercalciuria and hypermagnesiuria.

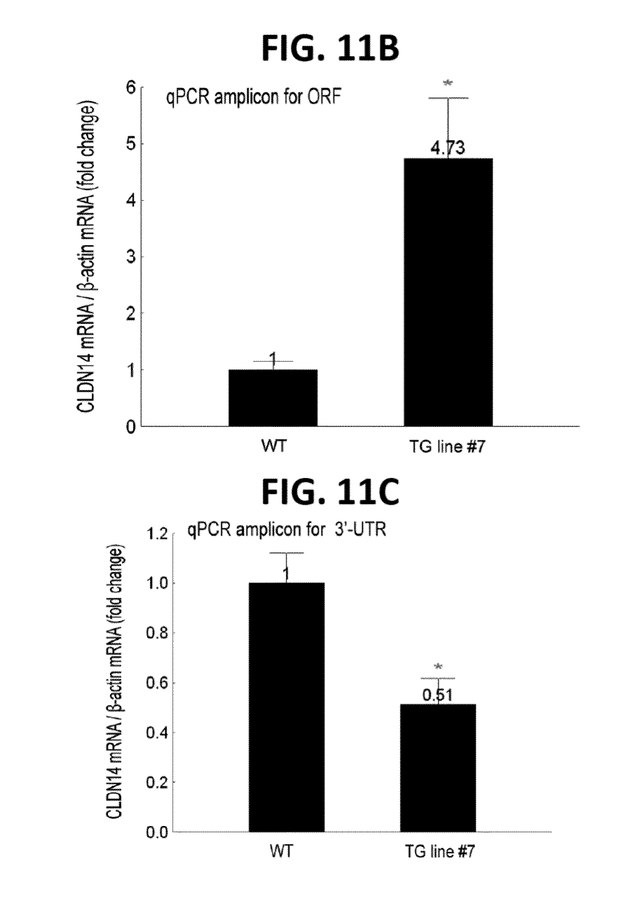

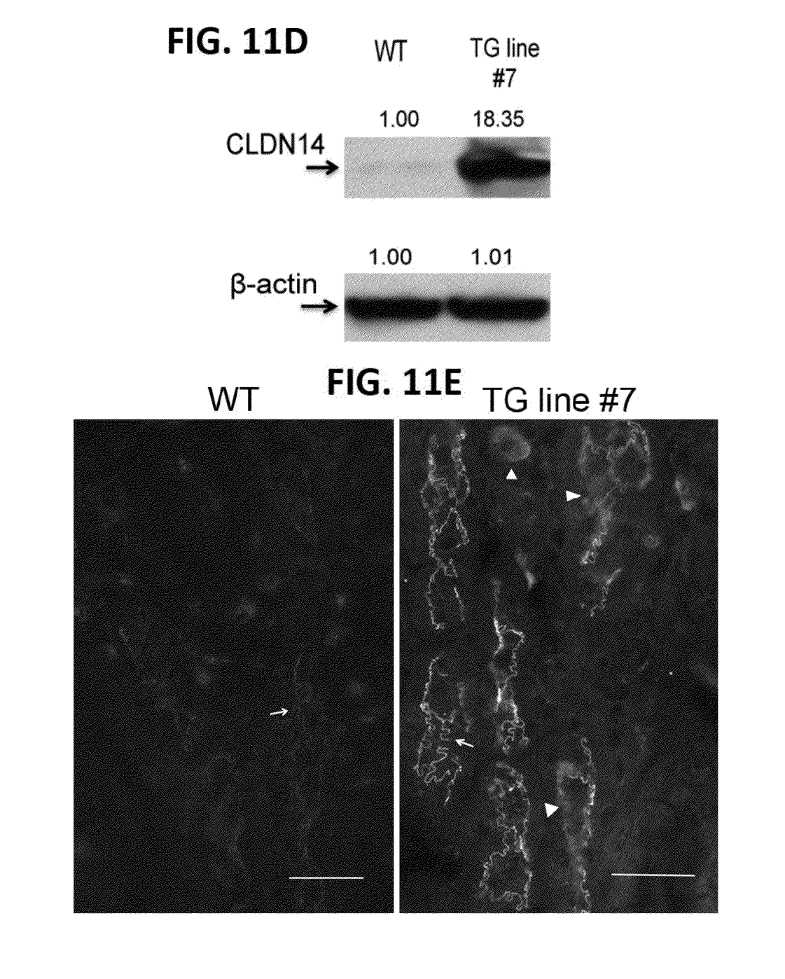

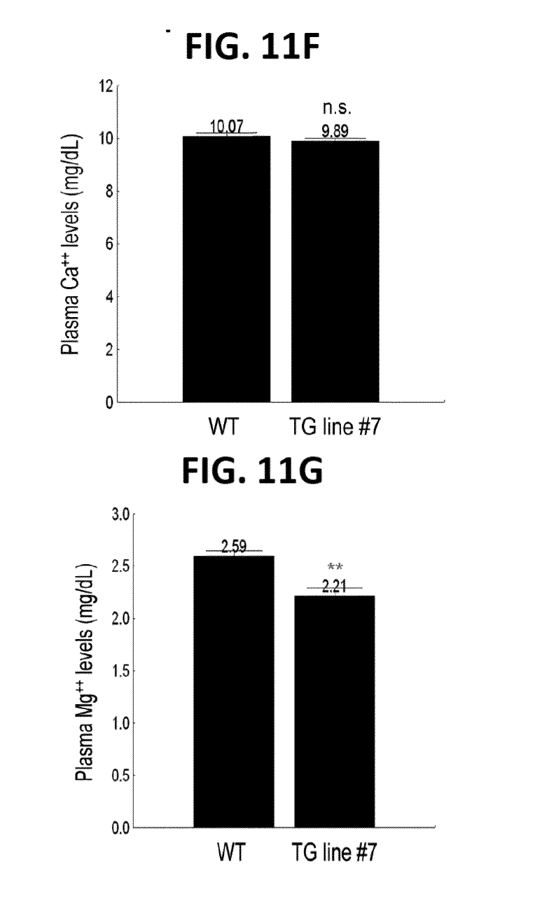

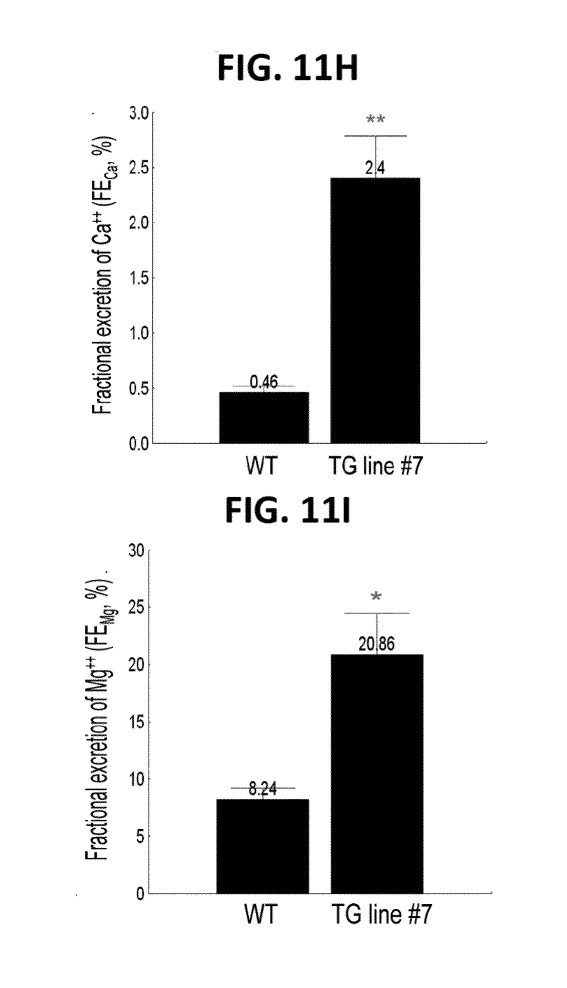

To provide in vivo evidence that manipulation of claudin-14 gene expression in the kidney per se is sufficient to cause calciuretic phenotypes, the inventors generated transgenic mouse models (established on C57BL/6x CBA hybrid background) to overexpress the claudin-14 gene selectively in the TALH epithelia of the kidney. The mouse claudin-14 gene was cloned downstream of a proven TALH-specific gene promoter—Tamm-Horsfall protein (THP; Stricklett et al., 2003) (FIG. 11A), allowing TALH-specific transgenic expression. After transgenesis (see Methods), the TALH tubules were immuno-isolated from mature hemizygous transgenic mouse kidneys for quantitative analyses of claudin-14 gene expression.

Because the transgene contained only the open reading frame (ORF) but no 5″- or 3″-untranslated region (UTR) of the claudin-14 gene, the inventors designed two pairs of primers to differentiate the transgenic from endogenous claudin-14 expression (FIG. 11A). The primer pair “qPCRorf” allowed amplifying the total claudin-14 transcripts while the primer pair “qPCR3utr” selectively amplified the endogenous claudin-14 transcripts (Table 2). The total claudin-14 mRNA level (normalized to β-actin mRNA) was significantly increased by 4.73-fold (p<0.05, n=6: FIG. 4B) in a transgenic line (TG line #7) compared to its WT littermates. The endogenous claudin-14 mRNA levels was instead significantly decreased by 49% (p<0.05, n=6 versus WT; FIG. 11C) in the same line, suggesting a feedback loop involving the CaSR signaling described elsewhere of this study. Thus, the transgenic expression on the mRNA level was estimated 9.27-fold relative to the endogenous level in WT. The claudin-14 proteins, pooled from the isolated TALH tubules of 6 animals in each group (N=6), were dramatically upregulated by 18.35-fold in TG animals compared to WT controls (FIG. 11D). Because the transgene contained no 3″-UTR of the claudin-14 gene where two microRNAs—miR-9 and miR-374 bind, the transgenic claudin-14 transcript would be more efficient in translation compared to its endogenous transcript, therefore causing the observed super-regulation on the protein level.

In WT mouse kidneys, claudin-14 was faintly immunostained in the TALH tubules showing interdigitated TJ pattern (arrow; FIG. 11E). In transgenic mouse kidneys, claudin-14 staining was profoundly strengthened in tight junctions of the TALH tubules (arrow; FIG. 11E), with occasional diffuse staining spotted in the subapical region of some TALH cells (arrowhead; FIG. 11E) suggestive of transgenic protein saturation. To investigate the functional effects of claudin-14 overexpression in the kidney, the inventors performed 24 h urinalysis on age (10-12 weeks old) and sex (male) matched claudin-14 TG mice and their littermate WT controls. The plasma Ca²⁺ level in TG mice was well defended (FIG. 11F; Table S2), but their circulating Mg²⁺ level was significantly lower than WT controls (P_{Mg}; TG: 2.21±0.08 mg/dL versus WT: 2.59±0.05 mg/dL; p<0.01, n=6; FIG. 11G: Table S2). The fractional excretion rates for Ca²⁺ (FE_{Ca}) and Mg² (FE_{Mg}) in TG animals were profoundly increased by 5.22-fold (p<0.01, n=6; FIG. 11H; Table S2) and 2.53-fold (p<0.05, n=6; FIG. 11I; Table S2) respectively compared to WT animals, indicating a direct renal tubular defect.

The glomerular filtration rate (GFR) based on creatinine clearance (Table S2) was not significantly different between TG and WT animals, nor was the urinary volume (UV) (Table S2). The phenotypes of plasma and urine electrolyte abnormalities of TG line #7 were recapitulated in a second transgenic line TG #24, whose total claudin-14 transcript level was 5.68-fold higher than WT (p<0.05, n=6).

These results have established the in vivo function of claudin-14 in the kidney that negatively regulates the paracellular divalent reabsorption, sharing characteristics with the renal phenotypes of claudin-16 KO and claudin-19 KO animals.

### Example 13

This example illustrates that microRNA transcription but not the claudin-14 promoter is directly regulated by CaSR.

MiR-9 is transcribed from 3 genomic loci in both human and mouse: miR-9-1, miR-9-2 and miR-9-3 (Ma et al., 2010). Human miR-374 has two isoforms—a and b, both sharing the same seed sequence and each having its own genomic locus. Mature human miR-374b is identical to mouse miR-374 that is transcribed from a single genomic locus, while human miR-374a has no mouse homologue.

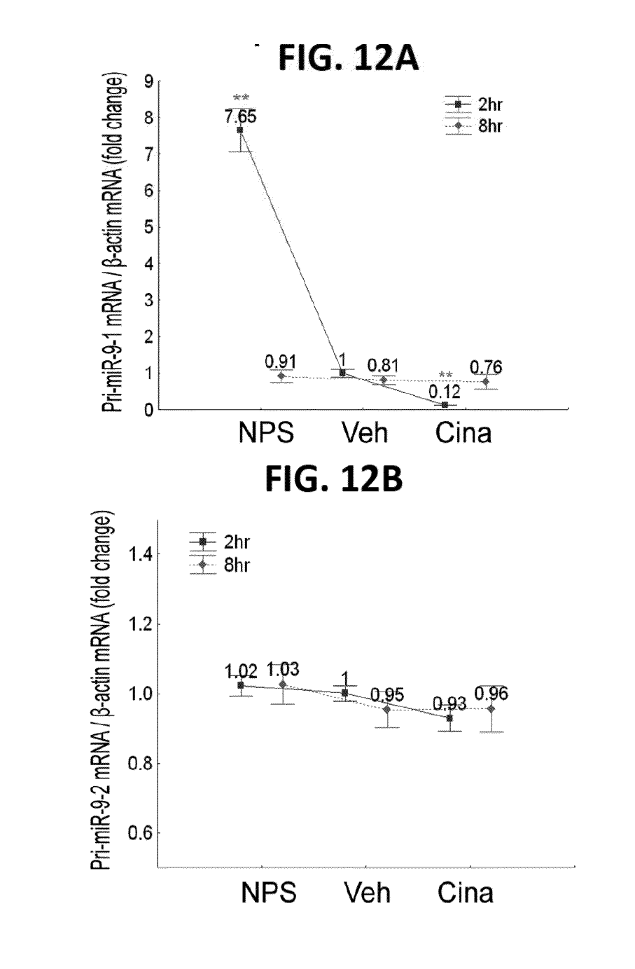

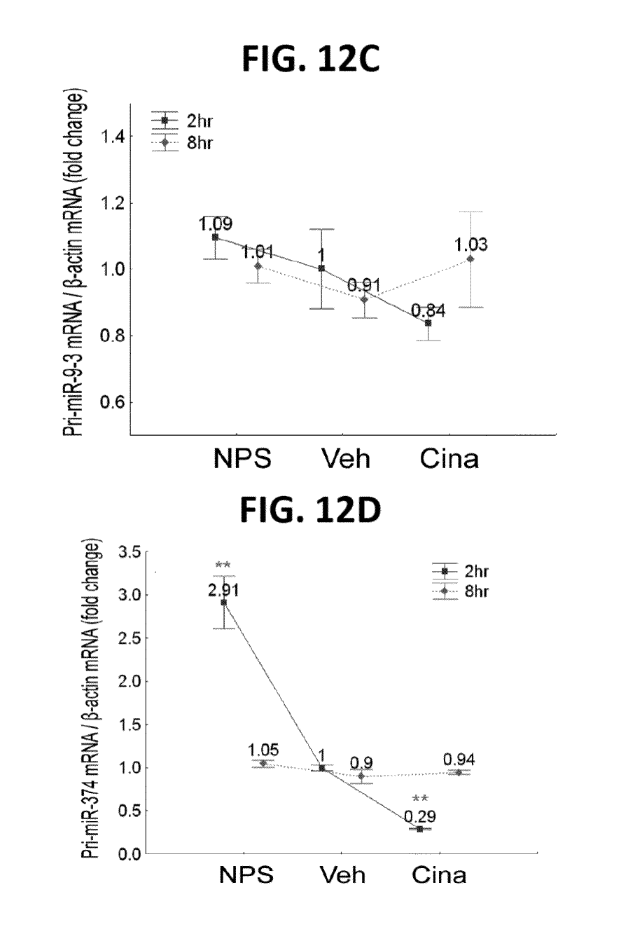

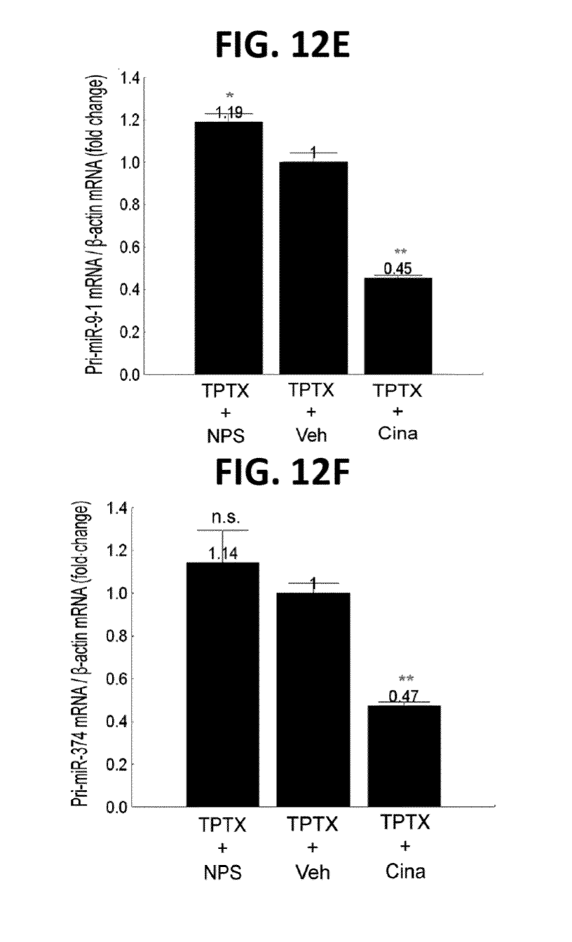

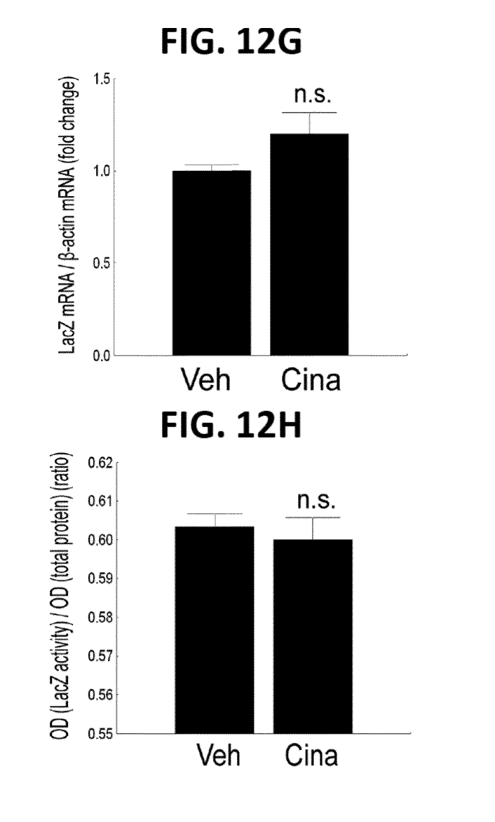

To measure the transcriptional level for microRNA gene in vivo in the kidney, the inventors designed primers (see Methods; Table 2) to amplify the pri-miRNA molecule, the original transcript for microRNA gene that had not yet been subjected to any nuclear or cellular processing. Because both miR-9 and miR-374 had broader localization profiles along the nephron, the inventors isolated the TALH tubular cells for pri-miRNA analyses from the mouse kidney described elsewhere in this study. WT mice (strain: C57BL/6) were treated with NPS2143 or cinacalcet as described for claudin-14 analyses and assayed for pri-miRNA levels in isolated TALH cells at 2 hrs and 8 hrs respectively. Among the three miR-9 genes, miR-9-1 was the most sensitive to CaSR modulation: showing a 7.65-fold increase in its pri-miRNA transcript level 2 hrs after single-dose 30 mg/kg BW⁻¹ NPS2143 treatment (p<0.01, n=4 versus vehicle; FIG. 12A); and 88% decrease 2 hrs after a 30 mg/kg BW⁻¹ cinacalcet treatment (p<0.01, n=4 versus vehicle; FIG. 12A). By 8 hrs, the changes in pri-miR-9-1 levels induced by NPS2143 or cinacalcet had dissipated completely (FIG. 12A). Neither pri-miR-9-2 (FIG. 12B) nor pri-miR-9-3 level (FIG. 12C) was significantly altered by NPS2143 or cinacalcet at 2 hrs or 8 hrs. NPS2143 and cinacalcet also profoundly regulated pri-miR-374. At 2 hrs, NPS2143 upregulated pri-miR-374 by 2.91-fold while cinacalcet downregulated it by 71% (p<0.01, n=4 versus vehicle; FIG. 12D). Similar to pri-miR-9-1, pri-miR-374 levels were not different from control 8 hrs after NPS2143 or cinacalcet treatment (FIG. 12D). The expression levels of miR-9-1 and miR-374 genes were both inversely correlated with that of claudin-14 gene in the kidney, compatible with the silencing role of microRNA in regulating its target gene.

miR-9-1 RNA
(SEQ ID NO: 40)
CGGGGUUGGUUGUUAUCUUUGGUUAUCUAGCUGUAUGAGUGGUGUGGAGU
CUUCAUAAAGCUAGAUAACCGAAAGUAAAAAUAACCCCA
miR-374 RNA
(SEQ ID NO: 41)
ACUCGGAUGGAUAUAAUACAACCUGCUAAGUGUCCUAGCACUUAGCAGGU
UGUAUUAUCAUUGUCCGUGUCU

The inventors investigated whether CaSR regulation of pri-miRNA depended upon PTH secretion. In TPTX treated mice (Methods), cinacalcet at a single dose of 30 mg/kg BW⁻¹ significantly reduced pri-miR-9-1 and pri-miR-374 levels by 55% and 53% respectively at 2 hrs (p<0.01, n=5 versus vehicle; FIG. 12E-F). NPS2143 at 30 mg/kg BW⁻¹ significantly increased the pri-miR-9-1 level by 19% in TPTX treated animals (p<0.05, n=5 versus vehicle; FIG. 12E) but failed to elicit a significant change in pri-miR-374 level, likely due to severe hypocalcemia (FIG. 9D) in TPTX animals that had already experienced several-fold higher basal levels of pri-miR-9-1 and pri-miR-374 compared to sham-operated animals.

The inventors also sought for alternative mechanisms for claudin-14 gene regulation. Because the claudin-14 KO mouse was generated with a lacZ reporter gene replacing the last exon of claudin-14 gene (including the coding region and 3″-UTR) (Ben-Yosef et al., 2003), the expression of lacZ gene would provide a faithful measurement for endogenous claudin-14 promoter activity regardless of any microRNA based regulation. The claudin-14^{+/lacZ} mice were treated with cinacalcet or NPS2143 as described for WT mice. At 2 hrs after single-dose 30 mg/kg BW⁻¹ treatment, cinacalcet caused no change in lacZ mRNA levels (normalized to β-actin mRNA; n=4: FIG. 12G) or β-galactosidase activity (normalized to total soluble proteins; n=4; FIG. 12H), assayed in claudin-14^{+/lacZ} mouse kidneys and isolated TALH cells respectively, nor did NPS2143 at the same dosage and time point. These data indicate that CaSR regulates claudin-14 gene expression not through cis-acting element. i.e. its promoter but through trans-acting factors, i.e. miR-9 and miR-374.

### Example 14

This example illustrates that a calcineurin inhibitor abrogates CaSR mediated regulation of claudin-14, microRNAs and urinary Ca²⁺ excretion.

The calcineurin inhibitor cyclosporine-A induces hypomagnesemia and hypercalciuria in laboratory animals, previously thought to occur because of disturbed TRPV5 and TRPM6 channel expression in the kidney (Nijenhuis et al., 2004). A recent study has found that cyclosporine-A reduced the paracellular but not transcellular divalent cation transport in cultured mouse TALH cells in vitro (Chang et al., 2007). To determine how cyclosporine-A affected claudin-14 gene expression and renal Ca²⁺ handling in vivo, the inventors pre-treated WT mice (strain: C57BL/6) with cyclosporine-A (Sandimmune) at a single I.P. dose of 25 mg/kg BW⁻¹ per day for 6 consecutive days followed by NPS2143 or cinacalcet treatment on the 6th day (Methods).

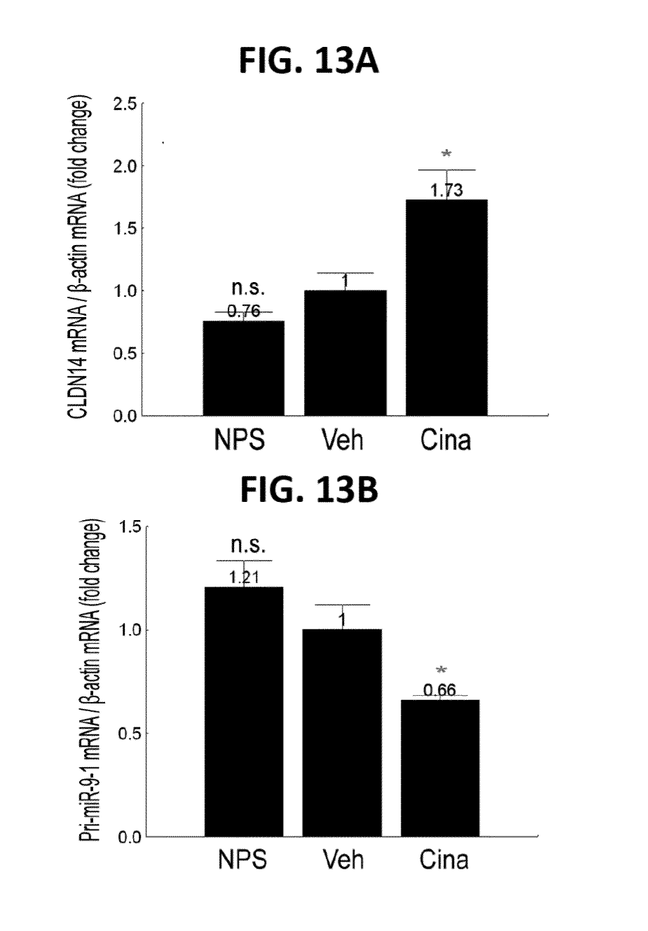

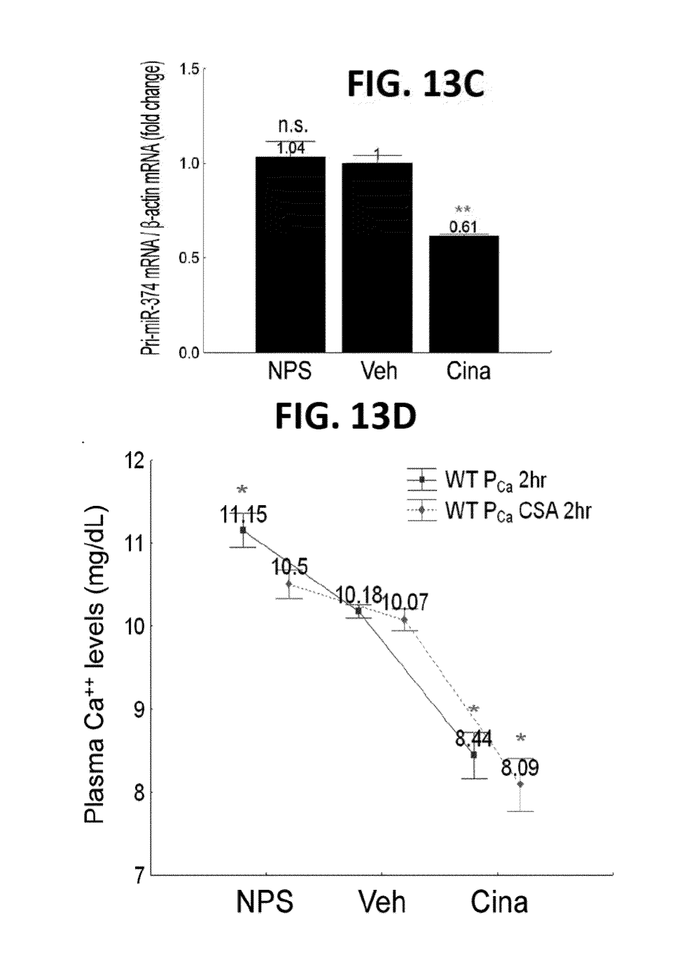

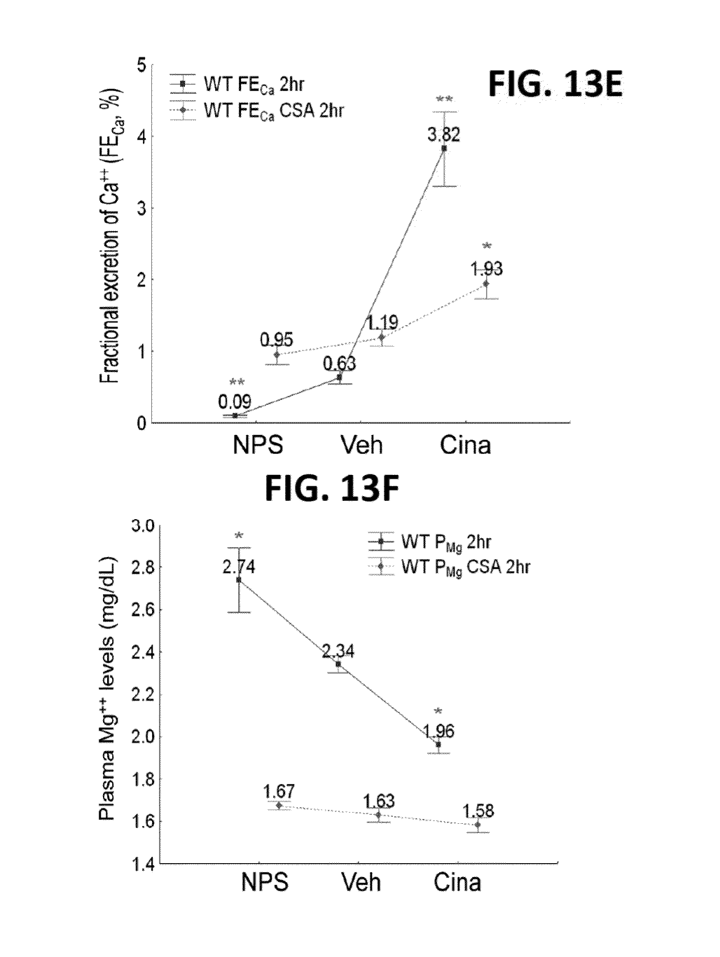

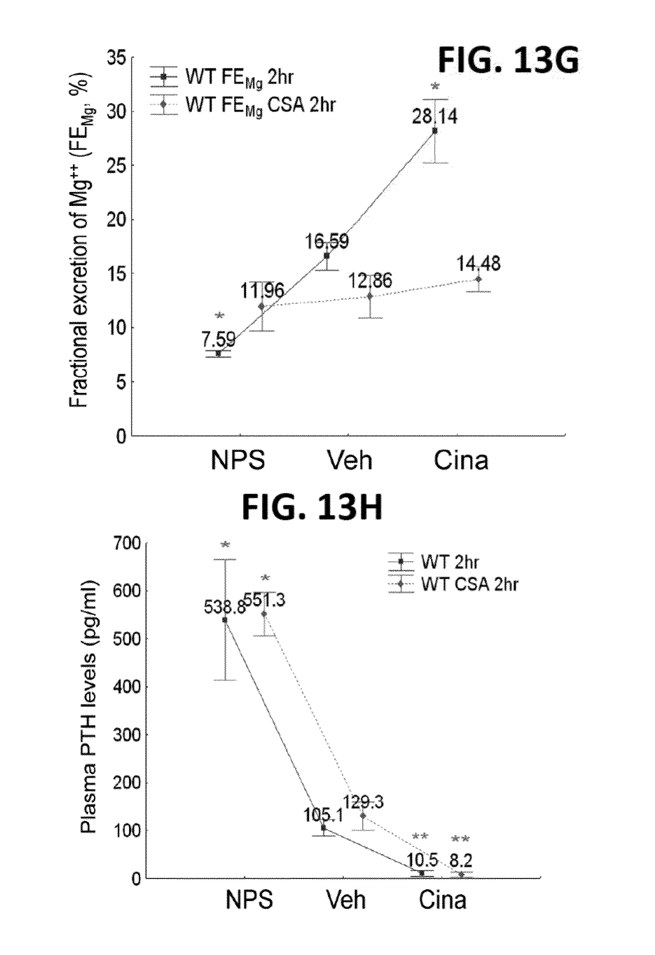

Cyclosporine-A pre-treatment did not affect the basal level of claudin-14, pri-miR-9-1 or pri-miR-374 in the TALH of the kidney. Calcimimetic and calcilytic effects were, however, significantly attenuated by cyclosporine-A pre-treatments. At 2 hrs after single-dose 30 mg/kg BW⁻¹ treatment, NPS2143 failed to elicit any significant change in claudin-14 (n=5 versus vehicle: FIG. 13A), pri-miR-9-1 (FIG. 13B) or pri-miR-374 mRNA levels (FIG. 13C) in cyclosporine-A pre-treated animals. Cinacalcet, at the same dosage and time point, still significantly increased claudin-14 mRNA levels by 1.73-fold (p<0.05, n=5 versus vehicle: FIG. 13A) and decreased pri-miR-9-1 and pri-miR-374 mRNA levels by 34% (p<0.05; FIG. 13B) and by 39% (p<0.01; FIG. 13C) respectively in cyclosporine-A pre-treated animals, nevertheless, its regulatory effects were much lower than those captured for animals without cyclosporine-A treatment (FIG. 8B; FIGS. 12A and 12D).

The inventors investigated whether the calciuretic response to CaSR modulation was also attenuated by cyclosporine-A. Because the control animals for cyclosporine-A treatment (injected with Sandimmune solvent; see Methods) showed no difference in their plasma and urinary electrolyte levels from WT animals, their physiological data were not shown, instead the WT animal data presented in FIG. 10 were superimposed onto the calciuretic and magnesiuretic curves of cyclosporine-A treated animals (FIG. 13D-H). Cyclosporine-A significantly increased the basal level of urinary Ca²⁺ excretion (FE_{Ca}: 1.19±0.12% versus 0.63±0.09% in WT; p<0.05, n=5-7; FIG. 13E) without affecting the basal plasma Ca⁺⁺ level (P_{Ca}; FIG. 13D). The basal level of P_{Mg} was dramatically reduced by cyclosporine-A to 1.63±0.03 mg/dL compared with 2.34±0.04 mg/dL in WT (p<0.05, n=5-7; FIG. 13F), accompanied by normomagnesiuria (FIG. 13G), hence pointing to an extrarenal origin of Mg²⁺ loss. At 2 hrs, the NPS2143 effects on PCa (FIG. 13D) and FE_{Ca} (FIG. 13E) were abolished by cyclosporine-A, while the cinacalcet effects were maintained (FIG. 13D-E) even though cinacalcet induced hypercalciuria was attenuated to a level of 1.93-fold higher than vehicle in cyclosporine-A pre-treated animals (p<0.05, n=5-7: FIG. 13E). Both NPS2143 and cinacalcet dependent regulation of P_{Mg} (FIG. 13F) and FE_{Mg} (FIG. 13G) was completely abrogated by cyclosporine-A despite an intact PTH response towards both drugs (FIG. 13H) at 2 hrs. These results show a role for calcineurin inhibitor that antagonizes the extracellular Ca²⁺ signaling in the kidney causing deranged claudin-14 expression and urinary Ca²⁺ excretion.

### Example 15

This example illustrates that CaSR signaling involves NFAT dependent regulation of microRNA promoters.

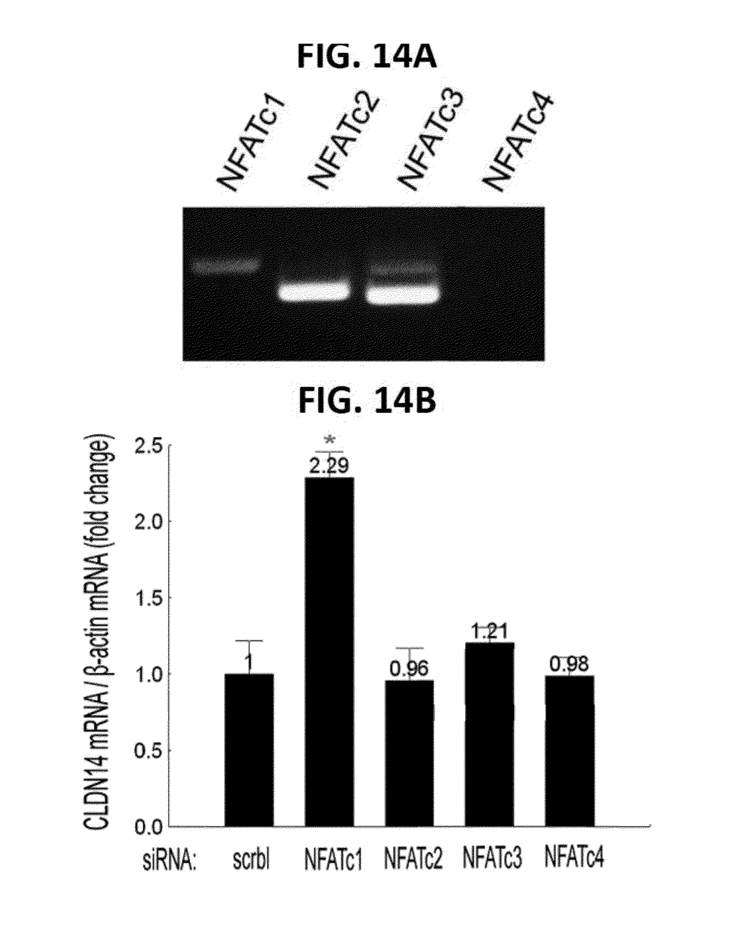

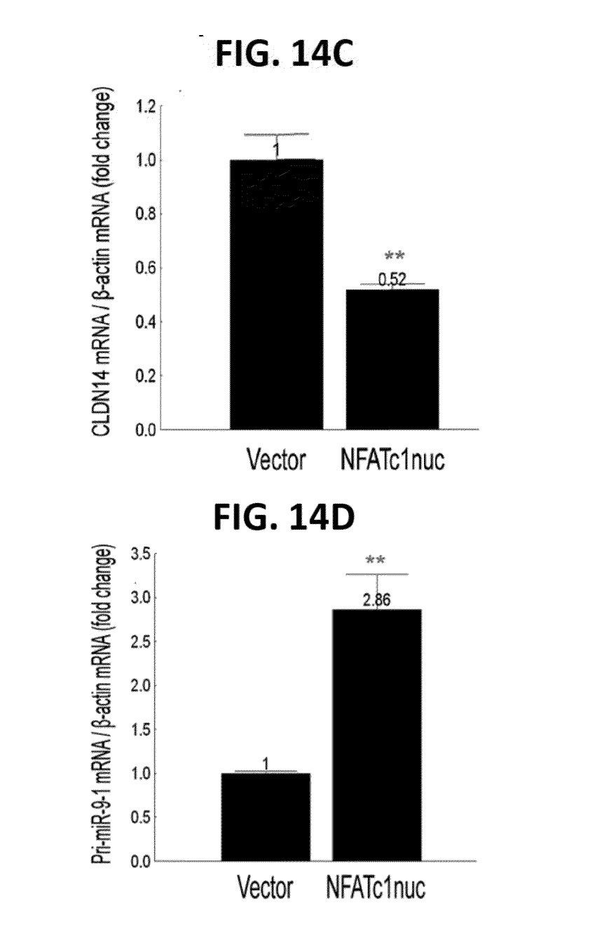

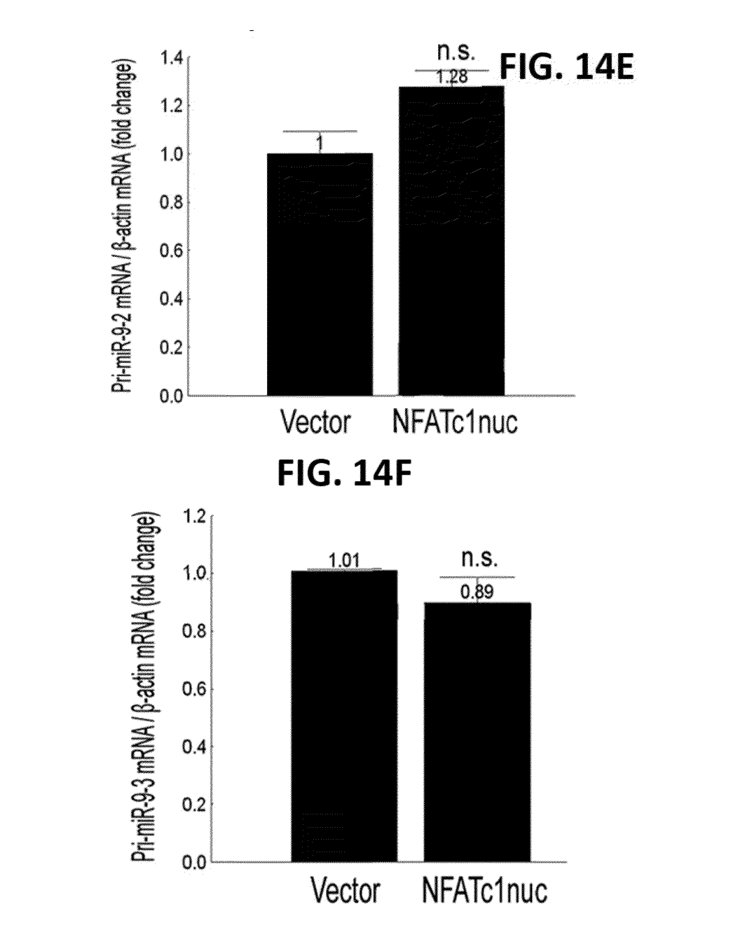

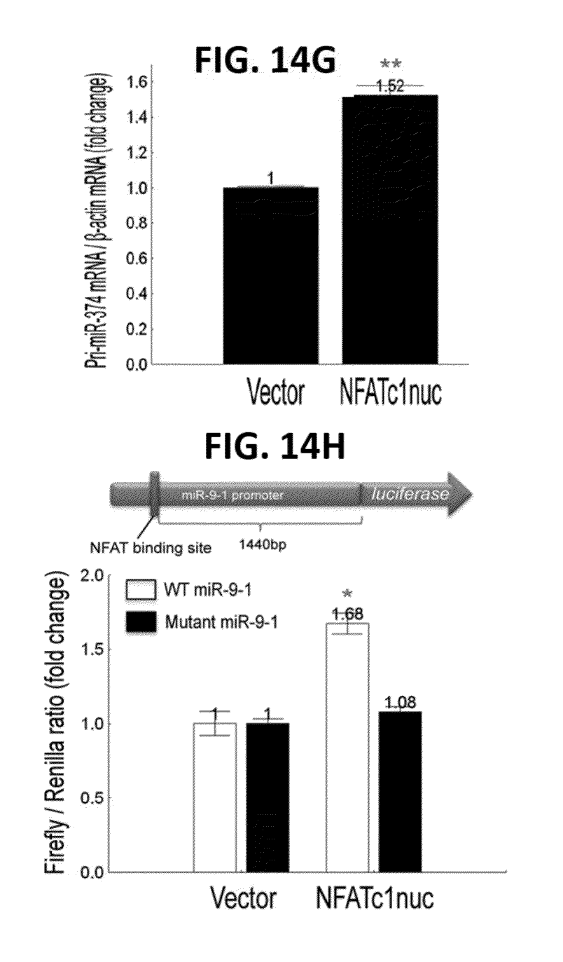

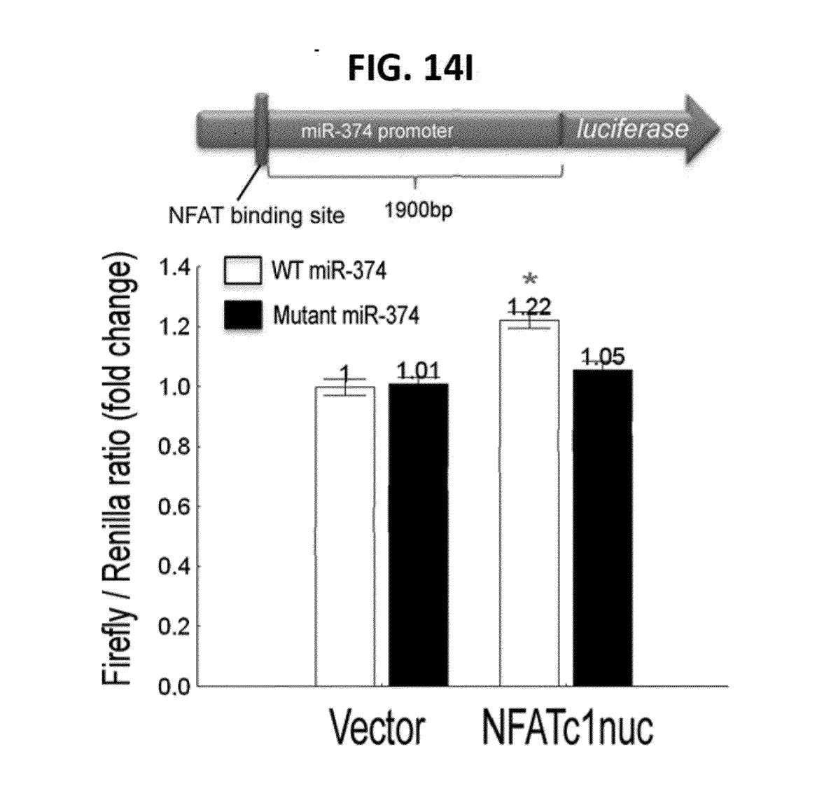

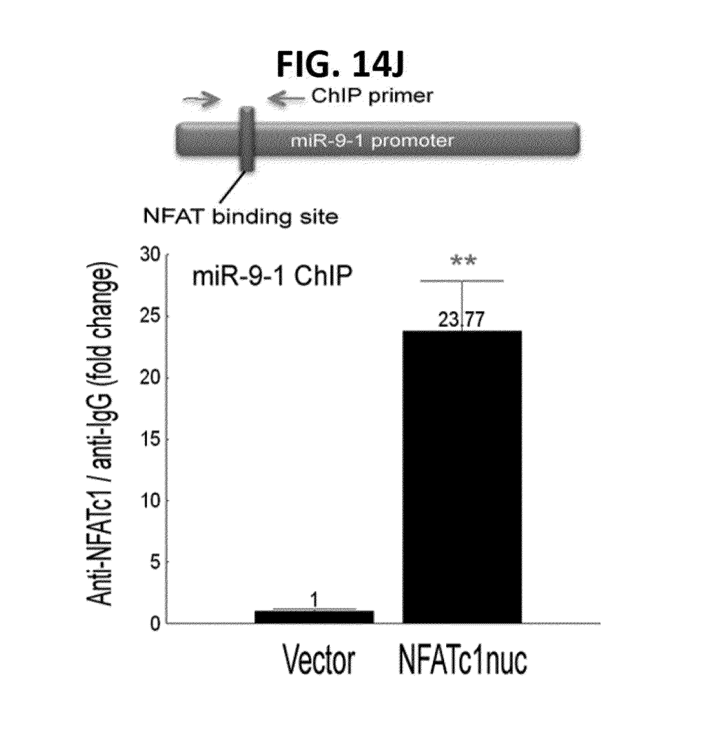

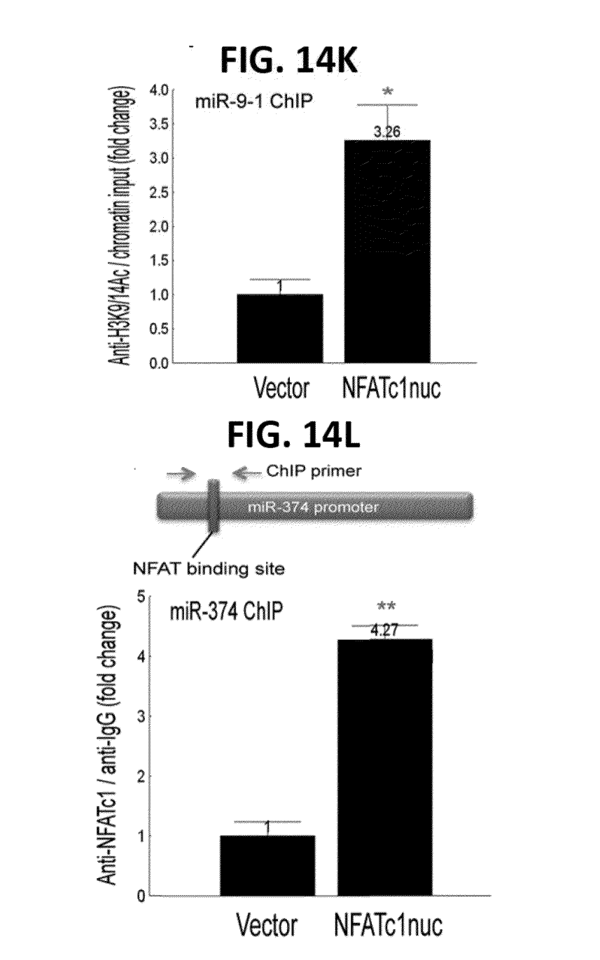

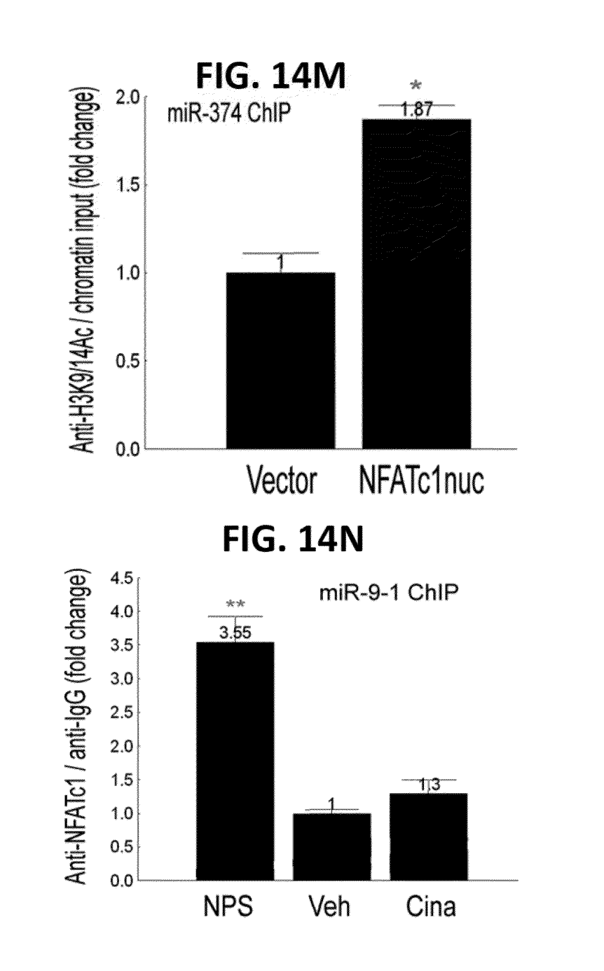

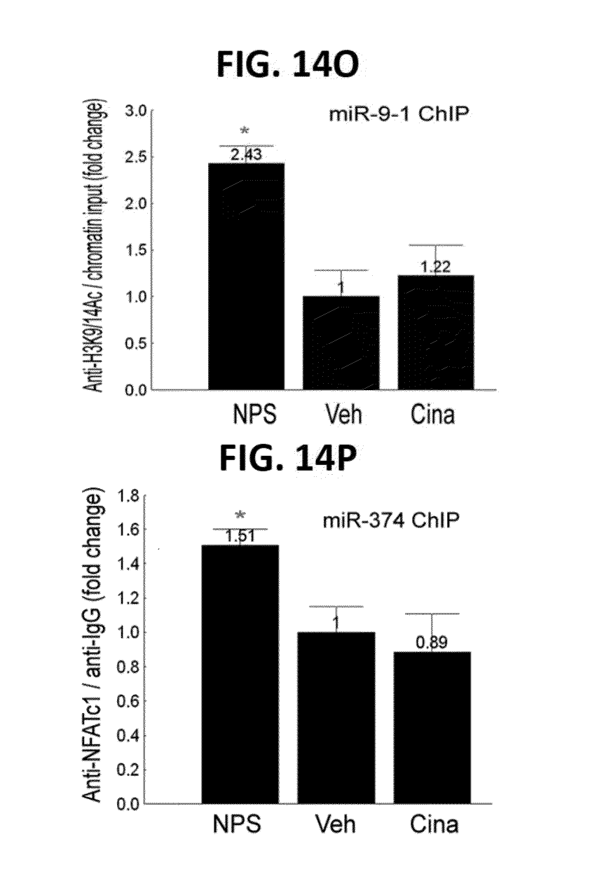

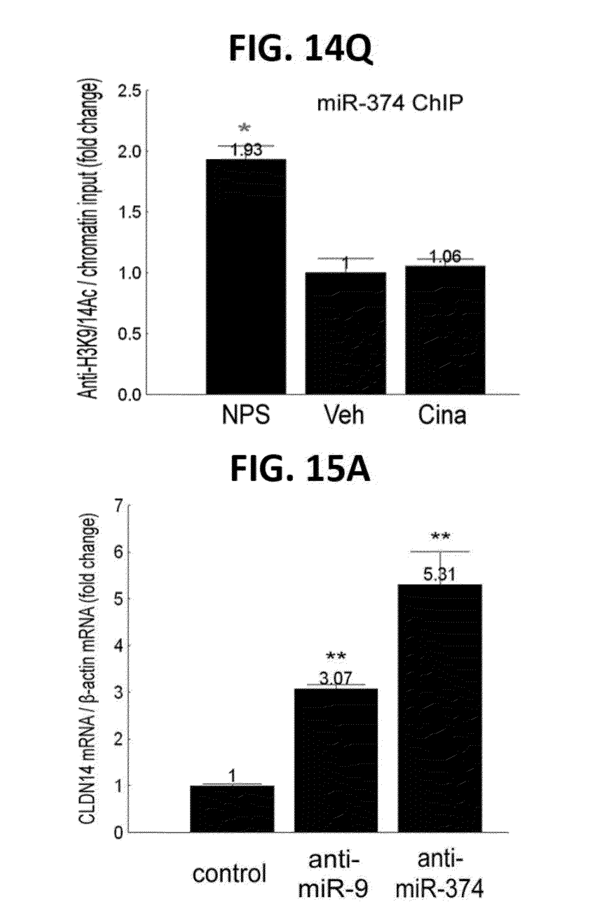

Calcineurin is a protein phosphatase that regulates a class of transcriptional factors known as NFATs. NFATs transduce the calcineurin signals to gene regulation in the nucleus (Crabtree and Olson, 2002). The TALH cell is sensitive to calcineurin signaling. Cyclosporine regulates the gene expression of NKCC2 and prostaglandin E2 in the mouse and rat TALH cells (Chang et al., 2005; Esteva-Font et al., 2007). The inventors investigated which isoform of NFATs was expressed in the TALH of the kidney. With primers designed against the four cellular isoforms of NFATs (NFATc 1-4; Table 2), the inventors detected predominant gene expression of NFATc1, c2 and c3 but not c4 in immuno-isolated mouse TALH cells (FIG. 14A). The inventors also investigated which NFAT played a functional role in claudin-14 gene regulation in the TALH cells. To selectively knock down NFAT gene expression, the inventors transfected a pre-validated siRNA reagent (see Methods) that contained a pool of three target-specific 21 nt siRNA duplexes designed against each mouse NFAT gene into the primary cultures of freshly isolated mouse TALH cells. A scrambled siRNA duplex was transfected as negative control. Compared to scrb1-siRNA, transfection with NFATc1-siRNA significantly increased the transcriptional level of claudin-14 by 2.29-fold (p<0.05, n=4; FIG. 14B) in primary TALH cells, while knockdown of other NFATs were without significant effect.

The inventors transfected a constitutively active mutant of NFATc 1 (with 21 Serine to Alanine mutations; named NFATc1nuc; see Methods; Winslow et al., 2006) into primary TALH cells. Transfection with NFATc1nuc significantly decreased the transcriptional level of claudin-14 by 48% (p<0.01, n=3 versus vector control; FIG. 14C), accompanied by 2.86-fold and 1.52-fold increases in miR-9-1 and miR-374 transcriptional levels respectively (p<0.01, n=3; FIGS. 14D and 14G). The transcription of miR-9-2 or miR-9-3 gene was not altered by NFATc1nuc (FIG. 14E-F). The inventors searched the miR-9-1 and miR-374 gene promoters for presence of NFAT consensus-binding sites ([A/T]GGAAA[A/N][A/T/C]N) (Kuwahara et al., 2006). In the mouse miR-9-1 promoter, there was a NFAT binding site (AGGAAAAT) 1440 bp upstream of the miR-9-1 hairpin sequence (FIG. 14H), which was also present in human miR-9-1 promoter (1660 bp upstream of human miR-9-1 hairpin). In the mouse miR-374 promoter, a similar NFAT binding site was found 1900 bp upstream of miR-374 hairpin (FIG. 14I) that was conserved in human miR-374 promoter. To determine if NFATc1 was able to regulate miR-9-1 and miR-374 promoters independent of cell context and chromatin environment, the inventors cloned the miRNA promoters (−2500 bp-0 bp; containing the NFAT binding site) upstream of a firefly luciferase reporter gene (FIGS. 14H-I) and co-transfected with NFATc1nuc into a model cell line—HEK293 that expressed no endogenous CaSR-claudin-14 pathway. A 68% increase in firefly luciferase activity (p<0.05, n=3; normalized to renilla luciferase activity; FIG. 14H) was found for miR-9-1 promoter; a 22% increase (p<0.05, n=3; FIG. 14I) for miR-374 promoter, compared with vector transfection. Deletion of the NFAT binding site in miR-9-1 and miR-374 promoters (mutant promoter) completely abolished the observed NFATc 1 effect (FIG. 14H-I).

The inventors sought for evidence of NFATc1 regulating endogenous miR-9-1 and miR-374 promoters in native TALH cells and in presence of intact chromatin. Chromatin immunoprecipitation (ChIP) was carried out in mouse primary TALH cells transfected with NFATc1nuc or its vector control. ChIP primers were designed to span the NFAT binding sites (FIGS. 14J and 14L; Table 2). NFATc1nuc transfection induced a dramatic 23.77-fold increase in its enrichment over miR-9-1 promoter (with anti-NFATc1 antibody; p<0.01, n=3 versus vector; normalized to anti-IgG antibody; FIG. 14J) and a 4.27-fold increase over miR-374 promoter (p<0.01, n=3 versus vector; FIG. 14L). The acetylation levels on histone H3 Lysine-9 (H3K9) and Lysine-14 (H3K14), well-documented chromatin markers for transcriptional activation, were concomitantly increased by 3.26-fold (with anti-H3K9/14Ac antibody; p<0.05, n=3 versus vector; normalized to chromatin input; FIG. 14K) and 1.87-fold (p<0.05, n=3 versus vector: FIG. 14M) respectively over the miR-9-1 and miR-374 promoter regions containing NFAT binding sites. The inventors established a direct regulatory role for NFATc 1 in microRNA transcription.

The inventors questioned if CaSR signaling induces NFATc1 dependent regulation of microRNA in vivo. To manipulate CaSR signaling in vivo in the kidney, the inventors treated WT mice with NPS2143 or cinacalcet at a single dose of 30 mg/kg BW⁻¹ as described elsewhere in this study, isolated the TALH cells at 2 hrs following each drug treatment and performed ChIP analyses on isolated TALH cells from each mouse. NPS2143 treatment significantly increased the fold enrichment of NFATc 1 over the promoter regions of miR-9-1 and miR-374 containing NFAT binding sites, by 3.55-fold (p<0.01, n=5 versus vehicle: FIG. 14N) and 1.51-fold (p<0.05, n=5 versus vehicle; FIG. 14P) respectively, while cinacalcet treatment was without significant effect. The acetylation levels on H3K9 and H3K14 surrounding the same miR-9-1 and miR-374 promoter regions were also increased by NPS2143 but not cinacalcet in mouse TALH cells, to 2.43-fold and 1.93-fold higher (p<0.05, n=5; FIGS. 14O and 14Q) than vehicle level respectively. These data have identified a transcriptional factor—NFATc1 that mediates CaSR signaling to transcriptional regulation of miR-9-1 and miR-374 genes through epigenetic mechanism.

### Example 16

This example illustrates that anti-microRNAs can treat hypercalcemia.

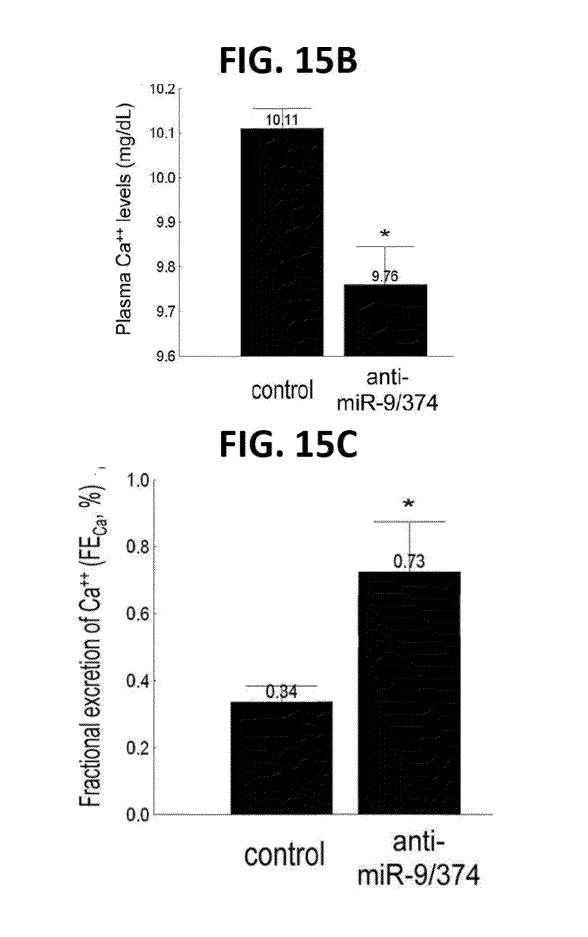

Anti-miR-9 and anti-miR-374 were tested in freshly isolated mouse TALH tubular cells. Transfection of 50 pmol anti-miR-9 or anti-miR-374 significantly increased the expression level of claudin-14 in mouse TALH cells by 3.07-fold and 5.31-fold respectively (p<0.01, n=5 versus control: (FIG. 15A). A 1:1 mixture of anti-miR-9 and anti-miR-374 was then injected to adult mice (8-10 weeks old; strain: C57BL/6) intravenously at the dose of 2.5 mg/kg for each anti-miR. The control anti-miR was injected at the same dose as negative control. Injected mice were analyzed for plasma and urinary Ca²⁺ levels in metabolic cages. Anti-miR-9/374 significantly lowered the plasma Ca²⁺ levels in treated mice (p<0.05; n=5 versus control; FIG. 15B), accompanied by a 2.15-fold increase in their urinary excretion levels of Ca²⁺ (p<0.05; n=5 versus control; FIG. 15C).

These data have provided evidence that manipulation at the level of microRNA can affect renal handling of calcium metabolism, in line with the signaling cascade of CaSR-NFAT-microRNA-claudin-14 the inventors discovered from hypercalcemic animals. An immediate clinic application of this technology will be to treat hypercalcemic patients without inducing unwanted changes in circulating PTH levels.

### The Following Methods are Applicable to Examples 17-21

### Reagents, Antibodies, Cell Lines and Animals

The following antibodies were used in this study: rabbit polyclonal anti-THP (Biomedical Technologies): rabbit polyclonal anti-CLDN14 (against RAPSVTSAAHSGYRLNDYV) (SEQ ID NO: 39); rabbit polyclonal anti-CLDN16 (against SYSAPRTETAKMYAVDTRV) (SEQ ID NO: 5); rabbit polyclonal anti-CLDN19 (against NSIPQPYRSGPSTAAREYV) (SEQ ID NO: 6). Human HEK293 cells were cultured in DMEM (Invitrogen) supplemented with 10% FBS, penicillin/streptomycin, and 1 mM sodium pyruvate. WT C57BL/6 mice were from Charles River Laboratory. The CLDN14^{+/lacZ} reporter/knockout mice were back-crossed to C57BL/6 background for 7 generations.

### Pharmacological Manipulation in Experimental Animals

SAHA and TsA were dissolved in 5% (w/v) ethanol/0.9% saline solution and fed to mice with gavage syringe. Furosemide was dissolved in 50% (w/v) DMSO/0.9% saline solution and I.P. injected to animals. All animals had free access to water and were housed under a 12 hr light cycle. Blood samples were taken by cardiac puncture rapidly after initiation of terminal anesthesia and centrifuged at 4° C. for 10 min. Kidney were dissected out and immediately frozen at −80° C.

### Animal Metabolic Studies

Mice were housed individually in metabolic cages (Harvard Apparatus) with free access to water and food for 24 hr. Urine was collected under mineral oil. The plasma and urine Ca⁺⁺ and Mg⁺⁺ levels were determined with atomic flame absorption spectrophotometer (PerkinElmer). The creatinine levels were measured with an enzymatic method that was independent of plasma chromogens. (Gong Y, et al., 2013) The fractional excretion of electrolytes was calculated using the following equation FEᵢₒₙ=V×Uᵢₒₙ/(GFR×Pᵢₒₙ), where GFR was calculated according to the clearance rate of creatinine (GFR=V×U_{creatinine}/P_{creatinine}).

### Establishing Primary Cultures of Mouse Thick Ascending Limb Cells

An immunomagnetic separation method was used to isolate the TALH cells from the mouse kidney. (Gong Y, et al. 2012 and Gong Y, et al. 2013) Antibodies against the TALH cell specific surface antigen, Tamm-Horsfall protein, were coated onto the paramagnetic polystyrene beads (Dynabeads M-280; Dynal), allowing immunoprecipitation of the TALH cells from collagenase digested mouse kidneys. The isolated cells were plated in DMEM medium supplemented with 10% FBS, penicillin/streptomycin, and 1 mM sodium pyruvate for 16 hours, followed by immediate treatment.

### Real-Time PCR Quantification

Total RNA including microRNA was extracted using Trizol (Invitrogen). Cellular mRNA was reverse transcribed using the Superscript-III kit (Invitrogen), followed by real-time PCR amplification using SYBR Green PCR Master Mix (Bio-Rad) and Eppendorf Realplex2S system. The design of claudin-14 primers was described before by Gong et al., 2012. The design of pri-miRNA primers and ChIP primers was according to Gong et al., 2013. Results were expressed as 2^{-ΔCt} values with ΔCT=Ct_{gene}−Ct_{β-actin}.

### Chromatin Immunoprecipitation (ChIP)

Freshly isolated TALH cells or primary TALH cell cultures were cross-linked in 1% paraformaldehyde (pH7.0) for 10 min at room temperature. The cross-linked cells were lysed following to instruction of MAGnify ChIP system (Invitrogen). Chromatin was sonicated into 500 bp fragments with Bioruptor UCD-200 (High power, 15 cycles of 30 seconds ON, 30 seconds OFF; Diagenode). Sheared chromatin was precipitated with antibody-bound Dynabeads (Invitrogen). Following reverse crosslink and DNA extraction, antibody-bound DNA was eluted and analyzed with real-time PCR. Two criteria for normalization were applied: fold enrichment over anti-IgG background signal and input control over chromatin input signal.

### Immunolabeling and Fluorescence Microscopy

For viewing claudin localization in the kidney, fresh cryostat sections (10 μm) were fixed with cold methanol at −20° C., followed by blocking with PBS containing 10% FBS, incubation with primary antibody (1:300) and FITC-labeled secondary antibody (1:200). After washing with PBS, slides were mounted with Mowiol (Calbiochem). Epifluorescence images were taken with a Nikon 80i photomicroscope equipped with a DS-Qi1Mc digital camera. All images were converted to TIFF format and arranged using Photoshop CS4 (Adobe).

### Antagomir Treatments

Antagomirs for miR-9, miR-374, and scrambled control miRNA were synthesized by Exiqon using locked nucleic acids (LNAs) using the following sequences: Anti-miR-9: A*T*+A*C*A*+G*C*T*+A*G*A*+T*A*A*+C*C*A*+A*A*G; Anti-miR-374: A*C*+T*T*A*+G*C*A*+G*G*T*+T*G*T*+A*T*T*+A*T*A; where DNA base: G, A, T, C: LNA™ base: +G, +A, +T, +C; Phosphorothioated DNA base: G*, A*, T*, C*. In vitro, 30 pmol of each antagomir was transfected to the primary cultures of TALH in 12-well culture dishes using Lipofectamine 2000 (Invitrogen). In vivo, each antagomir was mixed with the in vivo-jetPEI™ Delivery Reagent (VWR) according to the manufacturer's guideline, followed by I.P. injection to animals at the dose of 2.5 mg/kg BW⁻¹.

### Patients

Eight idiopathic calcium stone formers and seven healthy controls were studied. Stone formers and controls were enrolled in the year of 2013 in the Outpatient clinics of the Italian Centers participating to the GENIAL Network. All patients had idiopathic kidney stones that were radio-opaque and/or composed of calcium-oxalate at the chemical or infrared spectrometric analysis. They were not taking drugs affecting electrolyte handling. Serum and urinary concentrations of calcium were measured. Urinary pH was lower than 5.5 in spot morning collections. Controls had negative personal and familial history of kidney stones, normal serum creatinine and calcium and no evidence of diseases at physical examination.

### Urinary Exosome Analyses

Urine (50 ml) was collected from kidney stone patients and healthy controls from spot afternoon collections. The urinary exosomes were isolated from each urine sample using the Total Urine Exosome Isolation kit (Invitrogen) according to manufacturer's guideline. Following ultracentrifugation, the pellet was resuspended in SDS lysis buffer containing 150 mM NaCl; 1% SDS; 50 mM Tris, pH 8.0; and protease inhibitors, and then subjected to western blotting. A novel antibody against the human CLDN14 sequence (aa. 29-81 corresponding to the first extracellular loop) was raised to detect the CLDN14 protein in urinary exosomes.

### Statistical Analyses

The significance of differences between groups was tested by ANOVA (Statistica 6.0; Statsoft). When the all-effect F value was significant (p<0.05), post hoc analysis of differences between individual groups was made with the Newman-Keuls test. Values were expressed as mean±SEM, unless otherwise stated.

TABLE 5
Plasma and Urine Electrolyte Levels in WT and Claudin-14 KO Animals under SAHA and/or Furosemide Treatments

| Group | WT + Veh | WT + SAHA | CLDN14KO + Veh | CLDN14KO + SAHA | WT + Veh + Furo | AVT + SAHA + Furo |
|---|---|---|---|---|---|---|
| UV, | 44.95 ± 3.96 | 37.11 ± 4.54 | 39.83 ± 5.78 | 44.03 ± 2.07 | 137.11 ± 15.95** | 110.59 ± 9.03 |
| ul/24 | | | | | | |
| h · g | | | | | | |
| GFR, | 2.82 ± 0.36 | 2.09 ± 0.51 | 1.97 ± 0.32 | 2.42 ± 0.29 | 1.77 ± 0.44 | 1.69 ± 0.13 |
| ml/24 | | | | | | |
| h · g | | | | | | |
| P_{Ca}, | 10.40 ± 0.10 | 11.38 ± 0.44^{□} | 10.73 ± 0.59 | 11.13 ± 0.34 | 10.61 ± 0.43 | 9.95 ± 0.41 |
| mg/dL | | | | | | |
| P_{Mg}, | 2.43 ± 0.05 | 2.71 ± 0.12^{□} | 2.75 ± 0.05** | 2.76 ± 0.05 | 2.33 ± 0.11 | 2.20 ± 0.06 |
| mg/dL | | | | | | |
| FE_{Ca}, | 0.68 ± 0.08 | 0.27 ± 0.08^{□□} | 0.60 ± 0.18 | 0.45 ± 0.07 | 1.89 ± 0.67* | 1.97 ± 0.38 |
| % | | | | | | |
| E_{Ca}, | 1.96 ± 0.33 | 0.72 ± 0.26^{□} | 1.11 ± 0.20 | 1.20 ± 0.24 | 2.84 ± 0.78 | 3.37 ± 0.75 |
| ug/24 | | | | | | |
| hr · g | | | | | | |
| FE_{Mg}, | 14.01 ± 1.53 | 6.06 ± 1.71^{□□} | 10.68 ± 1.00 | 9.90 ± 0.83 | 32.83 ± 1.30** | 22.50 ± 6.40 |
| % | | | | | | |
| E_{Mg}, | 9.41 ± 1.18 | 3.16 ± 0.80^{□□} | 5.75 ± 1.01 | 6.50 ± 0.55 | 13.24 ± 3.13 | 8.34 ± 2.15 |
| ug/24 | | | | | | |
| hr · g | | | | | | |

Values are expressed as means ± SEM; N = 5-9, the number of animals; sex, male; age, 10-12 weeks.
UV, urine volume; GFR, glomerular filtration rate; P_{Ca}, P_{Mg}, plasma Ca⁺⁺ and Mg⁺⁺ concentrations; FE_{Ca}, FE_{Mg}, fractional excretion of Ca⁺⁺ and Mg⁺⁺.
^{□}p < 0.05,
^{□□}p < 0.01 versus Veh treatment in the same group;
*p < 0.05,
**p < 0.01 versus the WT group with Veh treatment.

TABLE 6
Plasma and Urine Electrolyte Levels in Antagomir Injected Animals

| Group | Scrbl | Anti-miR-374 | significance |
|---|---|---|---|
| UV, ul/24 h · g | 34.81 ± 3.22 | 41.65 ± 7.67 | n.s. |
| GFR, | 3.30 ± 0.56 | 2.75 ± 0.47 | n.s.. |
| ml/24 h · g | | | |
| P_{Ca}, mg/dL | 10.43 ± 0.23 | 10.50 ± 0.22 | n.s |
| P_{Mg}, mg/dL | 2.68 ± 0.20 | 2.10 ± 0.09 | p < 0.05 |
| FE_{Ca}, % | 0.93 ± 0.27 | 3.21 ± 0.16 | p < 0.01 |
| E_{Ca}, | 2.67 ± 0.36 | 9.11 ± 1.38 | p < 0.01 |
| ug/24 hr · g | | | |
| FE_{Mg}, % | 8.86 ± 2.23 | 20.94 ± 1.53 | p < 0.01 |
| E_{Mg}, | 7.61 ± 1.98 | 11.92 ± 2.25 | n.s. |
| ug/24 hr.g | | | |

Values are expressed as means ± SEM; N = 6, the number of animals; sex, male; age, 10-12 weeks.
UV, urine volume; n.s., not significant; GFR, glomerular filtration rate; P_{Ca}, P_{Mg}, plasma Ca⁺⁺ and Mg⁺⁺ concentrations; FE_{Ca}, FE_{Mg}, fractional excretion of Ca⁺⁺ and Mg⁺⁺.

### EXAMPLES 17-21

### Example 17

This example illustrates in vivo effects of histone deacetylase inhibitors on CLDN114.

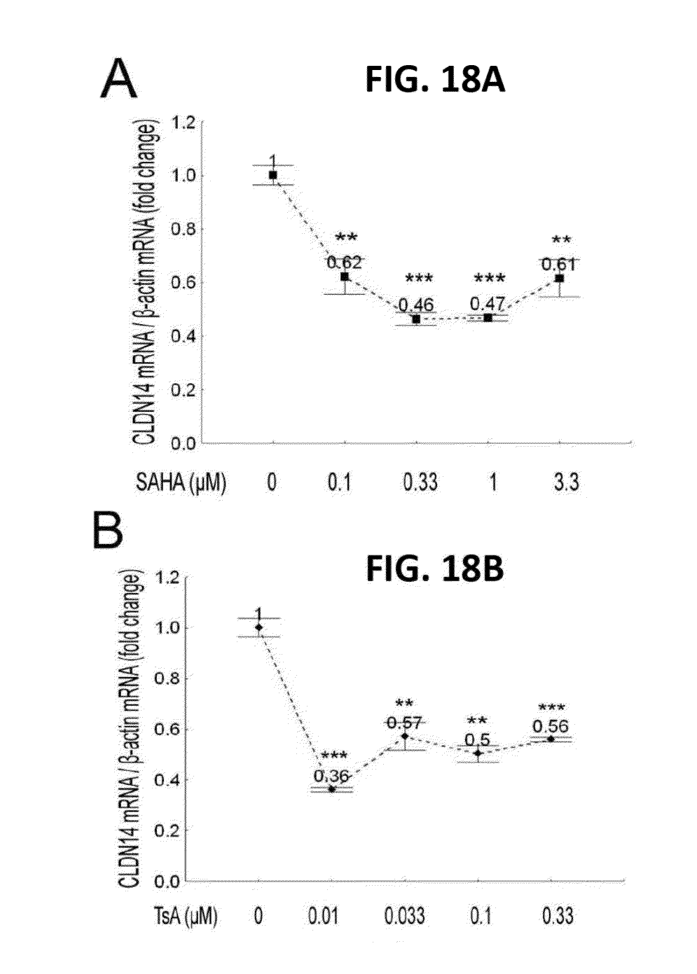

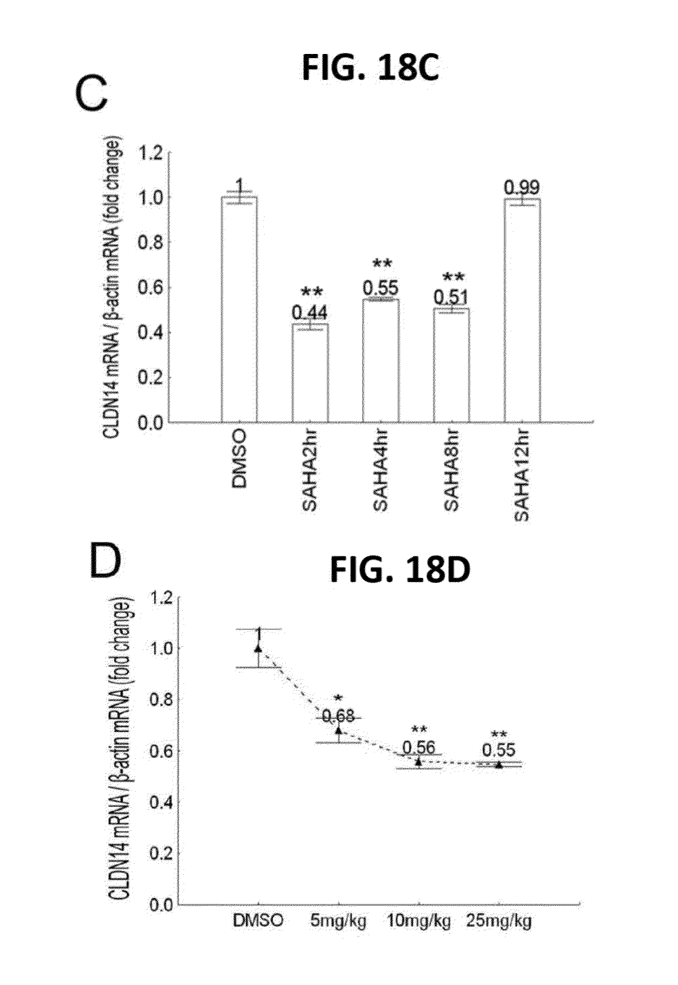

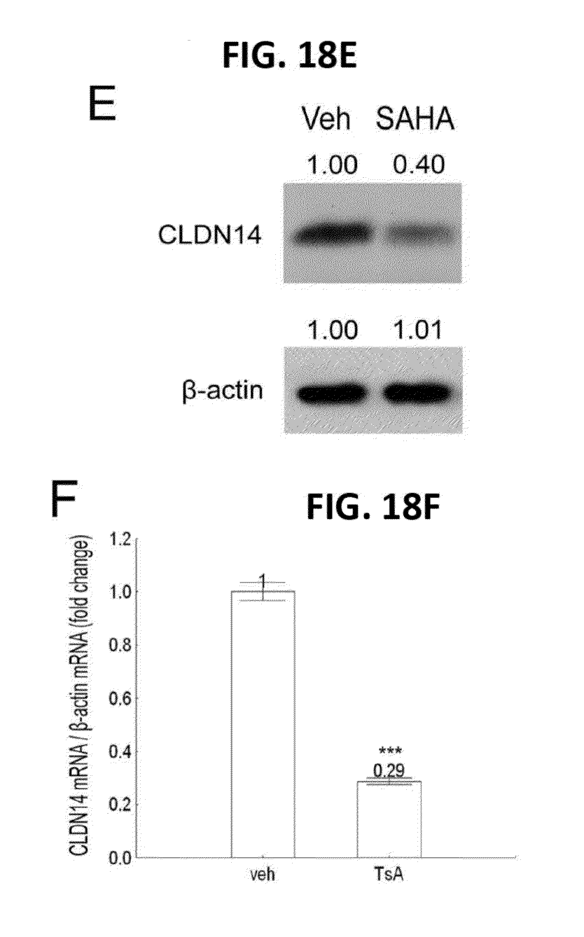

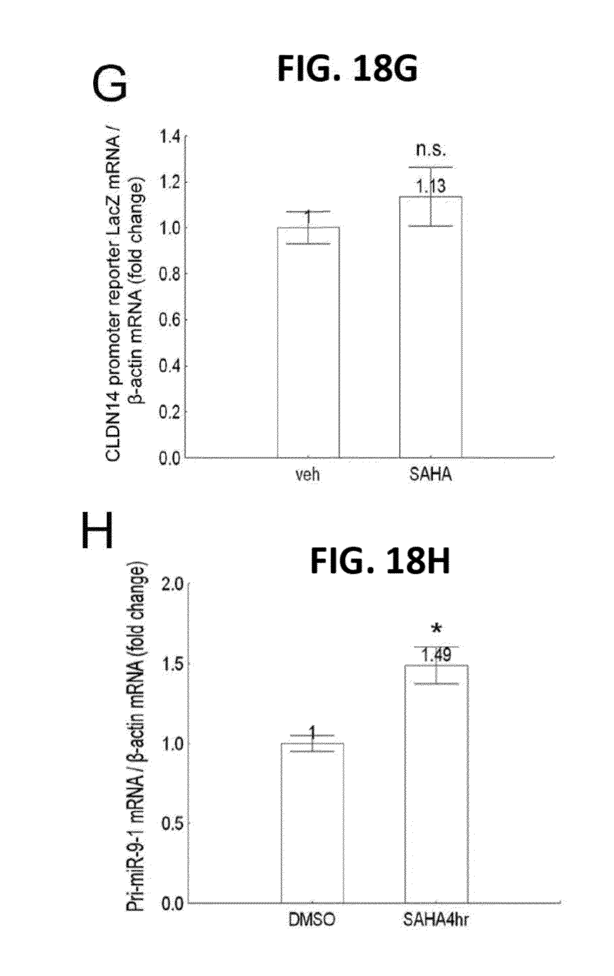

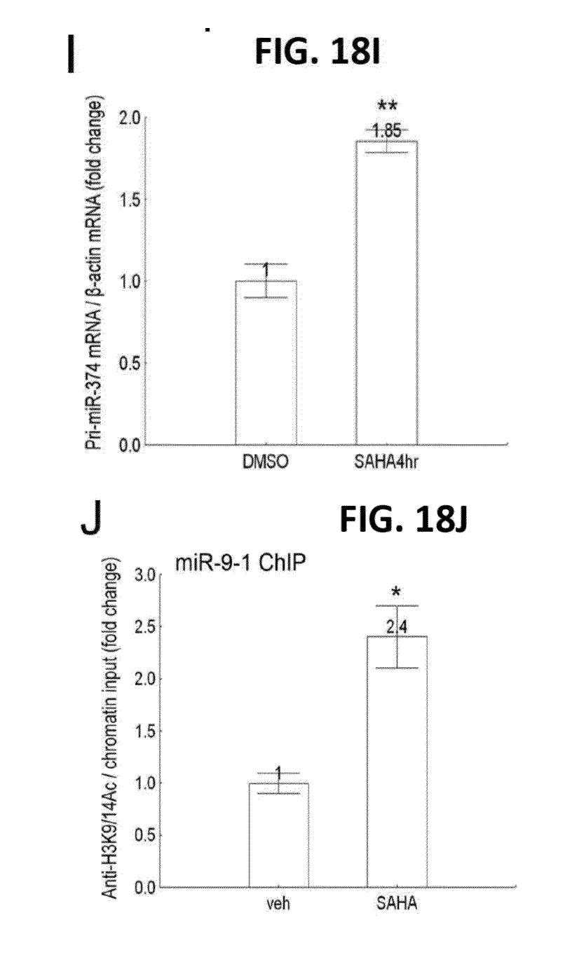

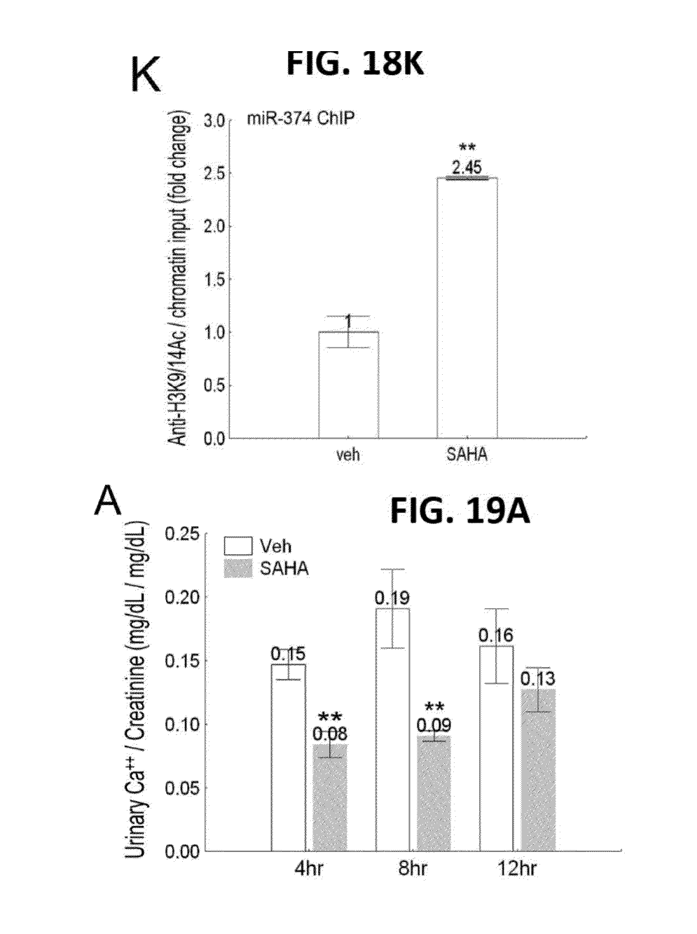

To examine the in vivo effects of histone deacetylase inhibitors on CLDN14, the inventors gave wild-type C57BL/U6 mice systemic treatments of SAHA or TsA over a range of doses and durations. A single dose of SAHA at 25 mg/kg BW⁻¹ reduced CLDN14 mRNA levels in the kidney by 56% (p<0.01, n=5 versus vehicle: FIG. 18C) at 2 hrs; by 4 hrs and through to at least 8 hrs, the reduction of CLDN14 expression persisted in the kidney (FIG. 18C); the effects of SAHA dissipated at 12 hrs (FIG. 18C).

The inventors varied the SAHA treatment doses from 5 mg/kg to 25 mg/kg; and found downregulation of CLDN14 at dosage as low as 5 mg/kg (p<0.05, n=5 versus vehicle) and through 10 mg/kg to 25 mg/kg at 4 hrs (FIG. 18D).

The CLDN14 protein levels were measured in freshly isolated TALH cells pooled from SAHA or vehicle treated mouse kidneys (n=5). SAHA treatment of 25 mg/kg BW⁻¹ reduced the CLDN14 protein levels by 60% when assayed at 4 hr. TsA was more effective in suppressing CLDN14 expression. At 1 mg/kg BW⁻¹ and 4 hr time point, TsA decreased CLDN14 mRNA levels in the kidney significantly, by 71% (p<0.001, n=5 versus vehicle: FIG. 18E).

Because the inventors had a claudin-14 KO/Reporter mouse line that was generated with a lacZ reporter gene replacing the last exon of claudin-14 gene (including the entire coding region and the 3′-UTR), (Ben-Yosef, T., et al., 2003) the inventors used the expression levels of lacZ gene as a faithful measurement for endogenous CLDN14 promoter activity regardless of any microRNA based regulation. The lacZ mRNA levels in the KO mouse kidneys were with no significant change 4 hrs after receiving 25 mg/kg BW⁻¹ SAHA treatment (FIG. 18G). By contrast, the CLDN14 targeting microRNAs—miR-9 and miR-374 were both upregulated by SAHA in the kidney. At the same dosage and time point, SAHA significantly increased the transcriptional levels of miR-9-1 gene by 1.49-fold (p<0.05, n=5 versus vehicle: FIG. 18H) and miR-374 gene by 1.85-fold (p<0.01, n=5 versus vehicle; FIG. 18I) respectively.

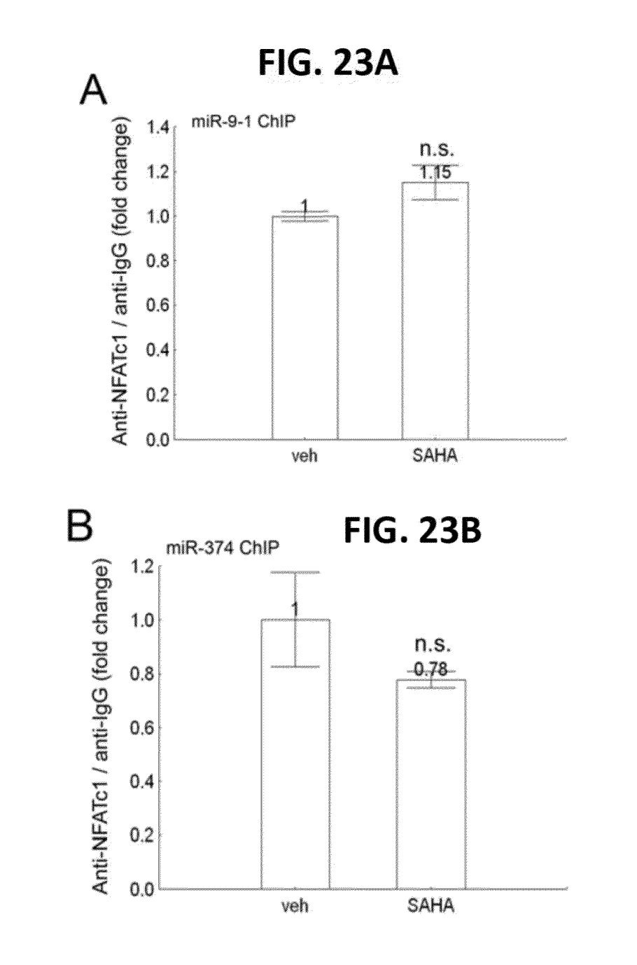

The inventors determined if SAHA caused direct histone acetylation on the microRNA promoters. The inventors previously discovered a NFAT binding site (AGGAAAAT) located 1-2 kb upstream of the miR-9-1 or miR-374 hairpin sequence (Gong Y, et al., 2013) where the histone acetylation was vividly regulated through CaSR signaling. The inventors found that the same promoter region experienced significant increases in histone H3 Lysine-9 (H3K9) and Lysine-14 (H3K14) acetylation 4 hrs after receiving SAHA at 25 mg/kg BW⁻¹, by 2.40-fold for miR-9-1 (p<0.05, n=5; FIG. 18J) and by 2.45-fold for miR-374 (p<0.01, n=5; FIG. 18K) respectively. The NFAT binding to miR-9-1 or miR-374 promoter was not affected by SAHA (FIG. 23). Without being limited by theory, these data establish a molecular basis for SAHA regulation of CLDN14 in the kidney.

### Example 18

This example illustrates that curbing CLDN14 expression lowers urinary Ca++ excretion.

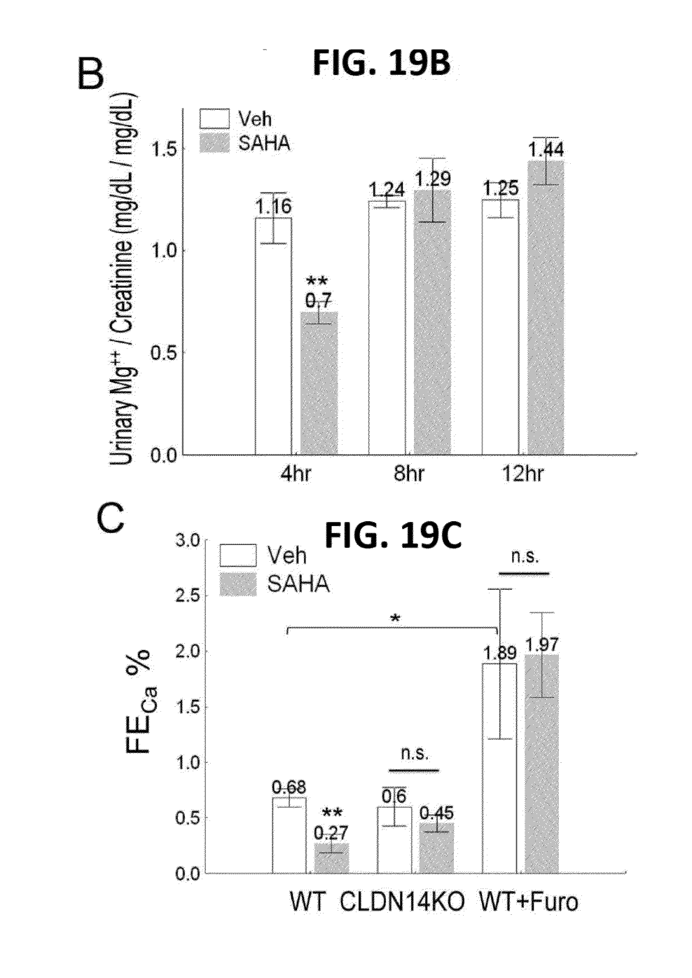

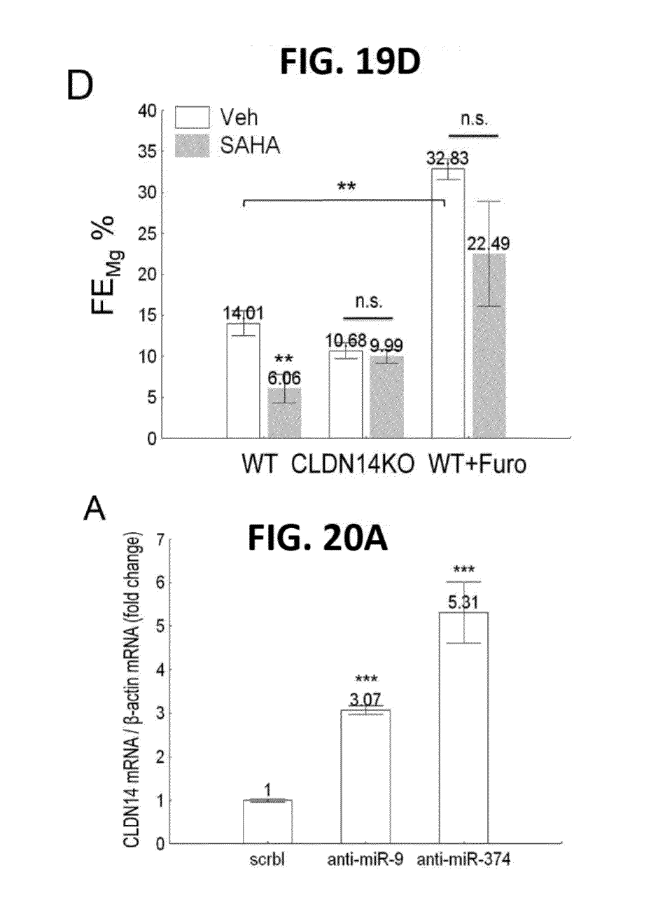

In these experiments, the inventors gave wild-type C57BL/6 mice SAHA treatments at 5 mg/kg BW⁻¹ and traced urinary Ca⁺⁺ and Mg⁺⁺ levels (as ratios to creatinine) from 4 hr to 12 hr in spot urine collections. Consistent with the downregulation of CLDN14 gene expression at 4 hrs, the urinary excretion of Ca⁺⁺ and Mg⁺⁺ was reduced by 47% and 40% respectively (p<0.01, n=5 versus vehicle, FIG. 19A-B). By 8 hrs, hypocalciuria was pronounced in SAHA treated animals (FIG. 19A) but urinary magnesium excretion recovered to the vehicle level (FIG. 19B). The SAHA effects on urinary Ca⁺⁺ and Mg⁺⁺ levels were completely lost after 12 hrs (FIG. 19A-B).

To investigate the renal transport function over a chronic phase, the inventors performed 24 hr urinalysis on age- and sex-matched wild-type mice receiving SAHA at 10 mg/kg BW⁻¹ per day. The plasma levels for Ca⁺⁺ (P_{Ca}) and Mg⁺⁺ (P_{Mg}) in SAHA-treated animals were both significantly higher than the vehicle group (p<0.05, n=9; Table 5), compatible with the observation of significantly reduced fractional excretion rates for Ca⁺⁺ (FE_{Ca}; FIG. 19C; Table 5) and Mg⁺⁺ (FE_{Mg}; FIG. 19D: Table 5) in the SAHA group. The total urinary Ca⁺⁺ (E_{Ca}) and Mg⁺⁺ (E_{Mg}) contents were also lowered by SAHA (p<0.05, n=9; Table 5), despite unchanged glomerular filtration rates (GFR) and urinary volume (UV) (Table 5). TsA, at 1 mg/kg BW⁻¹ per day, exerted similar effects on renal Ca⁺⁺ and Mg⁺⁺ metabolism. These data indicate a direct renal tubular effect for HDAC inhibitors.

To elucidate the genetic origin of SAHA effects, the inventors took used the CLDN14 KO mice (previously backcrossed to C57BL/6 background; Gong Y, et al., 2012) and treated them with the same SAHA regimen. In contrast to the wild-type mice, the CLDN14 KO animals were refractory towards SAHA induced hypocalciuria and hypomagnesiuria (FIG. 19C-D; Table 5). The plasma Ca⁺⁺ and Mg⁺⁺ changes were also abolished in CLDN14 KO (Table 5), suggesting that SAHA primarily acted upon the kidney where CLDN14 was exclusively localized. To elucidate the tubular origin of SAHA effects, the inventors pre-treated wild-type animals with furosemide at 40 mg/kg BW⁻¹ per day. Furosemide, by inhibiting the NKCC2 transporter in the TALH, induces natriuresis and diuresis, which in turn abolishes the transepithelial voltage for paracellular Mg⁺⁺ and Ca⁺⁺ reabsorption. (Greger, R. 1985) The furosemide treatment significantly increased UV, FE_{Ca} and FE_{Mg} (Table 5). In addition to the elevated baseline levels, furosemide also abolished the SAHA effects on FE_{Ca} and FE_{Mg} (FIG. 19C-D: Table 5), identifying the TALH as the major nephron segment for SAHA function.

### Example 19

This example illustrates the effects of an HDAC inhibitor on other Ca⁺⁺ related hormonal or genetic pathways in the kidney.

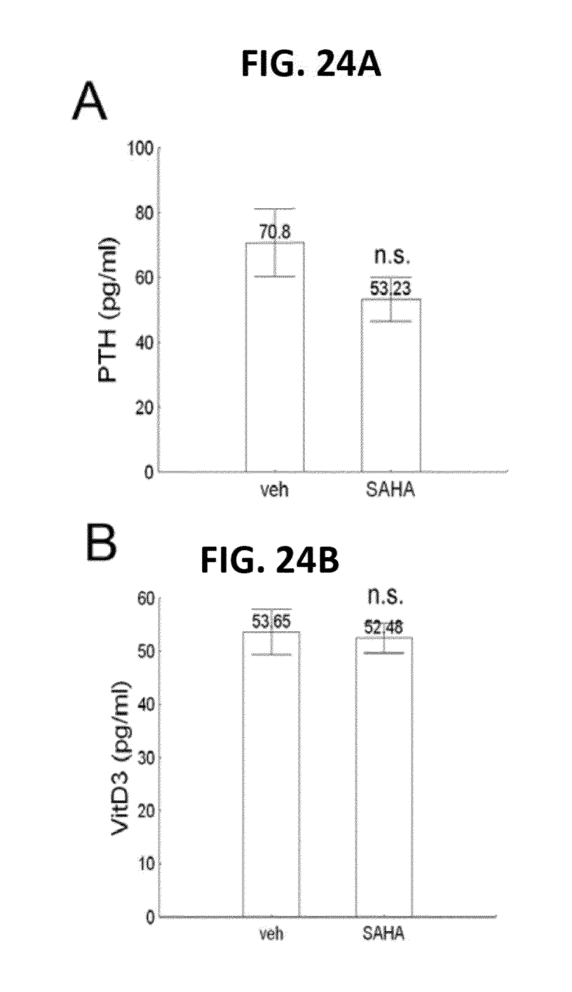

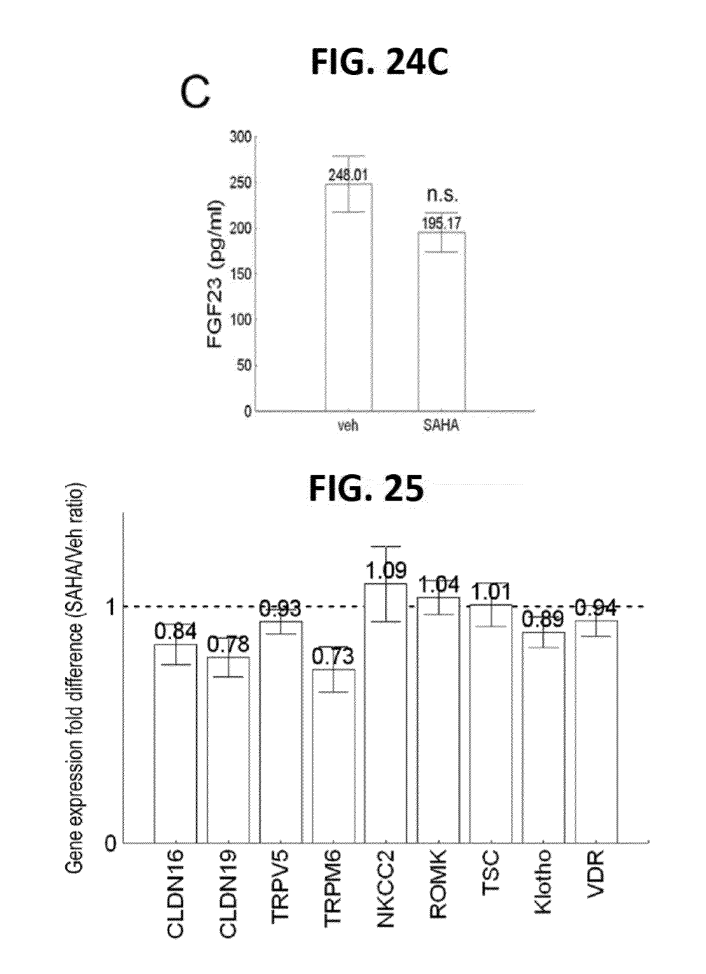

Chronic SAHA treatment can induce mild hypercalcemia, therefore the inventors measured the circulating PTH, 1,25-(OH)₂-vitD₃ and FGF23 levels to determine if hypercalcemia originated from changes in these hormonal systems. None of these hormones changed during SAHA treatments (FIG. 24), nor did the inventors find any significant change in the expression levels of other major Ca⁺⁺/Mg⁺⁺ transporters from the kidney including CLDN16, CLDN19, TRPV5, TRPM6. NKCC2, ROMK, TSC, Klotho and VDR (FIG. 25).

### Example 20

This example illustrates effects of microRNAs on renal calcium metabolism.

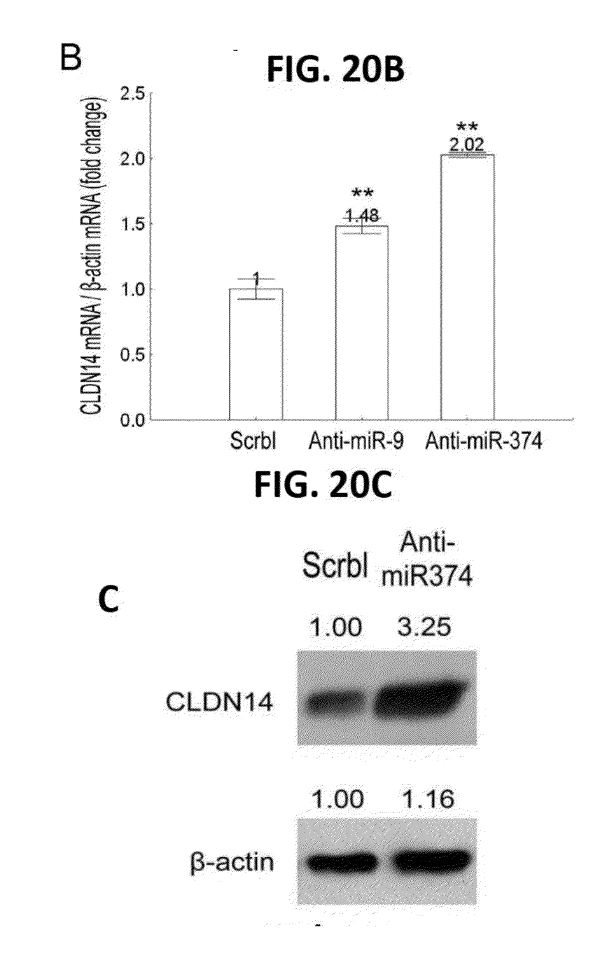

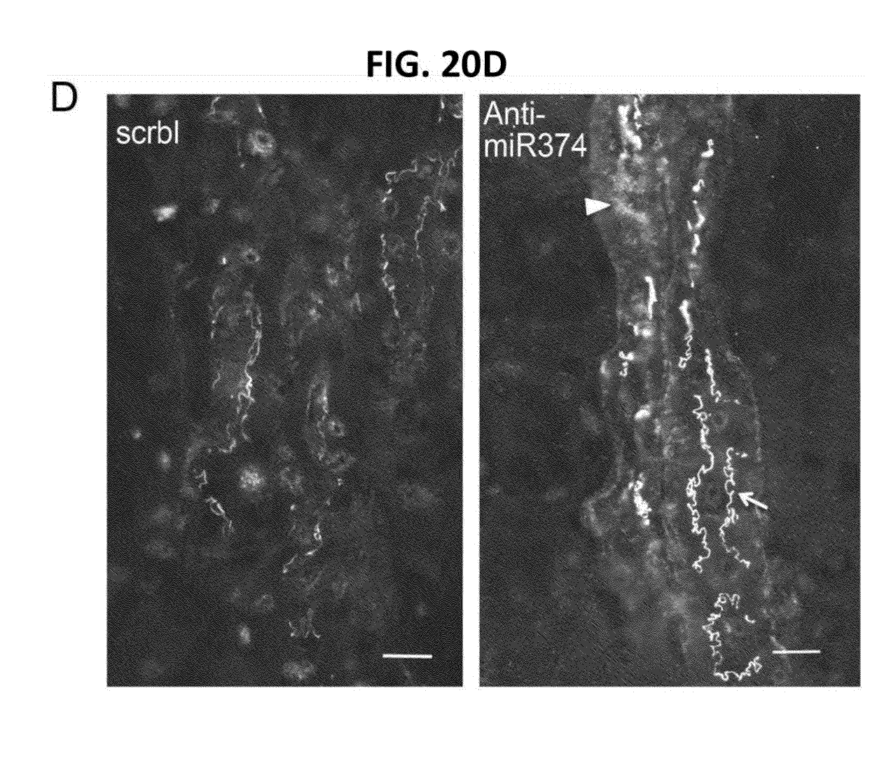

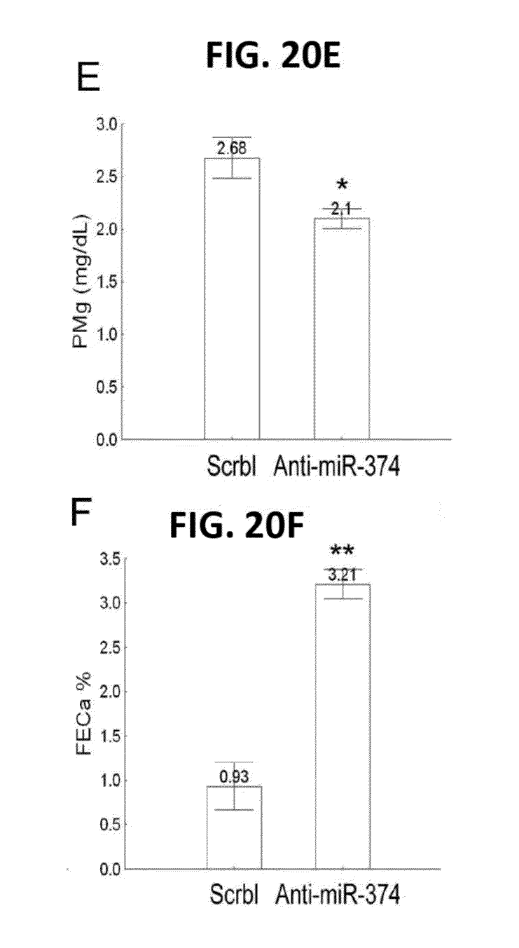

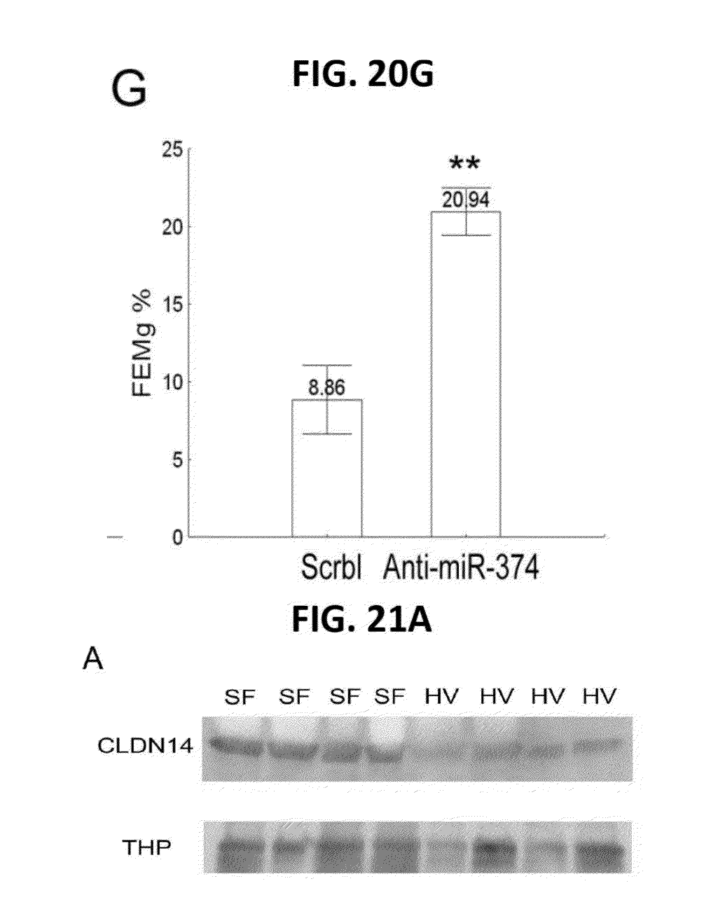

To investigate whether manipulation of microRNAs per se was sufficient to induce changes in renal calcium metabolism, the inventors screened for LNA sequences ranging from 8 nt to 23 nt long (Obad, S. et al., 2011; Elmén, J., et al., 2008; and Castoldi, M., et al., 2011) and found the most effective sequences for miR-9 and miR-374 (see Method). In the primary TALH cultures in vitro, transfection with anti-miR-9 or anti-miR-374 but not scrambled antagomir induced significant increases in CLDN14 gene expression by 3.07-fold and 5.31-fold respectively (p<0.001, n=−4; FIG. 20A). The in vivo knockdown appeared to be much more difficult. Although several studies have reported effective ablation of glomerular microRNAs from the mesangial cells (Kato, M., et al., 2009) or the podocytes (Gebeshuber, C. A., et al., 2013) with systemic injection of antagomirs, the inventors found that systemic injection of anti-miR-9 or anti-miR-374 was not able to knockdown microRNA in renal tubules throughout the dosage range of 1 mg/kg to 25 mg/kg BW⁻¹ in mice. Instead, when the LNA antagomirs were co-injected with the in vivo-jetPEI™ Delivery Reagent (Polyplus-transfection SA) (based upon the polyethylenimine cationic polymers), a single dose of anti-miR-9 or anti-miR-374 at 2.5 mg/kg BW⁻¹ significantly increased the CLDN14 mRNA level in the kidney 24 hr after injection, by 1.48-fold and 2.02-fold respectively relative to the scrambled antagomir (p<0.01, n=5; FIG. 20B). Because anti-miR-374 was more effective than anti-miR-9 and miR-9 had known tumorigenic implication through targeting E-cadherin, (Ma, L., et al., 2010) the inventors focused upon miR-374 to study its role in renal tubular functions.

Wild-type C57BL/6 mice were given anti-miR-374 injections at 2.5 mg/kg BW⁻¹ per day and measured CLDN14 protein abundance, localization, and plasma and urinary electrolytes levels. In freshly isolated TALH cells pooled from antagomir treated mouse kidneys (n=5), the CLDN14 protein level was 3.25-fold higher with anti-miR-374 than scrambled antagomir treatment (FIG. 20C). The change in CLDN14 levels at the tight junction was particularly striking. The thin interdigitated TJ lines faintly stained for CLDN14 in control mouse kidneys (FIG. 20D) were profoundly strengthened in anti-miR-374 treated mouse kidneys (arrow; FIG. 20D), with occasional subapical staining spotted in some TALH cells (arrowhead; FIG. 20D). The P_{Ca} was not changed by anti-miR-374 but the P_{Mg} was significantly lower in the anti-miR-374 group than the scrambled (p<0.05, n=6; FIG. 20E; Table 6). The fractional excretion rates for Ca⁺⁺ and Mg⁺⁺ were both significantly increased by anti-miR-374 in 24 hr urine collections (FE_{Ca}: 3.45-fold, FE_{Mg}: 2.36-fold versus scrambled: FIG. 20F-G; p<0.01, n=6; Table 6). Other renal parameters such as GFR and UV were not changed by anti-miR-374 (Table 6), indicating a direct tubular effect.

The anti-miR-374 knockdown animals phenocopied the CLDN14 transgenic overexpression animals described by us (Gong Y., et al., 2013) establishing a therapeutic principle for using microRNAs to manipulate CLDN14 expression and urinary calcium excretion.

### Example 21

This example illustrates that the renal expression levels of CLDN14 can serve as a reporter for a range of Ca⁺⁺ related pathophysiological abnormalities, including kidney stone diseases.

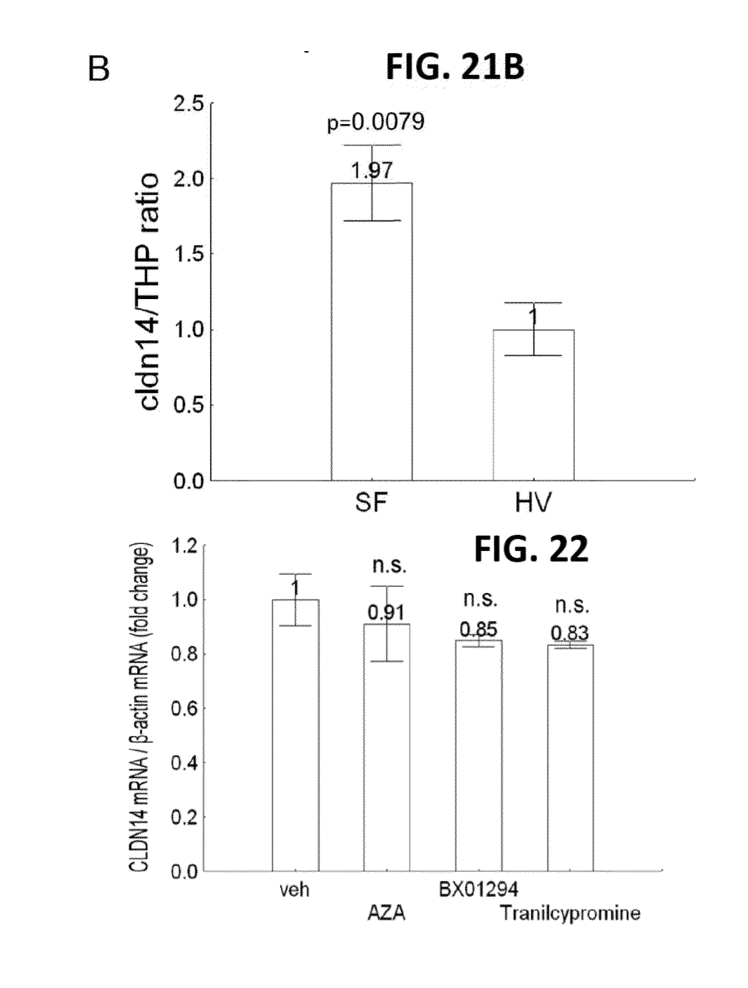

The inventors used an antibody against the first extracellular loop of CLDN14 (see Methods) to detect the CLDN14 protein from human urine exosomes. The inventors recruited 8 calcium oxalate stone formers (SF: n=3 hypercalciuric patients [37.5%]) and 7 healthy volunteers (HV) with no history of kidney stone. All stone formers had recurring kidney stones with the latest episode occurring in year 2013. Their 24 hr urinary Ca⁺⁺ excretion levels were 236.8±34.4 mg, serum Ca⁺⁺ 9.48±0.08 mg/dL; and blood pressure normal. The CLDN14 proteins levels from isolated urinary exosomes were consistently higher in SF than HV (FIG. 21A). When normalized to the Tamm-Horsfall protein (THP) levels, a well-established marker for urinary exosomes, (Fernández-Llama, P., et al., 2010) the increases in CLDN14 reached the significance of p=0.0079 (FIG. 21B). Intriguingly, after excluding the hypercalciuric patients, the CLDN14 difference between SF and HV was still significant: p=0.0156. These data extend the role of CLDN14 from to kidney stone reporter or modulator.

### The Following Methods are Applicable to Examples 22-27

### Reagents, Antibodies, Cell Lines and Animals

The reagents, kits, and antibodies were listed in Table 9. Human HEK293 cells were cultured in DMEM (Invitrogen) supplemented with 10% FBS, penicillin/streptomycin, and 1 mM sodium pyruvate. WT C57BL/6 mice were from Charles River Laboratory. The CLDN14^{+/lacZ} reporter/knockout mice were back-crossed to C57BL/6 background for 7 generations. Thyroparathyroidectomy (TPTX) was performed by Charles River Lab surgeons.

### Pharmacological and Dietary Manipulation in Experimental Animals

NPS2143 and cinacalcet (Table 9) were dissolved in vehicle −20% (w/v) (2-Hydroxypropyl)-β-cyclodextrin solution and fed to mice with gavage syringe. Cyclosporin-A (Sandimmune; Table 9) was diluted in 0.9% saline and I.P. injected to animals. Control animals received CNI vehicle injection (13% wiv Cremophor EL and 32.9% ethanol). FK506 was dissolved in CNI vehicle, diluted in 0.9% saline and I.P. injected to animals. For dietary Ca⁺ manipulation, animals were fed with the following diets for six consecutive days: basal diet: 0.610% Ca⁺⁺ (TestDiet #5755); low Ca⁺⁺ diet: 0 Ca⁺⁺ (TestDiet #5855); high Ca⁺⁺ diet: 5% Ca⁺ (TestDiet #5AVB). All animals had free access to water and were housed under a 12 hr light cycle. Blood samples were taken by cardiac puncture rapidly after initiation of terminal anesthesia and centrifuged at 4° C. for 10 min. Kidney were dissected out and immediately frozen at −80° C.

### Surgical Protocols and Renal Clearance

The method for performing renal clearance measurements in the mouse has been described by Hou, J., et al., 2007 and Hou, J., et al., 2009. Mice were anesthetized by i.p. injection of Inactin (Sigma: 100 mg/kg). The jugular vein was catheterized for i.v. infusion of 0.9% saline at 2 μL/min, with 1% FITC-inulin included in the infusate. After an equilibration period of 60 min, renal clearance measurements were carried out for a 60 min period. Urine was collected under mineral oil, and 30 μL blood sample was taken at hourly intervals. Urine and plasma Ca⁺⁺ and Mg⁺⁺ concentrations were measured by atomic flame absorption spectrophotometer (PerkinEhner). Urine and plasma FITC-inulin levels were measured in 100 mM HEPES buffer (pH7.0) with fluorescence spectrophotometer (BioTek). The fractional excretion of electrolytes was calculated using the following equation FEᵢₒₙ=V×Uᵢₒₙ/(GFR×Pᵢₒₙ), where GFR was calculated according to the clearance rate of FITC-inulin (GFR=V×Uᵢₙᵤₗᵢₙ/Pᵢₙᵤₗᵢₙ).

### Animal Metabolic Studies

Mice were housed individually in metabolic cages (Harvard Apparatus) with free access to water and food for 24 hr. Urine was collected under mineral oil. The plasma and urine Ca⁺⁺ and Mg⁺⁺ levels were determined with atomic flame absorption spectrophotometer (PerkinElmer). The creatinine levels were measured with an enzymatic method that was independent of plasma chromogens. (Himmerkus, N., et al., 2008) The fractional excretion of electrolytes was calculated using the following equation FEᵢₒₙ=V×Uᵢₒₙ/(GFR×Pᵢₒₙ), where GFR was calculated according to the clearance rate of creatinine (GFR=V×U_{creatinine}/P_{creatinine}).

### Generation of Transgenic Mice

The coding sequence of mouse claudin-14 gene was cloned into a bicistronic pIRES-GFP vector (Clontech). A 3.7 kb mouse Tamm-Horsfall protein (THP) promoter (Stricklett, P. K., et al., 2003) was cloned into the pIRES-GFP vector to replace the CMV promoter. Inclusion of GFP allowed rapid screening of transgene expression in the kidney. To generate claudin-14 overexpression transgenic (TG) mice, female donor mice (C57BL/6x CBA hybrid strain) were superovulated with a combination of pregnant mare serum (5 units) and human CG (5 units). The transgenic vector was injected into the pronucleus of single-cell mouse embryos and allowed to develop to two-cell embryo stage. Injected embryos were implanted into pseudopregnant females and carried to term. The transgenic founder mice were crossed to WT C57BL/6 mice, and F1 progeny were analyzed. Littermate WT mice were used as controls. Out of 41 transgenic founders, 9 had germ-line transmission of transgene; 4 had detectable transgene expression in the kidney.

### Establishing Primary Cultures of Mouse Thick Ascending Limb Cells

An immunomagnetic separation method was used to isolate the TALH cells from the mouse kidney. (Gong, Y., et al., 2012; Hou, J., et al., 2009) Antibodies against the TALH cell specific surface antigen, Tamm-Horsfall protein (THP; a GPI-anchored protein that is exclusively expressed in the TALH and the early part of the DCT,) (Stricklett, P. K., et al., 2003; Bernascone, I., et al., 2010) were coated onto the paramagnetic polystyrene beads (Dynabeads M-280; Dynal), allowing immunoprecipitation of the TALH cells from collagenase digested mouse kidneys. The isolated cells were plated in DMEM medium supplemented with 10% FBS, penicillin/streptomycin, and 1 mM sodium pyruvate for 16 hours, followed by immediate transfection with NFAT.

### Real-time PCR Quantification

Total RNA including microRNA was extracted using Trizol (Invitrogen). Cellular mRNA was reverse transcribed using the Superscript-III kit (Invitrogen), followed by real-time PCR amplification using SYBR Green PCR Master Mix (Bio-Rad) and Eppendorf Realplex2S system. The design of claudin-14 primers was described before by Gong. Y., et al., 2012. The design of pri-miRNA primers was according to Ma. L., et al., 2010. The PCR primers are listed in Table 2. Results were expressed as 2^{-ΔCt} values with ΔCT=Ct_{gene}−Ct_{β-actin}.

### Manipulation of NFAT Gene Expression

Pre-validated Silencer Select siRNAs were designed and synthesized by Ambion. A pool of 3 target-specific 21 nt siRNA duplexes was designed against the coding region of mouse NFATc1-c4 genes. A scrambled siRNA duplex was used as negative control. Transfection of siRNAs or NFATc1nuc was carried out with Lipofectamine LTX & Plus Reagent for primary cultures.

### Luciferase Reporter Assay

The miR-9-1 and miR-374 gene promoters were cloned into pGL4.10 luciferase reporter (Promega) with Sfi1 sites. Deletion of the NFAT binding sites (AGGAAAAT) in miR-9-1 and miR-374 promoters were generated using site-directed mutagenesis (Stratagene). The pGL4.10 reporter (500 ng), the pGL4.74 Renilla luciferase control vector (500 ng; Promega) and pcDNA3.1-NFATc1nuc vector were co-transfected to HEK293 cells in 12-well culture dishes using Lipofectamine-2000 (Invitrogen). Twenty-four hours after transfection, firefly and renilla luciferase activities were measured with a chemiluminescence reporter assay system—Dual Glo (Promega) in a GLOMAX Luminometer (Promega).

### Chromatin Immunoprecipitation (ChIP)

Freshly isolated TALH cells or primary TALH cell cultures were cross-linked in 1% paraformaldehyde (pH7.0) for 10 min at room temperature. The cross-linked cells were lysed following to instruction of MAGnify ChIP system (Invitrogen). Chromatin was sonicated into 500 bp fragments with Bioruptor UCD-200 (High power, 15 cycles of 30 seconds ON, 30 seconds OFF, Diagenode). Sheared chromatin was precipitated with antibody-bound Dynabeads (Invitrogen). Following reverse crosslink and DNA extraction, antibody-bound DNA was eluted and analyzed with real-time PCR. The primers for ChIP analyses are listed in Table 2. Two criteria for normalization were applied: fold enrichment over anti-IgG background signal and input control over chromatin input signal.

### Immunolabeling and Fluorescence Microscopy

For viewing claudin localization in the kidney, fresh cryostat sections (10 μm) were fixed with cold methanol at −20° C., followed by blocking with PBS containing 10% FBS, incubation with primary antibody (1:300) and FITC-labeled secondary antibody (1:200). After washing with PBS, slides were mounted with Mowiol (Calbiochem). Epifluorescence images were taken with a Nikon 80i photomicroscope equipped with a DS-Qi1Mc digital camera. All images were converted to TIFF format and arranged using Photoshop CS4 (Adobe).

### Statistical Analyses

The significance of differences between groups was tested by ANOVA (Statistica 6.0; Statsoft). When the all-effect F value was significant (p<0.05), post hoc analysis of differences between individual groups was made with the Newman-Keuls test. Values were expressed as mean±SEM, unless otherwise stated.

TABLE 7
Plasma and Urine Electrolyte Levels in WT and Claudin-14 KO Animals Under NPS2143 and Cinacalcet Treatments

| Group | | WT | | | WT | | |
|---|---|---|---|---|---|---|---|
| Treat- | | 2 hr | | | 8 hr | | |
| ment | NPS | Veh | Cina | NPS | Veh | Cina | NPS |
| Weight, | 22.10 ± 0.38 | 23.21 ± 1.34 | 22.88 ± 1.39 | 21.73 ± 0.27 | 21.90 ± 0.16 | 22.72 ± 0.20 | 23.86 ± 0.86 |
| g | | | | | | | |
| UV, | 7.91 ± 1.34 | 9.85 ± 1.68 | 6.65 ± 1.14 | 7.14 ± 1.25 | 9.03 ± 1.28 | 7.74 ± 2.13 | 3.63 ± 0.06 |
| ul/ | | | | | | | |
| hr · g | | | | | | | |
| GFR, | 0.51 ± 0.06 | 0.43 ± 0.08 | 0.44 ± 0.06 | 0.44 ± 0.08 | 0.43 ± 0.08 | 0.28 ± 0.03 | 0.34 ± 0.02 |
| ml/ | | | | | | | |
| hr · g | | | | | | | |
| P_{Ca}, | 11.15 ± 0.21* | 10.18 ± 0.08 | 8.44 ± 0.27* | 10.63 ± 0.43 | 10.59 ± 0.18 | 9.58 ± 0.21* | 10.22 ± 0.30 |
| mg/dL | | | | | | | |
| P_{Mg}, | 2.74 ± 0.15* | 2.34 ± 0.04 | 1.96 ± 0.04* | 2.37 ± 0.09 | 2.25 ± 0.04 | 2.44 ± 0.16 | 2.88 ± 0.12 |
| mg/dL | | | | | | | |
| FE_{Ca}, | 0.09 ± 0.01** | 0.63 ± 0.09 | 3.82 ± 0.52** | 0.58 ± 0.11 | 0.65 ± 0.10 | 0.88 ± 0.14 | 0.74 ± 0.26 |
| % | | | | | | | |
| E_{Ca}, | 0.06 ± 0.01* | 0.28 ± 0.09 | 1.33 ± 0.13** | 0.25 ± 0.04 | 0.27 ± 0.04 | 0.22 ± 0.02 | 0.25 ± 0.09 |
| ug/ | | | | | | | |
| hr · g | | | | | | | |
| FE_{Mg}, | 7.59 ± 0.32* | 16.59 ± 1.31 | 28.14 ± 2.93* | 14.88 ± 2.63 | 16.06 ± 1.62 | 18.80 ± 1.48 | 10.71 ± 2.16 |
| % | | | | | | | |
| E_{Mg}, | 1.04 ± 0.06* | 1.60 ± 0.20 | 2.32 ± 0.26# | 1.41 ± 0.18 | 1.54 ± 0.30 | 1.25 ± 0.09 | 1.02 ± 0.12 |
| ug/ | | | | | | | |
| hr · g | | | | | | | |

| Group | KO | | | WT + CsA | |
|---|---|---|---|---|---|
| Treat- | 2 hr | | | 2 hr | |
| ment | Veh | Cina | NPS | Veh | Cina |
| Weight, | 22.50 ± 2.87 | 21.80 ± 0.97 | 20.77 ± 0.58 | 21.65 ± 0.48 | 20.56 ± 0.18 |
| g | | | | | |
| UV, | 4.77 ± 1.60 | 4.38 ± 0.68 | 9.03 ± 3.47 | 10.39 ± 0.68 | 14.98 ± 3.97 |
| ul/ | | | | | |
| hr · g | | | | | |
| GFR, | 0.36 ± 0.09 | 0.36 ± 0.06 | 0.41 ± 0.06 | 0.54 ± 0.11 | 0.52 ± 0.08 |
| ml/ | | | | | |
| hr · g | | | | | |
| P_{Ca}, | 9.81 ± 0.11 | 8.47 ± 0.09* | 10.50 ± 0.18 | 10.07 ± 0.13 | 8.09 ± 0.32* |
| mg/dL | | | | | |
| P_{Mg}, | 2.71 ± 0.05¶ | 2.64 ± 0.10 | 1.67 ± 0.02 | 1.63 ± 0.03¶ | 1.58 ± 0.04 |
| mg/dL | | | | | |
| FE_{Ca}, | 0.59 ± 0.17 | 1.08 ± 0.26 | 0.95 ± 0.14 | 1.19 ± 0.12¶ | 1.93 ± 0.20* |
| % | | | | | |
| E_{Ca}, | 0.19 ± 0.04 | 0.31 ± 0.03 | 0.38 ± 0.02 | 0.64 ± 0.17 | 0.79 ± 0.07 |
| ug/ | | | | | |
| hr · g | | | | | |
| FE_{Mg}, | 11.67 ± 2.11\hat{ } | 12.35 ± 2.21 | 11.96 ± 2.27 | 12.86 ± 1.99 | 14.47 ± 1.13 |
| % | | | | | |
| E_{Mg}, | l.05 ± 0.10 | 1.12 ± 0.03 | 0.80 ± 0.19 | 1.02 ± 0.12 | 1.22 ± 0.2.4 |
| ug/ | | | | | |
| hr · g | | | | | |

Values are expressed as means ± SEM; N = 5-7, the number of animals; sex, male; age, 10-12 weeks.
UV, urine volume; GFR, glomerular filtration rate; P_{Ca}, P_{Mg}, plasma Ca⁺⁺ and Mg⁺⁺ concentrations; FE_{Ca}, FE_{Mg}, fractional excretion of Ca⁺⁺ and Mg⁺⁺.
*p < 0.05 versus Veh treatment in the same group (N = 5-7)
**p < 0.01 versus Veh treatment in the same group (N = 5-7)
#p = 0.07 versus Veh treatment in the same group (N = 5-7)
¶p < 0.05 versus the same treatment in WT 2 hr group (N = 5-7)
\hat{ }p = 0.09 versus the same treatment in the WT 2 hr group (N = 5-7)

TABLE 8
Plasma and Urine Electrolyte Levels in WT
and Claudin-14 Transgenic Animals.

| Group | WT | TG | Significance |
|---|---|---|---|
| Weight, g | 22.27 ± 1.40 | 20.88 ± 0.78 | n.s. |
| UV, ul/24 h · g | 48.53 ± 6.76 | 55.47 ± 11.86 | n.s. |
| GFR, ml/24 h · g | 3.48 ± 0.68 | 4.09 ± 0.88 | n.s. |
| P_{Ca}, mg/dL | 10.07 ± 0.14 | 9.89 ± 0.10 | n.s. |
| P_{Mg}, mg/dL | 2.59 ± 0.05 | 2.21 ± 0.08 | p < 0.01 |
| FE_{Ca}, % | 0.46 ± 0.06 | 2.40 ± 0.38 | p < 0.01 |
| E_{Ca}, ug/24 hr · g | 1.60 ± 0.37 | 8.53 ± 1.10 | p < 0.01 |
| FE_{Mg}, % | 8.24 ± 0.96 | 20.86 ± 3.59 | p < 0.05 |
| E_{Mg}, ug/24 hr · g | 7.50 ± 1.48 | 16.71 ± 2.63 | p < 0.05 |

Values are expressed as means ± SEM; N = 6, the number of animals; sex, male; age, 10-12 weeks. UV, urine volume; n.s, not significant: GFR, glomerular filtration rate; PCa, PMg, plasma Ca⁺⁺ and Mg⁺⁺ concentrations; FECa, FEMg, fractional excretion of Ca⁺⁺ and Mg⁺⁺.

TABLE 9
Reagents and Experimental Materials

| | Name | Supplier | Cat No |
|---|---|---|---|
| Chemicals | TRIzol ® Reagent | Invitrogen | 15596-018 |
| | (2-Hydroxypropy1)-β-cyclodextrin | Sigma | 332607 |
| | Advantage ® RT-for-PCR Kit | Clontech | 639506 |
| | iQ ™ SYBR ® Green Supermix | Bio Rad | 170-8882 |
| | Cinacalcet HCL | Amegen | |
| | NPS2143 hydrochloride | Sigma | SML0362- |
| | | | 25MG |
| | Cremophor EL | Sigma | C5135-500G |
| | FK-506 monohydrate | Sigma | F4679-5MG |
| | Sandimmune Injection (cyclosporine injection) | Novartis | |
| | Inulin-FITC | Sigma | F3272 |
| | Lipofectamine ® LTX & Plus Reagent | Invitrogen | 15338-100 |
| | Lipofectamine ® 2000 Transfection Reagent | Invitrogen | 11668019 |
| | Silencer ® Select Negative Control No. 1 siRNA | Invitrogen | 4390843 |
| | NFAT Silencer Selected siRNA | Invitrogen | 4390771 |
| | Collagenase | worthington | LS004196 |
| | MAGnifY ™ Chromatin Immunoprecipitation | Invitrogen | 49-2024 |
| | System | | |
| | β-Gal Assay Kit | Invitrogen | K1455-01 |
| | Dual-Glo Luciferase Assay System | Promega | E2940 |
| | DC Protein Assay Kit II | Bio Rad | 500-0112 |
| | Precision Plus Protein ™ WesternC ™ Pack | Bio Rad | 161-0385 |
| | MOUSE PTH 1-84 ELISA KIT | IMMUTOPICS | 60-2305 |
| | QuikChange Lightning Multi Site-Directed | Agilent | 210518 |
| | Mutagenesis Kit | Technologies | |
| Tissue | FBS Advantage | Atlanta Bio | S11050 |
| Culture | | | |
| | Penicillin-Streptomycin | Invitrogen | 15140-122 |
| | Trypsin EDTA | Cellgro | 25-053-CI |
| | Dulbecco's Modification of Eagle's Medium | Cellgro | 10-013-CV |
| | (DMEM) | | |
| Antibody | Anti-acetyl-Histone H3 Antibody | Millipore | 06-599 |
| | Anti-NF-ATcl Antibody | BD Bioscience | 556602 |
| | Anti-cldn14 | Lab Generated | |
| | anti-Tamm-Horsfall Glycoprotein | Biomedical | BT-590 |
| | | Technologies | |

### EXAMPLES 22-27

### Example 22

This example illustrates pharmacological manipulation with calcimimetic and calcilytic compounds demonstrating CaSR dependent regulation of claudin-14 gene expression in the kidney.

The CaSR agonist cinacalcet (calcimimetic) has been used for the treatment of secondary hyperparathyroidism in chronic kidney disease (CKD) and dialysis patients. (Lindberg. J. S., et al., 2005) NPS2143 is a novel CaSR antagonist that has been previously demonstrated to increase serum Ca⁺⁺ level independent of PTH secretion. (Loupy, A., et al., 2012) The kidney CaSR is predominantly expressed in the TALH, (Loupy, A., et al., 2012) co-localizing with claudin-14. To investigate how CaSR regulated claudin-14 in vive in the kidney, the inventors treated age (8-10 weeks old) and sex (male) matched mice (strain: C57BL/6) with NPS2143 and cinacalcet over a range of doses and durations, isolated kidneys at the end of each treatment and quantified claudin-14 mRNA and protein levels with real-time PCR and western blot respectively.

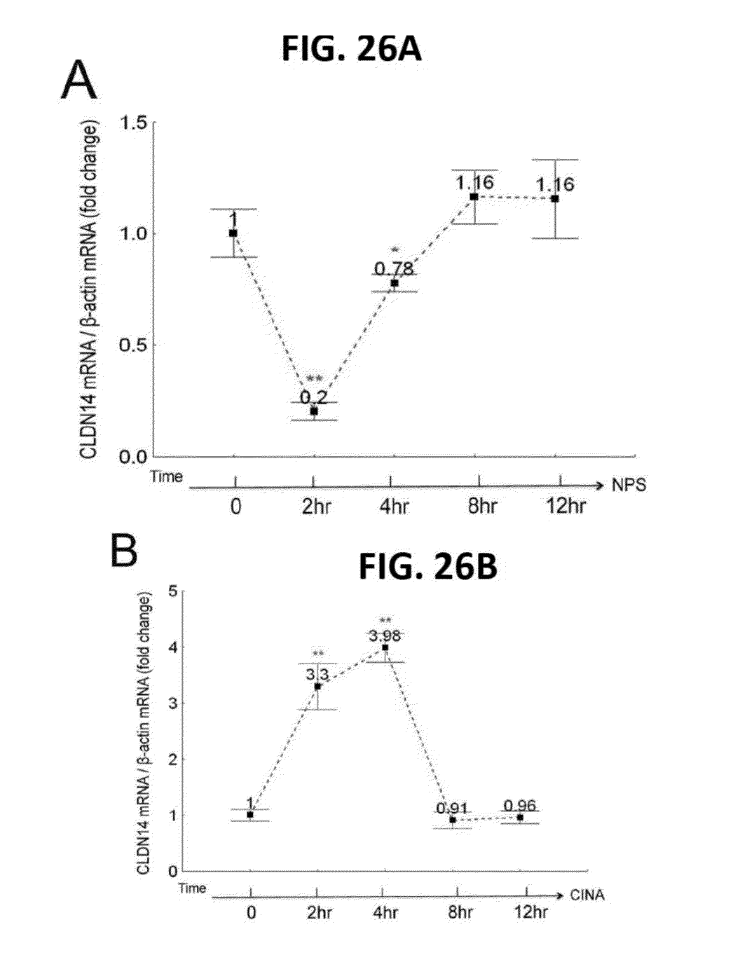

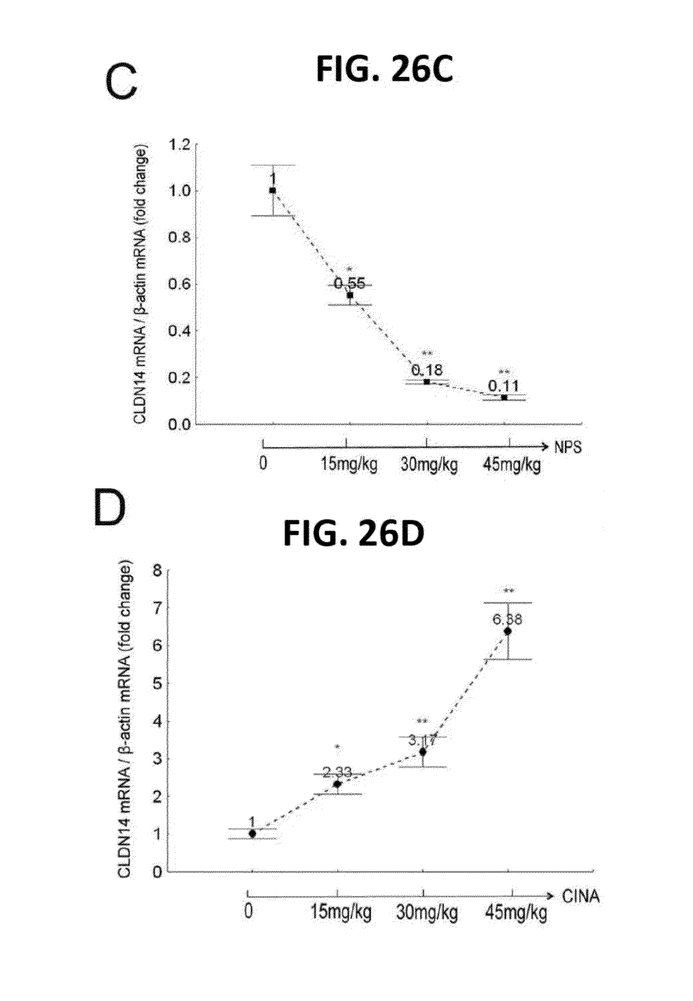

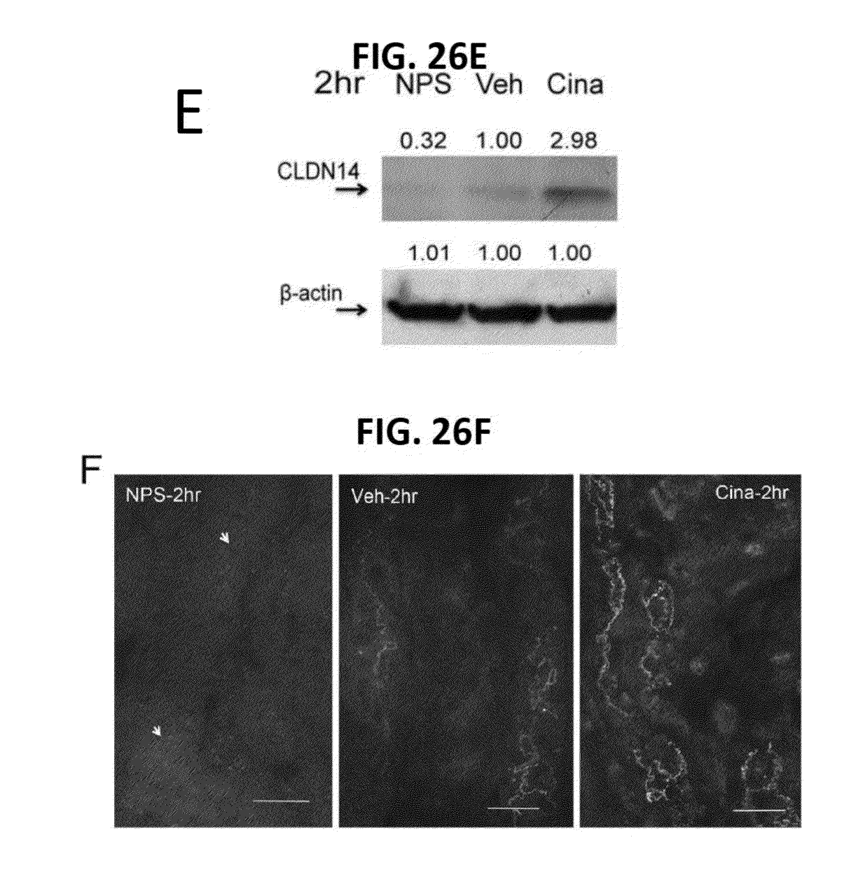

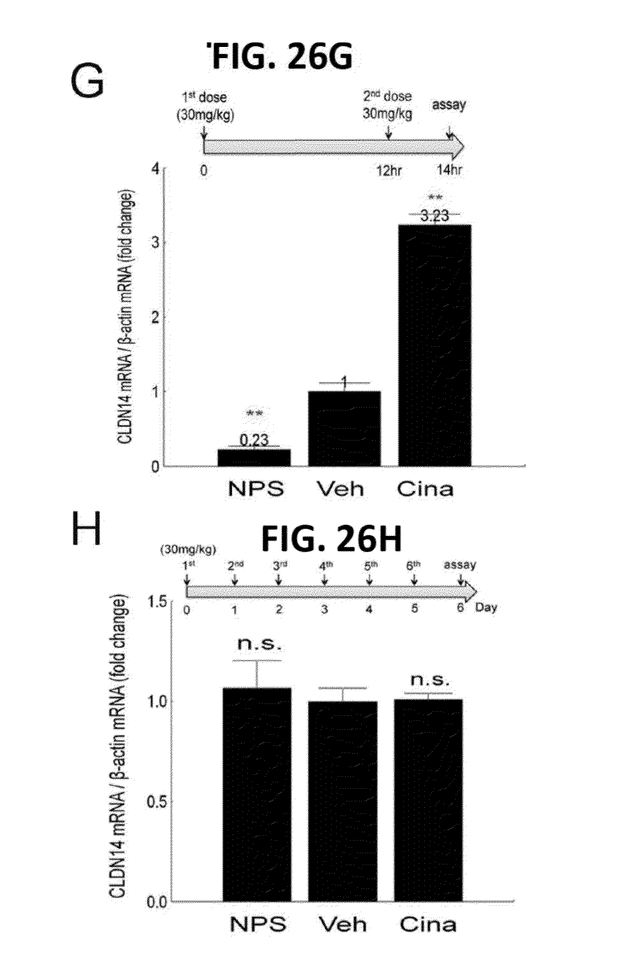

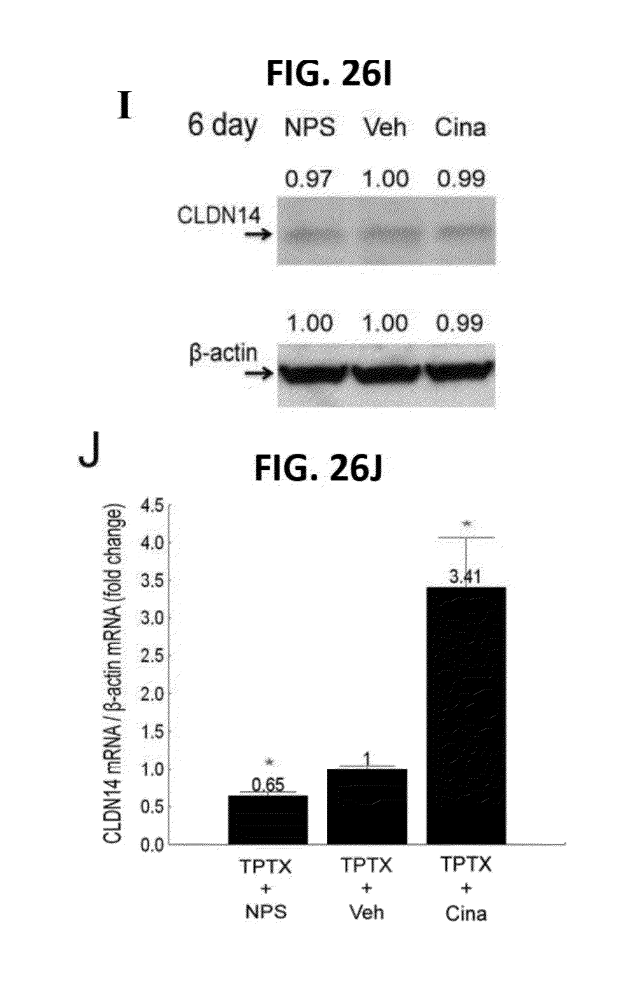

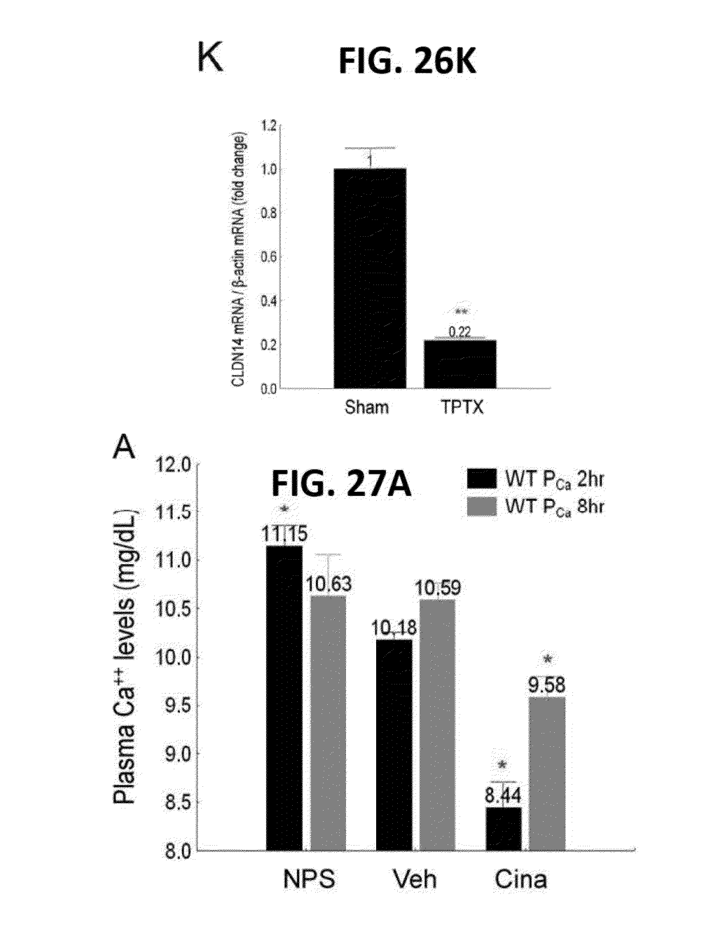

Both NPS2143 and cinacalcet rapidly regulated the mRNA and protein levels of claudin-14 in the kidney. A single oral dose of NPS2143 at 30 mg/kg BW⁻¹ downregulated the mRNA level of claudin-14 by 80% (normalized to β-actin mRNA) at 2 hrs (p<0.01, n=3 versus vehicle; FIG. 26A); the dowuregulation persisted through to 4 hrs although claudin-14 mRNA recovered to 78% of its control level (p<0.05, n=3 versus vehicle; FIG. 26A): by 8 hrs and through to 12 hrs, claudin-14 mRNA completely recovered to its control level. Cinacalcet at a single oral dose of 30 mg/kg BW⁻¹, on the other hand, significantly upregulated the mRNA level of claudin-14 by 3.3-fold at 2 hrs (p<0.01, n=3 versus vehicle; FIG. 26B); by 4 hrs the claudin-14 mRNA level reached the ceiling of 3.98-fold (p<0.01, n=3 versus vehicle; FIG. 26B); by 8 hrs and through to 12 hrs, claudin-14 mRNA recovered to its control level.

To determine the dosage effect of NPS2143 and cinacalcet, the inventors selected the time point for both drugs at 2 hrs. Across the doses of 15, 30 and 45 mg/kg BW⁻¹, NPS2143 treatments generated progressive reduction of claudin-14 mRNA levels, by 45%, 82% and 89% respectively (p<0.05, n=4 versus vehicle; FIG. 26C); while cinacalcet progressively induced claudin-14 expression by 2.33, 3.17 and 6.38-fold respectively (p<0.05, n=4 versus vehicle: FIG. 26D). The dose dependence of claudin-14 expression levels appeared to be linear within 30 mg/kg BW⁻¹ for both drugs.

Changes in claudin-14 protein levels were captured as early as 2 hrs following a single oral dose of NPS2143 or cinacalcet (30 mg/kg BW⁻¹) treatment. Because of the low abundance of claudin-14 proteins in the whole kidney, the inventors adapted an immunomagnetic separation method to freshly isolate the TALH tubular cells from the kidneys of each treated mouse (described in Gong, Y., et al., 2012 and Hou, J., et al. 2009; see Methods), pooled the TALH cells from all animals (N=5) within each treatment group, isolated plasma membrane proteins and quantified claudin-14 protein levels by western blot. While NPS2143 down-regulated claudin-14 protein levels to 32% of the vehicle treatment on densitometric scale (FIG. 26E; normalized to β-actin protein), cinacalcet upregulated claudin-14 protein levels by 2.98-fold (FIG. 26E). In vitro in primary cultures of TALH cells, our data showed a 30 min half-life of claudin-14 protein upon treatment with cycloheximide. Although claudins were previously considered static molecules sequestered in the tight junction, several recent studies have found similarly rapid turnover rate of less than 60 min for claudins (see Discussion). Claudin-14 proteins were inummuostained in mouse kidneys to investigate changes in TJ localization.

Using antibody methods (see Methods) the inventors detected claudin-14 proteins in tight junctions of vehicle treated mice that showed an interdigitated pattern characteristic of the TALH tubule (FIG. 26F). While NPS2143 reduced the staining signal for claudin-14 to punctuate foci (arrow head) apically located reminiscent of dissolved TJ strands, cinacalcet upregulated claudin-14 protein levels in the tight junction that now showed reinforced staining in the TALH tubules (FIG. 26F).

To determine the long-term effects of NPS2143 and cinacalcet on claudin-14 gene expression, the inventors explored whether pretreatment of NPS2143 or cinacalcet for 12 hrs would interfere with their short-term effects. Since the inventors determined that a first dose of NPS2143 or cinacalcet (30 mg/kg BW⁻¹) generated the most pronounced gene regulation of claudin-14 at 2 hrs that dissipated completely by 12 hrs, the inventors gave animals a second dose of NPS2143 or cinacalcet (30 mg/kg BW⁻¹) at 12 hrs and measured claudin-14 gene expression at 14 hrs. The second dose of NPS2143 reduced claudin-14 mRNA levels by 77% (p<0.01, n=3 versus vehicle; FIG. 26G), to a similar extent as a single dose treatment (FIG. 26A); whereas the second cinacalcet treatment increased claudin-14 mRNA levels by 3.23-fold (p<0.01, n=3 versus vehicle; FIG. 26G), not different from the first dose (FIG. 26B).

The inventors explored whether continuous treatments with NPS2143 or cinacalcet for 6 days would pre-program claudin-14 gene expression. To distinguish from the observed short-term effects, the inventors gave animals an oral dose of NPS2143 or cinacalcet (30 mg/kg BW⁻¹) per day for 6 days and measured claudin-14 gene expression 24 hrs following the last treatment. Neither NPS2143 nor cinacalcet caused changes in claudin-14 mRNA (FIG. 26H) or protein levels (FIG. 9I) during the 6-day treatment. The long-term NPS2143 or cinacalcet treatment (FIG. 26H-I) demonstrated a lack of plasticity in CaSR signaling.

The inventors explored whether CaSR regulation of claudin-14 depended upon the PTH secretion. In thyroparathyroidectomy (TPTX) mice with nominal absence of circulating PTH, cinacalcet at a single dose of 30 mg/kg BW⁻¹ significantly upregulated claudin-14 expression levels by 3.41-fold at 2 hrs (p<0.05, n=5 versus vehicle; FIG. 26J), and cinacalcet induced a comparable 3.3-fold upregulation in intact mice (FIG. 26B). NPS2143 was able to further downregulate claudin-14 gene expression by 35% at 2 hrs after a single dose of 30 mg/kg BW⁻¹ (p<0.05, n=5 versus vehicle; FIG. 26J) in TPTX treated mice, mindful that the basal level of claudin-14 in TPTX animals was already 78% lower than in sham controls (FIG. 26K). These results indicate that the CaSR regulation of claudin-14 in the kidney is rapid and independent of PTH.

### Example 23

This example illustrates physiological Ca⁺⁺ regulation abolished in claudin-14 knockout animals

The inventors explored whether claudin-14 underlay the physiological role of CaSR in renal Ca⁺⁺ transport. The inventors previously described a claudin-14 KO mouse model that showed hypomagnesiuria and hypocalciuria under high Ca⁺⁺ dietary condition. (Gong, Y., et al. 2012) To investigate the functional role of claudin-14 in CaSR signaling, the inventors treated age (10-12 weeks old) and sex (male) matched claudin-14 KO mice (established on C57BL/6 strain; Methods) and their littermate WT controls with NPS2143 or cinacalcet at a single dose of 30 mg/kg BW⁻¹ (N=5-7 for each treatment group). The time dependent change in CaSR mediated claudin-14 gene regulation (FIG. 26A-B) would suggest that urinary Ca⁺⁺ excretion was time sensitive, i.e. peaking at 2 hr but dissipating by 8 hr after NPS2143 or cinacalcet treatment.

To capture these changes in renal excretion function, the inventors performed 1 hr renal clearance measurements on mice infused with FITC-inulin (Methods) starting at 2 hrs and 8 hrs respectively. At 2 hrs, NPS2143 treatment significantly increased the plasma Ca⁺⁺ level (P_{Ca}) to 11.15±0.21 mg/dL in WT mice (versus 10.18±0.08 mg/dL in vehicle treated animals; p<0.05; FIG. 27A; Table 7), accompanied by a dramatic decrease in urinary excretion of Ca⁺⁺ (FE_{Ca}: 0.09±0.01% versus 0.63±0.09% in vehicle group; FIG. 27B; Table 7); whereas cinacalcet significantly decreased P_{Ca} (8.44±0.27 mg/dL; p<0.05 versus vehicle; FIG. 27A) and increased FE_{Ca} (3.82±0.52%: p<0.01 versus vehicle: FIG. 27B) in WT animals. At 8 hrs, the plasma Ca⁺⁺ level was still significantly lower in cinacalcet group but returned to normal in NPS2143 treated animals (FIG. 27A; Table 7). The urinary Ca⁺⁺ excretion levels at 8 hrs were not different across NPS2143, vehicle and cinacalcet groups (FIG. 27B; Table 7).

Renal handling of Mg⁺⁺ was similar to that of Ca⁺⁺ in WT mice. The plasma Mg⁺⁺ level (P_{Mg}) was significantly altered by NPS2143 and cinacalcet at 2 hrs but returned to baseline at 8 hrs (FIG. 27C; Table 7). The changes in urinary excretion of Mg⁺⁺ (FE_{Mg}) were inversely correlated with that in P_{Mg}, which was significant at 2 hrs (p<0.05 for NPS2146 and cinacalcet versus vehicle) but dissipated at 8 hrs (FIG. 27D; Table 7).

Plasma PTH levels were significantly affected by NPS2143 or cinacalcet at both 2 hrs and 8 hrs (p<0.05 versus vehicle: FIG. 27E). At 8 hrs, the PTH level in NPS2143 group started to return towards baseline (p=0.08 versus 2 hr) while cinacalcet continued to suppress plasma PTH levels through 2 hrs to 8 hrs. The claudin-14 KO animals were refractory towards CaSR mediated renal Ca⁺⁺ regulation. At 2 hrs, NPS2143 induced hypercalcemia (P_{Ca}; FIG. 27F; Table 7) and hypocalciuria (FE_{Ca}; FIG. 27G; Table 7) were abolished in KO animals despite significant increases in plasma PTH levels (p<0.05 versus vehicle; FIG. 27J). Cinacalcet-induced hypocalcemia was still present in KO animals (p<0.05 versus vehicle: FIG. 27F), which appeared to have originated from extrarenal sources due to suppressed PTH levels (FIG. 27J), because their urinary Ca⁺⁺ excretion was not different from the vehicle group (p=0.19; FIG. 27G).

The baseline plasma Mg⁺⁺ level was significantly higher in KO animals compared to that in WT animals (vehicle group; P_{Mg}: 2.71±0.05 mg/dL in KO versus 2.34±0.04 mg/dL in WT: p<0.01; FIG. 27H; Table 7), accompanied by a trend towards lower baseline urinary excretion of Mg⁺⁺ (FE_{Mg}: 11.67±2.11% in KO versus 16.59±1.31% in WT; p=0.09: FIG. 27I; Table 7). This is consistent with our previous in vitro recordings that claudin-14 is a negative regulator of the paracellular channel reabsorbing divalent cations in the kidney. (Gong, Y., et al. 2012) Both NPS2143 and cinacalcet dependent regulation of P_{Mg} (FIG. 27H) and FE_{Mg} (FIG. 27I) was abrogated in KO animals comparable to their effects on Ca⁺⁺ handling. The deregulation of urinary Ca⁺⁺ metabolism in claudin-14 KO animals demonstrates that claudin-14 is the functional “effector” underpinning the renal signaling pathway of CaSR.

### Example 24

This example illustrates transgenic overexpression of claudin-14 in the kidney induces hypercalciuria and hypermagnesiuria

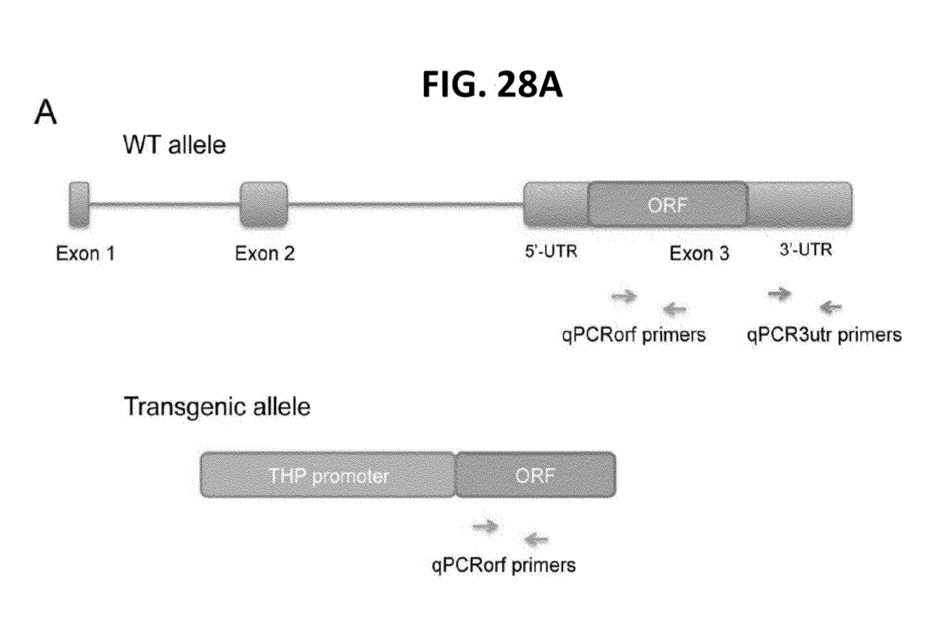

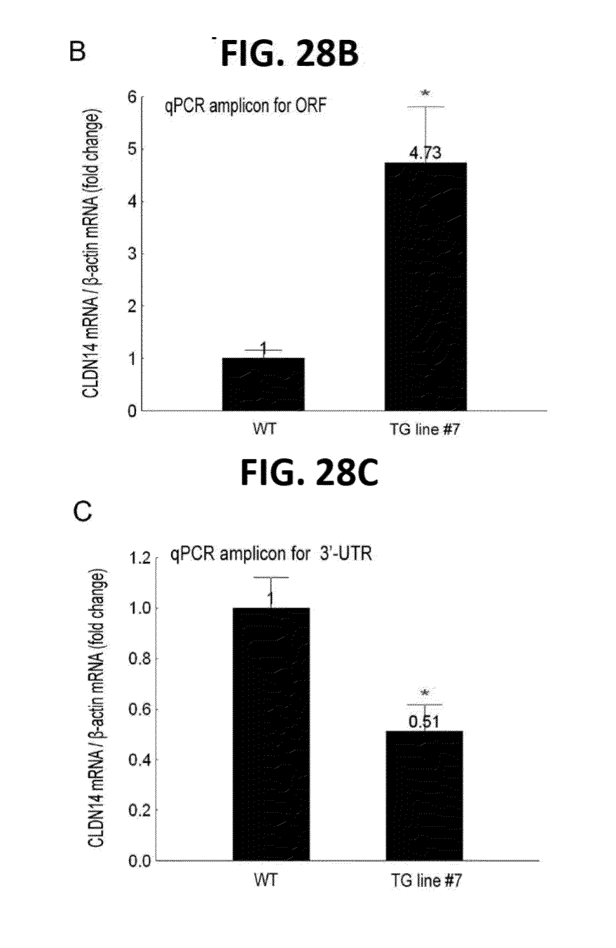

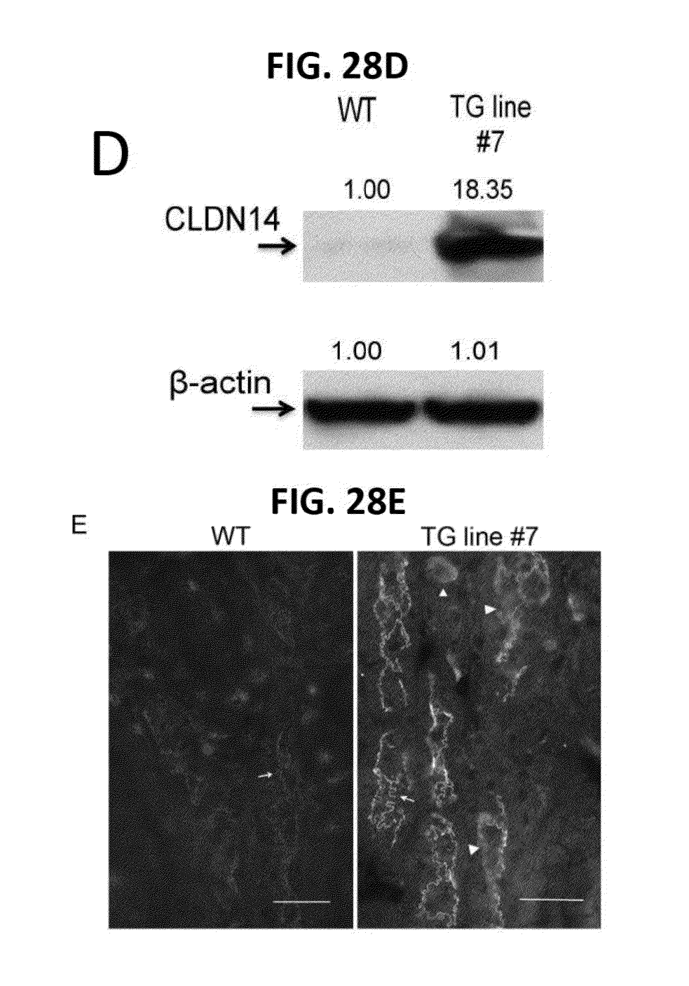

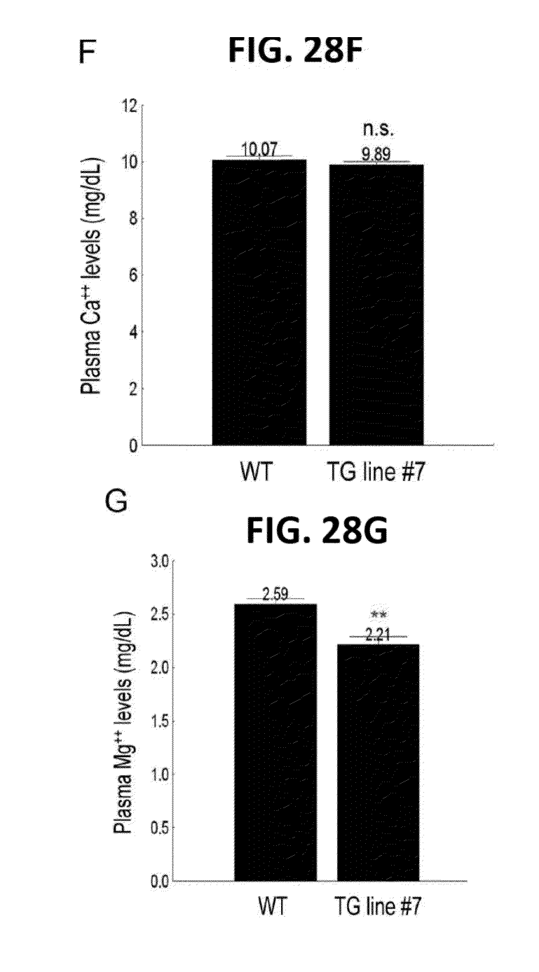

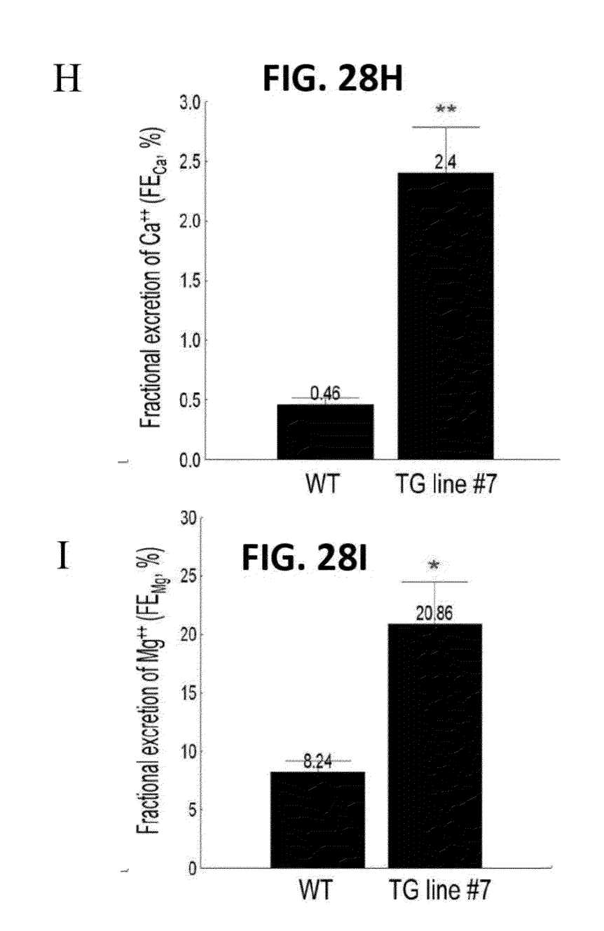

In a previous report, the inventors demonstrated that claudin-14 interacts with and inhibits claudin-16 channel permeability using several in vitro biochemical, biophysical and cellular criteria. (Gong, Y., et al., 2012) To provide in vivo evidence that manipulation of claudin-14 gene expression in the kidney per se is sufficient to disrupt Ca⁺⁺ transport, the inventors generated transgenic mouse models (established on C57BL/6x CBA hybrid background) to overexpress the claudin-14 gene selectively in the TALH epithelia of the kidney. The mouse claudin-14 gene was cloned downstream of a proven TALH-specific gene promoter—Tamm-Horsfall protein (THP) (Hou, J., et al., 2009) (FIG. 28A), allowing TALH-specific transgenic expression.

After transgenesis (see Methods), the TALH tubules were immuno-isolated from mature hemizygous transgenic mouse kidneys for quantitative analyses of claudin-14 gene expression. Because the transgene contained the open reading frame (ORF) but no 5′- or 3′-untranslated region (UTR) of the claudin-14 gene, the inventors designed two pairs of primers to differentiate the transgenic from endogenous claudin-14 expression (FIG. 28A). The primer pair “qPCRorf” allowed amplifying the total claudin-14 transcripts while the primer pair “qPCR3utr” selectively amplified the endogenous claudin-14 transcripts (see Methods).

The total claudin-14 mRNA level (normalized to j3-actin mRNA) was increased by 4.73-fold (p<0.05, n=6; FIG. 28B) in a transgenic line (TG line #7) compared to its WT littermates. The endogenous claudin-14 mRNA levels was instead decreased by 49% (p<0.05, n=6 versus WT; FIG. 28C) in the same line, implicating a feedback loop involving the CaSR signaling described elsewhere of this study. Thus, the transgenic expression on the mRNA level was estimated 9.27-fold relative to the endogenous level in WT. The claudin-14 proteins, pooled from the isolated TALH tubules of 6 animals in each group (N=6), were dramatically upregulated by 18.35-fold in TG animals compared to WT controls (FIG. 28D). Because the transgene contained no 3′-UTR of the claudin-14 gene where two microRNAs—miR-9 and miR-374 bind, (Gong, Y., et al., 2012) the transgenic claudin-14 transcript would be more efficient in translation compared to its endogenous transcript, therefore causing the observed super-regulation on the protein level.

In WT mouse kidneys, claudin-14 was faintly inmumostained in the TALH tubules showing interdigitated TJ pattern (arrow; FIG. 28E). In transgenic mouse kidneys, claudin-14 staining was profoundly strengthened in tight junctions of the TALH tubules (arrow; FIG. 28E), with occasional diffuse staining spotted in the subapical region of some TALH cells (arrowhead; FIG. 28E) suggestive of transgenic protein saturation. To investigate the functional effects of claudin-14 overexpression in the kidney, the inventors performed 24 h urinalysis on age (10-12 weeks old) and sex (male) matched claudin-14 TG mice and their littermate WT controls. The plasma Ca⁺⁺ level in TG mice was well defended (FIG. 28F; Table 8), but their circulating Mg⁺⁺ level was significantly lower than WT controls (P_{Mg}; TG: 2.21±0.08 mg/dL versus WT: 2.59±0.05 mg/dL: p<0.01, n=6; FIG. 28G; Table 8). The fractional excretion rates for Ca⁺⁺ (FE_{Ca}) and Mg⁺⁺ (FE_{Mg}) in TG animals were profoundly increased by 5.22-fold (p<0.01, n=6; FIG. 28H; Table 8) and 2.53-fold (p<0.05, n=6: FIG. 28I: Table 8) respectively compared to WT animals, indicating a direct renal tubular defect. The glomerular filtration rate (GFR) based on creatinine clearance (Table 8) was not significantly different between TG and WT animals, nor was the urinary volume (UV) (Table 8). The phenotypes of plasma and urine electrolyte abnormalities of TG line #7 were recapitulated in a second transgenic line TG #24, whose total claudin-14 transcript level was 5.68-fold higher than WT (p<0.05, n=6). These results established the in-vivo function of claudin-14 in the kidney that negatively regulates the paracellular divalent reabsorption, thus phenocopying the renal phenotypes of claudin-16 KO and claudin-19 KO animals.

### Example 25

This example illustrates that microRNA transcription but not the claudin-14 promoter is directly regulated by CaSR

The inventors identified a microRNA-based mechanism involving two microRNA molecules—miR-9 and miR-374 in the TALH of the kidney to regulate claudin-14 mRNA decay and translational repression through reciprocal changes of their own cellular abundance in response to CaSR signals. (Gong, Y., et al., 2012) MiR-9 is transcribed from 3 genomic loci in both human and mouse: miR-9-1, miR-9-2 and miR-9-3. (Ma, L., et al., 2010) Human miR-374 has two isoforms—a and b, both sharing the same seed sequence and each having its own genomic locus. Mature human miR-374b is identical to mouse miR-374 that is transcribed from a single genomic locus, while human miR-374a has no mouse homologue. To measure the transcriptional level for microRNA gene in vivo in the kidney, the inventors designed primers (Methods; Table 2) to amplify the pri-miRNA molecule, the original transcript for microRNA gene that had not yet been subjected to any nuclear or cellular processing.

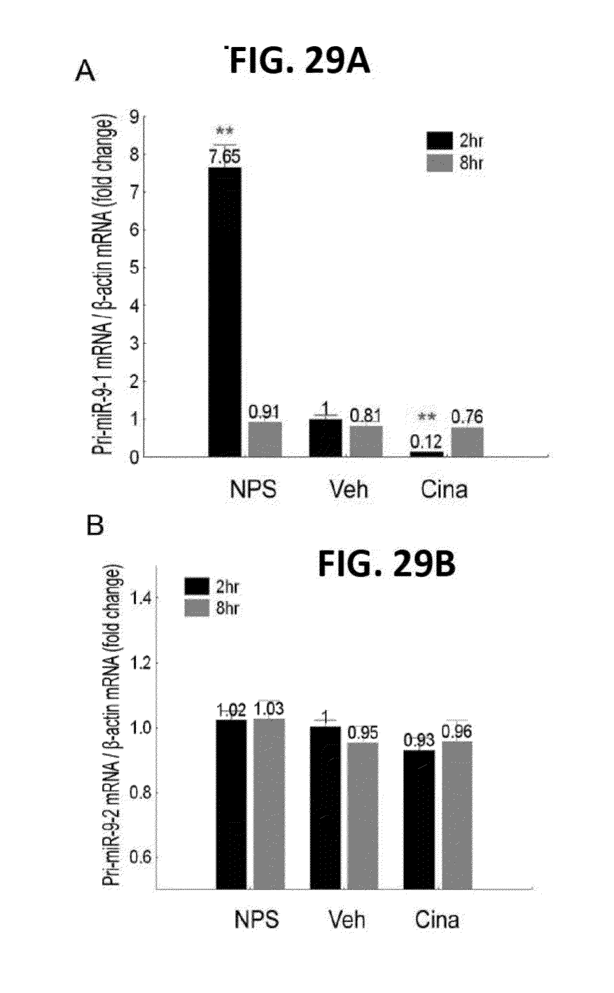

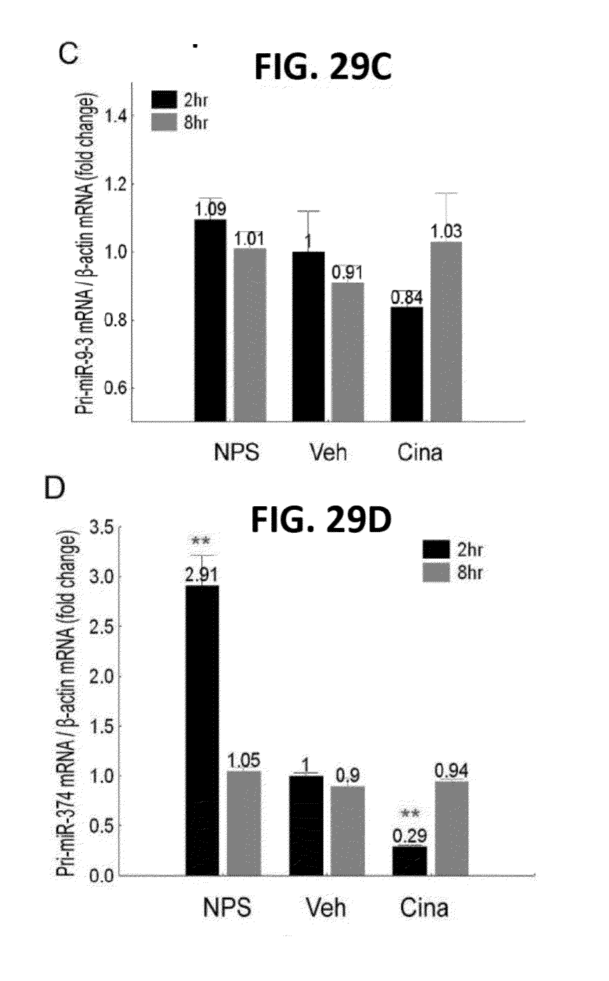

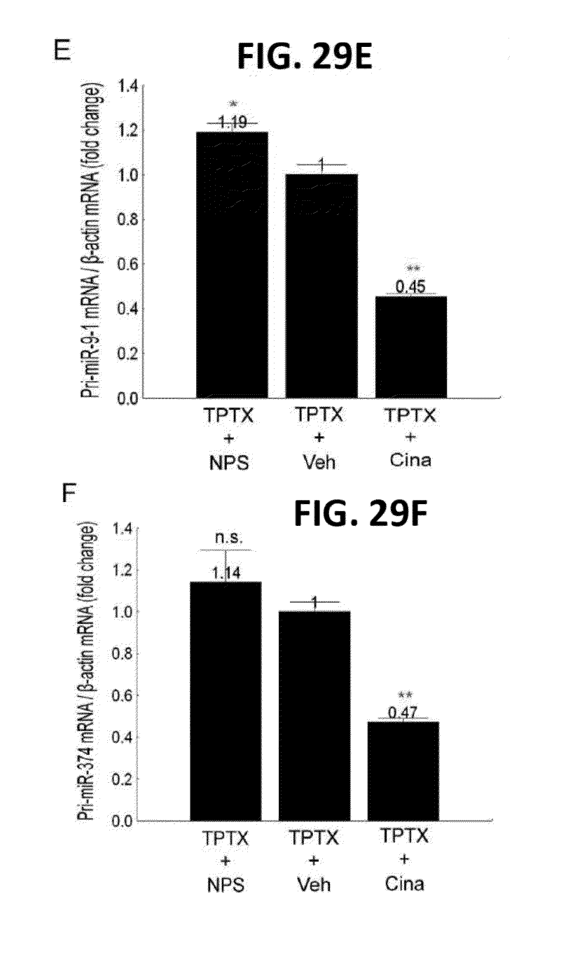

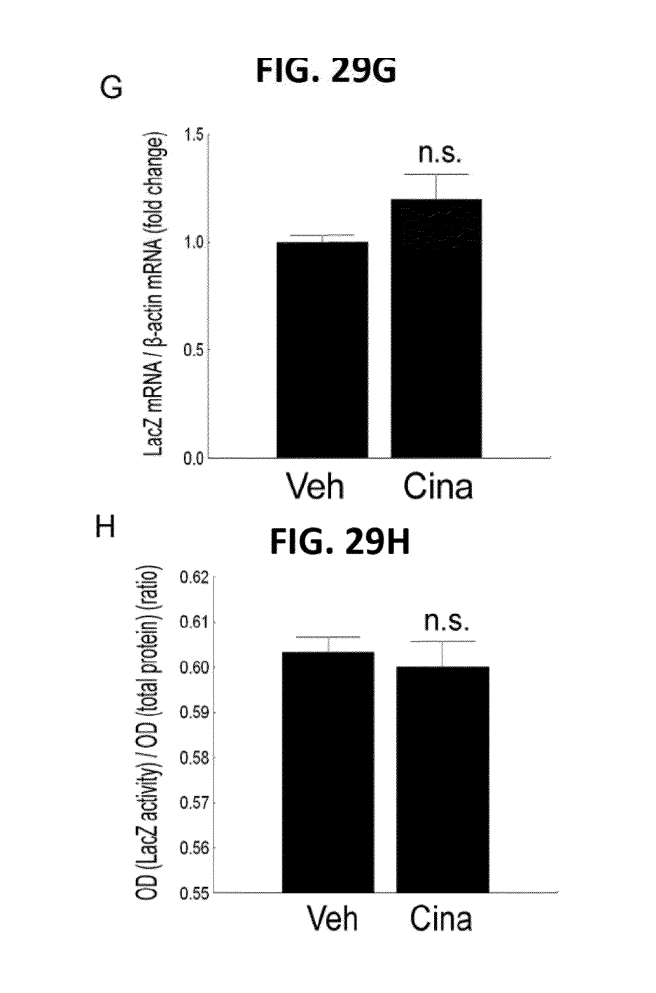

Because both miR-9 and miR-374 had broad localization profiles along the nephron (Gong, Y., et al., 2012), the inventors isolated the TALH tubular cells for pri-miRNA analyses from the mouse kidney described elsewhere in this study. WT mice (strain: C57BL/6) were treated with NPS2143 or cinacalcet as described for claudin-14 analyses and assayed for pri-miRNA levels in isolated TALH cells at 2 hrs and 8 hrs respectively. Among the three miR-9 genes, miR-9-1 was the most sensitive to CaSR modulation: showing a 7.65-fold increase in its pri-miRNA transcript level 2 hrs after single-dose 30 mg/kg BW⁻¹ NPS2143 treatment (p<0.01, n=4 versus vehicle; FIG. 29A): and 88% decrease 2 hrs after a 30 mg/kg BW⁻¹ cinacalcet treatment (p<0.01, n=4 versus vehicle; FIG. 29A). By 8 hrs, the changes in pri-miR-9-1 levels induced by NPS2143 or cinacalcet had dissipated completely (FIG. 29A). Neither pri-miR-9-2 (FIG. 29B) nor pri-miR-9-3 level (FIG. 29C) was significantly altered by NPS2143 or cinacalcet at 2 hrs or 8 hrs. NPS2143 and cinacalcet also markedly regulated pri-miR-374. At 2 hrs, NPS2143 upregulated pri-miR-374 by 2.91-fold while cinacalcet downregulated it by 71% (p<0.01, n=4 versus vehicle: FIG. 29D). Similar to pri-miR-9-1, pri-miR-374 levels were not different from control 8 hrs after NPS2143 or cinacalcet treatment (FIG. 29D). It was evident that the expression levels of miR-9-1 and miR-374 genes were both inversely correlated with that of claudin-14 gene in the kidney, compatible with the silencing role of microRNA in regulating its target gene.

The inventors investigated whether CaSR regulation of pri-miRNA depended upon PTH secretion. In TPTX treated mice (described above), cinacalcet at a single dose of 30 mg/kg BW⁻¹ significantly reduced pri-miR-9-1 and pri-miR-374 levels by 55% and 53% respectively at 2 hrs (p<0.01, n=5 versus vehicle; FIG. 29E-F). NPS2143 at 30 mg/kg BW⁻¹ significantly increased the pri-miR-9-1 level by 19% in TPTX treated animals (p<0.05, n=5 versus vehicle; FIG. 29E) but failed to elicit a significant change in pri-miR-374 level, which may have been due to severe hypocalcemia in TPTX animals that had already experienced several-fold higher basal levels of pri-miR-9-1 and pri-miR-374 compared to sham-operated animals.

The inventors explored alternative mechanisms for claudin-14 gene regulation. Because the claudin-14 KO mouse was generated with a lacZ reporter gene replacing the last exon of claudin-14 gene (including the coding region and 3′-UTR; Ben-Yosef, T., et al., 2003) the expression of lacZ gene would provide a faithful measurement for endogenous claudin-14 promoter activity regardless of any microRNA based regulation. The claudin-14^{+/lacZ} mice were treated with cinacalcet or NPS2143 as described for WT mice. At 2 hrs after single-dose 30 mg/kg BW⁻¹ treatment, cinacalcet caused no change in lacZ mRNA levels (normalized to β-actin mRNA: n=4; FIG. 29G) or β-galactosidase activity (normalized to total soluble proteins; n=4; FIG. 29H), assayed in claudin-14^{+/lacZ} mouse kidneys and isolated TALH cells respectively, nor did NPS2143 at the same dosage and time point. These data have demonstrate that CaSR regulates claudin-14 gene expression not through cis-acting element, i.e. its promoter but through trans-acting factors, i.e. miR-9 and miR-374.

### Example 26

This example illustrates a calcineurin inhibitor abrogating CaSR mediated regulation of claudin-14, microRNAs and urinary Ca⁺⁺ excretion.

The calcineurin inhibitor cyclosporine-A induces hypomagnesemia and hypercalciuria in laboratory animals, previously thought to occur because of disturbed TRPV5 and TRPM6 channel expression in the kidney. (Nijenhuis, T., et al., 2004) Cyclosporine-A reduces the paracellular but not transcellular divalent cation transport in cultured mouse TALH cells in vitro. (Chang, C. T., et al., 2007) To investigate how cyclosporine-A affects claudin-14 gene expression and renal Ca⁺⁺ handling in vivo, the inventors pre-treated WT mice (strain: C57BL/6) with cyclosporine-A (Sandimmune) at a single I.P. dose of 25 mg/kg BW⁻¹ per day for 6 consecutive days followed by NPS2143 or cinacalcet treatment on the 6^{th} day (as described above). Cyclosporine-A pre-treatment did not affect the basal level of claudin-14, pri-miR-9-1 or pri-miR-374 in the TALH of the kidney.

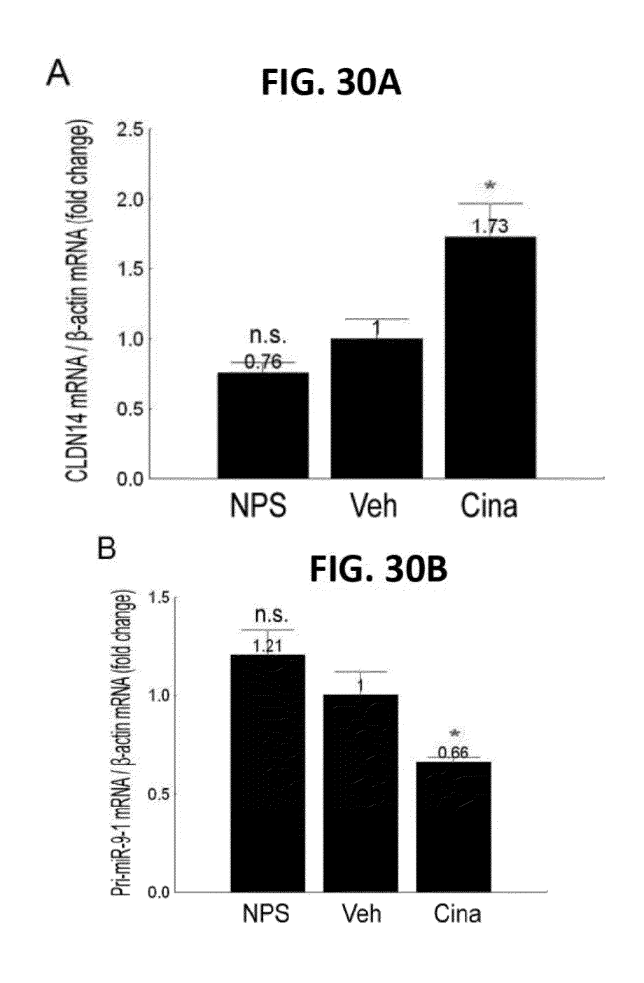

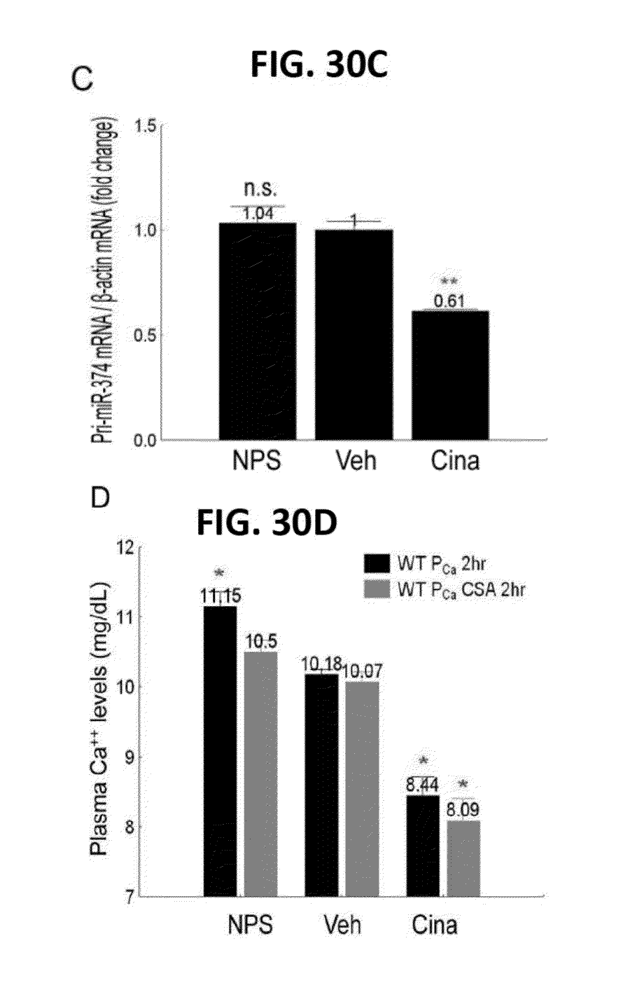

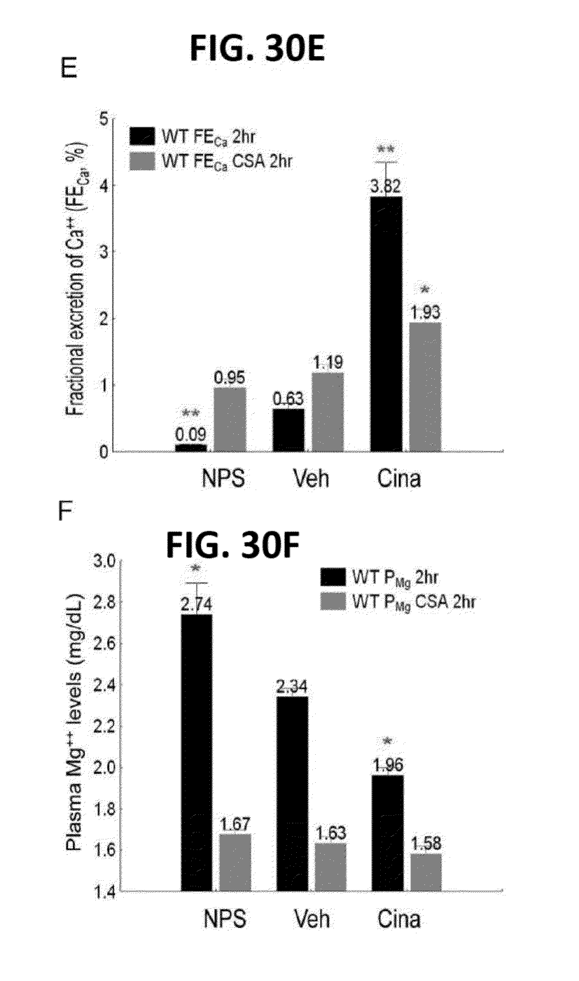

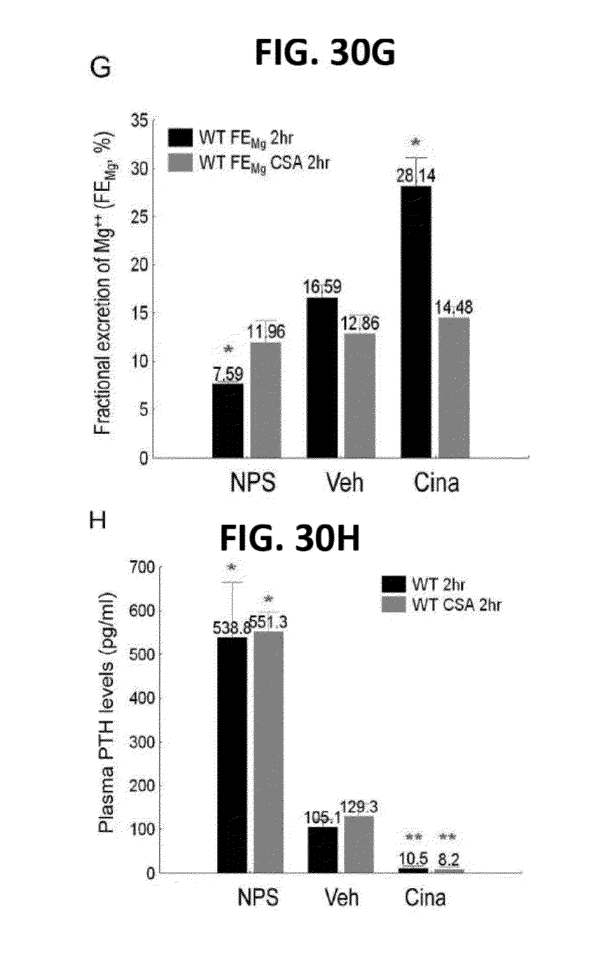

Calcimimetic and calcilytic effects were, however, significantly attenuated by cyclosporine-A pre-treatments. At 2 hrs after single-dose 30 mg/kg BW⁻¹ treatment, NPS2143 failed to elicit any significant change in claudin-14 (n=5 versus vehicle; FIG. 30A), pri-miR-9-1 (FIG. 30B) or pri-miR-374 mRNA levels (FIG. 30C) in cyclosporine-A pre-treated animals. Cinacalcet, at the same dosage and time point, still increased claudin-14 mRNA levels by 1.73-fold (p<0.05, n=5 versus vehicle; FIG. 30A) and decreased pri-miR-9-1 and pri-miR-374 mRNA levels by 34% (p<0.05; FIG. 30B) and by 39% (p<0.01; FIG. 30C) respectively in cyclosporine-A pre-treated animals, nevertheless, its regulatory effects were lower than those captured for animals without cyclosporine-A treatment (claudin-14: 3.3-fold in FIG. 26B; pri-miR-9-1: 88% and pri-miR-374: 71% in FIG. 29A and FIG. 21D respectively).

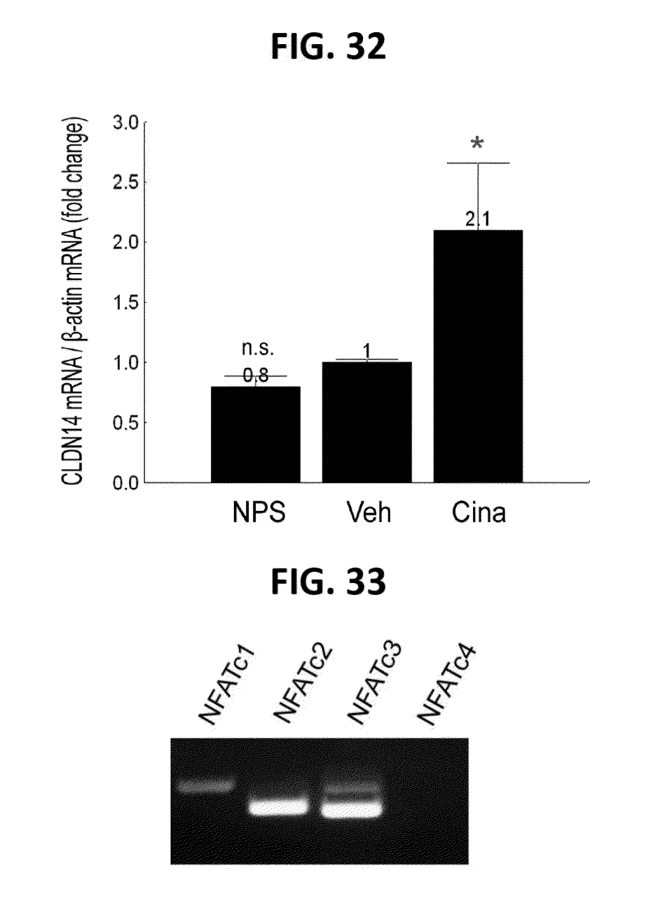

Pre-treatment with FK506 (Tacrolimus) at a single I.P. dose of 3 mg/kg BW⁻¹ per day for 6 consecutive days also attenuated the calcimimetic and calcilytic effects on claudin-14 gene expression (FIG. 32). The inventors investigated whether the calciuretic response to CaSR modulation was attenuated by calcineurin inhibitor. Because the control animals for cyclosporine-A treatment (injected with Sandinmmune solvent; Methods) showed no difference in their plasma and urinary electrolyte levels from WT animals, their physiological data were not included, instead the WT animal data presented in FIG. 27 were superimposed onto the calciuretic and magnesiuretic curves of cyclosporine-A treated animals (FIG. 30D-H). Cyclosporine-A significantly increased the basal level of urinary Ca⁺⁺ excretion (FE_{Ca}: 1.19+0.12% versus 0.63±0.09% in WT; p<0.05, n=5-7; FIG. 30E; Table 7) without affecting the basal plasma Ca⁺⁺ level (P_{Ca}; FIG. 30D). The basal level of P_{Mg} was dramatically reduced by cyclosporine-A to 1.63±0.03 mg/dL compared with 2.34±0.04 mg/dL in WT (p<0.05, n=5-7; FIG. 30F; Table 7), accompanied by nonuomagnesiuria (FIG. 30G). At 2 hrs, the NPS2143 effects on P_{Ca} (FIG. 30D) and FE_{Ca}(FIG. 30E) were abolished by cyclosporine-A, while the cinacalcet effects were maintained (FIG. 30D-E) even though cinacalcet induced hypercalciuria was attenuated to a level 1.93-fold higher than vehicle in cyclosporine-A pre-treated animals (p<0.05, n=5-7: FIG. 30E; Table 7). Both NPS2143 and cinacalcet dependent regulation of P_{Mg} (FIG. 30F) and FE_{Mg} (FIG. 30G) was completely abrogated by cyclosporine-A despite an intact PTH response towards both drugs (FIG. 30H) at 2 hrs.

These results demonstrate a role for calcineurin inhibitor that antagonizes the extracellular Ca⁺⁺ signaling in the kidney causing deranged claudin-14 expression and urinary Ca⁺⁺ excretion.

### Example 27

This example illustrates CaSR signaling involving NFAT dependent regulation of microRNA promoters.

The inventors investigated how calcineurin transduced its signal. Calcineurin is a protein phosphatase that employs a class of transcriptional factors known as NFATs to regulate target gene transcription. (Crabtree, G. R., et al., 2002) The inventors investigated which isoform of NFATs was expressed in the TALH of the kidney. Using primers designed against the four cellular isoforms of NFATs (NFATc1-4; Table 2), the inventors detected predominant gene expression of NFATc1, c2 and c3 but not c4 in immuno-isolated mouse TALH cells (FIG. 33).

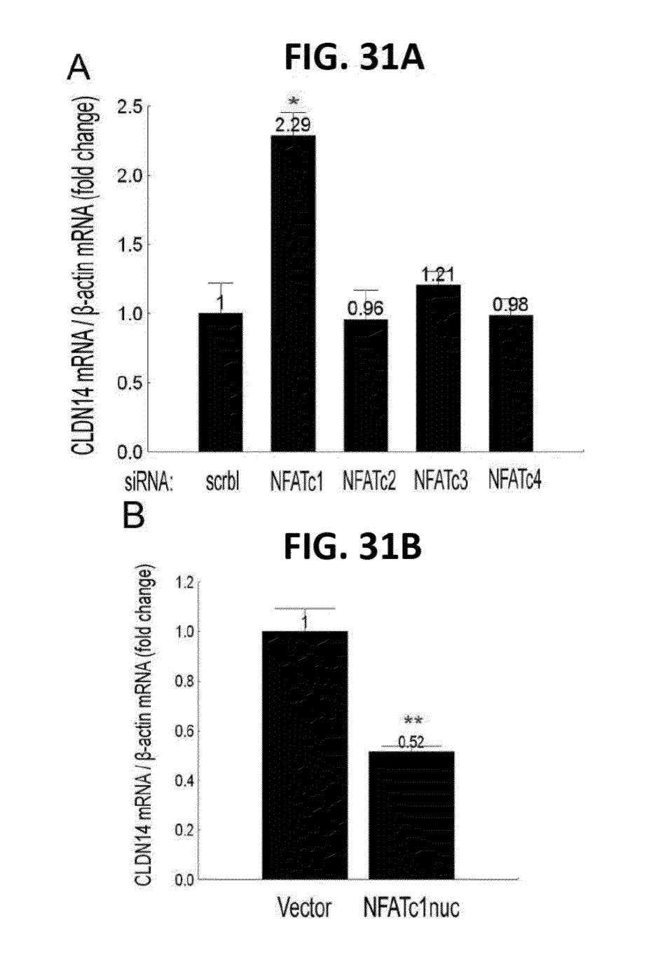

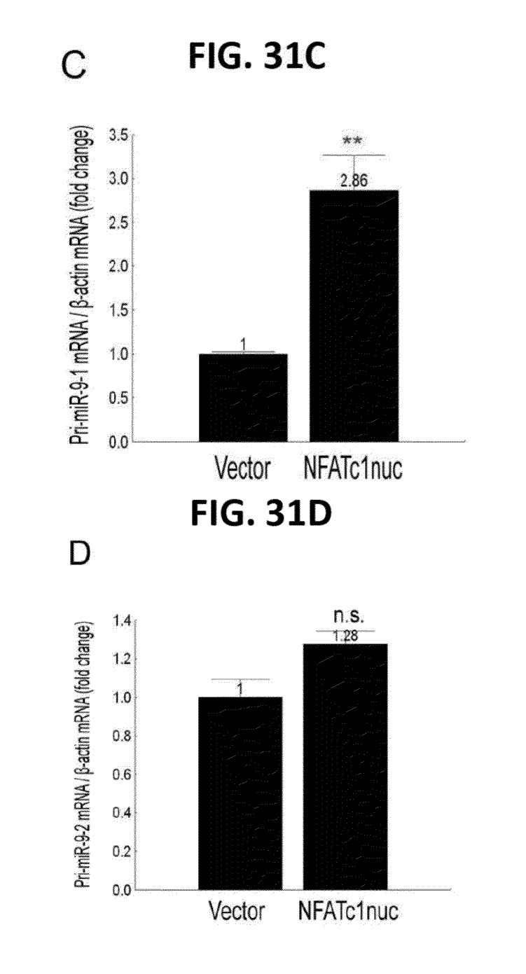

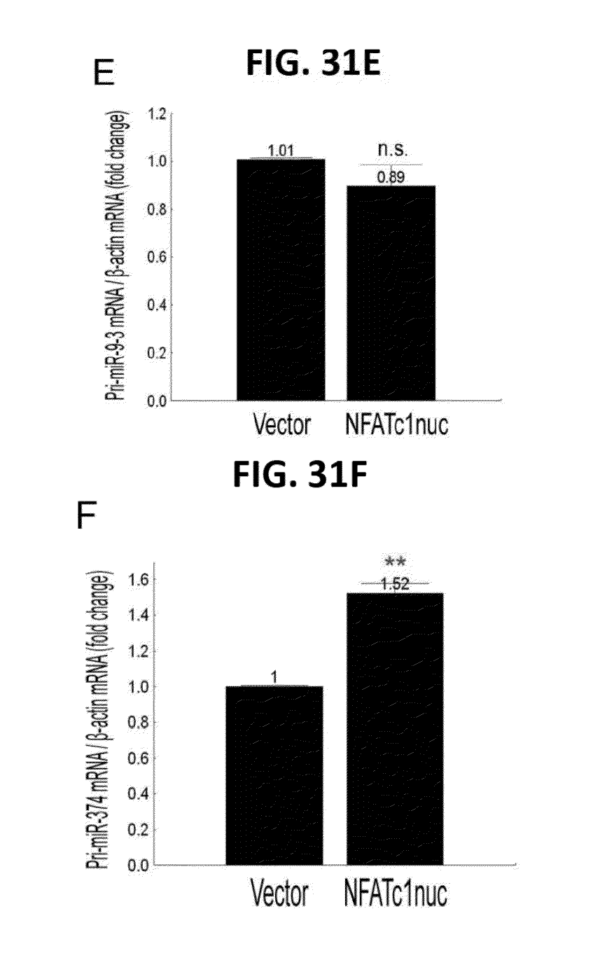

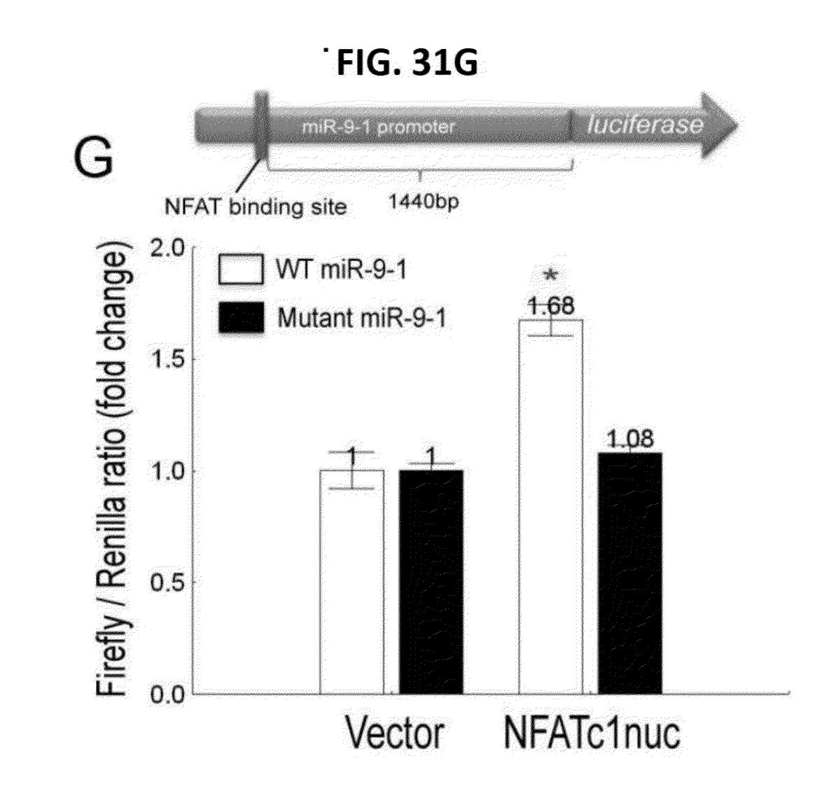

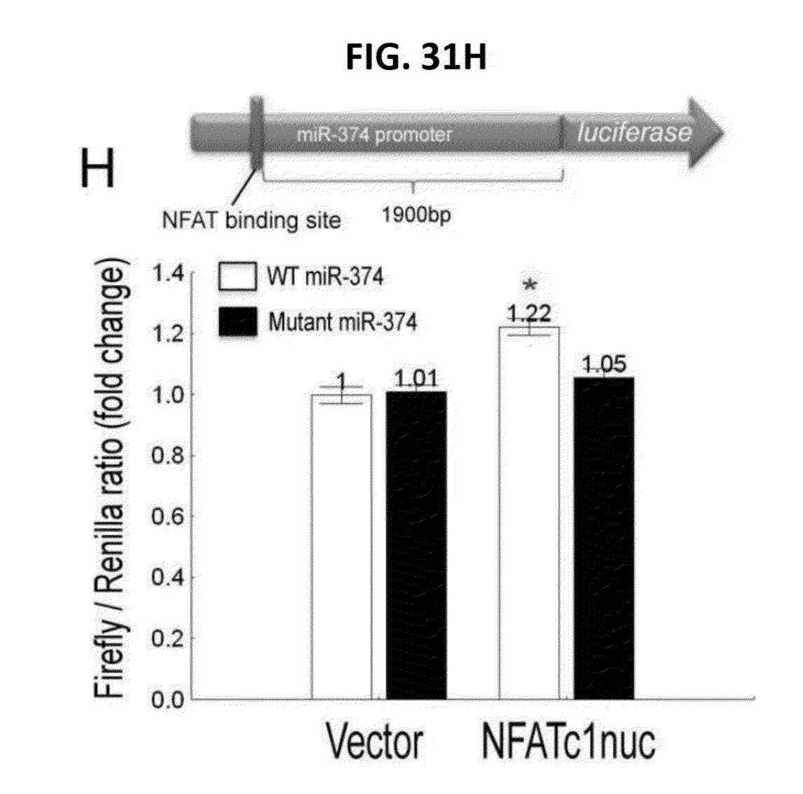

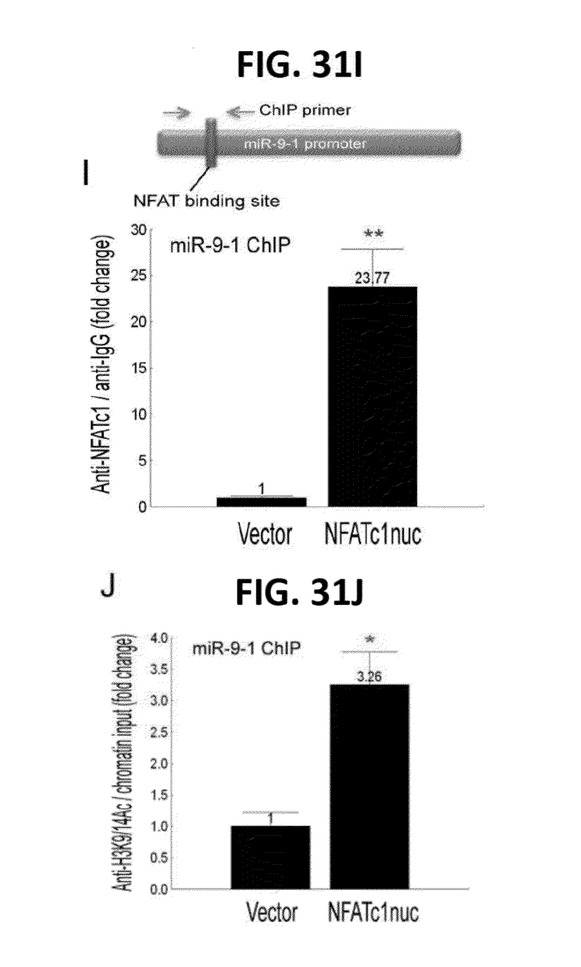

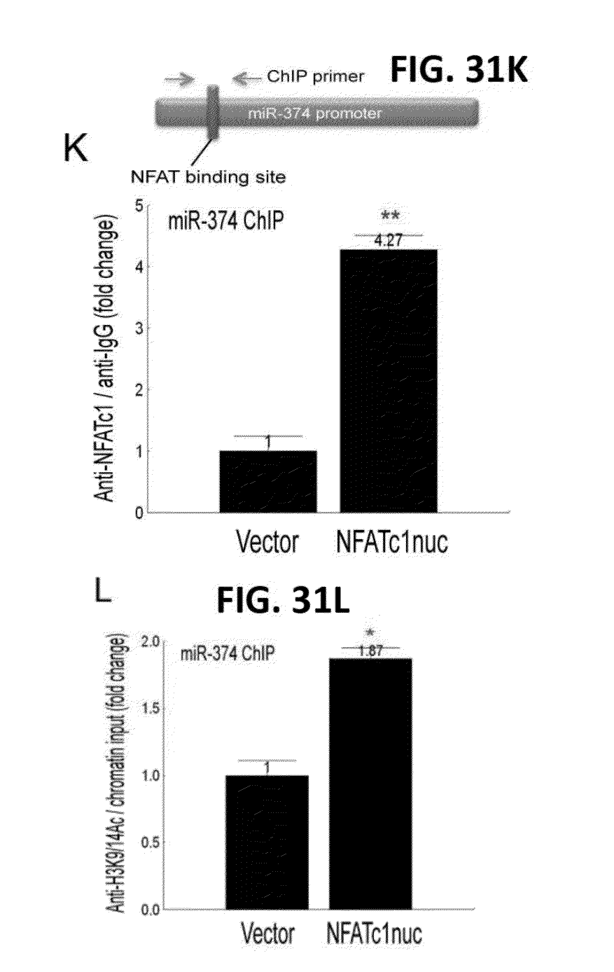

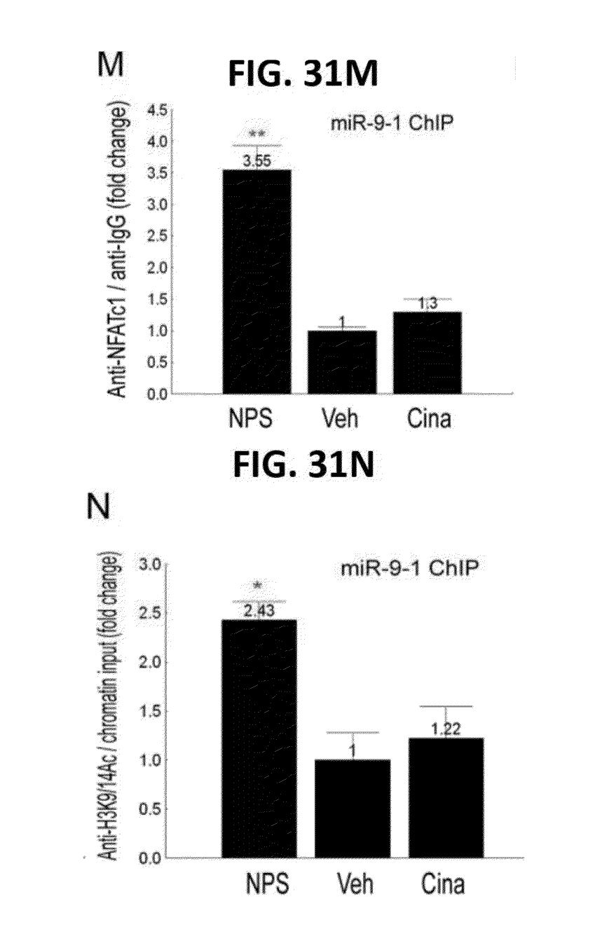

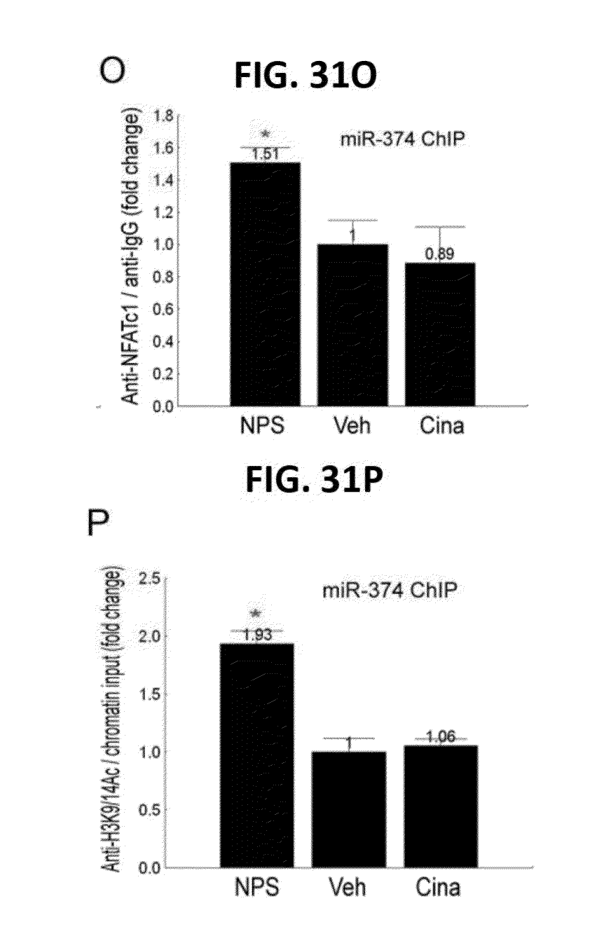

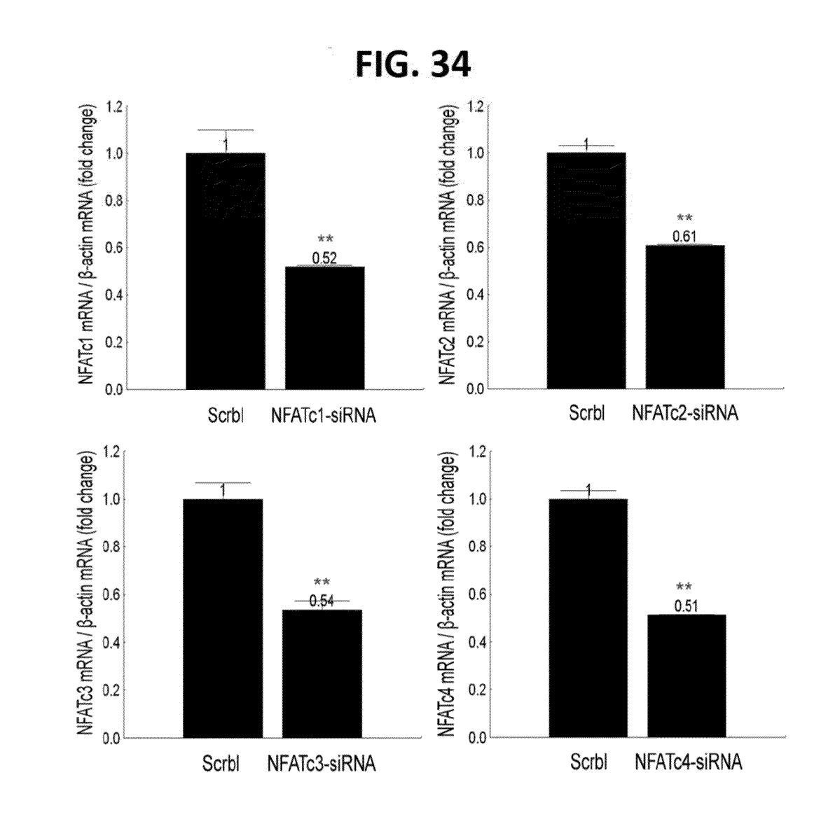

The inventors investigated which NFAT was functionally required for claudin-14 gene regulation in the TALH cells. To selectively knock down NFAT gene expression, the inventors transfected a pre-validated siRNA reagent (Methods) that contained a pool of three target-specific 21 nt siRNA duplexes designed against each mouse NFAT gene into the primary cultures of freshly isolated mouse TALH cells. A scrambled siRNA duplex was transfected as negative control. The efficacy of siRNA mediated NFAT knockdown was shown in FIG. 34. Compared to scrb1-siRNA, transfection with NFATc1-siRNA significantly increased the transcriptional level of claudin-14 by 2.29-fold (p<0.05, n=4; FIG. 31A) in primary TALH cells, while knockdown of other NFATs were without significant effect.

The inventors transfected a constitutively active mutant of NFATc1 (with 21 Serine to Alanine mutations (Winslow, M. M., et al., 2006); named NFATc1nuc; Methods) into primary TALH cells. Transfection with NFATc1nuc significantly decreased the transcriptional level of claudin-14 by 48% (p<0.01, n=3 versus vector control; FIG. 31B), accompanied by 2.86-fold and 1.52-fold increases in miR-9-1 and miR-374 transcriptional levels respectively (p<−0.01, n=3; FIGS. 31C and 23F). The transcription of miR-9-2 or miR-9-3 gene was not altered by NFATc1nuc transfection (FIG. 31D-E).

The inventors searched the miR-9-1 and miR-374 gene promoters for presence of NFAT consensus-binding sites ([A/T]GGAAA[A/N][A/T/C]N). (Kuwahara, K., et al., 2006) In the mouse miR-9-1 promoter, there was a NFAT binding site (AGGAAAAT) 1440 bp upstream of the miR-9-1 hairpin sequence (FIG. 31G), which was also present in human miR-9-I promoter (1660 bp upstream of human miR-9-1 hairpin). In the mouse miR-374 promoter, a similar NFAT binding site was found 1900 bp upstream of miR-374 hairpin (FIG. 31H) that was conserved in human miR-374 promoter. To determine if NFATc1 was able to regulate miR-9-1 and miR-374 promoters independent of cell context and chromatin environment, the inventors cloned the miRNA promoters (−2500 bp-0 bp; containing the NFAT binding site) upstream of a firefly luciferase reporter gene (FIG. 31G-H) and co-transfected with NFATc1nuc into a model cell line—HEK293 that expressed no endogenous CaSR-claudin-14 pathway. A 68% increase in firefly luciferase activity (p<0.05, n=3: normalized to renilla luciferase activity; FIG. 31G) was found for miR-9-1 promoter; a 22% increase (p<0.05, n=3; FIG. 31H) for miR-374 promoter, compared with vector transfection. Deletion of the NFAT binding site in miR-9-1 and miR-374 promoters (mutant promoter) completely abolished the observed NFATc1 effect (FIG. 31G-H).

The inventors investigated whether NFATc1 regulates endogenous miR-9-1 and miR-374 promoters in native TALH cells and in presence of intact chromatin. Chromatin immunoprecipitation (ChIP) was carried out in mouse primary TALH cells transfected with NFATc1nuc or its vector control. ChIP primers were designed to span the NFAT binding sites (FIGS. 31I and 23K; Table 2). NFATc1nuc transfection induced a 23.77-fold increase in its enrichment over miR-9-1 promoter (with anti-NFATc1 antibody, p<0.01, n=3 versus vector, normalized to anti-IgG antibody; FIG. 31I) and a 4.27-fold increase over miR-374 promoter (p<0.01, n=3 versus vector; FIG. 31K). The acetylation levels on histone H3 Lysine-9 (H3K9) and Lysine-14 (H3K14), well-documented chromatin markers for transcriptional activation, were concomitantly increased by 3.26-fold (with anti-H3K9/14Ac antibody; p<0.05, n=3 versus vector; normalized to chromatin input: FIG. 31J) and 1.87-fold (p<0.05, n=3 versus vector; FIG. 31L) respectively over the miR-9-1 and miR-374 promoter regions containing NFAT binding sites.

Having established a direct regulatory role for NFATc1 in microRNA transcription, the inventors investigated whether CaSR signaling induces NFATc1 dependent regulation of microRNA in vivo. To manipulate CaSR signaling in vivo in the kidney, the inventors treated WT mice with NPS2143 or cinacalcet at a single dose of 30 mg/kg BW⁻¹ as described above, isolated the TALH cells at 2 hrs following each drug treatment and performed Chip analyses on isolated TALH cells from each mouse. NPS2143 treatment significantly increased the fold enrichment of NFATc1 over the promoter regions of miR-9-1 and miR-374 containing NFAT binding sites, by 3.55-fold (p<0.01, n=5 versus vehicle: FIG. 31M) and 1.51-fold (p<0.05, n=5 versus vehicle; FIG. 31O) respectively: while cinacalcet treatment was without significant effect. The acetylation levels on H3K9 and H3K14 surrounding the same miR-9-1 and miR-374 promoter regions were also increased by NPS2143 but not by cinacalcet in mouse TALH cells, to 2.43-fold and 1.93-fold higher (p<0.05, n=5; FIG. 31N and FIG. 31P) than vehicle level respectively.

These data identified a transcriptional factor, NFATc1 that mediates CaSR signaling to transcriptional regulation of miR-9-1 and miR-374 genes through epigenetic mechanism.

### REFERENCES

Abdullah, H. L, Pedraza P L, McGiff J C, Ferreri N R. 2008. CaR activation increases TNF production by mTAL cells via a Gi-dependent mechanism. Am J Physiol Renal Physiol. 294:F345-54.
Archin, N. M., et al., Administration of vorinostat disrupts HIV-1 latency in patients on antiretroviral therapy. Nature. 2012; 487(7408):482-5.
Ben-Yosef, T., Belyantseva I A, Saunders T L, Hughes E D, Kawamoto K, Van Itallie C M, Beyer L A, Halsey K. Gardner D J, Wilcox E R, et al., 2003. Claudin 14 knockout mice, a model for autosomal recessive deafness DFNB29, are deaf due to cochlear hair cell degeneration. Hum Mol Genet. 12:2049-61.
Berezikov, E. Evolution of microRNA diversity and regulation in animals. Nature reviews Genetics 2011, 12:846-60.
Bemascone, I., et al., A transgenic mouse model for uromodulin-associated kidney diseases shows specific tubulo-interstitial damage, urinary concentrating defect and renal failure. Hum Mol Genet. 2010, 19(15):2998-3010.
Bourgeois, S., Rossignol, P., Grelac, F., Chalumeau, C., Klein. C., Laghmani, K., Chambrey, R., Bruneval. P., Duong, J. P., Poggioli, J., et al., (2003) Differentiated thick ascending limb (TAL) cultured cells derived from SV40 transgenic mice express functional apical NHE2 isoform: effect of nitric oxide. Pflugers Arch. 446:672-83.
Brown. E. M., Gamba, G., Riccardi, D., Lombardi, M., Butters. R., Kifor, O., Sun, A., Hediger. M. A., Lytton, J. Hebert, S. C. 1993. Cloning and characterization of an extracellular Ca2+-sensing receptor from bovine parathyroid. Nature. 366:575-580.
Buchert, M., Papin, M., Bonnans, C., Darido, C., Raye, W. S., Garambois, V., Pelegrin, A., Bourgaux, J. F., Pannequin, J., et al., (2010) Symplekin promotes tmnorigenicity by up-regulating claudin-2 expression. Proc Natl Acad Sci USA. 107:2628-33.
Buzza, M. S., et al., Membrane-anchored serine protease matriptase regulates epithelial barrier formation and permeability in the intestine. Proceedings of the National Academy of Sciences of the United States of America 2010, 107:4200-5.
Castoldi, M., et al., The liver-specific microRNA miR-122 controls systemic iron homeostasis in mice. J Clin Invest. 2011; 121(4):1386-96.
Chang, C. T., Hung, C. C., Yang, C. W., Vandewalle, A., Wu, M. S. 2005. Cyclosporine decreases prostaglandin E2 production in mouse medullary thick ascending limb cultured cells. Transpl Int. 18:871-8.
Chang, C. T., Hung, C. C., Tian, Y. C., Yang, C. W., Wu, M. S. 2007. Ciclosporin reduces paracellin-1 expression and magnesimn transport in thick ascending limb cells. Nephrol Dial Transplant. 22: 1033-40.
Coward. W. R., et al., Repression of IP-10 by interactions between histone deacetylation and hypermethylation in idiopathic pulmonary fibrosis. Mol Cell Biol. 2010; 30(12):2874-86.
Crabtree, G. R., Olson, E. N. 2002. NFAT signaling: choreographing the social lives of cells. Cell. 109 Suppl:S67-79.
Desfleurs, E., Wittner, M., Simeone, S., Pajaud, S., Moine, G., Rajerison, R., Di Stefano, A. 1998. CaSR: regulation of electrolyte transport in the thick ascending limb of Henle's loop. Kidney Blood Press Res 21:401-412.
Dimke, H., et al., Activation of the Ca(2+)-sensing receptor increases renal claudin-14 expression and urinary Ca(2+) excretion. Am J Physiol Renal Physiol. 2013; 304(6):F761-9.
Elkouby-Naor, L., Abassi, Z., Lagziel, A., Gow, A., Ben-Yosef, T. (2008) Double gene deletion reveals lack of cooperation between claudin 11 and claudin 14 tight junction proteins. Cell Tissue Res. 333(3):427-38.
Elmén. J., et al., Antagonism of microRNA-122 in mice by systemically administered LNA-antimiR leads to up-regulation of a large set of predicted target mRNAs in the liver. Nucleic Acids Res. 2008; 36(4):1153-62.
Esteva-Font. C., Ars. E., Guillen-Gomez, E., Campistol, J. M., Sanz, L. Jiminez, W., Knepper, M. A., Tones, F., Torra, R., Ballarin, J. A., et al., 2007. Ciclosporin-induced hypertension is associated with increased sodiumn transporter of the loop of Henle (NKCC2). Nephrol Dial Transplant. 22: 2810-6.
Feldman, G., et al., 2007. Role for TGF-beta in cyclosporine-induced modulation of renal epithelial barrier function. J Am Soc Nephrol. 18:1662-71.
Fernández-Llama, P., et al., Tamm-Horsfall protein and urinary exosome isolation. Kidney Int. 2010; 77(8):736-42.
Furuse, M., Sasaki, H. and Tsukita, S. (1999) Manner of interaction of heterogeneous claudin species within and between tight junction strands. J. Cell Biol. 147:891-903.
Furuse, M., et al., Claudin-based tight junctions play a role in the mammalian epidermal barrier: a lesson from claudin-1-deficient mice. J. Cell Biol. 2002, 156:1099-111.
Gamba, G., et al., (2009) Thick ascending limb: the Na(+):K (+):2C1 (−) co-transporter, NKCC2, and the calcium-sensing receptor, CaSR. Pflugers Arch. 458(1):61-76.
Gebeshuber, C. A., et al., Focal segmental glomerulosclerosis is induced by microRNA-193a and its downregulation of WT1. Nat Med. 2013; 19(4):481-7.
Gong, Y., et al., 2012. Claudin-14 regulates renal Ca²⁺ transport in response to CaSR signalling via a novel microRNA pathway. EMBO J. 31:1999-2012.
Gong, Y., Hou, J. Claudin-14 underlies CaSR mediated Ca++ metabolism via NFAT-microRNA based mechanisms. J Am Soc Nephrol. 2013 Dec. 12. [Epub ahead of print]
Gow, A., et al., CNS myelin and sertoli cell tight junction strands are absent in Osp/claudin-11 null mice. Cell 1999, 99:649-59.
Grant, M. P., et al., Agonist-driven maturation and plasma membrane insertion of calcium-sensing receptors dynamically control signal amplitude. Sci Signal. 2011, 4(200):ra78.
Greger, R. 1985. Ion transport mechanisms in thick ascending limb of Henle's loop of manualian nephron. Physiol. Rev. 65, 760-797.
Haberland. M., et al., The many roles of histone deacetylases in development and physiology: implications for disease and therapy. Nat Rev Genet. 2009: 10(1):32-42.
Harvey, S. J., et al., (2008) Podocyte-specific deletion of dicer alters cytoskeletal dynamics and causes glomerular disease. J Am Soc Nephrol. 19:2150-8.
Hebert, S. C., Andreoli, T. E. (1986) Ionic conductance pathways in the mouse medullary thick ascending limb of Henle. The paracellular pathway and electrogenic Cl⁻ absorption. J Gen Physiol. 87:567-90.
Hebert, S. C., Riccardi, D., Geibel, J. 2007. The calcium-sensing receptor. In: The Kidney: Physiology and Pathophysiology (4th ed.), edited by Alpern R J, Hebert S C. Amsterdam: Elsevier, p. 1785-1882.
Helin, K., et al., Chromatin proteins and modifications as drug targets. Nature. 2013; 502(7472):480-488.
Himmerkus, N., et al., (2008) Salt and acid-base metabolism in claudin-16 knockdown mice: impact for the pathophysiology of FHHNC patients. Am J Physiol Renal Physiol. 295:F1641-7.
Ho, C., et al., 1995. A mouse model of human familial hypocalciuric hypercalcemia and neonatal severe hyperparathyroidism. Nat Genet. 11:389-94.
Hoorn, E. J., et al., 2011. The calcineurin inhibitor tacrolinms activates the renal sodium chloride cotransporter to cause hypertension. Nat Med. 17:1304-9.
Hou J: Lecture: New light on the role of claudins in the kidney. Organogenesis 2012, 8:1-9.
Hou, J., et al., (2005) Paracellin-1 and the modulation of ion selectivity of tight junctions. J Cell Sci. 118:5109-18.
Hou, J., et al., 2007. Transgenic RNAi depletion of claudin-16 and the renal handling of magnesium. J Biol Chem. 282:17114-22.
Hou, J., et al., (2008) Claudin-16 and claudin-19 interact and form a cation-selective tight junction complex. J Clin Invest. 118:619-28.
Hou. J., et al., 2009. Claudin-16 and claudin-19 interaction is required for their assembly into tight junctions and for renal reabsorption of magnesium. Proc Natl Acad Sci USA. 106:15350-5.
Hou, J., et al., 2012. Claudins and the Kidney. Annu Rev Physiol. Annu Rev Physiol. 2013; 75:479-501.
Hu, H., et al., (2010) ATM is down-regulated by N-Myc-regulated microRNA-421. Proc Natl Acad Sci USA. 107:1506-11.
Huang, C., Miller, R. T. 2007. The calcium-sensing receptor and its interacting proteins. J Cell Mol Med 11:923-934.
Huntzinger, E., Izaumralde, E. (2011) Gene silencing by microRNAs: contributions of translational repression and mRNA decay. Nat Rev Genet. 12(2):99-110.
Johnstone, R. W. Histone-deacetylase inhibitors: novel drugs for the treatment of cancer. Nat Rev Drug Discov. 2002; 1(4):287-99.
Kantham, L., et al., 2009. The calcium-sensing receptor (CaSR) defends against hypercalcemia independently of its regulation of parathyroid hormone secretion. Am J Physiol Endocrinol Metab. 297:E915-23.
Kato, M., et al., TGF-beta activates Ak-t kinase through a microRNA-dependent amplifying circuit targeting PTEN. Nat Cell Biol. 2009; 11(7):881-9.
Kiuchi-Saishin, Y., et al., (2002) Differential expression patterns of claudins, tight junction membrane proteins, in mouse nephron segments. J Am Soc Nephrol. 13(4):875-86.
Konrad, M., et al., (2006) Mutations in the tightjunction gene claudin 19 (CLDN19) are associated with renal magnesium wasting, renal failure, and severe ocular involvement. Am. J. Hum. Genet. 79:949-957.
Kos, C. H., et al., The calcium-sensing receptor is required for normal calcium homeostasis independent of parathyroid hormone. J Clin Invest. 2003, 111(7):1021-8.
Krol, J., Loedige, I., Filipowicz, W. 2010. The widespread regulation of microRNA biogenesis, function and decay. Nat Rev Genet. 11:597-610.
Krutzfeldt, J., et al., (2005) Silencing of microRNAs in vivo with ‘antagomirs’. Nature 438, 685-689.
Kuwahara, K., et al., 2006. TRPC6 fulfills a calcineurin signaling circuit during pathologic cardiac remodeling. J Clin Invest. 116:3114-26.
Lee, M. G., et al., Histone H3 lysine 4 demethylation is a target of nonselective antidepressive medications. Chem Biol. 2006; 13(6):563-7.
Lindberg, J. S., et al., 2005. Cinacalcet HCl, an oral calcimimetic agent for the treatment of secondary hyperparathyroidism in hemodialysis and peritoneal dialysis: a randomized, double-blind, multicenter study. J Am Soc Nephrol. 16:800-7.
Loupy, A., et al., 2012. PTH-independent regulation of blood calcium concentration by the calcium-sensing receptor. J Clin Invest. 122:3355-67.
Ma, L., et al., (2010) miR-9, a MYC/MYCN-activated microRNA, regulates E-cadherin and cancer metastasis. Nat Cell Biol. 12:247-56.
Mandel, I., et al., The ubiquitin-proteasome pathway regulates claudin 5 degradation. Journal of cellular biochemistry 2012, 113:2415-23.
Marks, P. A., et al., Dimethyl sulfoxide to vorinostat: development of this histone deacetylase inhibitor as an anticancer drug. Nat Biotechnol. 2007; 25(1):84-90.
Mineta, K., et al., (2011) Predicted expansion of the claudin multigene family. FEBS Lett. 585(4):606-12.
Motoyama, H. I., Friedman, P. A. 2002. CaSR regulation of PTH dependent calcium absorption by mouse cortical ascending limbs. Am J Physiol Renal Physiol 283:F399-F406.
Nemeth, E. F., et al., 2004. Pharmacodynamics of the type II calcimimetic compound cinacalcet HCl. J Pharmacol Exp Ther. 308:627-35.
Nesbit, M. A., et al., Mutations in AP2S1 cause familial hypocalciuric hypercalcemia type 3. Nat Genet. 2013, 45(1):93-7.
Nijenhuis, T., Hoenderop, J. G., Bindels, R. J. 2004. Downregulation of Ca2+ and Mg2+ transport proteins in the kidney explains tacrolimus (FK506)-induced hypercalciuria and hypomagnesemia. J Am Soc Nephrol. 15: 549-57.
Nijenhuis, T., et al., Enhanced passive Ca2+ reabsorption and reduced Mg2+ channel abundance explains thiazide-induced hypocalciuria and hypomagnesemia. J Clin Invest. 2005; 115(6): 1651-8.
Nitta, T., et al., Size-selective loosening of the blood-brain barrier in claudin-5-deficient mice. The Journal of cell biology 2003, 161:653-60.
Nübel, T., et al., (2009) Claudin-7 regulates EpCAM-mediated functions in tumor progression. Mol Cancer Res. 7:285-99.
Obad, S., et al., Silencing of microRNA families by seed-targeting tiny LNAs. Nat Genet. 2011: 43(4):371-8.
Pietrzykowski, A. Z., et al., (2008) Posttranscriptional regulation of BK channel splice variant stability by miR-9 underlies neuroadaptation to alcohol. Neuron. 59:274-87.
Pipalia, N. H., et al., Histone deacetylase inhibitor treatment dramatically reduces cholesterol accumulation in Niemann-Pick type C1 mutant human fibroblasts. Proc Natl Acad Sci USA. 2011; 108(14):5620-5.
Pollak, M. R., et al., Mutations in the human Ca(2+)-sensing receptor gene cause familial hypocalciuric hypercalcemia and neonatal severe hyperparathyroidism. Cell 1993, 75:1297-303.
Riccardi, D., Brown, E. M. (2010) Physiology and pathophysiology of the calcium-sensing receptor in the kidney. Am J Physiol Renal Physiol. 298(3):F485-99.
Riccardi, D., Kemp, P. J. 2012. The calcium-sensing receptor beyond extracellular calcium homeostasis: conception, development, adult physiology, and disease. Annu Rev Physiol. 74:271-97.
Simon, D. B., et al., (1999) Paracellin-1, a renal tight junction protein required for paracellular Mg2+ resorption. Science. 285:103-106.
Stevenson, B. R., and Goodenough, D. A. 1984. Zonulae occludentes in junctional complex enriched fractions from mouse liver: morphological and biochemical characterization. J. Cell Biol. 98:1209-1221.
Stricklett, P. K., Taylor, D., Nelson, R. D., Kohan, D. E. 2003. Thick ascending limb-specific expression of Cre recombinase. Am J Physiol Renal Plrnsiol. 285:F33-9.
Thorleifsson, G., Holm, H., Edvardsson, V., Walters, G. B., Styrkarsdottir, U., Gudbjartsson, D. F., et al., (2009) Sequence variants in the CLDN14 gene associate with kidney stones and bone mineral density. Nat Genet. 41(8):926-30.
Thorleifsson. G., Walters, G. B., Gudbjartsson. D. F., Steinthorsdottir, V., Sulem, P., Helgadottir, A., et al., Genome-wide association yields new sequence variants at seven loci that associate with measures of obesity. Nat Genet 2009, 41:18-24.
Toka, H. R., et al., 2012. Deficiency of the calcium-sensing receptor in the kidney causes parathyroid hormone-independent hypocalciuria. J Am Soc Nephrol. 23:1879-90.
Tsukita, S., Furuse, M., and Itoh, M. (2001) Multifunctional strands in tight junctions. Nat. Rev. Mol. Cell. Biol. 2, 285-293.
Tu, Q., et al., Rescue of the skeletal phenotype in CasR-deficient mice by transfer onto the Gcm2 null background. J Clin Invest. 2003, 111(7):1029-37.
Van Itallie, C. M., et al., (2005) Pahnitoylation of claudins is required for efficient tight-junction localization. J Cell Sci. 118:1427-36.
Van Lint, C., et al., The expression of a small fraction of cellular genes is changed in response to histone hyperacetylation. Gene Expr. 1996; 5(4-5):245-53
Vargas-Poussou, R., et al., (2002) Functional characterization of a calcium-sensing receptor mutation in severe autosomal dominant hypocalcemia with a Bartter-like syndrome. J Am Soc Nephrol 13: 2259-2266.
Wang, K., et al., (2010) miR-9 and NFATc3 regulate myocardin in cardiac hypertrophy. J Biol Chem. 285:11903-12.
Wang. W. H., et al., (1996) Cytochrome P-450 metabolites mediate extracellular Ca²⁺-induced inhibition of apical K⁺ channels in the TAL. Am J Physiol Cell Physiol 271: C103-C111.
Wang, W. H., et al., (1997) Phospholipase A2 is involved in mediating the effect of extracellular Ca²⁺ on apical K⁺ channels in rat TAL. Am J Physiol Renal Physiol 273: F421-F429.
Weber, S., et al., (2001) Novel paracellin-1 mutations in 25 families with familial hypomagnesemia with hypercalciuria and nephrocalcinosis. J Am Soc Nephrol. 12:1872-81.
Wei, Q., et al., (2010) Targeted deletion of Dicer from proximal tubules protects against renal ischemia-reperfusion injury. J Am Soc Nephrol. 21:756-61.
Wilcox, E. R., et al., (2001) Mutations in the gene encoding tight junction claudin-14 cause autosomal recessive deafness DFNB29. Cell. 104:165-72.
Winslow, M. M., et al., 2006. Calcineurin/NFAT signaling in osteoblasts regulates bone mass. Dev Cell. 10:771-82.
Wong, V. (1997) Phosphorylation of occludin correlates with occludin localization and function at the tight junction. Am J Physiol. 273:C1859-67.
Yan, H., et al., (2002) Allelic variation in human gene expression. Science. 297:1143.
Yoo, A. S., et al., (2009) MicroRNA-mediated switching of chromatin-remodelling complexes in neural development. Nature. 460:642-6.
Yu, A. S., et al., (2009) Molecular basis for cation selectivity in claudin-2-based paracellular pores: identification of an electrostatic interaction site. J Gen Physiol. 133:111-27.
Zheng, B., Cantley, L. C. (2007) Regulation of epithelial tight junction assembly and disassembly by AMP-activated protein kinase. Proc Natl Acad Sci USA. 104:819-22.

All publications cited in this application are herein incorporated by reference in their entirety as if each individual publication, patent, patent application or other reference were specifically and individually indicated to be incorporated by reference.

What is claimed is:

1. A method of detecting, diagnosing or monitoring kidney stone disease in a subject, comprising:
providing a urine sample from a subject having or suspected of having kidney stone disease;
contacting the sample with an antibody that binds Claudin-14 polypeptide, under conditions sufficient for formation of a primary complex comprising the antibody and the Claudin-14 polypeptide if present;
measuring quantity of the primary complex;
comparing the quantity of the primary complex to that of a control complex formed from the antibody and a urine sample of an individual who does not have kidney stone disease; and
detecting kidney stone disease if the quantity of the primary complex from the subject is statistically significantly greater than that of an individual who does not have kidney stone disease.

2. A method of detecting, diagnosing or monitoring kidney stone disease in accordance with claim 1, wherein the antibody is a monoclonal antibody.

3. A method of detecting, diagnosing or monitoring kidney stone disease in accordance with claim 1, wherein the antibody is a polyclonal antibody.

4. A method of detecting, diagnosing or monitoring kidney stone disease in accordance with claim 1, wherein the antibody is a single chain antibody.

5. A method in accordance with claim 1, wherein the antibody is directed against the first extracellular loop of CLDN14.

6. A method of detecting, diagnosing or monitoring kidney stone disease in accordance with claim 1, wherein the measuring comprises an immunoprecipitation assay, an ELISA, a radioimmunoassay, a Western blot assay, a dip stick assay, a microarray, or a bead assay.

7. A method of detecting, diagnosing or monitoring kidney stone disease in accordance with claim 1, wherein the measuring comprises an ELISA or a Western blot assay.

8. A method of detecting, diagnosing or monitoring kidney stone disease in accordance with claim 1, wherein the antibody comprises a label, and the measuring quantity of the primary complex comprises quantifying the label.

9. A method of detecting, diagnosing or monitoring kidney stone disease in accordance with claim 8, wherein the label is selected from the group consisting of an enzyme, a radioisotope, a fluorogen, fluorophore, a chromogen and a chromophore.

10. A method of detecting, diagnosing or monitoring kidney stone disease in accordance with claim 1, wherein the measuring quantity of the primary complex comprises contacting the complex with a secondary probe that binds the antibody that binds Claudin-14 polypeptide under conditions sufficient for formulation of a second complex comprising the secondary probe, the antibody and the Claudin-14 polypeptide if present; and
determining quantity of the second complex.

11. A method of detecting, diagnosing or monitoring kidney stone disease in accordance with claim 10, wherein the secondary probe is selected from the group consisting of an antibody directed against the antibody that binds Claudin-14, an aptamer that binds the antibody that binds Claudin-14, an avimer that binds the antibody that binds Claudin-14, an avidin and a streptavidin.

12. A method in accordance with claim 1, wherein the subject is a mammal.

13. A method in accordance with claim 1, wherein the subject is a human.

14. A method of monitoring kidney stone disease in a subject, comprising:
providing a first sample from a subject at a first time point;
contacting the sample with an antibody that binds Claudin-14 polypeptide, under conditions sufficient for formation of a first primary complex comprising the first antibody and Claudin-14 polypeptide if present;
measuring quantity of the first primary complex;
providing a second sample from the subject at a second time point;
contacting the sample with the antibody that binds Claudin-14 polypeptide, under conditions sufficient for formation of a second primary complex comprising the second antibody and Claudin-14 polypeptide if present; and
measuring quantity of the second primary complex wherein an increase compared to the first sample is diagnostic for increased kidney stone disease.

15. A method of monitoring kidney stone disease in accordance with claim 14, wherein the antibody is a monoclonal antibody.

16. A method of monitoring kidney stone disease in accordance with claim 14, wherein the antibody is a polyclonal antibody.

17. A method of monitoring kidney stone disease in accordance with claim 14, wherein the antibody is a single chain antibody.

18. A method of monitoring kidney stone disease in accordance with claim 14, wherein the antibody is directed against the first extracellular loop of CLDN 4.

19. A method of monitoring kidney stone disease in accordance with claim 14 in accordance with claim 14, wherein the measuring quantity of the first complex and the measuring quantity of the second complex each comprises an immunoprecipitation assay, an ELISA, a radioimmunoassay, a Western blot assay, a dip stick assay, a microarray, or a bead assay.

20. A method of monitoring kidney stone disease in accordance with claim 14 in accordance with claim 14, wherein the measuring quantity of the first complex and the measuring quantity of the second complex each comprises an ELISA or a Western blot assay.


SEQUENCE LISTING (41 sequences)

SEQ ID NO: 1 - DNA, 171 bp, Mus musculus
gttccttccccgggcttctgccagggatgctgggccccaaaggaccaatgatggatgtgggaaggatgcagagagcaagcccggaacacagggaaggaggtgctcttcaaagcaaagacttctaaaaagtgctggttttttatttattatatgtatttatgcgggtggctt

SEQ ID NO: 2 - DNA, 183 bp, Homo sapiens
agcacaaagtttacttctgggcaatttttgtatccaaggaaataatgtgaatgcgaggaaatgtctttagagcacagggacagagggggaaataagaggaggagaaagctctctataccaaagactgaaaaaaaaaatcctgtctgtttttgtatttattatatatatttatgtgggtgattt

SEQ ID NO: 3 - DNA, 1024 bp, Homo sapiens
ccccacccgaaacacactcagcccttgcactgacctgccttctgattggaggctggttgcttcggataatgacctccaggaccccactgttggttacagcctgtttgtattattcttactgcaactcaagacacctgcagcagggcgtgagaaaaagtaaaagaccagtattttcacattgccaggtaccagaaacacagaagactgacacccgccacttaagtggggccagggctggtgtctgcccatgttgccatcctgatgggctgcttgccacaatgagggatcttcttcaatacatcgcttgcttctttgcctttttctctgctgggtttttgattgtggccacctggactgactgttggatggtgaatgctgatgactctctggaggtgagcacaaaatgccgaggcctctggtgggaatgcgtcacaaatgcttttgatgggattcgcacctgtgatgagtacgattccatacttgcggagcatcccttgaagctggtggtaactcgagcgttgatgattactgcagatattctagctgggtttggatttctcaccctgctccttggtcttgactgcgtgaaattcctccctgatgagccgtacattaaagtccgcatctgctttgttgctggagccacgttactaatagcaggtaccccaggaatcattggctctgtgtggtatgctgttgatgtgtatgtggaacgttctactttggttttgcacaatatatttcttggtatccaatataaatttggttggtcctgttggctcggaatggctgggtctctgggttgctttttggctggagctgttctcacctgctgcttatatctttttaaagatgttggacctgagagaaactatccttattccttgaggaaagcctattcagccgcgggtgtttccatggccaagtcatactcagcccctcgcacagagacggccaaaatgtatgctgtagacacaagggtgtaaaatgcacgtttcagggtgtgtttgcatatgatttaatc

SEQ ID NO: 4 - DNA, 1616 bp, Homo sapiens
ctggccatgaccaaagcccctgctggcaccctggcccagctctgagtcctgggaccctcggtcctctctcctgggccatggccaactcaggcctccagctcctgggctacttcttggccctgggtggctgggtgggcatcattgctagcacagccctgccacagtggaagcagtcttcctacgcaggcgacgccatcatcactgccgtgggcctctatgaagggctctggatgtcctgcgcctcccagagcactgggcaagtgcagtgcaagctctacgactcgctgctcgccctggacggtcacatccaatcagcgcgggccctgatggtggtggccgtgctcctgggcttcgtggccatggtcctcagcgtagttggcatgaagtgtacgcgggtgggagacagcaaccccattgccaagggccgtgttgccatcgccgggggagccctcttcatcctggcaggcctctgcactttgactgctgtctcgtggtatgccaccctggtgacccaggagttcttcaacccaagcacacctgtcaatgccaggtatgaatttggcccagccctgttcgtgggctgggcctcagctggcctggccgtgctgggcggctccttcctctgctgcacatgcccggagccagagagacccaacagcagcccacagccctatcggcctggaccctctgctgctgcccgagagtacgtctgagctctgcctgccctggccagccccccacccagtggcccccttgcccagcatccagccagcctcgcagcaccctgggcagggccactggggcataggatgggcataggtgctctgagcagcttgtcctcaacacaagcacccaccctgcaatctgagacccagatcctcagagagacaccagaggcaggacccagcccccaggcatacacacagatgcaggtccaggcacggtcttgtctgcacagcctggtgggcaccagcatgcatccctggagacaggccctcaggcaccagcccggctgtttactcactgaagagctgcgtgggtgtctgctacgtgctgggccctagagatagagcagtggccaagacgtaccttagtacccaggtccttggggtgagcagaaaccttcaccctccccagtcccatgggctcctcacagcaaccccacaagggcagtgccgggatgctgaacgttcacacaaggacagggagggtctgagtttaggtctcaggttcttccagtgcgcccagggctgggggccacctacacagatggtgaggtcggaccatggcgcccctgcccccgggaatgggccccaggcagggctgctgtgagggccaaggtctggccacgctggccagtacccatgtccgggcctgaatgcacagcccctgcccccgaccccacagctcactccactaaccagctctctctcttttgactttcagaccagttgttaaattgcccgcctccgccaagggccccctgggtgtgtaatgtccagtccccagccaggctctgtcccctgccatacctagactgtgtgtttcatatttttttggaaagagaagtgaacatccagccccaaaaaaaaaaaaaaaaaaaaaaaaaaaaaaaa

SEQ ID NO: 5 - protein, 19 aa, Mus musculus
SYSAPRTETAKMYAVDTRV

SEQ ID NO: 6 - protein, 19 aa, Mus musculus
NSIPQPYRSGPSTAAREYV

SEQ ID NO: 7 - DNA, 18 bp, Artificial Sequence
  Primer
accctgctctgcttatcc

SEQ ID NO: 8 - DNA, 18 bp, Artificial Sequence
  Primer
gcacggttgtccttgtag

SEQ ID NO: 9 - DNA, 21 bp, Artificial Sequence
  Primer
caaacgcttttgatgggattc

SEQ ID NO: 10 - DNA, 20 bp, Artificial Sequence
  Primer
tttgtgggtcatcaggtagg

SEQ ID NO: 11 - DNA, 19 bp, Artificial Sequence
  Primer
cgttgacatccgtaaagac

SEQ ID NO: 12 - DNA, 18 bp, Artificial Sequence
  Primer
tggaaggtggacagtgag

SEQ ID NO: 13 - DNA, 20 bp, Artificial Sequence
  Primer
cacacccgccaagaccacct

SEQ ID NO: 14 - DNA, 20 bp, Artificial Sequence
  Primer
aggtacagggcctggccgat

SEQ ID NO: 15 - DNA, 21 bp, Artificial Sequence
  Primer
gaagtttgagattggccaggc

SEQ ID NO: 16 - DNA, 20 bp, Artificial Sequence
  Primer
gtgcggtgtttgcagtggtc

SEQ ID NO: 17 - DNA, 20 bp, Artificial Sequence
  Primer
ggaggagggagtgggaaatg

SEQ ID NO: 18 - DNA, 20 bp, Artificial Sequence
  Primer
acctcacgcactatcacgac

SEQ ID NO: 19 - DNA, 20 bp, Artificial Sequence
  Primer
cagcttgctgcaccttagtc

SEQ ID NO: 20 - DNA, 20 bp, Artificial Sequence
  Primer
tgtgcggctagaacatccaa

SEQ ID NO: 21 - DNA, 20 bp, Artificial Sequence
  Primer
ggggaactggggatggagaa

SEQ ID NO: 22 - DNA, 19 bp, Artificial Sequence
  Primer
cagaggggaccgacagaca

SEQ ID NO: 23 - DNA, 24 bp, Artificial Sequence
  Primer
actcgatgattcttccattcatgc

SEQ ID NO: 24 - DNA, 20 bp, Artificial Sequence
  Primer
cggcttcaggtcaactgtga

SEQ ID NO: 25 - DNA, 20 bp, Artificial Sequence
  Primer
atgggtaacagtcttggcgg

SEQ ID NO: 26 - DNA, 20 bp, Artificial Sequence
  Primer
ggcgtatcgccaaaatcacc

SEQ ID NO: 27 - DNA, 22 bp, Artificial Sequence
  Primer
gtttttggaatagctgacaatg

SEQ ID NO: 28 - DNA, 20 bp, Artificial Sequence
  Primer
ctgttcaggaactggctttg

SEQ ID NO: 29 - DNA, 20 bp, Artificial Sequence
  Primer
cctggtgctgagattacaga

SEQ ID NO: 30 - DNA, 20 bp, Artificial Sequence
  Primer
ctgccacgtctataatccat

SEQ ID NO: 31 - DNA, 20 bp, Artificial Sequence
  Primer
gtgttcccagcggtcagccc

SEQ ID NO: 32 - DNA, 20 bp, Artificial Sequence
  Primer
gtggagcttcgggcttgcgt

SEQ ID NO: 33 - DNA, 20 bp, Artificial Sequence
  Primer
aggacgacagcatccccgct

SEQ ID NO: 34 - DNA, 20 bp, Artificial Sequence
  Primer
acggctgccctccgtctcat

SEQ ID NO: 35 - DNA, 20 bp, Artificial Sequence
  Primer
gcagcttccccagcagcctc

SEQ ID NO: 36 - DNA, 20 bp, Artificial Sequence
  Primer
ccggacccgggagaggagtg

SEQ ID NO: 37 - DNA, 20 bp, Artificial Sequence
  Primer
gcaggcagcatcagtgccca

SEQ ID NO: 38 - DNA, 20 bp, Artificial Sequence
  Primer
tcctgctccgaagccccctc

SEQ ID NO: 39 - protein, 19 aa, Mus musculus
RAPSVTSAAHSGYRLNDYV

SEQ ID NO: 40 - RNA, 89 nt, Homo sapiens
cgggguugguuguuaucuuugguuaucuagcuguaugagugguguggagucuucauaaagcuagauaaccgaaaguaaaaauaacccca

SEQ ID NO: 41 - RNA, 72 nt, Homo sapiens
acucggauggauauaauacaaccugcuaaguguccuagcacuuagcagguuguauuaucauuguccgugucu